SEQUENCE LISTING (50 sequences)

SEQ ID NO: 1 - protein, 457 aa, Artificial Sequence
  Description of Artificial Sequence: Synthetic polypeptide
QVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 2 - protein, 214 aa, Artificial Sequence
  Description of Artificial Sequence: Synthetic polypeptide
EIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 457 aa, Artificial Sequence
  Description of Artificial Sequence: Synthetic polypeptide
QVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 4 - protein, 214 aa, Artificial Sequence; identical to SEQ ID NO: 2
  Description of Artificial Sequence: Synthetic polypeptide

SEQ ID NO: 5 - protein, 127 aa, Artificial Sequence
  Description of Artificial Sequence: Synthetic polypeptide
QVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSS

SEQ ID NO: 6 - protein, 107 aa, Artificial Sequence [sequence omitted]

SEQ ID NO: 7 - protein, 8 aa, Artificial Sequence
  Description of Artificial Sequence: Synthetic peptide
GYPFTSYG

SEQ ID NO: 8 - protein, 8 aa, Artificial Sequence
  Description of Artificial Sequence: Synthetic peptide
ISTYQGNT

SEQ ID NO: 9 - protein, 20 aa, Artificial Sequence
  Description of Artificial Sequence: Synthetic peptide
ARDYTRGAWFGESLIGGFDN

SEQ ID NO: 10 - protein, 7 aa, Artificial Sequence
  Description of Artificial Sequence: Synthetic peptide
QTVSSTS

SEQ ID NO: 11 - protein, 3 aa, Artificial Sequence
  Description of Artificial Sequence: Synthetic peptide
GAS

SEQ ID NO: 12 - protein, 8 aa, Artificial Sequence
  Description of Artificial Sequence: Synthetic peptide
QQHDTSLT

SEQ ID NO: 13 - protein, 107 aa, Artificial Sequence [sequence omitted]

SEQ ID NO: 14 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 15 - protein, 329 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 16 - skipped

SEQ ID NO: 17 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 18 - protein, 329 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 19 - DNA, 1431 bp, Artificial Sequence
  Description of Artificial Sequence: Synthetic polynucleotide
  modified_base (795)..(795): a, t, g, or c
  modified_base (1077)..(1077): a, t, g, or c
atggaattcggcctgtcctgggtctttctggtggccatcctgaagggcgtgcagtgccaggtccagctggttcagagcggcgccgaggttaagaaacctggcgccagcgtgaaagtgtcctgcaaggccagcggctaccccttcaccagctacggcatctcttgggtgcggcaggcccctggacaaggactggagtggatgggatggatcagcacttaccagggcaataccaactacgcccagaaattccagggcagagtgacaatgaccaccgacaccagcacaaccacaggctacatggaactgcggagactgagaagcgatgatacagccgtgtactactgcgccagagattatacaagaggtgcttggttcggcgagagcctgatcggcggattcgacaactggggacaaggcaccctggtgaccgtgtcatccgcctctaccaagggccctagcgtgtttccactggcccctagctctaaaagcacaagcggcggcaccgccgctctgggatgtctggtgaaggactacttcccagagcccgtgaccgtgagctggaacagcggcgctctcacatctggggtgcatacctttcccgccgtgctgcagtcttctggactgtacagcctgagcagcgtggtgaccgtgccctccagcagcctgggcacacagacctacatctgcaacgtgaaccacaagccatctaataccaaggtggataagaaggtggaacctaagagctgtgacaagacacacacatgccccccctgccctgctcctgagctgctgggnggcccctccgtgtttctcttccctcctaaacccaaggacacactgatgattagccggaccccagaggtgacctgtgtggtggttgacgtgagtcacgaagatcctgaagtgaagttcaactggtacgtggacggcgtggaggtgcataacgccaaaaccaagcctcgggaagagcagtacaacagcacctatagagtggtgagcgtgcttacagtgctgcatcaggactggctgaacggcaaggaatacaagtgcaaggtgtccaacaaagccctgcctgcncctathgaaaagaccatcagcaaggccaagggccaaccaagagagcctcaagtgtacaccctgccccccagcagagatgagctgaccaagaatcaggtgtccctgacctgcctggtcaaaggcttctaccctagcgacatcgccgtcgagtgggagagcaatggccagcctgagaacaactacaagaccacccctcctgtgctggacagcgacggcagcttcttcctgtatagcaagctgaccgtggacaagtccaggtggcagcagggcaatgtgttcagctgtagcgtgctgcacgaggccctgcacagccactacacacagaagtctctgagcctgtctcctggcaagtga

SEQ ID NO: 20 - DNA, 1374 bp, Artificial Sequence
  Description of Artificial Sequence: Synthetic polynucleotide
  modified_base (738)..(738): a, t, g, or c
  modified_base (1020)..(1020): a, t, g, or c
caggtccagctggttcagagcggcgccgaggttaagaaacctggcgccagcgtgaaagtgtcctgcaaggccagcggctaccccttcaccagctacggcatctcttgggtgcggcaggcccctggacaaggactggagtggatgggatggatcagcacttaccagggcaataccaactacgcccagaaattccagggcagagtgacaatgaccaccgacaccagcacaaccacaggctacatggaactgcggagactgagaagcgatgatacagccgtgtactactgcgccagagattatacaagaggtgcttggttcggcgagagcctgatcggcggattcgacaactggggacaaggcaccctggtgaccgtgtcatccgcctctaccaagggccctagcgtgtttccactggcccctagctctaaaagcacaagcggcggcaccgccgctctgggatgtctggtgaaggactacttcccagagcccgtgaccgtgagctggaacagcggcgctctcacatctggggtgcatacctttcccgccgtgctgcagtcttctggactgtacagcctgagcagcgtggtgaccgtgccctccagcagcctgggcacacagacctacatctgcaacgtgaaccacaagccatctaataccaaggtggataagaaggtggaacctaagagctgtgacaagacacacacatgccccccctgccctgctcctgagctgctgggnggcccctccgtgtttctcttccctcctaaacccaaggacacactgatgattagccggaccccagaggtgacctgtgtggtggttgacgtgagtcacgaagatcctgaagtgaagttcaactggtacgtggacggcgtggaggtgcataacgccaaaaccaagcctcgggaagagcagtacaacagcacctatagagtggtgagcgtgcttacagtgctgcatcaggactggctgaacggcaaggaatacaagtgcaaggtgtccaacaaagccctgcctgcncctathgaaaagaccatcagcaaggccaagggccaaccaagagagcctcaagtgtacaccctgccccccagcagagatgagctgaccaagaatcaggtgtccctgacctgcctggtcaaaggcttctaccctagcgacatcgccgtcgagtgggagagcaatggccagcctgagaacaactacaagaccacccctcctgtgctggacagcgacggcagcttcttcctgtatagcaagctgaccgtggacaagtccaggtggcagcagggcaatgtgttcagctgtagcgtgctgcacgaggccctgcacagccactacacacagaagtctctgagcctgtctcctggcaagtga

SEQ ID NO: 21 - DNA, 1431 bp, Artificial Sequence
  Description of Artificial Sequence: Synthetic polynucleotide
atgggctggtcctgtatcatcctgttcctggtcgccacagccaccggagtgcacagccaagtgcagctggtccagagcggcgccgaggtgaagaagcccggcgctagcgtgaaggtgtcctgtaaagccagcggatatccttttaccagctacggcatctcctgggtgcggcaggcccctggccagggcctggaatggatgggctggatcagcacctaccagggaaataccaactacgcccagaagttccagggaagagtgacaatgaccacagatacatctacaaccaccggctacatggaactgaggcggctgagaagcgacgacaccgccgtgtactactgcgccagagattacaccagaggcgcttggttcggcgagagcctgatcggcggcttcgacaactggggccagggaaccctggtgacagtgtctagcgcttctaccaaaggcccttctgtctttcctctggccccttctagcaagtctacaagcggaggcaccgccgccctgggctgcctggtgaaggactacttccccgagcccgtgaccgtgagctggaatagcggcgccctgacaagcggcgtgcacaccttcccagctgtgctgcagagcagcggcctgtatagcctgagcagcgtggtcaccgtgcccagcagcagcctgggaacacagacctacatctgcaacgtgaaccacaagccttctaataccaaggtggataagaaggtggaacctaagagctgcgacaaaacacacacatgccctccatgtcctgctccagagctgctggccggccccagcgtttttctgttcccccccaaacctaaagacaccctgatgatcagcagaacccctgaggtgacctgtgtggtggtggacgtgtcccacgaagatcctgaggtgaagttcaactggtacgtggatggagtggaagtgcacaacgccaagaccaaacctagagaagagcagtacaacagcacatatagagtcgtgtccgtgcttacagtgctgcaccaggactggctgaatggaaaggaatacaagtgcaaggtgtccaacaaggccctgcctctgcctgaggagaagacaatctctaaagccaagggccaacctcgggaacctcaggtgtacacactgccccccagccgggacgagctgaccaagaaccaggtgtccctgacctgcctggtcaagggcttctacccctctgatatcgccgtggaatgggagagcaacggccaacctgagaacaactacaagaccacccctccagtgctggacagcgacggcagcttcttcctgtacagcaagctgaccgttgacaagtccagatggcagcagggcaacgtgttcagctgtagcgtcctgcacgaggccctgcattctcactacacccagaagagcctgtccctcagccctggcaagtga

SEQ ID NO: 22 - DNA, 1374 bp, Artificial Sequence
  Description of Artificial Sequence: Synthetic polynucleotide
caagtgcagctggtccagagcggcgccgaggtgaagaagcccggcgctagcgtgaaggtgtcctgtaaagccagcggatatccttttaccagctacggcatctcctgggtgcggcaggcccctggccagggcctggaatggatgggctggatcagcacctaccagggaaataccaactacgcccagaagttccagggaagagtgacaatgaccacagatacatctacaaccaccggctacatggaactgaggcggctgagaagcgacgacaccgccgtgtactactgcgccagagattacaccagaggcgcttggttcggcgagagcctgatcggcggcttcgacaactggggccagggaaccctggtgacagtgtctagcgcttctaccaaaggcccttctgtctttcctctggccccttctagcaagtctacaagcggaggcaccgccgccctgggctgcctggtgaaggactacttccccgagcccgtgaccgtgagctggaatagcggcgccctgacaagcggcgtgcacaccttcccagctgtgctgcagagcagcggcctgtatagcctgagcagcgtggtcaccgtgcccagcagcagcctgggaacacagacctacatctgcaacgtgaaccacaagccttctaataccaaggtggataagaaggtggaacctaagagctgcgacaaaacacacacatgccctccatgtcctgctccagagctgctggccggccccagcgtttttctgttcccccccaaacctaaagacaccctgatgatcagcagaacccctgaggtgacctgtgtggtggtggacgtgtcccacgaagatcctgaggtgaagttcaactggtacgtggatggagtggaagtgcacaacgccaagaccaaacctagagaagagcagtacaacagcacatatagagtcgtgtccgtgcttacagtgctgcaccaggactggctgaatggaaaggaatacaagtgcaaggtgtccaacaaggccctgcctctgcctgaggagaagacaatctctaaagccaagggccaacctcgggaacctcaggtgtacacactgccccccagccgggacgagctgaccaagaaccaggtgtccctgacctgcctggtcaagggcttctacccctctgatatcgccgtggaatgggagagcaacggccaacctgagaacaactacaagaccacccctccagtgctggacagcgacggcagcttcttcctgtacagcaagctgaccgttgacaagtccagatggcagcagggcaacgtgttcagctgtagcgtcctgcacgaggccctgcattctcactacacccagaagagcctgtccctcagccctggcaagtga

SEQ ID NO: 23 - DNA, 701 bp, Artificial Sequence
  Description of Artificial Sequence: Synthetic polynucleotide
tgggctggtcctgcatcatcctgttcctggtggccacagccaccggcgtgcacagcgagatcgtcctgacacagagccccggcacactgagcctctccccaggcgagcgggctacactgtcttgtagagcttctcagaccgtgtccagcaccagcctggcctggtatcagcagaaacctggccaggcccctagactgctgatctacggcgccagcagcagagccaccggcatccctgatagattcagcggcagcggatctggaaccgacttcaccctgaccatcagccggctggaacccgaggactttgccgtgtactactgccagcaacacgacaccagcctgaccttcggcggcggaacaaaggtggaaatcaagagaaccgtggccgcccctagcgtgttcatcttcccccccagcgacgagcagctgaagagcggtacagcttctgtggtgtgcctgctgaacaacttctacccgcgggaagccaaggtgcagtggaaggtggacaacgccctgcagagcggcaacagccaggagagcgtgacagagcaggacagcaaggacagcacctacagcctgagcagcaccctgaccctgagcaaggccgactacgagaagcacaaggtgtacgcctgtgaagtgacccaccagggcctgtctagccctgtgaccaagtcttttaacagaggcgagtgctga

SEQ ID NO: 24 - DNA, 645 bp, Artificial Sequence
  Description of Artificial Sequence: Synthetic polynucleotide
gagatcgtcctgacacagagccccggcacactgagcctctccccaggcgagcgggctacactgtcttgtagagcttctcagaccgtgtccagcaccagcctggcctggtatcagcagaaacctggccaggcccctagactgctgatctacggcgccagcagcagagccaccggcatccctgatagattcagcggcagcggatctggaaccgacttcaccctgaccatcagccggctggaacccgaggactttgccgtgtactactgccagcaacacgacaccagcctgaccttcggcggcggaacaaaggtggaaatcaagagaaccgtggccgcccctagcgtgttcatcttcccccccagcgacgagcagctgaagagcggtacagcttctgtggtgtgcctgctgaacaacttctacccgcgggaagccaaggtgcagtggaaggtggacaacgccctgcagagcggcaacagccaggagagcgtgacagagcaggacagcaaggacagcacctacagcctgagcagcaccctgaccctgagcaaggccgactacgagaagcacaaggtgtacgcctgtgaagtgacccaccagggcctgtctagccctgtgaccaagtcttttaacagaggcgagtgctga

SEQ ID NO: 25 - DNA, 2574 bp, Artificial Sequence
  Description of Artificial Sequence: Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatcgcggccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggtacccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttgggtttaaaccgcagccaccatggacatgagagtgcccgcccagctgctgggcctgctgctgctgtggctgtccggagccagatgcgaaatcgtgctgacccagagccctggcaccctgagcctgtcacccggcgaacgggctaccctgtcttgtagagcctctcagaccgtgtccagcaccagcctggcctggtaccagcagaaacctggacaggctcctagactgctgatctacggagcttctagtagagccaccggcatccccgatagattcagcggcagcggcagcggcactgatttcaccctgacaattagccggctggaacctgaggactttgccgtgtattactgccagcaacacgacaccagcctgacattcggcggcggaaccaaagttgagatcaagcggaccgtggccgctccatctgtgttcatctttccacctagcgacgagcagctgaagtccggcacagcctctgtggtgtgcctgctcaacaacttctaccctcgcgaggccaaggtgcagtggaaggtggacaacgccctgcaaagcggcaacagccaggagagcgtcacagaacaggacagcaaggactctacatacagcctgagcagcacactgaccctcagcaaggccgattacgagaagcacaaggtttacgcctgcgaggtgacccaccagggcctgtccagccctgtgacaaagagcttcaatagaggcgaatgttgatagttaattaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 26 - DNA, 3293 bp, Artificial Sequence
  Description of Artificial Sequence: Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatcgcggccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggtacccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttgggtttaaaccgcagccaccatggaattcggcctgtcctgggtctttctggtggccatcctgaagggcgtgcagtgccaggtccagctggttcagagcggcgccgaggttaagaaacctggcgccagcgtgaaagtgtcctgcaaggccagcggctaccccttcaccagctacggcatctcttgggtgcggcaggcccctggacaaggactggagtggatgggatggatcagcacttaccagggcaataccaactacgcccagaaattccagggcagagtgacaatgaccaccgacaccagcacaaccacaggctacatggaactgcggagactgagaagcgatgatacagccgtgtactactgcgccagagattatacaagaggtgcttggttcggcgagagcctgatcggcggattcgacaactggggacaaggcaccctggtgaccgtgtcatccgcctctaccaagggccctagcgtgtttccactggcccctagctctaaaagcacaagcggcggcaccgccgctctgggatgtctggtgaaggactacttcccagagcccgtgaccgtgagctggaacagcggcgctctcacatctggggtgcatacctttcccgccgtgctgcagtcttctggactgtacagcctgagcagcgtggtgaccgtgccctccagcagcctgggcacacagacctacatctgcaacgtgaaccacaagccatctaataccaaggtggataagaaggtggaacctaagagctgtgacaagacacacacatgccccccctgccctgctcctgagctgctggccggcccctccgtgtttctcttccctcctaaacccaaggacacactgatgattagccggaccccagaggtgacctgtgtggtggttgacgtgagtcacgaagatcctgaagtgaagttcaactggtacgtggacggcgtggaggtgcataacgccaaaaccaagcctcgggaagagcagtacaacagcacctatagagtggtgagcgtgcttacagtgctgcatcaggactggctgaacggcaaggaatacaagtgcaaggtgtccaacaaagccctgcctctgcctgaagaaaagaccatcagcaaggccaagggccaaccaagagagcctcaagtgtacaccctgccccccagcagagatgagctgaccaagaatcaggtgtccctgacctgcctggtcaaaggcttctaccctagcgacatcgccgtcgagtgggagagcaatggccagcctgagaacaactacaagaccacccctcctgtgctggacagcgacggcagcttcttcctgtatagcaagctgaccgtggacaagtccaggtggcagcagggcaatgtgttcagctgtagcgtgctgcacgaggccctgcacagccactacacacagaagtctctgagcctgtctcctggcaagtgatagttaattaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctctagagatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 27 - DNA, 6156 bp, Artificial Sequence
  Description of Artificial Sequence: Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcatatgtaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttaattaactatcaacattcgcctctattgaagctctttgtcacagggctggacaggccctggtgggtcacctcgcaggcgtaaaccttgtgcttctcgtaatcggccttgctgagggtcagtgtgctgctcaggctgtatgtagagtccttgctgtcctgttctgtgacgctctcctggctgttgccgctttgcagggcgttgtccaccttccactgcaccttggcctcgcgagggtagaagttgttgagcaggcacaccacagaggctgtgccggacttcagctgctcgtcgctaggtggaaagatgaacacagatggagcggccacggtccgcttgatctcaactttggttccgccgccgaatgtcaggctggtgtcgtgttgctggcagtaatacacggcaaagtcctcaggttccagccggctaattgtcagggtgaaatcagtgccgctgccgctgccgctgaatctatcggggatgccggtggctctactagaagctccgtagatcagcagtctaggagcctgtccaggtttctgctggtaccaggccaggctggtgctggacacggtctgagaggctctacaagacagggtagcccgttcgccgggtgacaggctcagggtgccagggctctgggtcagcacgatttcgcatctggctccggacagccacagcagcagcaggcccagcagctgggcgggcactctcatgtccatggtggctgcggtttaaactcagttccaaaggttggaatctaaaagagagaaacaattagaatcagtagtttaacacattatacacttaaaaattttatatttaccttagagtaattattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgtacctggctcagaaaccacagcgtcctgtgtccaaggtggagggggtggcgtgagtcagacagtctctgggagagtaccacttagctggccctctgctctcactgcagaatccttagtggctgttccactggtagcaagatccagtacctctagaccagcctgaccaacatggtgaaaccccgtccctgtcaaaaatagaaaaaattagccgggtgtggtggcacaggcctgtaatcccagttacttgggaggctgaggctgaccaacatggagaaacccgtctctactaaaaatagaaaattagccaggtgtggtggcacatgcttgtaatcccagctacttgggaggctgaggcaggggaaggttgtggtgagatgagattgtgtcactgcactccagcctgggcgacagagcaaacctccatctcaaaaaacaaaacaaaacaaaacaaaaaaaccaaatgtttatttgccacaaaaaccctatcagatgggcgtctttatcatttccattgtacagatggggaaacaggcttcggggtcggggcatagccacttactgacgactccccacccagcaagtggttttgaacccggaccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctctgagcctacattcttaactaccctccgccctactcctgtccctcccccatttcctgtttgcagtacccaaggcaaatattagtctaagtaggacagagggacaaagagcaggaacacggggaggcacaagttctcgcatcgattgtaccaaagtacaagcgttaatgattaactgtaccaaagtacaagcgttaatgattaactgtaccaaagtacaagcgttaatgattaacgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggtacccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggaccaggtcgcagccaccatggaattcggcctgtcctgggtctttctggtggccatcctgaagggcgtgcagtgccaggtccagctggttcagagcggcgccgaggttaagaaacctggcgccagcgtgaaagtgtcctgcaaggccagcggctaccccttcaccagctacggcatctcttgggtgcggcaggcccctggacaaggactggagtggatgggatggatcagcacttaccagggcaataccaactacgcccagaaattccagggcagagtgacaatgaccaccgacaccagcacaaccacaggctacatggaactgcggagactgagaagcgatgatacagccgtgtactactgcgccagagattatacaagaggtgcttggttcggcgagagcctgatcggcggattcgacaactggggacaaggcaccctggtgaccgtgtcatccgcctctaccaagggccctagcgtgtttccactggcccctagctctaaaagcacaagcggcggcaccgccgctctgggatgtctggtgaaggactacttcccagagcccgtgaccgtgagctggaacagcggcgctctcacatctggggtgcatacctttcccgccgtgctgcagtcttctggactgtacagcctgagcagcgtggtgaccgtgccctccagcagcctgggcacacagacctacatctgcaacgtgaaccacaagccatctaataccaaggtggataagaaggtggaacctaagagctgtgacaagacacacacatgccccccctgccctgctcctgagctgctggccggcccctccgtgtttctcttccctcctaaacccaaggacacactgatgattagccggaccccagaggtgacctgtgtggtggttgacgtgagtcacgaagatcctgaagtgaagttcaactggtacgtggacggcgtggaggtgcataacgccaaaaccaagcctcgggaagagcagtacaacagcacctatagagtggtgagcgtgcttacagtgctgcatcaggactggctgaacggcaaggaatacaagtgcaaggtgtccaacaaagccctgcctctgcctgaagaaaagaccatcagcaaggccaagggccaaccaagagagcctcaagtgtacaccctgccccccagcagagatgagctgaccaagaatcaggtgtccctgacctgcctggtcaaaggcttctaccctagcgacatcgccgtcgagtgggagagcaatggccagcctgagaacaactacaagaccacccctcctgtgctggacagcgacggcagcttcttcctgtatagcaagctgaccgtggacaagtccaggtggcagcagggcaatgtgttcagctgtagcgtgctgcacgaggccctgcacagccactacacacagaagtctctgagcctgtctcctggcaagtgatagcttaaggagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 28 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 29 - DNA, 16 bp, Adeno-associated virus 2
gcgcgctcgctcgctc

SEQ ID NO: 30 - DNA, 143 bp, Adeno-associated virus 1
ttgcccactccctctctgcgcgctcgctcgctcggtggggcctgcggaccaaaggtccgcagacggcagaggtctcctctgccggccccaccgagcgagcgacgcgcgcagagagggagtgggcaactccatcactagggtaa

SEQ ID NO: 31 - DNA, 141 bp, Adeno-associated virus 2
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 32 - DNA, 144 bp, Adeno-associated virus 3
ttggccactccctctatgcgcactcgctcgctcggtggggcctggcgaccaaaggtcgccagacggacgtgggtttccacgtccggccccaccgagcgagcgagtgcgcatagagggagtggccaactccatcactagaggtat

SEQ ID NO: 33 - DNA, 127 bp, Adeno-associated virus 4
ttggccactccctctatgcgcgctcgctcactcactcggccctggagaccaaaggtctccagactgccggcctctggccggcagggccgagtgagtgagcgagcgcgcatagagggagtggccaact

SEQ ID NO: 34 - DNA, 166 bp, Adeno-associated virus 5
tcccccctgtcgcgttcgctcgctcgctggctcgtttgggggggcgacggccagagggccgtcgtctggcagctctttgagctgccacccccccaaacgagccagcgagcgagcgaacgcgacaggggggagagtgccacactctcaagcaagggggttttgtaag

SEQ ID NO: 35 - DNA, 144 bp, Adeno-associated virus 6
ttgcccactccctctaatgcgcgctcgctcgctcggtggggcctgcggaccaaaggtccgcagacggcagaggtctcctctgccggccccaccgagcgagcgagcgcgcatagagggagtgggcaactccatcactaggggtat

SEQ ID NO: 36 - DNA, 143 bp, Adeno-associated virus 1
ttaccctagtgatggagttgcccactccctctctgcgcgcgtcgctcgctcggtggggccggcagaggagacctctgccgtctgcggacctttggtccgcaggccccaccgagcgagcgagcgcgcagagagggagtgggcaa

SEQ ID NO: 37 - DNA, 141 bp, Adeno-associated virus 2
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 38 - DNA, 144 bp, Adeno-associated virus 3
atacctctagtgatggagttggccactccctctatgcgcactcgctcgctcggtggggccggacgtggaaacccacgtccgtctggcgacctttggtcgccaggccccaccgagcgagcgagtgcgcatagagggagtggccaa

SEQ ID NO: 39 - DNA, 127 bp, Adeno-associated virus 4
agttggccacattagctatgcgcgctcgctcactcactcggccctggagaccaaaggtctccagactgccggcctctggccggcagggccgagtgagtgagcgagcgcgcatagagggagtggccaa

SEQ ID NO: 40 - DNA, 166 bp, Adeno-associated virus 5
cttacaaaacccccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacagggggga

SEQ ID NO: 41 - DNA, 144 bp, Adeno-associated virus 6
atacccctagtgatggagttgcccactccctctatgcgcgctcgctcgctcggtggggccggcagaggagacctctgccgtctgcggacctttggtccgcaggccccaccgagcgagcgagcgcgcattagagggagtgggcaa

SEQ ID NO: 42 - DNA, 12 bp, Artificial Sequence
  Description of Artificial Sequence: Synthetic oligonucleotide
atcgaacgatcg

SEQ ID NO: 43 - DNA, 12 bp, Artificial Sequence
  Description of Artificial Sequence: Synthetic oligonucleotide
cgatcgttcgat

SEQ ID NO: 44 - DNA, 12 bp, Artificial Sequence
  Description of Artificial Sequence: Synthetic oligonucleotide
atcgaaccatcg

SEQ ID NO: 45 - DNA, 165 bp, Adeno-associated virus 2
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 46 - DNA, 140 bp, Adeno-associated virus 2
cccctagtgatggagttggccactccctctctgcgcgctcgctcgctcactgaggccgcccgggcaaagcccgggcgtcgggcgacctttggtcgcccggcctcagtgagcgagcgagcgcgcagagagatcactagggg

SEQ ID NO: 47 - DNA, 91 bp, Adeno-associated virus 2
gcgcgctcgctcgctcactgaggccgcccgggcaaagcccgggcgtcgggcgacctttggtcgcccggcctcagtgagcgagcgagcgcgc

SEQ ID NO: 48 - DNA, 80 bp, Artificial Sequence
  Description of Artificial Sequence: Synthetic oligonucleotide
gcgcgctcgctcgctcactgaggccgcccgggcgtcgggcgacctttggtcgcccggcctcagtgagcgagcgagcgcgc

SEQ ID NO: 49 - DNA, 91 bp, Adeno-associated virus 2
gcgcgctcgctcgctcactgaggccgggcgaccaaaggtcgcccgacgcccgggctttgcccgggcggcctcagtgagcgagcgagcgcgc

SEQ ID NO: 50 - DNA, 80 bp, Artificial Sequence
  Description of Artificial Sequence: Synthetic oligonucleotide
gcgcgctcgctcgctcactgaggccgggcgaccaaaggtcgcccgacgcccgggcggcctcagtgagcgagcgagcgcgc

(19) United States
(12) Patent Application Publication
(10) Pub. No.: US 2024/0216535 A1
(43) Pub. Date: Jul. 4, 2024

(54) NON-VIRAL DNA VECTORS EXPRESSING ANTI-CORONAVIRUS ANTIBODIES AND USES THEREOF

(71) Applicants: Generation Bio Co., Cambridge, MA (US); Vir Biotechnology, Inc., San Francisco, CA (US)

(72) Inventors: Nathaniel Silver, Cambridge, MA (US); Fabio Benigni, Bellinzona (CH)

(21) Appl. No.: 18/288,622

(22) PCT Filed: Apr. 27, 2022
(86) PCT No.: PCT/US2022/026554

§ 371 Date: Oct. 27, 2023

Related U.S. Application Data
(60) Provisional application No. 63/182,563, filed on Apr. 30, 2021.
(60) Provisional application No. 63/180,460, filed on Apr. 27, 2021.

(51) Int. Cl. A61K 48/00; A61K 9/127; A61K 39/00; A61K 45/06; A61P 31/14; C07K 16/10; C12N 15/85

(52) U.S. Cl. CPC A61K 48/005; A61K 9/127; A61K 45/06; A61K 48/0075; A61P 31/14; C07K 16/1003; C12N 15/85; A61K 2039/505

(57) ABSTRACT

The application describes methods and compositions comprising ceDNA vectors useful for the expression of anti-CoV-2 S antibodies and antigen-binding fragments thereof in a cell, tissue or subject, and methods of treatment of COVID-19 with said ceDNA vectors.

### CROSS REFERENCE TO RELATED APPLICATIONS

This application claims the benefit of priority to U.S. Provisional Application No. 63/180,460, filed on Apr. 27, 2021, and U.S. Provisional Application No. 63/182,563, filed on Apr. 30, 2021, the contents of each of which are incorporated herein by reference in their entireties.

### TECHNICAL FIELD

The present disclosure relates to the field of antibody therapeutics, including non-viral vectors for expressing antibodies, or an antigen-binding fragments thereof, in a subject or a cell. The disclosure also relates to nucleic acid constructs, promoters, vectors, and host cells comprising the nucleic acids, as well as methods of delivering transgenes encoding the antibodies, or the antigen-binding fragments thereof, to a target cell, tissue, organ or organism. For example, the present disclosure provides methods for using non-viral ceDNA vectors to express an antibody, or an antigen-binding fragment thereof, from a cell, e.g., expressing the antibody, or the antigen-binding fragment thereof, for the treatment of a subject with a Coronavirus infection. The methods and compositions can be applied e.g., for a therapeutic or a prophylactic purpose in a subject in need thereof.

### BACKGROUND

Infectious existing and emerging pathogens continue to cause significant morbidity and mortality worldwide. While progress has been made on developing vaccines against some infectious pathogens to decrease mortality and/or spread, many remain a threat to human health, and treatments are needed. Certain infectious diseases are considered particularly important, e.g., because they showed a 100% lethality rate when they emerged, for example, HIV/AIDS; or because the infectious viral agent causes disease beyond the principal person of infection, for example, the emergence of birth defects from infection with Zika virus.

Coronaviruses (CoVs), a large family of single-stranded RNA viruses, can infect a wide variety of animals, including humans, causing respiratory, enteric, hepatic and neurological diseases. (Yin, Y., Wunderink, R G, Respirology (2018) 23 (2): 130-37, citing Weiss, SR, Leibowitz, IL, Coronavirus pathogenesis. Adv. Virus Res. (2011) 81: 85-164). Human coronaviruses, which were considered to be relatively harmless respiratory pathogens in the past, have now received worldwide attention as important pathogens in respiratory tract infection. As the largest known RNA viruses, CoVs are further divided into four genera: alpha-, beta-, gamma- and delta-coronavirus; the β-coronaviruses are further divided into A, B, C, and D lineages (Woo et al., J Virol. 2012 April; 86(7):3995-4008).

Coronaviruses are enveloped with a non-segmented, positive sense, single strand RNA, with size ranging from 26,000 to 37,000 bases; this is the largest known genome among RNA viruses. (Yang, Y. et al., J. Autoimmunity (2020), citing Weiss, S R et al. Microbiol. Mol. Biol. Rev. (2005) 69 (4): 635-64]. The viral RNA encodes structural proteins, and genes interspersed with in the structural genes, some of which play important roles in viral pathogenesis (Yang, Y. et al., J. Autoimmunity (2020), citing Fehr, A R, Perlman, S. Methods Mol. Biol. (2015) 1282: 1-23; Zhao, L. et al. Cell Host Microbe (2012) 11(6): 607-16]. The surface spike glycoprotein protein (S) is responsible for receptor binding and subsequent viral entry into host cells; it consists of S1 and S2 subunits. The membrane (M) and envelope (E) proteins play important roles in viral assembly; the E protein is required for pathogenesis (DeDiego, M L, et al. J. Virol. (2007) 81(4): 1701-13; Nieto-Torres, J L et al. PLOS Pathog. (2014) 10(5)). The nucleocapsid (N) protein contains two domains, both of which can bind virus RNA genomes via different mechanisms, and are necessary for RNA synthesis and packaging the encapsulated genome into virions. (Fehr, A R, Perlman, S. Methods Mol. Biol. (2015) 1282: 1-23; Song. Z. et al. Viruses (2019) 11(1): 59; Chang, C K et al., J. Biomed. Sci. (2006) 13(1): 59-72; Hurst, K R, et al. J. Virol. (2009) 83 (14): 7221-34). The N protein also is an antagonist of interferon and viral encoded repressor (VSR) of RNA interference (RNAi), which benefits viral replication (Cui, L. et al. J. Virol. (2015) 89 (17): 9029-43).

CoVs can co-infect humans and other vertebrate animals. Previously, seven CoVs were known to infect humans (HCoVs), including HCoV-229E and HCoV-NL63 in the α-coronaviruses, HCoV-OC43 and HCoV-HKU1 in the β-coronaviruses lineage A, SARS-CoV and SARS-CoV-2 in the β-coronaviruses lineage B (β-B coronaviruses), and MERS-CoV in the β-coronaviruses lineage C. SARS-CoV-2 shares a highly similar gene sequence and behavior pattern with SARS-CoV (Chan et al., Emerg Microbes Infect. 2020; 9(1):221-236). Both SARS-CoV-2 and SARS-CoV are in the coronavirus family. β-coronavirus genera. The genome of SARS-CoV-2 is more than 85% similar to the genome of the SARS-like virus ZC45 (bat-SL-CoVZC45, MG772933.1), and together these types of viruses form a unique Orthocoronavirinae subfamily with another SARS-like virus ZXC21 in the sarbecovirus subgenus (Zhu et al., N Engl J Med. 2020 Feb. 20; 382(8):727-733). All three viruses show typical β-coronavirus gene structure.

Beginning in December 2019, pneumonia cases of unknown origin were identified in Wuhan, China. The cause was later identified as severe acute respiratory syndrome coronavirus 2 (SARS-CoV-2) and the virus-infected pneumonia was later designated coronavirus disease 2019 (COVID-19) by the World Health Organization. The WHO declared COVID-19 a global health emergency at the end of January 2020. The outbreak of SARS-CoV-2 has killed more than 3 million people worldwide, including over half a million deaths in the United States attributed to COVID-19, as reported to the National Center for Health Statistics as of Apr. 16, 2021.

Antibody based therapeutics (e.g., mAbs) have been shown to be one of the most successful strategies to treat immune disorders, cancer and infectious diseases. In order to achieve a sufficiently high concentration of antibody for long lasting therapeutic effects, antibody therapies are traditionally delivered by repeated administration, e.g., by multiple injections. However, this dosing regimen results in an inconsistent level of antibody throughout the treatment period, a limited efficiency per administration, a high cost of administration and consumption of the antibody.

Recombinant AAV (rAAV) is perhaps the best studied vector for gene transfer in humans, with hundreds of clinical trials demonstrating safety of transduction. Adeno-associated viruses (AAVs) belong to the Parvoviridae family and more specifically constitute the Dependoparvovirus genus. Vectors derived from AAV (i.e., rAVV or AAV vectors) are attractive for delivering genetic material because (i) they are able to infect (transduce) a wide variety of non-dividing and dividing cell types including myocytes and neurons; (ii) they are devoid of the virus structural genes, thereby diminishing the host cell responses to virus infection, e.g., interferon-mediated responses; (iii) wild-type viruses are considered non-pathologic in humans; (iv) in contrast to wild type AAV, which are capable of integrating into the host cell genome, replication-deficient AAV vectors lack the replication (rep) gene and generally persist as episomes, thus limiting the risk of insertional mutagenesis or genotoxicity; and (v) in comparison to other vector systems, AAV vectors are generally considered to be relatively poor immunogens and therefore do not trigger a significant immune response (see (ii)), thus gaining persistence of the vector DNA and potentially, long-term expression of the therapeutic transgenes.

However, there are several major deficiencies in using AAV particles as a gene delivery vector. One major drawback associated with rAAV is its limited viral packaging capacity of about 4.5 kb of heterologous DNA (Dong et al., 1996; Athanasopoulos et al., 2004; Lai et al., 2010), and as a result, use of AAV vectors has been limited to less than 150,000 Da protein coding capacity. Particularly related to antibody delivery, the packaging limitation of AAV represents a significant challenge for the efficient delivery of both heavy and light chains that form the natural antibody structure. The second drawback is that as a result of the prevalence of wild-type AAV infection in the population, candidates for rAAV gene therapy have to be screened for the presence of neutralizing antibodies that eliminate the vector from the patient. A third drawback is related to the capsid immunogenicity that prevents re-administration to patients that were not excluded from an initial treatment. The immune system in the patient can respond to the vector which effectively acts as a “booster” shot to stimulate the immune system generating high titer anti-AAV antibodies that preclude future treatments. Preexisting immunity can severely limit the efficiency of transduction. Some recent reports indicate concerns with immunogenicity in high dose situations. Another notable drawback is that the onset of AAV-mediated gene expression is relatively slow, given that single-stranded AAV DNA must be converted to double-stranded DNA prior to heterologous gene expression.

Additionally, conventional AAV virions with capsids are produced by introducing a plasmid or plasmids containing the AAV genome, rep genes, and cap genes (Grimm et al., 1998). However, such encapsidated AAV virus vectors were found to inefficiently transduce certain cell and tissue types and the capsids also induce an immune response.

Accordingly, use of adeno-associated virus (AAV) vectors for delivery of antibody therapeutics is limited due to the single administration to patients (owing to the patient immune response), the limited range of transgene genetic material suitable for delivery in AAV vectors due to minimal viral packaging capacity (about 4.5 kb), and slow AAV-mediated gene expression.

There remains a need in the art for the development of treatments that prevent the spread of, treat, reduce the severity of life-threatening infectious diseases, such as SARS-CoV and SARS-CoV-2. Further, a need remains for the delivery of antibodies and antibody-based therapeutics through alternative routes or modalities of administration.

### BRIEF DESCRIPTION

The technology described herein relates to capsid-free (e.g., non-viral) DNA vectors with covalently-closed ends (referred to herein as a “closed-ended DNA vector” or a “ceDNA vector”), where the ceDNA vector comprises a nucleic acid sequence that encodes one or more polypeptides selected from the group consisting of an antibody heavy chain and an antibody light chain. These ceDNA vectors can be used to produce antibodies, or antigen-binding fragment thereof, for treatment of COVID-19. The application of ceDNA vectors expressing one or more nucleic acid sequences that encode one or more polypeptides selected from the group consisting of an antibody heavy chain and an antibody light chain to a subject is useful to: treat, prevent or reduce the severity of COVID-19 in a subject, be minimally invasive in delivery, be repeatable and dosed-to-effect, have rapid onset of therapeutic effect, and/or result in sustained expression of antibody or antigen-binding fragment thereof. Moreover, by employing a ceDNA vector to deliver a transgene (e.g., a nucleic acid sequence) encoding an antibody or an antigen-binding fragment thereof to cells or tissues, the adaptive immune response is bypassed, and the desired antibody specificities are produced without the use of immunization or passive transfer. That is, the ceDNA vector enters the cell via endocytosis, then escapes from the endosomal compartment and is transported to the nucleus. The transcriptionally active ceDNA episome results in the expression of encoded antibodies that may then be secreted from the cell into the circulation. The ceDNA vector may therefore enable continuous, sustained and long-term delivery of antibodies (e.g., the therapeutic antibodies, or antigen-binding fragments therein, described herein) administered by a single injection.

According to some embodiments, a ceDNA-vector comprising one or more nucleic acid sequences that encode one or more polypeptides selected from the group consisting of an antibody heavy chain and an antibody light chain is present in a liposome nanoparticle formulation (LNP).

The ceDNA vectors comprising one or more nucleic acid sequences that encode one or more polypeptides selected from the group consisting of an antibody heavy chain and an antibody light chain as described herein are capsid-free, linear duplex DNA molecules formed from a continuous strand of complementary DNA with covalently-closed ends (linear, continuous and non-encapsidated structure), which comprise a 5′ inverted terminal repeat (ITR) sequence and a 3′ ITR sequence, where the 5′ ITR and the 3′ ITR can have the same symmetrical three-dimensional organization with respect to each other. (i.e., symmetrical or substantially symmetrical), or alternatively, the 5′ ITR and the 3′ ITR can have different three-dimensional organization with respect to each other (i.e., asymmetrical ITRs). In addition, the ITRs can be from the same or different serotypes. According to some embodiments, a ceDNA vector can comprise ITR sequences that have a symmetrical three-dimensional spatial organization such that their structure is the same shape in geometrical space, or have the same A, C-C′ and B-B′ loops in 3D space (i.e., they are the same or are mirror images with respect to each other). According to some embodiments, one ITR can be from one AAV serotype, and the other ITR can be from a different AAV serotype.

Accordingly, some aspects of the technology described herein relate to a ceDNA vector for improved protein expression and/or production of the above described antibodies or antigen-binding fragments thereof that comprise ITR sequences that flank a nucleic acid sequence that encodes one or more polypeptides selected from the group consisting of an antibody heavy chain and an antibody light chain, wherein the ITR sequences being selected from any of: (i) at least one WT ITR and at least one modified AAV inverted terminal repeat (ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization. The ceDNA vectors disclosed herein can be produced in eukaryotic cells, thus devoid of prokaryotic DNA modifications and bacterial endotoxin contamination in insect cells.

According to a first aspect, the disclosure provides a capsid-free closed ended DNA (ceDNA) vector composition comprising a ceDNA vector comprising at least one nucleic acid sequence between flanking inverted terminal (ITRs), wherein the at least one nucleic acid sequence encodes a heavy chain (HC) and/or a light chain (LC) of an anti-CoV-2 S antibody or an antigen-binding fragment thereof. According to embodiments, the at least one nucleic acid sequence encodes the HC of the anti-SARS-CoV-2 S antibody, wherein the at least one nucleic acid sequence encoding the HC of the anti-SARS-CoV-2 S antibody is selected from the group consisting of SEQ ID NO: 1 and SEQ ID NO: 3. According to embodiments, the at least one nucleic acid sequence encodes the LC of the anti-SARS-CoV-2 S antibody, wherein the at least one nucleic acid sequence encoding the LC of the anti-SARS-CoV-2 S antibody is selected from the group consisting of SEQ ID NO: 2 and SEQ ID NO: 4. According to embodiments, the at least one nucleic acid sequence encodes both the HC and LC of the anti-SARS-CoV-2 S antibody, wherein the nucleic acid sequence encoding the HC is selected from SEQ ID NO: 1 and SEQ ID NO: 3, and the nucleic acid sequence encoding the LC is selected from SEQ ID NO: 2 and SEQ ID NO: 4.

In another aspect, the disclosure provides a capsid-free closed-ended DNA (ceDNA) vector combination comprising a first ceDNA vector comprising at least one nucleic acid sequence between flanking inverted terminal repeats (ITRs), wherein the at least one nucleic acid sequence encodes a heavy chain (HC) of an anti-CoV-2 S antibody or an antigen-binding fragment thereof; and a second ceDNA vector comprising at least one nucleic acid sequence between flanking inverted terminal repeats (ITRs), wherein the at least one nucleic acid sequence encodes a light chain (LC) of an anti-CoV-2 S antibody or an antigen-binding fragment thereof. According to embodiments, the at least one nucleic acid sequence encodes an anti-SARS-CoV-2 S antibody HC comprising SEQ ID NO: 1 or SEQ ID NO: 3. According to embodiments, the at least one nucleic acid sequence encodes an anti-SARS-CoV-2 S antibody LC comprising SEQ ID NO: 2 or SEQ ID NO: 4. According to embodiments of the above aspects and embodiments, the first ceDNA vector comprises at least one nucleic acid sequence encoding an anti-SARS-CoV-2 S antibody HC comprising SEQ ID NO: 1; and the second ceDNA vector comprises at least one nucleic acid sequence encoding an anti-SARS-CoV-2 S antibody LC comprising SEQ ID NO: 2. According to embodiments, the first ceDNA vector comprises at least one nucleic acid sequence encoding an anti-SARS-CoV-2 S antibody HC comprising SEQ ID NO: 3; and the second ceDNA vector comprises at least one nucleic acid sequence encoding an anti-SARS-CoV-2 S antibody LC comprising SEQ ID NO: 4. According to embodiments of the above aspects and embodiments, the first ceDNA vector comprises an open reading frame that is at least 85% identical to SEQ ID NO:25. According to embodiments of the above aspects and embodiments, the second ceDNA vector comprises an ORF that is at least 85% identical to SEQ ID NO:26. According to embodiments of the above aspects and embodiments, the first ceDNA vector and the second ceDNA vector are present at a molar ratio of 1:1. According to embodiments of the above aspects and embodiments, the first ceDNA vector and the second ceDNA vector each comprise a promoter sequence, operatively linked to the least one nucleic acid sequence. According to embodiments, the promoters are the same, or the promoters are different. According to embodiments of the above aspects and embodiments, the at least one ITR comprises a functional terminal resolution site and a Rep binding site. According to embodiments, the first and the second ceDNA vector are encapsulated in a lipid nanoparticle.

According to another aspect, the disclosure provides a capsid-free close-ended DNA (ceDNA) vector formulation comprising a first ceDNA vector comprising an open reading frame (ORF) at least 85% identical to SEQ ID NO:25; and a second ceDNA vector comprising an ORF at least 85% identical to SEQ ID NO:26.

According to another aspect, the disclosure provides a capsid-free close-ended DNA (ceDNA) vector composition comprising a first ceDNA vector comprising an open reading frame (ORF) consisting of SEQ ID NO:25; and a second ceDNA vector comprising an ORF consisting of SEQ ID NO:26.

According to another aspect, the disclosure provides a method of expressing an anti-CoV-2 S antibodies and antigen-binding fragments thereof in a cell comprising contacting the cell with the ceDNA vector formulation of any one of the aspects and embodiments herein. According to embodiments, the cell is in vitro or in vivo. According to embodiments, the at least one nucleic acid sequence is codon optimized for expression in the eukaryotic cell.

According to another aspect, the disclosure provides a method of treating a subject with COVID-19, comprising administering to the subject a ceDNA vector composition of the aspects and embodiments herein.

According to another aspect, the disclosure provides a method of preventing infection of a subject with SARS-CoV-2, comprising administering to the subject a ceDNA vector composition of the aspects and embodiments herein. According to embodiments of the above aspects and embodiments, the subject is administered one or more additional therapeutic agents. According to embodiments of the above aspects and embodiments, the ceDNA vector formulation is administered by intravenous, subcutaneous or intramuscular injection.

According to another aspect, the disclosure provides an anti-SARS-CoV-2 S antibody, or an antigen binding fragment thereof, wherein the antibody, or the antigen binding fragment thereof, comprises a heavy chain (HC) and a light chain (LC), wherein the HC comprises an amino acid sequence that is at least 85% identical to SEQ ID NO: 1 or SEQ ID NO: 3; and the LC comprises an amino acid sequence that is at least 85% identical to SEQ ID NO: 2 or SEQ ID NO: 4, wherein the anti-SARS-CoV-2 S antibody is expressed from one or more ceDNA vectors containing a nucleic acid sequence encoding the HC and/or LC. According to embodiments, the HC comprises SEQ ID NO: 1 and the LC comprises SEQ ID NO: 2; or the HC comprises SEQ ID NO: 3 and the LC comprises SEQ ID NO: 4. According to embodiments, the one or more ceDNA vectors are expressed in a cell. According to embodiments, the cell is in vitro or in vivo.

According to another aspect, the disclosure provides a pharmaceutical composition comprising the ceDNA vector composition of the aspects and embodiments herein. According to embodiments, the pharmaceutical composition further comprises an additional therapeutic agent.

According to another aspect, the disclosure provides a cell containing the ceDNA vector composition of the aspects and embodiments herein.

According to another aspect, the disclosure provides a composition comprising the ceDNA vector composition of the aspects and embodiments herein. According to embodiments, the lipid is a lipid nanoparticle (LNP).

According to another aspect, the disclosure provides a kit comprising the ceDNA vector composition, the pharmaceutical composition or the cell of the aspects and embodiments herein.

These and other aspects of the disclosure are described in further detail below.

### DESCRIPTION OF DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1A illustrates an exemplary structure of a ceDNA vector for expression of an antibody, or antigen-binding fragment thereof (e.g., HC or LC), as disclosed herein, comprising asymmetric ITRs. In this embodiment, the exemplary ceDNA vector comprises an expression cassette containing CAG promoter, WPRE, and BGHpA. An open reading frame (ORF) encoding the transgene (e.g., nucleic acid sequence encoding the antibody or the antigen-binding fragment thereof) can be inserted into the cloning site (R3/R4) between the CAG promoter and WPRE. The expression cassette is flanked by two inverted terminal repeats (ITRs)—the wild-type AAV2 ITR on the upstream (5′-end) and the modified ITR on the downstream (3′-end) of the expression cassette, therefore the two ITRs flanking the expression cassette are asymmetric with respect to each other.

FIG. 1B illustrates an exemplary structure of a ceDNA vector for expression of an antibody, or antigen-binding fragment thereof (e.g., HC or LC), as disclosed herein comprising asymmetric ITRs with an expression cassette containing CAG promoter, WPRE, and BGHpA. An open reading frame (ORF) encoding the transgene (e.g., nucleic acid sequence encoding the antibody or the antigen-binding fragment thereof) can be inserted into the cloning site between CAG promoter and WPRE. The expression cassette is flanked by two inverted terminal repeats (ITRs)—a modified ITR on the upstream (5′-end) and a wild-type ITR on the downstream (3′-end) of the expression cassette.

FIG. 1C illustrates an exemplary structure of a ceDNA vector for expression of an antibody, or an antigen-binding fragment thereof, as disclosed herein comprising asymmetric ITRs, with an expression cassette containing an enhancer/promoter, the transgene (e.g., nucleic acid sequence encoding the antibody or the antigen-binding fragment thereof), a post transcriptional element (WPRE), and a polyA signal. An open reading frame (ORF) allows insertion of the transgene into the cloning site between CAG promoter and WPRE. The expression cassette is flanked by two inverted terminal repeats (ITRs) that are asymmetrical with respect to each other; a modified ITR on the upstream (5′-end) and a modified ITR on the downstream (3′-end) of the expression cassette, where the 5′ ITR and the 3′ITR are both modified ITRs but have different modifications (i.e., they do not have the same modifications).

FIG. 1D illustrates an exemplary structure of a ceDNA vector for expression of an antibody, or an antigen-binding fragment thereof (e.g., HC or LC), as disclosed herein, comprising symmetric modified ITRs, or substantially symmetrical modified ITRs as defined herein, with an expression cassette containing CAG promoter. WPRE, and BGHpA. An open reading frame (ORF) encoding the transgene (e.g., nucleic acid sequence encoding the antibody or antigen-binding fragment thereof) is inserted into the cloning site between CAG promoter and WPRE. The expression cassette is flanked by two modified inverted terminal repeats (ITRs), where the 5′ modified ITR and the 3′ modified ITR are symmetrical or substantially symmetrical.

FIG. 1E illustrates an exemplary structure of a ceDNA vector for expression of an antibody, or an antigen-binding fragment thereof (e.g., HC or LC), as disclosed herein comprising symmetric modified ITRs, or substantially symmetrical modified ITRs as defined herein, with an expression cassette containing an enhancer/promoter, a transgene, a post transcriptional element (WPRE), and a polyA signal. An open reading frame (ORF) allows insertion of a transgene (e.g., nucleic acid sequence encoding the antibody or antigen-binding fragment thereof) into the cloning site between CAG promoter and WPRE. The expression cassette is flanked by two modified inverted terminal repeats (ITRs), where the 5′ modified ITR and the 3′ modified ITR are symmetrical or substantially symmetrical.

FIG. 1F illustrates an exemplary structure of a ceDNA vector for expression of an antibody, or an antigen-binding fragment thereof (e.g., HC or LC), as disclosed herein, comprising symmetric WT-ITRs, or substantially symmetrical WT-ITRs as defined herein, with an expression cassette containing CAG promoter, WPRE, and BGHpA. An open reading frame (ORF) encoding a transgene (e.g., nucleic acid sequence encoding the antibody or antigen-binding fragment thereof) is inserted into the cloning site between CAG promoter and WPRE. The expression cassette is flanked by two wild type inverted terminal repeats (WT-ITRs), where the 5′ WT-ITR and the 3′ WT ITR are symmetrical or substantially symmetrical.

FIG. 1G illustrates an exemplary structure of a ceDNA vector for expression of an antibody, or an antigen-binding fragment thereof, as disclosed herein, comprising symmetric modified ITRs, or substantially symmetrical modified ITRs as defined herein, with an expression cassette containing an enhancer/promoter, a transgene, a post transcriptional element (WPRE), and a polyA signal. An open reading frame (ORF) allows insertion of a transgene into the cloning site between CAG promoter and WPRE. The expression cassette is flanked by two wild type inverted terminal repeats (WT-ITRs), where the 5′ WT-ITR and the 3′ WT ITR are symmetrical or substantially symmetrical.

FIG. 2A provides the T-shaped stem-loop structure of a wild-type left ITR of AAV2 with identification of A-A′ arm, B-B′ arm, C-C′ arm, two Rep binding sites (RBE and RBE′) and also shows the terminal resolution site (trs). The RBE contains a series of 4 duplex tetramers that are believed to interact with either Rep 78 or Rep 68. In addition, the RBE′ is also believed to interact with Rep complex assembled on the wild-type ITR or mutated ITR in the construct. The D and D′ regions contain transcription factor binding sites and other conserved structure. FIG. 2B shows proposed Rep-catalyzed nicking and ligating activities in a wild-type left ITR, including the T-shaped stem-loop structure of the wild-type left ITR of AAV2 with identification of A-A′ arm, B-B′ arm, C-C′ arm, two Rep Binding sites (RBE and RBE′) and also shows the terminal resolution site (trs), and the D and D′ region comprising several transcription factor binding sites and other conserved structure.

FIG. 3A provides the primary structure (polynucleotide sequence) (left) and the secondary structure (right) of the RBE-containing portions of the A-A′ arm, and the C-C′ and B-B′ arm of the wild type left AAV2 ITR. FIG. 3B shows an exemplary mutated ITR (also referred to as a modified ITR) sequence for the left ITR. Shown is the primary structure (left) and the predicted secondary structure (right) of the RBE portion of the A-A′ arm, the C arm and B-B′ arm of an exemplary mutated left ITR (ITR-1, left). FIG. 3C shows the primary structure (left) and the secondary structure (right) of the RBE-containing portion of the A-A′ loop, and the B-B′ and C-C′ arms of wild type right AAV2 ITR. FIG. 3D shows an exemplary right modified ITR. Shown is the primary structure (left) and the predicted secondary structure (right) of the RBE containing portion of the A-A′ arm, the B-B′ and the C arm of an exemplary mutant right ITR (ITR-1, right). Any combination of left and right ITR (e.g., AAV2 ITRs or other viral serotype or synthetic ITRs) can be used as taught herein. Each of FIGS. 3A-3D polynucleotide sequences refer to the sequence used in the plasmid or bacmid/baculovirus genome used to produce the ceDNA as described herein. Also included in each of FIGS. 3A-3D are corresponding ceDNA secondary structures inferred from the ceDNA vector configurations in the plasmid or bacmid/baculovirus genome and the predicted Gibbs free energy values.

FIG. 4A is a schematic illustrating an upstream process for making baculovirus infected insect cells (BIICs) that are useful in the production of a ceDNA vector for expression of the antibody, or antigen-binding fragment thereof, disclosed herein in the process described in the schematic in FIG. 4B. FIG. 4B is a schematic of an exemplary method of ceDNA production and FIG. 4C illustrates a biochemical method and process to confirm ceDNA vector production. FIG. 4D and FIG. 4E are schematic illustrations describing a process for identifying the presence of ceDNA in DNA harvested from cell pellets obtained during the ceDNA production processes in FIG. 4B. FIG. 4D shows schematic expected bands for an exemplary ceDNA either left uncut or digested with a restriction endonuclease and then subjected to electrophoresis on either a native gel or a denaturing gel. The leftmost schematic is a native gel, and shows multiple bands suggesting that in its duplex and uncut form ceDNA exists in at least monomeric and dimeric states, visible as a faster-migrating smaller monomer and a slower-migrating dimer that is twice the size of the monomer. The schematic second from the left shows that when ceDNA is cut with a restriction endonuclease, the original bands are gone and faster-migrating (e.g., smaller) bands appear, corresponding to the expected fragment sizes remaining after the cleavage. Under denaturing conditions, the original duplex DNA is single-stranded and migrates as a species twice as large as observed on native gel because the complementary strands are covalently linked. Thus, in the second schematic from the right, the digested ceDNA shows a similar banding distribution to that observed on native gel, but the bands migrate as fragments twice the size of their native gel counterparts. The rightmost schematic shows that uncut ceDNA under denaturing conditions migrates as a single-stranded open circle, and thus the observed bands are twice the size of those observed under native conditions where the circle is not open. In this figure “kb” is used to indicate relative size of nucleotide molecules based, depending on context, on either nucleotide chain length (e.g., for the single stranded molecules observed in denaturing conditions) or number of basepairs (e.g., for the double-stranded molecules observed in native conditions). FIG. 4E shows DNA having a non-continuous structure. The ceDNA can be cut by a restriction endonuclease, having a single recognition site on the ceDNA vector, and generate two DNA fragments with different sizes (1 kb and 2 kb) in both neutral and denaturing conditions. FIG. 4E also shows a ceDNA having a linear and continuous structure. The ceDNA vector can be cut by the restriction endonuclease, and generate two DNA fragments that migrate as 1 kb and 2 kb in neutral conditions, but in denaturing conditions, the stands remain connected and produce single strands that migrate as 2 kb and 4 kb.

FIG. 5 is an exemplary picture of a denaturing gel running examples of ceDNA vectors with (+) or without (−) digestion with endonucleases (EcoRI for ceDNA construct 1 and 2; BamH1 for ceDNA construct 3 and 4; SpeI for ceDNA construct 5 and 6; and XhoI for ceDNA construct 7 and 8) Constructs 1-8 are described in Example 1 of International Application PCT PCT/US18/49996, which is incorporated herein in its entirety by reference. Sizes of bands highlighted with an asterisk were determined and provided on the bottom of the picture.

FIG. 6 is a graph that shows detection of antibody expression by the ceDNA constructs tested in Example 6. Anti-spike human IgG was used to detect antibody expression, which was quantified by ng/ml anti-spike hIgG detected at up to 35 days after injection with the ceDNA construct.

FIG. 7 is a graph that shows serum levels of the antibodies expressed by the ceDNA constructs tested in Example 7, at day 3 and day 7 post-injection.

FIG. 8 shows neutralization of SARS-CoV-2 by the antibodies expressed by the ceDNA construct 1856 (LC)/1859 (HC) following hydrodynamic delivery in vitro. Virus neutralization is shown at Days 4 and 7.

FIG. 9 shows neutralization of SARS-CoV-2 by the antibodies expressed by the ceDNA construct 1856 (LC)/1859 (HC) following hydrodynamic delivery in vitro.

FIG. 10 is a graph that shows LNP delivery of dual vectors (ceDNA-1; ceDNA constructs 1856 (LC) and 1859 (HC) coformulated in LNP formulation 1) achieved persistent, therapeutically relevant, anti-Spike hIgG concentrations of 8 μg/mL in mice as compared to expression of the single vector of ceDNA (dual ORF “ceDNA-2”).

FIG. 11 is a graph that shows a dose dependent increase in antibody expression in ceDNA-1 dual vector (ceDNA constructs 1856 (LC) and 1859 (HC)) or ceDNA-2 single vector (dual ORFs) designed to express the antibody HC and LC from a single ceDNA molecule, following hydrodynamic delivery.

FIG. 12A is a graph showing the dissociation constant (K_{D}) values of cell line-derived (recombinant LS) or ceDNA-derived Antibody 1 (serum purified ceDNA derived LS) and cell line-derived (recombinant LS-GAALIE) or ceDNA-derived (serum purified ceDNA derived LS-GAALIE) Antibody 2 when associated with then dissociated from the FcγRIIIa-V receptor. FIG. 12B is a graph showing the dissociation constant (K_{D}) values of cell line-derived (recombinant LS) or ceDNA-derived (serum purified ceDNA derived LS) Antibody 1 and cell line-derived (recombinant LS-GAALIE) or ceDNA-derived (serum purified ceDNA derived LS-GAALIE) Antibody 2 when associated with then dissociated from the FcγRIIIaF receptor.

FIG. 13A is a graph showing activation levels of Jurkat FcγRIIIa of cell line-derived (recombinant LS) Antibody 1, cell line-derived (recombinant LS-GAALIE) Antibody 2, or ceDNA-derived (serum purified ceDNA derived LS-GAALIE) Antibody 2. FIG. 13B is a graph showing activation levels of Jurkat FcγRIIa of cell line-derived (recombinant LS) Antibody 1, cell line-derived (recombinant LS-GAALIE) Antibody 2, or ceDNA-derived (serum purified ceDNA derived LS-GAALIE) Antibody 2.

### DETAILED DESCRIPTION

According to embodiments of the present disclosure, compositions for delivering antibodies or antigen-binding fragments thereof by ceDNA vectors are provided. In particular, the present disclosure provides compositions for delivering anti-SARS-CoV-2 S antibodies or antigen-binding fragments thereof by ceDNA vectors. According to some embodiments, the anti-SARS-CoV-2 antibodies or antigen-binding fragments thereof, are capable of binding to a SARS-CoV-2 surface glycoprotein (S) expressed on a cell surface of a host cell and/or on a SARS-CoV-2 virion. The antibodies or antigen-binding fragments described herein are useful for therapeutic purposes, e.g., inhibiting or neutralizing SARS-CoV-2 activity, blocking attachments SARS-CoV-2 to a host cell and/or for preventing the invasion of SARS-CoV-2 into the host cell, inhibiting cell-cell transmission of SARS-CoV-2 or by killing SARS-CoV-2-infected cells, and reducing the production of pathogenic virus. According to some embodiments, the antibodies or the antigen-binding fragments thereof, are useful in preventing, treating, or ameliorating at least one symptom of a SARS-CoV-2 infection (COVID-19) in a subject. According to some embodiments, the antibodies, or the antigen-binding fragments thereof, can be administered prophylactically or therapeutically to a subject who has is at risk of being infected with SARS-CoV-2. According to some embodiments, the antibodies, or the antigen-binding fragments thereof, can be administered prophylactically or therapeutically to a subject who has or is at risk of developing COVID-19. According to some embodiments, the antibodies, or the antigen-binding fragments thereof, can be administered as a first-line treatment to a subject who has already been exposed to SARS-CoV-2.

### I. Definitions

Unless otherwise defined herein, scientific and technical terms used in connection with the present application shall have the meanings that are commonly understood by those of ordinary skill in the art to which this disclosure belongs. It should be understood that this disclosure is not limited to the particular methodology, protocols, and reagents, etc., described herein and as such can vary. The terminology used herein is for the purpose of describing particular embodiments only and is not intended to limit the scope of the present disclosure, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), Fields Virology, 6^{th} Edition, published by Lippincott Williams & Wilkins, Philadelphia, PA. USA (2013), Knipe, D. M. and Howley, P. M. (ed.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the term “SARS-CoV-2” (also known as 2019-nCoV and Wuhan coronavirus) refers to the newly-emerged coronavirus which was identified as the cause of a serious outbreak starting in Wuhan, China.

As used herein, the term “CoV-2-S”, also called “S” or “S protein” is meant to refer to the surface glycoprotein (S) (also referred to as the “spike protein”) of a Coronavirus, and can refer to specific S proteins such as SARS-CoV-2-S. The SARS-CoV-2-S protein is a 1273 amino acid type I membrane glycoprotein which assembles into trimers that constitute the spikes or peplomers on the surface of the enveloped coronavirus particle. The protein has two essential functions, host receptor binding and membrane fusion, which are attributed to the N-terminal (S1) and C-terminal (S2) halves of the S protein. CoV-S binds to its cognate receptor via a receptor binding domain (RBD) present in the S1 subunit. The term “CoV-2-S” includes protein variants of CoV S protein isolated from different CoV isolates as well as recombinant CoV spike protein or a fragment thereof.

The term “coronavirus infection” or “CoV infection,” as used herein, refers to infection with a coronavirus such as SARS-CoV-2. The term includes coronavirus respiratory tract infections, often in the lower respiratory tract. Symptoms can include high fever, dry cough, shortness of breath, pneumonia, gastro-intestinal symptoms such as diarrhea, organ failure (kidney failure and renal dysfunction), septic shock, and death in severe cases.

As used herein, the term “antibody”, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The term “monoclonal antibody”, as used herein, refers to a population of substantially homogeneous antibodies, i.e., the antibody molecules comprising the population are identical in amino acid sequence except for possible naturally occurring mutations that may be present in minor amounts. A “plurality” of such monoclonal antibodies and fragments in a composition refers to a concentration of identical (i.e., in amino acid sequence except for possible naturally occurring mutations that may be present in minor amounts) antibodies and fragments which is above that which would normally occur in nature, e.g., in the blood of a host organism such as a mouse or a human.

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region that can bind the antigen. The exact boundaries of these CDRs have been defined differently according to different systems. The system described by Kabat (Kabat et al. (1987; 1991) Sequences of Proteins of Immunological Interest (National Institutes of Health, Bethesda, Md.) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk (1987) J. Mol. Biol. 196: 901-917; and Chothia et al. (1989) Nature 342: 877-883) found that certain sub-portions within Kabat CDRs adopt nearly identical peptide backbone conformations, despite having great diversity at the level of amino acid sequence. These sub-portions were designated as L1, L2 and L3 or H1, H2 and H3, or, L-CDR1, L-CDR2 and L-CDR3 or H-CDR1, H-CDR2 and H-CDR3, where the “L” and the “H” designate the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9: 133-139 and MacCallum (1996) J. Mol. Biol. 262(5): 732-45. Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding (see, for example: Lu X et al., MAbs. 2019 January; 11(1):45-57). The methods used herein may utilize CDRs defined according to any of these systems, although certain embodiments use Kabat or Chothia defined CDRs.

As used herein, the term “antigen-binding fragment” or “antigen-binding portion” of an antibody (or simply “antibody fragment”), refers to one or more fragments of an antibody that retain the ability to specifically bind to an antigen. It has been shown that the antigen-binding function of an antibody can be performed by fragments of a full-length antibody. Binding fragments include Fab, Fab′, F(ab′)2, Fabc, Fv, single chains, and single-chain antibodies. Examples of binding fragments encompassed within the term “antigen-binding fragment” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody. (v) a dAb fragment (Ward et al. (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. USA 90:6444-6448; Poljak et al. (1994) Structure 2:1121-1123). The antibody portions of the disclosure are described in further detail in U.S. Pat. Nos. 6,090,382, 6,258,562, 6,509,015, each of which is incorporated herein by reference in its entirety.

The term “CL” refers to an “immunoglobulin light chain constant region” or a “light chain constant region.” i.e., a constant region from an antibody light chain. The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH2. CH3, and CH4 domains (IgE, IgM). The Fc region of an antibody heavy chain is described further herein. In any of the presently disclosed embodiments, an antibody or antigen-binding fragment of the present disclosure comprises any one or more of CL, a CH1, a CH2, and a CH3. In certain embodiments, a CL comprises an amino acid sequence having 90%, 91%, 92%, 93%, 94%, 95%, 96%, 975, 98%, 99%, or 100% identity to the amino acid sequence of SEQ ID NO.: 13 or SEQ ID NO.: 193. In certain embodiments, a CH1-CH2-CH3 comprises an amino acid sequence having 90%, 91%, 92%. 93%, 94%, 95%, 96%, 975, 98%, 99%, or 100% identity to the amino acid sequence of SEQ ID NO.: 14 or SEQ ID NO.: 17 or SEQ ID NO.: 15 or SEQ ID NO.: 18. It will be understood that, for example, production in a mammalian cell line can remove one or more C-terminal lysine of an antibody heavy chain (see, e.g., Liu et al. mAbs 6(5):1145-1154 (2014)). Accordingly, an antibody or antigen-binding fragment of the present disclosure can comprise a heavy chain, a CH1-CH3, a CH3, or an Fc polypeptide wherein a C-terminal lysine residue is present or is absent; in other words, encompassed are embodiments where the C-terminal residue of a heavy chain, a CH1-CH3, or an Fc polypeptide is not a lysine, and embodiments where a lysine is the C-terminal residue. In certain embodiments, a composition comprises a plurality of an antibody and/or an antigen-binding fragment of the present disclosure, wherein one or more antibody or antigen-binding fragment does not comprise a lysine residue at the C-terminal end of the heavy chain, CH1-CH3, or Fc polypeptide, and wherein one or more antibody or antigen-binding fragment comprises a lysine residue at the C-terminal end of the heavy chain, CH1-CH3, or Fc polypeptide.

“Chimeric antibodies” refers to antibodies wherein some portion of each of the amino acid sequences of heavy and light chains is homologous to corresponding sequences in antibodies derived from a particular species or belonging to a particular class, while the remaining segment of the chains is homologous to corresponding sequences from another species.

“Humanized antibodies” refer to antibodies which comprise at least one chain comprising variable region framework residues substantially from a human antibody chain (referred to as the acceptor immunoglobulin or antibody) and at least one complementarity determining region (CDR) substantially from a non-human-antibody (e.g., mouse). In addition, humanized antibodies typically undergo further alterations in order to improve affinity and/or immunogenicity.

The term “multivalent antibody” refers to an antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” or “dual specific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope

The term “human antibody”, as used herein, is intended to include antibodies having variable and constant regions derived from human germline immunoglobulin sequences. Human antibodies of may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell.

A “neutralizing antibody”, as used herein, is one that can neutralize, i.e., prevent, inhibit, reduce, impede, or interfere with, the ability of a pathogen to initiate and/or perpetuate an infection in a host. This inhibition can be assessed by measuring one or more indicators of biological activity (either in vitro or in vivo), cellular activation, and/or receptor binding. According to some embodiments, the antibody or antigen-binding fragment thereof is capable of preventing and/or neutralizing a SARS-CoV-2 infection in an in vitro model of infection and/or in an in vivo animal model of infection and/or in a human.

The term “antigen” as used herein, is meant to refer to a molecule containing one or more epitopes (either linear, conformational or both) that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, inclusive, such as, 9, 10, 11, 12, 13, 14 or 15 amino acids. The term includes polypeptides which include modifications, such as deletions, additions and substitutions (generally conservative in nature) as compared to a native sequence, as long as the protein maintains the ability to elicit an immunological response, as defined herein. These modifications may be deliberate, as through site-directed mutagenesis, or may be accidental, such as through mutations of hosts which produce the antigens.

The term “epitope” may be also referred to as an antigenic determinant, is a molecular determinant (e.g., polypeptide determinant) that can be specifically bound by a binding agent, immunoglobulin or T-cell receptor. Epitope determinants include chemically active surface groupings of molecules, such as amino acids, sugar side chains, phosphoryl, or sulfonyl, and, in certain embodiments, may have specific three-dimensional structural characteristics, and/or specific charge characteristics. Epitopes may be defined as structural or functional. Functional epitopes are generally a subset of the structural epitopes and have those residues that directly contribute to the affinity of the interaction. Epitopes may be linear or conformational, that is, composed of non-linear amino acids. An epitope recognized by an antibody or an antigen-binding fragment of an antibody is a structural element of an antigen that interacts with CDRs (e.g., the complementary site) of the antibody or the fragment. An epitope may be formed by contributions from several amino acid residues, which interact with the CDRs of the antibody to produce specificity. An antigenic fragment can contain more than one epitope. In certain embodiments, an antibody specifically binds an antigen when it recognizes its target antigen in a complex mixture of proteins and/or macromolecules. For example, antibodies are said to “bind to the same epitope” if the antibodies cross-compete (one prevents the binding or modulating effect of the other). In certain embodiments, an antibody or antigen-binding fragment of the present disclosure associates with or unites with a SARS-CoV-2 surface glycoprotein epitope or antigen comprising the epitope, while not significantly associating or uniting with any other molecules or components in a sample. In certain embodiments, an antibody or antigen-binding fragment of the present disclosure associates with or unites (e.g., binds) to a SARS-CoV-2 surface glycoprotein epitope and can also associate with or unite with an epitope from another coronavirus (e.g., SARS CoV) present in the sample, but not significantly associating or uniting with any other molecules or components in the sample. In other words, in certain embodiments, an antibody or antigen binding fragment of the present disclosure is cross-reactive for SARS-CoV-2 and one or more additional coronavirus.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example 1 of U.S. Pat. No. 6,258,562 and Jönsson et al. (1993) Ann. Biol. Clin. 51:19; Jönsson et al. (1991) Biotechniques 11:620-627; Johnsson et al. (1995) J. Mol. Recognit. 8:125; and Johnnson et al. (1991) Anal. Biochem. 198:268.

As used herein, “specifically binds” refers to an association or union of an antibody or antigen-binding fragment to an antigen with an affinity or Kₐ (i.e., an equilibrium association constant of a particular binding interaction with units of 1/M) equal to or greater than 10⁵ M⁻¹ (which equals the ratio of the on-rate [Kₒₙ] to the off rate [K_{off}] for this association reaction), while not significantly associating or uniting with any other molecules or components in a sample. Alternatively, affinity may be defined as an equilibrium dissociation constant (K_{d}) of a particular binding interaction with units of M (e.g., 10⁻⁵ M to 10⁻¹³ M). Antibodies may be classified as “high-affinity” antibodies or as “low-affinity” antibodies. “High-affinity” antibodies refer to those antibodies having a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹²M⁻¹, or at least 10¹³ M⁻¹. “Low-affinity” antibodies refer to those antibodies having a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M).

The term “K_{off}”, as used herein, is intended to refer to the off-rate constant for dissociation of an antibody from the antibody/antigen complex.

The term “K_{d}”, as used herein, is intended to refer to the dissociation constant of a particular antibody-antigen interaction.

The term “IC₅₀” as used herein, is intended to refer to the concentration of the inhibitor required to inhibit the biological endpoint of interest.

As used herein, the terms “heterologous nucleic acid sequence” and “transgene” are used interchangeably and refer to a nucleic acid of interest (other than a nucleic acid encoding a capsid polypeptide) that is incorporated into and may be delivered and expressed by a ceDNA vector as disclosed herein. According to some embodiments, the term “heterologous nucleic acid” is meant to refer to a nucleic acid (or transgene) that is not present in, expressed by, or derived from the cell or subject to which it is contacted.

As used herein, the terms “expression cassette” and “transcription cassette” are used interchangeably and refer to a linear stretch of nucleic acids that includes a transgene that is operably linked to one or more promoters or other regulatory sequences sufficient to direct transcription of the transgene, but which does not comprise capsid-encoding sequences, other vector sequences or inverted terminal repeat regions. An expression cassette may additionally comprise one or more cis-acting sequences (e.g., promoters, enhancers, or repressors), one or more introns, and one or more post-transcriptional regulatory elements.

The terms “polynucleotide” and “nucleic acid.” used interchangeably herein, refer to a polymeric form of nucleotides of any length, either ribonucleotides or deoxyribonucleotides. Thus, this term includes single, double, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

DNA may be in the form of, e.g., antisense molecules, plasmid DNA, DNA-DNA duplexes, pre-condensed DNA, PCR products, vectors (P1, PAC, BAC, YAC, artificial chromosomes), expression cassettes, chimeric sequences, chromosomal DNA, or derivatives and combinations of these groups. DNA may be in the form of minicircle, plasmid, bacmid, minigene, ministring DNA (linear covalently closed DNA vector), closed-ended linear duplex DNA (CELiD or ceDNA), doggybone (dbDNA™) DNA, dumbbell shaped DNA, minimalistic immunological-defined gene expression (MIDGE)-vector, viral vector or nonviral vectors. RNA may be in the form of small interfering RNA (siRNA), Dicer-substrate dsRNA, small hairpin RNA (shRNA), asymmetrical interfering RNA (aiRNA), microRNA (miRNA), mRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphorodiamidate morpholino oligomer (morpholino), phosphoramidates, methyl phosphonates, chiral-methyl phosphonates, 2′-O-methyl ribonucleotides, locked nucleic acid (LNA™), and peptide nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

“Nucleotides” contain a sugar deoxyribose (DNA) or ribose (RNA), a base, and a phosphate group. Nucleotides are linked together through the phosphate groups.

“Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure. An “expression cassette” includes a DNA coding sequence operably linked to a promoter.

By “hybridizable” or “complementary” or “substantially complementary” it is meant that a nucleic acid (e.g., RNA) includes a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C). In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a subject DNA-targeting RNA molecule is considered complementary to a uracil (U), and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary.

The terms “peptide.” “polypeptide.” and “protein” are used interchangeably herein, and refer to a polymeric form of amino acids of any length, which can include coded and non-coded amino acids, chemically or biochemically modified or derivatized amino acids, and polypeptides having modified peptide backbones.

A DNA sequence that “encodes” a particular anti-CoV-2 S HC and/or LC is a DNA nucleic acid sequence that is transcribed into the particular RNA and/or protein. A DNA polynucleotide may encode an RNA (mRNA) that is translated into protein, or a DNA polynucleotide may encode an RNA that is not translated into protein (e.g., tRNA, rRNA, or a DNA-targeting RNA; also called “non-coding” RNA or “ncRNA”).

As used herein, the term “terminal repeat” or “TR” includes any viral terminal repeat or synthetic sequence that comprises at least one minimal required origin of replication and a region comprising a palindrome hairpin structure. A Rep-binding sequence (“RBS”) (also referred to as RBE (Rep-binding element)) and a terminal resolution site (“TRS”) together constitute a “minimal required origin of replication” and thus the TR comprises at least one RBS and at least one TRS. TRs that are the inverse complement of one another within a given stretch of polynucleotide sequence are typically each referred to as an “inverted terminal repeat” or “ITR”. In the context of a virus, ITRs mediate replication, virus packaging, integration and provirus rescue. As was unexpectedly found. TRs that are not inverse complements across their full length can still perform the traditional functions of ITRs, and thus the term ITR is used herein to refer to a TR in a ceDNA genome or ceDNA vector that is capable of mediating replication of ceDNA vector. It will be understood by one of ordinary skill in the art that in complex ceDNA vector configurations more than two ITRs or asymmetric ITR pairs may be present. The ITR can be an AAV ITR or a non-AAV ITR, or can be derived from an AAV ITR or a non-AAV ITR. For example, the ITR can be derived from the family Parvoviridae, which encompasses Parvoviruses and Dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies: Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

A “wild-type ITR” or “WT-ITR” refers to the sequence of a naturally occurring ITR sequence in an AAV or other dependovirus that retains, e.g., Rep binding activity and Rep nicking ability. The nucleic acid sequence of a WT-ITR from any AAV serotype may slightly vary from the canonical naturally occurring sequence due to degeneracy of the genetic code or drift, and therefore WT-ITR sequences encompassed for use herein include WT-ITR sequences as result of naturally occurring changes taking place during the production process (e.g., a replication error).

As used herein, the term “substantially symmetrical WT-ITRs” or a “substantially symmetrical WT-ITR pair” refers to a pair of WT-ITRs within a single ceDNA genome or ceDNA vector that are both wild type ITRs that have an inverse complement sequence across their entire length. For example, an ITR can be considered to be a wild-type sequence, even if it has one or more nucleotides that deviate from the canonical naturally occurring sequence, so long as the changes do not affect the properties and overall three-dimensional structure of the sequence. According to some aspects, the deviating nucleotides represent conservative sequence changes. As one non-limiting example, a sequence that has at least 95%, 96%, 97%, 98%, or 99% sequence identity to the canonical sequence (as measured, e.g., using BLAST at default settings), and also has a symmetrical three-dimensional spatial organization to the other WT-ITR such that their 3D structures are the same shape in geometrical space. The substantially symmetrical WT-ITR has the same A, C-C′ and B-B′ loops in 3D space. A substantially symmetrical WT-ITR can be functionally confirmed as WT by determining that it has an operable Rep binding site (RBE or RBE′) and terminal resolution site (trs) that pairs with the appropriate Rep protein. One can optionally test other functions, including transgene expression under permissive conditions.

As used herein, the phrases of “modified ITR” or “mod-ITR” or “mutant ITR” are used interchangeably herein and refer to an ITR that has a mutation in at least one or more nucleotides as compared to the WT-ITR from the same serotype. The mutation can result in a change According to some or more of A. C. C′, B, B′ regions in the ITR, and can result in a change in the three-dimensional spatial organization (i.e., its 3D structure in geometric space) as compared to the 3D spatial organization of a WT-ITR of the same serotype.

As used herein, the term “asymmetric ITRs” also referred to as “asymmetric ITR pairs” refers to a pair of ITRs within a single ceDNA genome or ceDNA vector that are not inverse complements across their full length. As one non-limiting example, an asymmetric ITR pair does not have a symmetrical three-dimensional spatial organization to their cognate ITR such that their 3D structures are different shapes in geometrical space. Stated differently, an asymmetrical ITR pair have the different overall geometric structure, i.e., they have different organization of their A, C-C′ and B-B′ loops in 3D space (e.g., one ITR may have a short C-C′ arm and/or short B-B′ arm as compared to the cognate ITR). The difference in sequence between the two ITRs may be due to one or more nucleotide addition, deletion, truncation, or point mutation. According to some embodiment, one ITR of the asymmetric ITR pair may be a wild-type AAV ITR sequence and the other ITR a modified ITR as defined herein (e.g., a non-wild-type or synthetic ITR sequence). In another embodiment, neither ITRs of the asymmetric ITR pair is a wild-type AAV sequence and the two ITRs are modified ITRs that have different shapes in geometrical space (i.e., a different overall geometric structure). According to some embodiments, one mod-ITRs of an asymmetric ITR pair can have a short C-C′ arm and the other ITR can have a different modification (e.g., a single arm, or a short B-B′ arm etc.) such that they have different three-dimensional spatial organization as compared to the cognate asymmetric mod-ITR.

As used herein, the term “symmetric ITRs” refers to a pair of ITRs within a single ceDNA genome or ceDNA vector that are mutated or modified relative to wild-type dependoviral ITR sequences and are inverse complements across their full length. Neither ITRs are wild type ITR AAV2 sequences (i.e., they are a modified ITR, also referred to as a mutant ITR), and can have a difference in sequence from the wild type ITR due to nucleotide addition, deletion, substitution, truncation, or point mutation. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

As used herein, the terms “substantially symmetrical modified-ITRs” or a “substantially symmetrical mod-ITR pair” refers to a pair of modified-ITRs within a single ceDNA genome or ceDNA vector that are both that have an inverse complement sequence across their entire length. For example, the modified ITR can be considered substantially symmetrical, even if it has some nucleotide sequences that deviate from the inverse complement sequence so long as the changes do not affect the properties and overall shape. As one non-limiting example, a sequence that has at least 85%, 90%. 91%, 92%, 93%, 94%, 95%, 96%, 97%, 98%, or 99% sequence identity to the canonical sequence (as measured using BLAST at default settings), and also has a symmetrical three-dimensional spatial organization to their cognate modified ITR such that their 3D structures are the same shape in geometrical space. Stated differently, a substantially symmetrical modified-ITR pair have the same A. C-C′ and B-B′ loops organized in 3D space. According to some embodiments, the ITRs from a mod-ITR pair may have different reverse complement nucleotide sequences but still have the same symmetrical three-dimensional spatial organization—that is both ITRs have mutations that result in the same overall 3D shape. For example, one ITR (e.g., 5′ ITR) in a mod-ITR pair can be from one serotype, and the other ITR (e.g., 3′ ITR) can be from a different serotype, however, both can have the same corresponding mutation (e.g., if the 5′ITR has a deletion in the C region, the cognate modified 3′ITR from a different serotype has a deletion at the corresponding position in the C′ region), such that the modified ITR pair has the same symmetrical three-dimensional spatial organization. In such embodiments, each ITR in a modified ITR pair can be from different serotypes (e.g., AAV1, 2, 3, 4, 5, 6, 7, 8, 9, 10, 11, and 12) such as the combination of AAV2 and AAV6, with the modification According to some ITR reflected in the corresponding position in the cognate ITR from a different serotype. According to some embodiment, a substantially symmetrical modified ITR pair refers to a pair of modified ITRs (mod-ITRs) so long as the difference in nucleotide sequences between the ITRs does not affect the properties or overall shape and they have substantially the same shape in 3D space. As a non-limiting example, a mod-ITR that has at least 90%, 91%, 92%, 93%, 94%, 95%, 96%, 97%, 98% or 99% sequence identity to the canonical mod-ITR as determined by standard means well known in the art such as BLAST (Basic Local Alignment Search Tool), or BLASTN at default settings, and also has a symmetrical three-dimensional spatial organization such that their 3D structure is the same shape in geometric space. A substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR.

The term “flanking” refers to a relative position of one nucleic acid sequence with respect to another nucleic acid sequence. Generally, in the sequence ABC, B is flanked by A and C. The same is true for the arrangement A×B×C. Thus, a flanking sequence precedes or follows a flanked sequence but need not be contiguous with, or immediately adjacent to the flanked sequence. According to some embodiment, the term flanking refers to terminal repeats at each end of the linear duplex ceDNA vector.

As used herein, the term “ceDNA genome” refers to an expression cassette that further incorporates at least one inverted terminal repeat region. A ceDNA genome may further comprise one or more spacer regions. According to some embodiments the ceDNA genome is incorporated as an intermolecular duplex polynucleotide of DNA into a plasmid or viral genome.

As used herein, the term “ceDNA spacer region” refers to an intervening sequence that separates functional elements in the ceDNA vector or ceDNA genome. According to some embodiments, ceDNA spacer regions keep two functional elements at a desired distance for optimal functionality. According to some embodiments, ceDNA spacer regions provide or add to the genetic stability of the ceDNA genome within e.g., a plasmid or baculovirus. According to some embodiments, ceDNA spacer regions facilitate ready genetic manipulation of the ceDNA genome by providing a convenient location for cloning sites and the like. For example, in certain aspects, an oligonucleotide “polylinker” containing several restriction endonuclease sites, or a non-open reading frame sequence designed to have no known protein (e.g., transcription factor) binding sites can be positioned in the ceDNA genome to separate the cis-acting factors, e.g., inserting a 6mer, 12mer, 18mer, 24mer, 48mer, 86mer, 176mer, etc. between the terminal resolution site and the upstream transcriptional regulatory element. Similarly, the spacer may be incorporated between the polyadenylation signal sequence and the 3′-terminal resolution site.

As used herein, the terms “Rep binding site, “Rep binding element, “RBE” and “RBS” are used interchangeably and refer to a binding site for Rep protein (e.g., AAV Rep 78 or AAV Rep 68) which upon binding by a Rep protein permits the Rep protein to perform its site-specific endonuclease activity on the sequence incorporating the RBS. An RBS sequence and its inverse complement together form a single RBS. RBS sequences are known in the art, and include, for example, 5′-GCGCGCTCGCTCGCTC-3′, an RBS sequence identified in AAV2. Any known RBS sequence may be used in the embodiments of the disclosure, including other known AAV RBS sequences and other naturally known or synthetic RBS sequences. Without being bound by theory it is thought that the nuclease domain of a Rep protein binds to the duplex nucleic acid sequence GCTC, and thus the two known AAV Rep proteins bind directly to and stably assemble on the duplex oligonucleotide, 5′-(GCGC)(GCTC)(GCTC)(GCTC)-3′. In addition, soluble aggregated conformers (i.e., undefined number of inter-associated Rep proteins) dissociate and bind to oligonucleotides that contain Rep binding sites. Each Rep protein interacts with both the nitrogenous bases and phosphodiester backbone on each strand. The interactions with the nitrogenous bases provide sequence specificity whereas the interactions with the phosphodiester backbone are non- or less-sequence specific and stabilize the protein-DNA complex.

As used herein, the terms “terminal resolution site” and “TRS” are used interchangeably herein and refer to a region at which Rep forms a tyrosine-phosphodiester bond with the 5′ thymidine generating a 3′ OH that serves as a substrate for DNA extension via a cellular DNA polymerase, e.g., DNA pol delta or DNA pol epsilon. Alternatively, the Rep-thymidine complex may participate in a coordinated ligation reaction. According to some embodiments, a TRS minimally encompasses a non-base-paired thymidine. According to some embodiments, the nicking efficiency of the TRS can be controlled at least in part by its distance within the same molecule from the RBS. When the acceptor substrate is the complementary ITR, then the resulting product is an intramolecular duplex. TRS sequences are known in the art, and include, for example, 5′-GGTTGA-3′, the hexanucleotide sequence identified in AAV2. Any known TRS sequence may be used in the embodiments of the disclosure, including other known AAV TRS sequences and other naturally known or synthetic TRS sequences such as AGTT, GGTTGG, AGTTGG, AGTTGA, and other motifs such as RRTTRR.

As used herein, the term “ceDNA” refers to capsid-free closed-ended linear double stranded (ds) duplex DNA for non-viral gene transfer, synthetic or otherwise. Detailed description of ceDNA is described in International application of PCT/US2017/020828, filed Mar. 3, 2017, the entire contents of which are expressly incorporated herein by reference. Certain methods for the production of ceDNA comprising various inverted terminal repeat (ITR) sequences and configurations using cell-based methods are described in Example 1 of International applications PCT/US18/49996, filed Sep. 7, 2018, and PCT/US2018/064242, filed Dec. 6, 2018 each of which is incorporated herein in its entirety by reference. Certain methods for the production of synthetic ceDNA vectors comprising various ITR sequences and configurations are described, e.g., in International application PCT/US2019/14122, filed Jan. 18, 2019, the entire content of which is incorporated herein by reference. As used herein, the terms “ceDNA vector” and “ceDNA” are used interchangeably and refer to a closed-ended DNA vector comprising at least one terminal palindrome. According to some embodiments, the ceDNA comprises two covalently-closed ends.

As used herein, the term “ceDNA-plasmid” refers to a plasmid that comprises a ceDNA genome as an intermolecular duplex.

As used herein, the term “ceDNA-bacmid” refers to an infectious baculovirus genome comprising a ceDNA genome as an intermolecular duplex that is capable of propagating in E. coli as a plasmid, and so can operate as a shuttle vector for baculovirus.

As used herein, the term “ceDNA-baculovirus” refers to a baculovirus that comprises a ceDNA genome as an intermolecular duplex within the baculovirus genome.

As used herein, the terms “ceDNA-baculovirus infected insect cell” and “ceDNA-BIIC” are used interchangeably, and refer to an invertebrate host cell (including, but not limited to an insect cell (e.g., an Sf9 cell)) infected with a ceDNA-baculovirus.

As used herein, the term “closed-ended DNA vector” refers to a capsid-free DNA vector with at least one covalently closed end and where at least part of the vector has an intramolecular duplex structure.

As defined herein, “reporters” refer to proteins that can be used to provide detectable read-outs. Reporters generally produce a measurable signal such as fluorescence, color, or luminescence. Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase, and others well known in the art.

As used herein, the term “effector protein” refers to a polypeptide that provides a detectable read-out, either as, for example, a reporter polypeptide.

Transcriptional regulators refer to transcriptional activators and repressors that either activate or repress transcription of a transgene (e.g., a nucleic acid encoding an antibody or antigen-binding fragment thereof as described herein). Promoters are regions of nucleic acid that initiate transcription of a particular gene. Transcriptional activators typically bind nearby to transcriptional promoters and recruit RNA polymerase to directly initiate transcription. Repressors bind to transcriptional promoters and sterically hinder transcriptional initiation by RNA polymerase. Other transcriptional regulators may serve as either an activator or a repressor depending on where they bind and cellular and environmental conditions. Non-limiting examples of transcriptional regulator classes include, but are not limited to homeodomain proteins, zinc-finger proteins, winged-helix (forkhead) proteins, and leucine-zipper proteins.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

The term “in vivo” refers to assays or processes that occur in or within an organism, such as a multicellular animal. According to some of the aspects described herein, a method or use can be said to occur “in vivo” when a unicellular organism, such as a bacterium, is used. The term “ex vivo” refers to methods and uses that are performed using a living cell with an intact membrane that is outside of the body of a multicellular animal or plant, e.g., explants, cultured cells, including primary cells and cell lines, transformed cell lines, and extracted tissue or cells, including blood cells, among others. The term “in vitro” refers to assays and methods that do not require the presence of a cell with an intact membrane, such as cellular extracts, and can refer to the introducing of a programmable synthetic biological circuit in a non-cellular system, such as a medium not comprising cells or cellular systems, such as cellular extracts.

The term “promoter.” as used herein, refers to any nucleic acid sequence that regulates the expression of another nucleic acid sequence by driving transcription of the nucleic acid sequence, which can be a heterologous target gene encoding a protein or an RNA. Promoters can be constitutive, inducible, repressible, tissue-specific, or any combination thereof. A promoter is a control region of a nucleic acid sequence at which initiation and rate of transcription of the remainder of a nucleic acid sequence are controlled. A promoter can also contain genetic elements at which regulatory proteins and molecules can bind, such as RNA polymerase and other transcription factors. According to some embodiments of the aspects described herein, a promoter can drive the expression of a transcription factor that regulates the expression of the promoter itself. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the expression of transgenes in the ceDNA vectors disclosed herein. A promoter sequence may be bounded at its 3′ terminus by the transcription initiation site and extends upstream (5′ direction) to include the minimum number of bases or elements necessary to initiate transcription at levels detectable above background.

The term “enhancer” as used herein refers to a cis-acting regulatory sequence (e.g., 10-1,500 base pairs) that binds one or more proteins (e.g., activator proteins, or transcription factor) to increase transcriptional activation of a nucleic acid sequence. Enhancers can be positioned up to 1,000,000 base pars upstream of the gene start site or downstream of the gene start site that they regulate. An enhancer can be positioned within an intronic region, or in the exonic region of an unrelated gene.

A promoter can be said to drive expression or drive transcription of the nucleic acid sequence that it regulates. The phrases “operably linked.” “operatively positioned.” “operatively linked,” “under control.” and “under transcriptional control” indicate that a promoter is in a correct functional location and/or orientation in relation to a nucleic acid sequence it regulates to control transcriptional initiation and/or expression of that sequence. An “inverted promoter.” as used herein, refers to a promoter in which the nucleic acid sequence is in the reverse orientation, such that what was the coding strand is now the non-coding strand, and vice versa. Inverted promoter sequences can be used in various embodiments to regulate the state of a switch. In addition, in various embodiments, a promoter can be used in conjunction with an enhancer.

A promoter can be one naturally associated with a gene or sequence, as can be obtained by isolating the 5′ non-coding sequences located upstream of the coding segment and/or exon of a given gene or sequence. Such a promoter can be referred to as “endogenous.” Similarly, according to some embodiments, an enhancer can be one naturally associated with a nucleic acid sequence, located either downstream or upstream of that sequence.

According to some embodiments, a coding nucleic acid segment is positioned under the control of a “recombinant promoter” or “heterologous promoter.” both of which refer to a promoter that is not normally associated with the encoded nucleic acid sequence it is operably linked to in its natural environment. A recombinant or heterologous enhancer refers to an enhancer not normally associated with a given nucleic acid sequence in its natural environment. Such promoters or enhancers can include promoters or enhancers of other genes; promoters or enhancers isolated from any other prokaryotic, viral, or eukaryotic cell; and synthetic promoters or enhancers that are not “naturally occurring.” i.e., comprise different elements of different transcriptional regulatory regions, and/or mutations that alter expression through methods of genetic engineering that are known in the art. In addition to producing nucleic acid sequences of promoters and enhancers synthetically, promoter sequences can be produced using recombinant cloning and/or nucleic acid amplification technology, including PCR, in connection with the synthetic biological circuits and modules disclosed herein (see, e.g., U.S. Pat. Nos. 4,683,202, 5,928,906, each incorporated herein by reference). Furthermore, it is contemplated that control sequences that direct transcription and/or expression of sequences within non-nuclear organelles such as mitochondria, chloroplasts, and the like, can be employed as well.

As described herein, an “inducible promoter” is one that is characterized by initiating or enhancing transcriptional activity when in the presence of, influenced by, or contacted by an inducer or inducing agent. An “inducer” or “inducing agent.” as defined herein, can be endogenous, or a normally exogenous compound or protein that is administered in such a way as to be active in inducing transcriptional activity from the inducible promoter. According to some embodiments, the inducer or inducing agent, i.e., a chemical, a compound or a protein, can itself be the result of transcription or expression of a nucleic acid sequence (i.e., an inducer can be an inducer protein expressed by another component or module), which itself can be under the control or an inducible promoter. According to some embodiments, an inducible promoter is induced in the absence of certain agents, such as a repressor. Examples of inducible promoters include but are not limited to, tetracycline, metallothionine, ecdysone, mammalian viruses (e.g., the adenovirus late promoter; and the mouse mammary tumor virus long terminal repeat (MMTV-LTR)) and other steroid-responsive promoters, rapamycin responsive promoters and the like.

The term “open reading frame (ORF)” as used herein is meant to refer to a sequence of several nucleotide triplets which may be translated into a peptide or protein. An open reading frame preferably contains a start codon, i.e. a combination of three subsequent nucleotides coding usually for the amino acid methionine (ATG), at its 5′-end and a subsequent region which usually exhibits a length which is a multiple of 3 nucleotides. An ORF is preferably terminated by a stop-codon (e.g., TAA, TAG, TGA). Typically, this is the only stop-codon of the open reading frame. Thus, an open reading frame in the context of the present disclosure is preferably a nucleotide sequence, consisting of a number of nucleotides that may be divided by three, which starts with a start codon (e.g., ATG) and which preferably terminates with a stop codon (e.g., TAA, TGA, or TAG). The open reading frame may be isolated or it may be incorporated in a longer nucleic acid sequence, for example in a ceDNA vector as described herein.

“Operably linked” refers to a juxtaposition wherein the components so described are in a relationship permitting them to function in their intended manner. For instance, a promoter is operably linked to a coding sequence if the promoter affects its transcription or expression. An “expression cassette” includes a DNA sequence that is operably linked to a promoter or other regulatory sequence sufficient to direct transcription of the transgene in the ceDNA vector. Suitable promoters include, for example, tissue specific promoters. Promoters can also be of AAV origin.

The term “subject” as used herein refers to a human or animal, to whom treatment, including prophylactic treatment, with the ceDNA vector according to the present disclosure, is provided. As used herein, the term “subject” includes humans and other animals. Typically, the subject is a human. For example, the subject may be an adult, a teenager, a child (2 years to 14 years of age), an infant (birth to 2 year), or a neonate (up to 2 months). In particular aspects, the subject is up to 4 months old, or up to 6 months old. According to some aspects, the adults are seniors about 65 years or older, or about 60 years or older. According to some aspects, the subject is a pregnant woman or a woman intending to become pregnant. In other aspects, subject is not a human; for example, a non-human primate such as a baboon, a chimpanzee, a gorilla, or a macaque. In certain aspects, the subject may be a pet, such as a dog or a cat.

As used herein, the term “host cell”, includes any cell type that is susceptible to transformation, transfection, transduction, and the like with a nucleic acid construct or ceDNA expression vector of the present disclosure. A host cell can be an in situ or in vivo cell in a tissue, organ or organism.

The term “exogenous” refers to a substance present in a cell other than its native source. The term “exogenous” when used herein can refer to a nucleic acid (e.g., a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell.

The term “sequence identity” refers to the relatedness between two nucleotide sequences. For purposes of the present disclosure, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows: (Identical Deoxyribonucleotides.times. 100)/(Length of Alignment-Total Number of Gaps in Alignment). The length of the alignment is preferably at least 10 nucleotides, preferably at least 25 nucleotides more preferred at least 50 nucleotides and most preferred at least 100 nucleotides.

The term “homology” or “homologous” as used herein is defined as the percentage of nucleotide residues that are identical to the nucleotide residues in the corresponding sequence on the target chromosome, after aligning the sequences and introducing gaps, if necessary, to achieve the maximum percent sequence identity. Alignment for purposes of determining percent nucleotide sequence homology can be achieved in various ways that are within the skill in the art, for instance, using publicly available computer software such as BLAST, BLAST-2, ALIGN, ClustalW2 or Megalign (DNASTAR) software. Those skilled in the art can determine appropriate parameters for aligning sequences, including any algorithms needed to achieve maximal alignment over the full length of the sequences being compared. According to some embodiments, a nucleic acid sequence (e.g., DNA sequence), for example of a homology arm, is considered “homologous” when the sequence is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to the corresponding native or unedited nucleic acid sequence (e.g., genomic sequence) of the host cell.

The term “heterologous.” as used herein, means a nucleotide or polypeptide sequence that is not found in the native nucleic acid or protein, respectively. A heterologous nucleic acid sequence may be linked to a naturally-occurring nucleic acid sequence (or a variant thereof) (e.g., by genetic engineering) to generate a chimeric nucleotide sequence encoding a chimeric polypeptide. A heterologous nucleic acid sequence may be linked to a variant polypeptide (e.g., by genetic engineering) to generate a nucleic acid sequence encoding a fusion variant polypeptide. Alternatively, the term “heterologous” may refer to a nucleic acid sequence which is not naturally present in a cell or subject.

A “vector” or “expression vector” is a replicon, such as plasmid, bacmid, phage, virus, virion, or cosmid, to which another DNA segment, i.e., an “insert”, may be attached so as to bring about the replication of the attached segment in a cell. A vector can be a nucleic acid construct designed for delivery to a host cell or for transfer between different host cells. As used herein, a vector can be viral or non-viral in origin and/or in final form, however for the purpose of the present disclosure, a “vector” generally refers to a ceDNA vector, as that term is used herein. The term “vector” encompasses any genetic element that is capable of replication when associated with the proper control elements and that can transfer gene sequences to cells. According to some embodiments, a vector can be an expression vector or recombinant vector.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

By “recombinant vector” is meant a vector that includes a heterologous nucleic acid sequence, or “transgene” that is capable of expression in vivo. It should be understood that the vectors described herein can, according to some embodiments, be combined with other suitable compositions and therapies. According to some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the terms, “administration,” “administering” and variants thereof refers to introducing a composition or agent (e.g., a ceDNA as described herein) into a subject and includes concurrent and sequential introduction of one or more compositions or agents. “Administration” can refer, e.g., to therapeutic, pharmacokinetic, diagnostic, research, placebo, and experimental methods. “Administration” also encompasses in vitro and ex vivo treatments. The introduction of a composition or agent into a subject is by any suitable route, including orally, pulmonarily, intranasally, parenterally (intravenously, intramuscularly, intraperitoneally, or subcutaneously), rectally, intralymphatically, intratumorally, or topically. Administration includes self-administration and the administration by another. Administration can be carried out by any suitable route. A suitable route of administration allows the composition or the agent to perform its intended function. For example, if a suitable route is intravenous, the composition is administered by introducing the composition or agent into a vein of the subject.

The term “infection” as used herein refers to the initial entry of a pathogen into a host; and the condition in which the pathogen has become established in or on cells or tissues of a host; such a condition does not necessarily constitute or lead to a disease.

The term “immune response” as used herein is meant to refer to any functional expression of a subject's immune system, against either foreign or self-antigens, whether the consequences of these reactions are beneficial or harmful to the subject.

The term “dose” as used herein refers to the quantity of a substance (e.g., a ceDNA as described herein) to be taken or administered to the subject at one time.

The term “dosing”, as used herein, refers to the administration of a substance (e.g., a ceDNA as described herein) to achieve a therapeutic objective (e.g., treatment).

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present disclosure, therefore, includes methods of combination therapeutic treatment and combination pharmaceutical compositions.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may to administer to the subject a second agent, and the administering steps may be executed at the same time, or nearly the same time, or at distant times, so long as the first agent (and additional agents) are after administration in the presence of the second agent (and additional agents). The actor and the subject may be the same entity (e.g., human).

The term “combination therapy”, as used herein, refers to the administration of two or more therapeutic substances, e.g., an antibody, or antigen-binding fragment as described herein, and another drug. The other drug(s) may be administered concomitant with, prior to, or following the administration of the antibody, or antigen-binding fragment as described herein.

As used herein, the phrases “nucleic acid therapeutic”, “therapeutic nucleic acid” and “TNA” are used interchangeably and refer to any modality of therapeutic using nucleic acids as an active component of therapeutic agent to treat a disease or disorder. As used herein, these phrases refer to RNA-based therapeutics and DNA-based therapeutics. Non-limiting examples of RNA-based therapeutics include mRNA, antisense RNA and oligonucleotides, ribozymes, aptamers, interfering RNAs (RNAi), Dicer-substrate dsRNA, small hairpin RNA (shRNA), asymmetrical interfering RNA (aiRNA), microRNA (miRNA). Non-limiting examples of DNA-based therapeutics include minicircle DNA, minigene, viral DNA (e.g., Lentiviral or AAV genome) or non-viral synthetic DNA vectors, closed-ended linear duplex DNA (ceDNA/CELiD), plasmids, bacmids, Doggybone™ DNA vectors, minimalistic immunological-defined gene expression (MIDGE)-vector, nonviral ministring DNA vector (linear-covalently closed DNA vector), or dumbbell-shaped DNA minimal vector (“dumbbell DNA”). According to some embodiments, the therapeutic nucleic acid is a ceDNA.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation. For any therapeutic agent described herein therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to therapeutic window, additional guidance for dosage modification can be obtained.

As used herein, “viral infection” is meant to refer to the invasion and multiplication of a virus in the body of a subject.

As used herein, the terms “treat.” “treating.” and/or “treatment” include abrogating, substantially inhibiting, slowing or reversing the progression of a condition, substantially ameliorating clinical symptoms of a condition, or substantially preventing the appearance of clinical symptoms of a condition, obtaining beneficial or desired clinical results. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

Those “in need of treatment” include mammals, such as humans, already having a Coronavirus infection, e.g., a SARS-CoV-2 infection, including those in which the disease or disorder caused by the Coronavirus infection (e.g., COVID-19) is to be prevented, or those in which progression of the disease or disorder caused by the Coronavirus infection (e.g., COVID-19) is to be prevented.

As used herein, the term “increase,” “enhance.” “raise” (and like terms) generally refers to the act of increasing, either directly or indirectly, a concentration, level, function, activity, or behavior relative to the natural, expected, or average, or relative to a control condition.

As used herein, the term “suppress,” “decrease.” “interfere,” “inhibit” and/or “reduce” (and like terms) generally refers to the act of reducing, either directly or indirectly, a concentration, level, function, activity, or behavior relative to the natural, expected, or average, or relative to a control condition.

As used herein, a “control” is meant to refer to a reference standard. According to some embodiments, the control is a negative control sample obtained from a healthy patient. In other embodiments, the control is a positive control sample obtained from a patient diagnosed with COVID-19. In still other embodiments, the control is a historical control or standard reference value or range of values (such as a previously tested control sample, such as a group of COVID-19 patients with known prognosis or outcome, or group of samples that represent baseline or normal values). A difference between a test sample and a control can be an increase or conversely a decrease. The difference can be a qualitative difference or a quantitative difference, for example a statistically significant difference. According to some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The use of “comprising” indicates inclusion rather than limitation.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus, for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other than in the operating examples, or where otherwise indicated, all numbers expressing quantities of ingredients or reaction conditions used herein should be understood as modified in all instances by the term “about.” The term “about” when used in connection with percentages can mean±1%. The present disclosure is further explained in detail by the following examples, but the scope of the disclosure should not be limited thereto.

Groupings of alternative elements or embodiments of the disclosure disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Other terms are defined herein within the description of the various aspects of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It should be understood that this disclosure is not limited to the particular methodology, protocols, and reagents, etc., described herein and as such can vary. The terminology used herein is for the purpose of describing particular embodiments only, and is not intended to limit the scope of the present disclosure, which is defined solely by the claims.

### II. Expression of Antibody or Antigen-Binding Protein from a ceDNA Vector

The technology described herein is directed in general to the expression and/or production of an antibody, or an antigen-binding fragment thereof, in a cell from one or more non-viral DNA vectors, e.g., ceDNA vectors as described herein. ceDNA vectors for expression of an antibody, or an antigen-binding fragment thereof, are described herein in the section entitled “ceDNA vectors in general”. In particular, ceDNA vectors for expression of an antibody, or an antigen binding portion thereof, comprise a pair of ITRs (e.g., symmetric or assymetric as described herein) and between the ITR pair, a nucleic acid encoding an antibody heavy chain (HC) and/or an antibody light chain (LC), or a portion thereof, as described herein, operatively linked to a promoter or regulatory sequence. A distinct advantage of ceDNA vectors for expression of an antibody, or an antigen-binding fragment thereof, over traditional AAV vectors, and even lentiviral vectors, is that there is no size constraint for the nucleic acid sequences encoding the desired HC or LC. It is a feature of the present disclosure that in some embodiments, the HC and LC may be expressed from different ceDNAs.

As one will appreciate, the ceDNA vector technologies described herein can be adapted to any level of complexity or can be used in a modular fashion, where expression of different components of an antibody (e.g., a HC, a LC) can be controlled in an independent manner. For example, it is contemplated that the ceDNA vector technologies designed herein use multiple ceDNA vectors, where each vector expresses a HC or a LC, or portions thereof, or another component, that are each independently controlled by the same or different promoters. The following embodiments are specifically contemplated herein and can adapted by one of skill in the art as desired.

According to some embodiment, a single ceDNA vector can be used to express a LC of an anti-SARS-CoV-2-S antibody, or antigen-binding fragment thereof under the control of a first promoter. According to some embodiment, a single ceDNA vector can be used to express a HC of an anti-SARS-CoV-2-S antibody, or antigen-binding fragment thereof under the control of a second promoter. According to some embodiments, the first promoter and the second promoter are the same. According to some embodiments, the first promoter and the second promoter are different.

It is a finding of the present disclosure that, it is often desirable to express components of an antibody (e.g., the HC or LC) at different expression levels, thus controlling the stoichiometry of the individual components expressed to ensure efficient combination in the cell, as described in more detail hereinbelow.

### A. SARS CoV-2

Provided herein are compositions and methods comprising one or more ceDNA vectors for expression of an antibody, or an antigen-binding fragment thereof, for the treatment or prevention of a coronavirus infection in a subject. As described herein, the coronavirus infection is SARS-CoV-2 (severe acute respiratory syndrome coronavirus 2). According to some embodiments, the antibodies or antigen-binding fragments provided herein can bind to and/or neutralize SARS-CoV-2.

Coronavirus virions are spherical with diameters of approximately 125 nm. The most prominent feature of coronaviruses is the club-shape spike projections emanating from the surface of the virion. These spikes are a defining feature of the virion and give them the appearance of a solar corona, prompting the name, coronaviruses. Within the envelope of the virion is the nucleocapsid. Coronaviruses have helically symmetrical nucleocapsids, which is uncommon among positive-sense RNA viruses, but far more common for negative-sense RNA viruses. SARS-CoV-2 and SARS-CoV belong to the coronavirus family. The spike protein contains an S1 subunit that facilitates binding of the coronavirus to cell surface proteins. Accordingly, the S1 subunit of the spike protein controls which cells are infected by the coronavirus. The spike protein also contains a S2 subunit, which is a transmembrane subunit that facilitates viral and cellular membrane fusion.

The complete genome of severe acute respiratory syndrome coronavirus 2 isolate Wuhan-Hu-1 is set forth as GenBank Accession No. MN908947.3. SARS-Cov-2 comprises 29903 base pairs of single stranded RNA. Structural proteins create the outer envelope of the virus, including a surface glycoprotein (S) that is used for infecting cells, a nucleocapsid (N), and an envelope protein (E).

The amino acid sequence of the wild type surface glycoprotein (S), is set forth below as SEQ ID NO: 28

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGENCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

Upon binding cell surface proteins and membrane fusion, the coronavirus enters the cell and its singled-stranded RNA genome is released into the cytoplasm of the infected cell. The single-stranded RNA genome is a positive strand and thus, can be translated into an RNA polymerase, which produces additional viral RNAs that are minus strands. The viral minus RNA strands are transcribed into smaller, subgenomic positive RNA strands, which are used to translate other viral proteins, for example, nucleocapsid (N) protein, envelope (E) protein, and matrix (M) protein.

### B. Antibodies, and Antigen-Binding Fragments Thereof

According to some aspects, the present disclosure provides one or more ceDNA vectors comprising one or more nucleic acid sequences that encode an anti-SARS-CoV-2 S antibody, or antigen-binding fragment thereof, capable of binding to coronavirus surface glycoprotein (S) protein. According to some embodiments, the antibodies are anti-SARS-CoV-2 antibodies, or antigen-binding fragments thereof, that are capable of binding to a SARS-CoV-2 surface glycoprotein (S) expressed on a cell surface of a host cell and/or on a SARS-CoV-2 virion.

According to some embodiments, the ceDNA vector comprises variable heavy chain (VH) comprising the amino acid sequence set forth below:

(SEQ ID NO: 5)
QVQLVQSGAEVKKPGASVKVSCKASGYPFTSYGISWVRQAPGQGLEWMGW
ISTYQGNTNYAQKFQGRVTMTTDTSTTTGYMELRRLRSDDTAVYYCARDY
TRGAWFGESLIGGFDNWGQGTLVTVSS

According to some embodiments, the heavy chain variable domain of the antibody is at least 90%, 91%, 92%, 93%, 94%, 95%, 96%, 97%, 98%, or 99% identical to the VH sequence set forth in SEQ ID NO:5.

According to some embodiments, the ceDNA vector comprises a variable light chain (VL) comprising the amino acid sequence set forth below:

(SEQ ID NO.: 6)
EIVLTQSPGTLSLSPGERATLSCRASQTVSSTSLAWYQQKPGQAPRLLIY
GASSRATGIPDRFSGSGSGTDFTLTISRLEPEDFAVYYCQQHDTSLTFGG
GTKVEIK

According to some embodiments, the heavy chain variable domain of the antibody is at least 90%, 91%, 92%, 93%, 94%, 95%, 96%, 97%, 98%, or 99% identical to the VH sequence set forth in SEQ ID NO:6.

According to some embodiments, the one or more ceDNA vectors comprise an anti-SARS-CoV-2 antibody, or an antigen-binding fragment thereof, comprising a heavy chain variable domain and a light chain variable domain, wherein, the heavy chain variable domain is at least 90% identical (e.g., at least 90%, 91%, 92%, 93%, 94%, 95%, 96%, 97%, 98% 99% or 100% identical) to SEQ ID NO:5; and, wherein the light chain variable domain is at least 90% identical (e.g., at least 90%, 91%, 92%, 93%, 94%, 95%, 96%, 97%, 98% 99% or 100% identical) to SEQ ID NO:6.

Typically, the variable domains of both the heavy and light immunoglobulin chains comprise three hypervariable regions, also called complementarity determining regions (CDRs), located within relatively conserved framework regions (FR). In general, from N-terminal to C-terminal, both light chain and heavy chain variable domains comprise FR1, CDR1, FR2, CDR2, FR3, CDR3 and FR4.

Each set of the H-CDRs (CDRH1, CDRH2 and CDRH3) listed in Table 1 can be combined with the L-CDRs (CDRL1, CDRL2 and CDRL3) provided in Table 1.

TABLE 1

| | |
|---|---|
| CDRH1 | GYPFTSYG (SEQ ID NO.: 7) |
| CDRH2 | ISTYQGNT (SEQ ID NO.: 8) |
| CDRH3 | ARDYTRGAWFGESLIGGFDN (SEQ ID NO.: 9) |
| CDRL1 | QTVSSTS (SEQ ID NO.: 10) |
| CDRL2 | GAS (SEQ ID NO.: 11) |
| CDRL3 | QQHDTSLT (SEQ ID NO.: 12) |

According to some embodiments, the disclosure provides one or more ceDNA vectors comprising a nucleic acid sequence that encodes an anti-SARS-CoV-2 S antibody comprising six CDRs (e.g., a CDRH1, a CDRH2, a CDRH3, a CDRL1, a CDRL2, and a CDRL3), wherein CDRH1 comprises SEQ ID NO:7, CDRH2 comprises SEQ ID NO:8, CDRH3 comprises SEQ ID NO:9, CDRL1 comprises SEQ ID NO:10, CDRL2 comprises SEQ ID NO:11, and CDRL3 comprises SEQ ID NO:12.

According to some embodiments, any of the antibodies, or antigen-binding fragment thereof, of the disclosure include any antibody (including antigen binding fragments thereof) having one or more CDR (e.g., CDRH or CDRL) sequences identical to, or substantially similar to CDRH1, CDRH2, CDRH3, CDRL1, CDRL2, and/or CDRL3 set forth in Table 1 above. For example, the antibodies may include one or more CDR sequences as shown in Table 1 containing up to 5, 4, 3, 2, or 1 amino acid residue variations as compared to the corresponding CDR region in any one of SEQ ID NOs: 7, 8, 9, 10, 11 and 12.

According to some embodiments, the disclosure provides one or more ceDNA vectors comprising one or more nucleic acid sequences that encode an anti-SARS-CoV-2 S antibody heavy chain variable domain (VH) comprising a CDRH1, a CDRH2, and a CDRH3, and a light chain variable domain (VL) comprising a CDRL1, a CDRL2, and a CDRL3, and is capable of binding to a surface glycoprotein (S) of SARS-CoV-2. In certain embodiments, the antibody or the antigen-binding fragment is capable of binding to a SARS-CoV-2 surface glycoprotein (S) expressed on a cell surface of a host cell and/or on a SARS-CoV-2 virion.

### AB1

According to some embodiments, the disclosure provides one or more ceDNA vectors comprising one or more nucleic acid sequences that encode an anti-SARS-CoV-2 S antibody heavy chain (HC) comprising SEQ ID NO:1 set forth below:

(SEQ ID NO: 1)
QVQLVQSGAEVKKPGASVKVSCKASGYPFTSYGISWVRQAPGQGLEWMGW
ISTYQGNTNYAQKFQGRVTMTTDTSTTTGYMELRRLRSDDTAVYYCARDY
TRGAWFGESLIGGFDNWGQGTLVTVSSASTKGPSVFPLAPSSKSTSGGTA
ALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENN
YKTTPPVLDSDGSFFLYSKLTVDKSRWQQGNVFSCSVLHEALHSHYTQKS
LSLSPGK

According to some embodiments, the disclosure provides one or more ceDNA vectors comprising one or more nucleic acid sequences that encode an anti-SARS-CoV-2 S antibody light chain (LC) comprising SEQ ID NO:2 set forth below:

(SEQ ID NO: 2)
EIVLTQSPGTLSLSPGERATLSCRASQTVSSTSLAWYQQKPGQAPRLLIY
GASSRATGIPDRESGSGSGTDFTLTISRLEPEDFAVYYCQQHDTSLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

According to some embodiments, the anti-SARS-CoV-2 S antibody, or the antigen-binding fragment thereof, comprises a heavy chain and a light chain, wherein, the heavy chain is at least 90% identical (e.g., at least 90%, 91%, 92%, 93%, 94%, 95%, 96%, 97%, 98% 99% or 100% identical) to SEQ ID NO: 1; and, wherein the light chain is at least 90% identical (e.g., at least 90%, 91%, 92%, 93%, 94%, 95%, 96%, 97%, 98% 99% or 100% identical) to SEQ ID NO:2.

According to some embodiments, AB1 comprises a heavy chain variable domain and a light chain variable domain, wherein, the heavy chain variable domain is at least 90% identical (e.g., at least 90%, 91%, 92%, 93%, 94%, 95%, 96%, 97%, 98% 99% or 100% identical) to SEQ ID NO:5; and, wherein the light chain variable domain is at least 90% identical (e.g., at least 90%, 91%, 92%, 93%, 94%, 95%, 96%, 97%, 98% 99% or 100% identical) to SEQ ID NO:6.

According to some embodiments, AB1 comprises the CDR sequences set forth in Table 1 above.

According to some embodiments, sequences corresponding to CL and CH1-CH3 of AB1 are shown below in Table 2A.

TABLE 2A

| Description | SEQ ID NO: | Sequence |
|---|---|---|
| SARS-COV-2 | 13 | RTVAAPSVFIFPPSDEQLKSGTASVVCLLNNFYPREAKVQWKV |
| mAb CL (Ck) | | DNALQSGNSQESVTEQDSKDSTYSLSSTLTLSKADYEKHKVYA |
| IgKC*01 k1m3 | | CEVTHQGLSSPVTKSFNRGEC |
| (aa) | | |
| SARS-COV-2 | 14 | ASTKGPSVFPLAPSSKSTSGGTAALGCLVKDYFPEPVTVSWNSG |
| CH1 CH3 | | ALTSGVHTFPAVLQSSGLYSLSSVVTVPSSSLGTQTYICNVNHK |
| G1m17; | | PSNTKVDKKVEPKSCDKTHTCPPCPAPELLGGPSVFLFPPKPKD |
| IgG1*01 LS | | TLMISRTPEVTCVVVDVSHEDPEVKFNWYVDGVEVHNAKTKP |
| (aa) | | REEQYNSTYRVVSVLTVLHQDWLNGKEYKCKVSNKALPAPIE |
| | | KTISKAKGQPREPQVYTLPPSRDELTKNQVSLTCLVKGFYPSDI |
| | | AVEWESNGQPENNYKTTPPVLDSDGSFFLYSKLTVDKSRWQQ |
| | | GNVFSCSVLHEALHSHYTQKSLSLSPGK |
| SARS-COV-2 | 15 | ASTKGPSVFPLAPSSKSTSGGTAALGCLVKDYFPEPVTVSWNSG |
| CH1 CH3 | | ALTSGVHTFPAVLQSSGLYSLSSVVTVPSSSLGTQTYICNVNHK |
| G1m17; | | PSNTKVDKKVEPKSCDKTHTCPPCPAPELLGGPSVFLFPPKPKD |
| IgG1*01 LS no | | TLMISRTPEVTCVVVDVSHEDPEVKFNWYVDGVEVHNAKTKP |
| C-term Lys (aa) | | REEQYNSTYRVVSVLTVLHQDWLNGKEYKCKVSNKALPAPIE |
| | | KTISKAKGQPREPQVYTLPPSRDELTKNQVSLTCLVKGFYPSDI |
| | | AVEWESNGQPENNYKTTPPVLDSDGSFFLYSKLTVDKSRWQQ |
| | | GNVFSCSVLHEALHSHYTQKSLSLSPG |

### AB2

According to some embodiments, the disclosure provides one or more ceDNA vectors comprising one or more nucleic acid sequences that encode an anti-SARS-CoV-2 S antibody heavy chain (HC) comprising SEQ ID NO:3 set forth below:

(SEQ ID NO: 3)
QVQLVQSGAEVKKPGASVKVSCKASGYPFTSYGISWVRQAPGQGLEWMGW
ISTYQGNTNYAQKFQGRVTMTTDTSTTTGYMELRRLRSDDTAVYYCARDY
TRGAWFGESLIGGFDNWGQGTLVTVSSASTKGPSVFPLAPSSKSTSGGTA
ALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLAGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPLPEEKTISKAK
GQPREPQVYTLPPSRDELTKNQVSLTCLVKGFYPSDIAVEWESNGQPENN
YKTTPPVLDSDGSFFLYSKLTVDKSRWQQGNVFSCSVLHEALHSHYTQKS
LSLSPGK

According to some embodiments, the disclosure provides one or more ceDNA vectors comprising one or more nucleic acid sequences that encode an anti-SARS-CoV-2 S antibody light chain (LC) comprising SEQ ID NO:4 set forth below:

(SEQ ID NO: 4)
EIVLTQSPGTLSLSPGERATLSCRASQTVSSTSLAWYQQKPGQAPRLLIY
GASSRATGIPDRFSGSGSGTDFTLTISRLEPEDFAVYYCQQHDTSLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

According to some embodiments, the anti-SARS-CoV-2 S antibody, or the antigen-binding fragment thereof, comprises a heavy chain and a light chain, wherein, the heavy chain is at least 90% identical (e.g., at least 90%, 91%, 92%, 93%, 94%, 95%, 96%, 97%, 98% 99% or 100% identical) to SEQ ID NO: 3; and, wherein the light chain is at least 90% identical (e.g., at least 90%, 91%, 92%, 93%, 94%, 95%, 96%, 97%, 98% 99% or 100% identical) to SEQ ID NO: 4.

According to some embodiments, AB2 comprises a heavy chain variable domain and a light chain variable domain, wherein, the heavy chain variable domain is at least 90% identical (e.g., at least 90%, 91%, 92%, 93%, 94%, 95%, 96%, 97%, 98% 99% or 100% identical) to SEQ ID NO:5; and, wherein the light chain variable domain is at least 90% identical (e.g., at least 90%, 91%, 92%, 93%, 94%, 95%, 96%, 97%, 98% 99% or 100% identical) to SEQ ID NO:6.

According to some embodiments, AB2 comprises the CDR sequences set forth in Table 1 above.

According to some embodiments, the CL and CH1-CH3 sequences and sequence identifiers of AB2 are shown below in Table 2B.

TABLE 2B

| Description | SEQ ID NO: | Sequence |
|---|---|---|
| SARS-COV-2 | 13 | RTVAAPSVFIFPPSDEQLKSGTASVVCLLNNFYPREAKVQWKVD |
| mAb CL (Ck) | | NALQSGNSQESVTEQDSKDSTYSLSSTLTLSKADYEKHKVYACE |
| IgKC*01 k1m3 | | VTHQGLSSPVTKSFNRGEC |
| (aa) | | |
| SARS-COV-2 | 17 | ASTKGPSVFPLAPSSKSTSGGTAALGCLVKDYFPEPVTVSWNSGA |
| CH1 CH3 | | LTSGVHTFPAVLQSSGLYSLSSVVTVPSSSLGTQTYICNVNHKPSN |
| G1m17; | | TKVDKKVEPKSCDKTHTCPPCPAPELLAGPSVFLFPPKPKDTLMIS |
| IgG1*01 LS | | RTPEVTCVVVDVSHEDPEVKFNWYVDGVEVHNAKTKPREEQYN |
| GAALIE (aa) | | STYRVVSVLTVLHQDWLNGKEYKCKVSNKALPLPEEKTISKAKG |
| | | QPREPQVYTLPPSRDELTKNQVSLTCLVKGFYPSDIAVEWESNGQ |
| | | PENNYKTTPPVLDSDGSFFLYSKLTVDKSRWQQGNVFSCSVLHE |
| | | ALHSHYTQKSLSLSPGK |
| SARS-COV-2 | 18 | ASTKGPSVFPLAPSSKSTSGGTAALGCLVKDYFPEPVTVSWNSGA |
| CH1 CH3 | | LTSGVHTFPAVLQSSGLYSLSSVVTVPSSSLGTQTYICNVNHKPSN |
| G1m17; | | TKVDKKVEPKSCDKTHTCPPCPAPELLAGPSVFLFPPKPKDTLMIS |
| IgG1*01 LS | | RTPEVTCVVVDVSHEDPEVKFNWYVDGVEVHNAKTKPREEQYN |
| GAALIE no C- | | STYRVVSVLTVLHQDWLNGKEYKCKVSNKALPLPEEKTISKAKG |
| term Lys (aa) | | QPREPQVYTLPPSRDELTKNQVSLTCLVKGFYPSDIAVEWESNGQ |
| | | PENNYKTTPPVLDSDGSFFLYSKLTVDKSRWQQGNVFSCSVLHE |
| | | ALHSHYTQKSLSLSPG |

Table 3 below sets forth nucleic acid sequences and codon optimized nucleic acid sequences of exemplary constructs.

TABLE 3

| Description | SEQ ID NO: | Sequence |
|---|---|---|
| SARS-COV-2 | 19 | ATGGAATTCGGCCTGTCCTGGGTCTTTCTGGTGGCCATCCTGA |
| S309-v1.1 mAb | | AGGGCGTGCAGTGCCAGGTCCAGCTGGTTCAGAGCGGCGCC |
| CH1 CH3 G1m17; | | GAGGTTAAGAAACCTGGCGCCAGCGTGAAAGTGTCCTGCAA |
| IgGHG1*01 LS; | | GGCCAGCGGCTACCCCTTCACCAGCTACGGCATCTCTTGGGT |
| signal peptide | | GCGGCAGGCCCCTGGACAAGGACTGGAGTGGATGGGATGG |
| (nt-CO) | | ATCAGCACTTACCAGGGCAATACCAACTACGCCCAGAAATTC |
| Full length, codon | | CAGGGCAGAGTGACAATGACCACCGACACCAGCACAACCACA |
| optimized AB1 | | GGCTACATGGAACTGCGGAGACTGAGAAGCGATGATACAGC |
| heavy chain with | | CGTGTACTACTGCGCCAGAGATTATACAAGAGGTGCTTGGTT |
| signal sequence | | CGGCGAGAGCCTGATCGGCGGATTCGACAACTGGGGACAAG |
| Signal sequence | | GCACCCTGGTGACCGTGTCATCCGCCTCTACCAAGGGCCCTA |
| underlined | | GCGTGTTTCCACTGGCCCCTAGCTCTAAAAGCACAAGCGGCG |
| VH in ital | | GCACCGCCGCTCTGGGATGTCTGGTGAAGGACTACTTCCCAG |
| CDRs in bold | | AGCCCGTGACCGTGAGCTGGAACAGCGGCGCTCTCACATCTG |
| CH1 CH3 double | | GGGTGCATACCTTTCCCGCCGTGCTGCAGTCTTCTGGACTGTA |
| underline | | CAGCCTGAGCAGCGTGGTGACCGTGCCCTCCAGCAGCCTGG |
| Stop codon at end | | GCACACAGACCTACATCTGCAACGTGAACCACAAGCCATCTA |
| | | ATACCAAGGTGGATAAGAAGGTGGAACCTAAGAGCTGTGAC |
| | | AAGACACACACATGCCCCCCCTGCCCTGCTCCTGAGCTGCTG |
| | | GGXGGCCCCTCCGTGTTTCTCTTCCCTCCTAAACCCAAGGACA |
| | | CACTGATGATTAGCCGGACCCCAGAGGTGACCTGTGTGGTGG |
| | | TTGACGTGAGTCACGAAGATCCTGAAGTGAAGTTCAACTGGT |
| | | ACGTGGACGGCGTGGAGGTGCATAACGCCAAAACCAAGCCT |
| | | CGGGAAGAGCAGTACAACAGCACCTATAGAGTGGTGAGCGT |
| | | GCTTACAGTGCTGCATCAGGACTGGCTGAACGGCAAGGAAT |
| | | ACAAGTGCAAGGTGTCCAACAAAGCCCTGCCTGCXCCTATYG |
| | | AAAAGACCATCAGCAAGGCCAAGGGCCAACCAAGAGAGCCT |
| | | CAAGTGTACACCCTGCCCCCCAGCAGAGATGAGCTGACCAAG |
| | | AATCAGGTGTCCCTGACCTGCCTGGTCAAAGGCTTCTACCCTA |
| | | GCGACATCGCCGTCGAGTGGGAGAGCAATGGCCAGCCTGAG |
| | | AACAACTACAAGACCACCCCTCCTGTGCTGGACAGCGACGGC |
| | | AGCTTCTTCCTGTATAGCAAGCTGACCGTGGACAAGTCCAGG |
| | | TGGCAGCAGGGCAATGTGTTCAGCTGTAGCGTGCTGCACGA |
| | | GGCCCTGCACAGCCACTACACACAGAAGTCTCTGAGCCTGTC |
| | | TCCTGGCAAGTGA |
| | | wherein X is A, T, G, or C; and |
| | | wherein Y is A, T, or C |
| SARS-COV-2 | 20 | CAGGTCCAGCTGGTTCAGAGCGGCGCCGAGGTTAAGAAACCT |
| S309-v1.1 mAb | | GGCGCCAGCGTGAAAGTGTCCTGCAAGGCCAGCGGCTACCCC |
| CH1 CH3 G1m17; | | TTCACCAGCTACGGCATCTCTTGGGTGCGGCAGGCCCCTGGA |
| IgGHG1*01 LS | | CAAGGACTGGAGTGGATGGGATGGATCAGCACTTACCAGGG |
| (nt-CO) | | CAATACCAACTACGCCCAGAAATTCCAGGGCAGAGTGACAAT |
| Full length, codon | | GACCACCGACACCAGCACAACCACAGGCTACATGGAACTGCG |
| optimized AB1 | | GAGACTGAGAAGCGATGATACAGCCGTGTACTACTGCGCCAG |
| heavy chain | | AGATTATACAAGAGGTGCTTGGTTCGGCGAGAGCCTGATCG |
| without signal | | GCGGATTCGACAACTGGGGACAAGGCACCCTGGTGACCGTG |
| sequence | | TCATCCGCCTCTACCAAGGGCCCTAGCGTGTTTCCACTGGCCC |
| VH in ital | | CTAGCTCTAAAAGCACAAGCGGCGGCACCGCCGCTCTGGGAT |
| CDRs in bold | | GTCTGGTGAAGGACTACTTCCCAGAGCCCGTGACCGTGAGCT |
| CH1 CH3 double | | GGAACAGCGGCGCTCTCACATCTGGGGTGCATACCTTTCCCG |
| underline | | CCGTGCTGCAGTCTTCTGGACTGTACAGCCTGAGCAGCGTGG |
| Stop codon at end | | TGACCGTGCCCTCCAGCAGCCTGGGCACACAGACCTACATCT |
| | | GCAACGTGAACCACAAGCCATCTAATACCAAGGTGGATAAGA |
| | | AGGTGGAACCTAAGAGCTGTGACAAGACACACACATGCCCCC |
| | | CCTGCCCTGCTCCTGAGCTGCTGGGXGGCCCCTCCGTGTTTCT |
| | | CTTCCCTCCTAAACCCAAGGACACACTGATGATTAGCCGGACC |
| | | CCAGAGGTGACCTGTGTGGTGGTTGACGTGAGTCACGAAGA |
| | | TCCTGAAGTGAAGTTCAACTGGTACGTGGACGGCGTGGAGG |
| | | TGCATAACGCCAAAACCAAGCCTCGGGAAGAGCAGTACAAC |
| | | AGCACCTATAGAGTGGTGAGCGTGCTTACAGTGCTGCATCAG |
| | | GACTGGCTGAACGGCAAGGAATACAAGTGCAAGGTGTCCAA |
| | | CAAAGCCCTGCCTGCKCCTATYGAAAAGACCATCAGCAAGGC |
| | | CAAGGGCCAACCAAGAGAGCCTCAAGTGTACACCCTGCCCCC |
| | | CAGCAGAGATGAGCTGACCAAGAATCAGGTGTCCCTGACCTG |
| | | CCTGGTCAAAGGCTTCTACCCTAGCGACATCGCCGTCGAGTG |
| | | GGAGAGCAATGGCCAGCCTGAGAACAACTACAAGACCACCC |
| | | CTCCTGTGCTGGACAGCGACGGCAGCTTCTTCCTGTATAGCA |
| | | AGCTGACCGTGGACAAGTCCAGGTGGCAGCAGGGCAATGTG |
| | | TTCAGCTGTAGCGTGCTGCACGAGGCCCTGCACAGCCACTAC |
| | | ACACAGAAGTCTCTGAGCCTGTCTCCTGGCAAGTGA |
| | | wherein X is A, T, G, or C; and |
| | | wherein Y is A, T, or C |
| SARS-COV-2 | 21 | atgggctggtcctgtatcatcctgttcctggtcgccacagccaccggagtgcacag |
| S309-v1.1 mAb | | ccaagtgcagctggtccagagcggcgccgaggtgaagaagcccggcgctag |
| CH1—CH3 G1m17; | | cgtgaaggtgtcctgtaaagccagcggatatccttttaccagctacggcatctcct |
| IgGHG1*01 LS; | | gggtgcggcaggcccctggccagggcctggaatggatgggctggatcagcac |
| GAALIE; signal | | ctaccagggaaataccaactacgcccagaagttccagggaagagtgacaatg |
| peptide (nt-CO) | | accacagatacatctacaaccaccggctacatggaactgaggcggctgagaa |
| Full length, codon | | gcgacgacaccgccgtgtactactgcgccagagattacaccagaggcgcttgg |
| optimized VIR- | | ttcggcgagagcctgatcggcggcttcgacaactgggccagggaaccctggt |
| AB2 heavy chain | | gacagtgtctagcgcttctaccaaaggcccttctgtctttcctctggccccttctagc |
| with signal | | aagtctacaagcggaggcaccgccgccctgggctgcctggtgaaggactacttc |
| sequence | | cccgagcccgtgaccgtgagctggaatagcggcgccctgacaagcggcgtgca |
| Signal sequence | | caccttcccagctgtgctgcagagcagcggcctgtatagcctgagcagcgtggtc |
| underlined | | accgtgcccagcagcagcctgggaacacagacctacatctgcaacgtgaaccac |
| VH in ital | | aagccttctaataccaaggtggataagaaggtggaacctaagagctgcgacaaaa |
| CDRs in bold | | cacacacatgccctccatgtcctgctccagagctgctggccggccccagcgtttttc |
| CH1 CH3 double | | tgttcccccccaaacctaaagacaccctgatgatcagcagaacccctgaggtgac |
| underline | | ctgtgtggtggtggacgtgtcccacgaagatcctgaggtgaagttcaactggtacg |
| Stop codon at end | | tggatggagtggaagtgcacaacgccaagaccaaacctagagaagagcagtac |
| | | aacagcacatatagagtcgtgtccgtgcttacagtgctgcaccaggactggctgaa |
| | | tggaaaggaatacaagtgcaaggtgtccaacaaggccctgcctctgcctgagga |
| | | gaagacaatctctaaagccaagggccaacctcgggaacctcaggtgtacacatg |
| | | ccccccagccgggacgagctgaccaagaaccaggtgtccctgacctgcctggtc |
| | | aagggcttctacccctctgatatcgccgtggaatgggagagcaacggccaacctg |
| | | agaacaactacaagaccacccctccagtgctggacagcgacggcagcttcttcct |
| | | gtacagcaagctgaccgttgacaagtccagatggcagcgggcaacgtgttcag |
| | | ctgtagcgtcctgcacgaggccctgcattctcactacacccagaagagcctgtccc |
| | | tcagccctggcaagtga |
| SARS-COV-2 | 22 | caagtgcagctggtccagagcggcgccgaggtgaagaagcccggcgctagc |
| S309-v1.1 mAb | | gtgaaggtgtcctgtaaagccagcggatatccttttaccagctacggcatctcctg |
| CH1 CH3 G1m17; | | ggtgcggcaggcccctggccagggcctggaatggatgggctggatcagcacc |
| IgGHG1*01 LS; | | taccagggaaataccaactacgcccagaagttccagggaagagtgacaatga |
| GAALIE; (nt-CO) | | ccacagatacatctacaaccaccggctacatggaactgaggcggctgagaag |
| Full length, codon | | cgacgacaccgccgtgtactactgcgccagagattacaccagaggcgcttggtt |
| optimized AB2 | | cggcgagagcctgatcggcggcttcgacaactggggccagggaaccctggtg |
| heavy chain | | acagtgtctagcgcttctaccaaaggcccttctgtotttcctctggccccttctagca |
| without signal | | agtctacaagcggaggcaccgccgccctgggctgcctggtgaaggactacttcc |
| sequence | | ccgagcccgtgaccgtgagctggaatagcggcgccctgacaagcggcgtgcac |
| VH in ital | | accttcccagctgtgctgcagagcagcggcctgtatagcctgagcagcgtggtca |
| CDRs in bold | | ccgtgcccagcagcagcctgggaacacagacctacatctgcaacgtgaaccaca |
| CH1 CH3 double | | agccttctaataccaaggtggataagaaggtggaacctaagagctgcgacaaaac |
| underline | | acacacatgccctccatgtcctgctccagagctgctggccggccccagcgtttttct |
| Stop codon at end | | gttcccccccaaacctaaagacaccctgatgatcagcagaacccctgaggtgacc |
| | | tgtgtggtggtggacgtgtcccacgaagatcctgaggtgaagttcaactggtacgt |
| | | ggatggagtggaagtgcacaacgccaagaccaaacctagagaagagcagtaca |
| | | acagcacatatagagtcgtgtccgtgcttacagtgctgcaccaggactggctgaat |
| | | ggaaaggaatacaagtgcaaggtgtccaacaaggccctgcctctgcctgaggag |
| | | aagacaatctctaaagccaagggccaacctcgggaacctcaggtgtacacactgc |
| | | cccccagccgggacgagctgaccaagaaccaggtgtccctgacctgcctggtca |
| | | agggcttctacccctctgatatcgccgtggaatgggagagcaacggccaacctga |
| | | gaacaactacaagaccacccctccagtgctggacagcgacggcagcttcttcctg |
| | | tacagcaagctgaccgttgacaagtccagatggcagcagggcaacgtgttcagct |
| | | gtagcgtcctgcacgaggccctgcattctcactacacccagaagagcctgtccctc |
| | | agccctggcaagtga |
| S309-v13 Light | 23 | tgggctggtcctgcatcatcctgttcctggtggccacagccaccggcgtgcacagc |
| chain k1m3; | | gagatcgtcctgacacagagccccggcacactgagcctctccccaggcgagc |
| IgKC*01; signal | | gggctacactgtcttgtagagcttctcagaccgtgtccagcaccagcctggcctg |
| peptide (nt-CO) | | gtatcagcagaaacctggccaggcccctagactgctgatctacggcgccagca |
| Full length, codon | | gcagagccaccggcatccctgatagattcagcggcagcggatctggaaccga |
| optimized light | | cttcaccctgaccatcagccggctggaacccgaggactttgccgtgtactactgc |
| chain with signal | | cagcaacacgacaccagcctgaccttcggcggcggaacaaaggtggaaatc |
| sequence | | aagagaaccgtggccgcccctagcgtgttcatcttcccccccagcgacgagcag |
| Signal sequence | | ctgaagagcggtacagcttctgtggtgtgcctgctgaacaacttctacccgcggga |
| underlined | | agccaaggtgcagtggaaggtggacaacgccctgcagagcggcaacagccag |
| VL in ital | | gagagcgtgacagagcaggacagcaaggacagcacctacagcctgagcagca |
| CDRs in bold | | ccctgaccctgagcaaggccgactacgagaagcacaaggtgtacgcctgtgaag |
| CL in double | | tgacccaccagggcctgtctagccctgtgaccaagtcttttaacagaggcgagtgc |
| underline | | tga |
| Stop codon at end | | |
| S309-v13 Light | 24 | gagatcgtcctgacacagagccccggcacactgagcctctccccaggcgagc |
| chain k1m3; | | gggctacactgtcttgtagagcttctcagaccgtgtccagcaccagcctggcctg |
| IgKC*01; (nt-CO) | | gtatcagcagaaacctggccaggcccctagactgctgatctacggcgccagca |
| Full length, codon | | gcagagccaccggcatccctgatagattcagcggcagcggatctggaaccga |
| optimized light | | cttcaccctgaccatcagccggctggaacccgaggactttgccgtgtactactgc |
| chain without | | cagcaacacgacaccagcctgaccttcggcggeggaacadaggtggaaatc |
| signal sequence | | aagagaaccgtggccgcccctagcgtgttcatcttcccccccagcgacgagcag |
| VL in ital | | ctgaagagcggtacagcttctgtggtgtgcctgctgaacaacttctacccgcggga |
| CDRs in bold | | agccaaggtgcagtggaaggtggacaacgccctgcagagcggcaacagccag |
| CL in double | | gagagcgtgacagagcaggacagcaaggacagcacctacagcctgagcagca |
| underline | | ccctgaccctgagcaaggccgactacgagaagcacaaggtgtacgcctgtgaag |
| Stop codon at end | | tgacccaccagggcctgtctagccctgtgaccaagtcttttaacagaggcgagtgc |
| | | tga |

According to some embodiments, the disclosure provides one or more ceDNA vectors comprising one or more nucleic acid sequences that encode an anti-SARS-CoV-2 S antibody, or an antigen-binding fragment thereof, wherein the nucleic acid sequence is selected from any one in Table 3.

According to some embodiments, the disclosure provides one or more ceDNA vectors comprising one or more nucleic acid sequences that encode an anti-SARS-CoV-2 S antibody, or an antigen-binding fragment thereof, that comprise (i) a heavy chain comprising (i)(1) a VH that comprises or consists of the amino acid sequence set forth in SEQ ID NO.: 5, and (i)(2) a CH1-CH3 that comprises or consists of the amino acid sequence set forth in SEQ ID NO.: 14 or SEQ ID NO.: 15; and (ii) a light chain comprising (ii)(1) a VL that comprises or consists of the amino acid sequence set forth in SEQ ID NO.: 6, and (ii)(2) a CL that comprises or consists of the amino acid sequence set forth in SEQ ID NO.: 13. According to some embodiments, the disclosure provides one or more ceDNA vectors comprising one or more anti-SARS-CoV-2 S antibodies, or an antigen-binding fragments thereof, that comprise (i) a heavy chain comprising (i)(1) a VH that comprises or consists of the amino acid sequence set forth in SEQ ID NO.: 5, and (i)(2) a CH1-CH3 that comprises or consists of the amino acid sequence set forth in SEQ ID NO.: 17 or SEQ ID NO.: 18; and (ii) a light chain comprising (ii)(1) a VL that comprises or consists of the amino acid sequence set forth in SEQ ID NO.: 6, and (ii)(2) a CL that comprises or consists of the amino acid sequence set forth in SEQ ID NO.: 13.

According to some embodiments, an antibody or an antigen-binding fragment of the disclosure which is modified According to some way retains the ability to specifically bind to CoV-S. e.g., retains at least 10% of its CoV-S binding activity (when compared to the parental antibody) when that activity is expressed on a molar basis. Preferably, an antibody or antigen-binding fragment of the disclosure retains at least 20%, 50%, 70%, 80%, 90%, 95% or 100% or more of the CoV-S binding affinity as the parental antibody. It is also intended that an antibody or antigen-binding fragment of the disclosure can include conservative or non-conservative amino acid substitutions (referred to as “conservative variants” or “function conserved variants” of the antibody) that do not substantially alter its biologic activity.

According to some embodiments, the antibodies, or the antigen-binding fragments thereof, exhibit one or more of the following properties: capable of binding to a SARS-CoV-2 surface glycoprotein (S) expressed on a cell surface of a host cell and/or on a SARS-CoV-2 virion; associates with or unites with (e.g., binds) a SARS-CoV-2 surface glycoprotein epitope or antigen comprising the epitope, while not significantly associating or uniting with any other molecules or components in a sample; is cross-reactive for SARS-CoV-2 and one or more additional coronavirus; inhibits growth of coronavirus (e.g., SARS-CoV-2, SARS-CoV) in vitro and/or in vivo; inhibits spread of coronavirus (e.g., SARS-CoV-2, SARS-CoV) in vitro and/or in vivo.

According to some aspects, the present disclosure provides a ceDNA comprising one or more nucleic acids encoding an antibody, or an antigen-binding fragment thereof, that comprises a heavy chain variable domain (VH) comprising a CDRH1, a CDRH2, and a CDRH3, and a light chain variable domain (VL) comprising a CDRL1, a CDRL2, and a CDRL3, and is capable of binding to a surface glycoprotein (S) of SARS-CoV-2. In certain embodiments, the antibody or antigen-binding fragment is capable of binding to a SARS-CoV-2 surface glycoprotein (S) expressed on a cell surface of a host cell and/or on a SARS-CoV-2 virion.

In some embodiments, an antibody or an antigen-binding fragment of the present disclosure associates with or unites with (e.g., binds) a SARS-CoV-2 surface glycoprotein epitope or antigen comprising the epitope, while not significantly associating or uniting with any other molecules or components in a sample.

In certain embodiments, an antibody or an antigen-binding fragment of the present disclosure associates with or unites (e.g., binds) to a SARS-CoV-2 surface glycoprotein epitope, and can also associate with or unite with an epitope from another coronavirus (e.g., SARS CoV) present in the sample, but not associating or uniting with any other molecules or components in the sample. In other words, in certain embodiments, an antibody or an antigen binding fragment of the present disclosure is cross-reactive for SARS-CoV-2 and one or more additional coronavirus.

In certain embodiments, an antibody or antigen-binding fragment of the present disclosure specifically binds to a SARS-CoV-2 surface (S) glycoprotein. According to some embodiments, an antibody or antigen-binding fragment is capable of binding to a Receptor Binding Domain (RBD) of the SARS-CoV-2 surface glycoprotein.

As used herein, “specifically binds” refers to an association or union of an antibody or an antigen-binding fragment to an antigen with an affinity or Ka (i.e., an equilibrium association constant of a particular binding interaction with units of 1/M) equal to or greater than 10⁵ M⁻¹ (which equals the ratio of the on-rate [Kₒₙ] to the off rate [K_{off}] for this association reaction), while not significantly associating or uniting with any other molecules or components in a sample. Alternatively, affinity may be defined as an equilibrium dissociation constant (Kd) of a particular binding interaction with units of M (e.g., 10⁻⁵ M to 10⁻¹³ M).

Antibodies may be classified as “high-affinity” antibodies or as “low-affinity” antibodies. “High-affinity” antibodies refer to those antibodies having a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low-affinity” antibodies refer to those antibodies having a Ka of up to 10⁷ M⁻¹, up to 10⁶ M⁻¹, up to 10⁵ M⁻¹. Alternatively, affinity may be defined as an equilibrium dissociation constant (Kd) of a particular binding interaction with units of M (e.g., 10⁻⁵ M to 10⁻¹³ M), as measured by real-time, label free bio-layer interferometry assay, for example, at 25° C. or 37° C., e.g., an Octet® HTX biosensor, or by surface plasmon resonance, e.g., BIACORE™, or by solution-affinity ELISA.

In certain embodiments, an antibody of the present disclosure is capable of neutralizing infection by SARS-CoV-2. As used herein, a “neutralizing antibody” is one that can neutralize, i.e., prevent, inhibit, reduce, impede, or interfere with, the ability of a pathogen to initiate and/or perpetuate an infection in a host. Neutralization may be quantified by, for example, assessing SARS-CoV-2 RNA levels in a sample (e.g., a lung sample), assessing SARS-CoV-2 viral load in a sample (e.g., a lung sample), assessing histopathology of a sample (e.g., a lung sample), or the like. The terms “neutralizing antibody” and “an antibody that neutralizes” or “antibodies that neutralize” are used interchangeably herein. In any of the presently disclosed embodiments, the antibody or antigen-binding fragment is capable of preventing and/or neutralizing a SARS-CoV-2 infection in an in vitro model of infection and/or in an in vivo animal model of infection (e.g., using a Syrian hamster model with intranasal delivery of SARS-CoV-2) and/or in a human. According to some embodiments, an antibody or antigen-binding fragment of the present disclosure is capable of neutralizing a SARS-CoV-2 infection with an IC90 of about 9 μg/mL. According to some embodiments, an antibody or antigen-binding fragment of the present disclosure is capable of neutralizing a SARS-CoV-2 infection with an IC50 of about 16 to about 20 μg/mL, for example, about 16 μg/ml, about 17 μg/mL, about 18 μg/mL, about 19 μg/mL or about 20 μg/mL. According to some embodiments, an antibody or antigen-binding fragment is capable of neutralizing a SARS-CoV-2 infection, or a virus pseudotyped with SARS-CoV-2, with an IC50 of about 0.3 μg/mL to about 0.4 μg/mL. According to some embodiments, an antibody or antigen-binding fragment, or a composition comprising two or more antibodies or antigen-binding fragments, of the present disclosure is capable of neutralizing a SARS-CoV-2 infection, or a virus pseudotyped with SARS-CoV-2, with an IC50 of about 0.07 μg/mL to about 0.08 μg/mL.

In any of the presently disclosed embodiments, the antibody or antigen-binding fragment is capable of preventing and/or neutralizing a SARS-CoV-2 infection in an in vitro model of infection and/or in an in vivo animal model of infection and/or in a human.

### (i) Fc Mutations (Including LS and GAALIE)

According to some embodiments, the antibody or antigen-binding fragment comprises a Fc polypeptide, or a fragment thereof. The “Fc” comprises the carboxy-terminal portions (i.e., the CH2 and CH3 domains of IgG) of both antibody H chains held together by disulfides. Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation. As discussed herein, modifications (e.g., amino acid substitutions) may be made to an Fc domain in order to modify (e.g., improve, reduce, or ablate) one or more functionality of an Fc-containing polypeptide (e.g., an antibody of the present disclosure). Such functions include, for example, Fc receptor (FcR) binding, antibody half-life modulation (e.g., by binding to FcRn), ADCC function, protein A binding, protein G binding, and complement binding. Amino acid modifications that modify (e.g., improve, reduce, or ablate) Fc functionalities include, for example, M428L/N434S and G236A/A330L/I332E mutations. Unless the context indicates otherwise, Fc amino acid residues are numbered herein according to the EU numbering system.

According to some embodiments of the present disclosure, the antibodies, or antigen-binding fragments described herein, are provided comprising an Fc domain comprising one or more mutations, which, for example, enhance or diminish antibody binding to the FcRn receptor, e.g., at acidic pH as compared to neutral pH. Such mutations may result in an increase in serum half-life of the antibody when administered to an animal.

In any of the herein disclosed embodiments, an antibody or antigen-binding fragment can comprise a Fc polypeptide or fragment thereof comprising a mutation selected from G236A; S239D; A330L; and I332E; or a combination comprising any two or more of the same; e.g., S239D/I332E; S239D/A330L/I332E; G236A/S239D/I332E; G236A/A330L/I332E (also referred to herein as “GAALIE”); or G236A/S239D/A330L/I332E. According to some embodiments, the Fc polypeptide or fragment thereof does not comprise S239D. According to some embodiments, the Fc polypeptide or fragment thereof comprises S at position 239.

According to some embodiments, the Fc polypeptide or fragment thereof may comprise or consist of at least a portion of an Fc polypeptide or fragment thereof that is involved in binding to FcRn binding. In certain embodiments, the Fc polypeptide or fragment thereof comprises one or more amino acid modifications that improve binding affinity for (e.g., enhance binding to) FcRn (e.g., at a pH of about 6.0) and, according to some embodiments, thereby extend in vivo half-life of a molecule comprising the Fc polypeptide or fragment thereof (e.g., as compared to a reference (e.g., wild-type) Fc polypeptide or fragment thereof or antibody that is otherwise the same but does not comprise the modification(s)). In certain embodiments, the Fc polypeptide or fragment thereof comprises or is derived from an IgG Fc and a half-life-extending mutation comprises any one or more of: M428L and N434S (EU numbering). In certain embodiments, a half-life-extending mutation comprises M428L/N434S (also referred to herein as “MLNS” or “LS”).

According to some embodiments, an antibody or antigen-binding fragment includes a Fc moiety that comprises the substitution mutations M428L/N434S. According to some embodiments, an antibody or antigen-binding fragment includes a Fc polypeptide or fragment thereof that comprises the substitution mutations G236A/A330L/I332E. According to some embodiments, an antibody or antigen-binding fragment includes a (e.g., IgG) Fc moiety that comprises a G236A mutation, an A330L mutation, and a I332E mutation (GAALIE), and does not comprise a S239D mutation (e.g., comprises a native S at position 239). In particular embodiments, an antibody or antigen-binding fragment includes an Fc polypeptide or fragment thereof that comprises the substitution mutations: M428L/N434S and G236A/A330L/I332E, and optionally does not comprise S239D. According to some embodiments, an antibody or an antigen-binding fragment includes a Fc polypeptide or fragment thereof that comprises the substitution mutations: M428L/N434S and G236A/S239D/A330L/I332E.

In certain embodiments, the antibody or the antigen-binding fragment is capable of eliciting continued protection in vivo in a subject even once no detectable levels of the antibody or antigen-binding fragment can be found in the subject (i.e., when the antibody or antigen-binding fragment has been cleared from the subject following administration). Such protection is referred to herein as a vaccinal effect.

Without wishing to be bound by theory, it is believed that dendritic cells can internalize complexes of antibody and antigen and thereafter induce or contribute to an endogenous immune response against antigen. In certain embodiments, an antibody or an antigen-binding fragment comprises one or more modifications, such as, for example, mutations in the Fc comprising G236A. A330L, and I332E, that are capable of activating dendritic cells that may induce, e.g., T cell immunity to the antigen.

A distinct advantage of ceDNA vectors over traditional AAV vectors, and even lentiviral vectors, is that there is no size constraint for the one or more nucleic acid sequences that encode an anti-SARS-CoV-2 S antibody, or antigen-binding fragment thereof. In addition, depending on the necessary stiochemistry one can vary ratios of the HC and LC (or HCVR or LCVR), and can use the same or different promoters, for optimal expression.

According to some embodiments, a ceDNA vector for expression of anti-CoV-2 S antibodies and antigen-binding fragments thereof, as described herein, comprises a nucleic acid sequence encoding the HC of the anti-CoV-2 S antibody or antigen-binding fragments thereof in a first ceDNA vector and a nucleic acid sequence encoding the LC of the anti-CoV-2 S antibody or antigen-binding fragments thereof in a second ceDNA vector. According to some embodiments, the nucleic acid sequence encoding the LC is under the control of a first promoter. According to some embodiments, the nucleic acid sequence encoding the HC is under the control of a second promoter. According to some embodiments, the first and the second promoter are the same. According to some embodiments, the first and the second promoter are different.

According to some embodiments, the first ceDNA vector comprising a nucleic acid sequence encoding the HC of the anti-CoV S antibody or antigen-binding fragments thereof and the second ceDNA vector comprising a nucleic acid sequence encoding the LC of the anti-CoV S antibody or antigen-binding fragments thereof are mixed at a 1:1 (HC:LC) molar ratio and co-formulated.

It is well within the abilities of one of skill in the art to take a known and/or publically available protein sequence of e.g., an antibody disclosed herein (e.g., a HC, LC, HV, LV), and reverse engineer a cDNA sequence to encode such a protein. The cDNA can then be codon optimized to match the intended host cell and inserted into a ceDNA vector as described herein.

### III. ceDNA Vector in General for Use in Production of Anti-SARS-CoV-2 S Antibodies and Antigen-Binding Fragments Thereof

Embodiments of the disclosure are based on methods and compositions comprising close ended linear duplexed (ceDNA) vectors that can express anti-CoV-2 S antibodies and antigen-binding fragments thereof. According to some embodiments, the ceDNA comprises a nucleic acid sequence encoding a HC and LC of an anti-CoV-2 S antibody, or an antigen-binding portion thereof. According to some embodiments, the ceDNA comprises a nucleic acid sequence encoding a LC of anti-CoV-2 S antibody, or an antigen-binding portion thereof. The ceDNA vector is preferably duplex, e.g., self-complementary, over at least a portion of the molecule, such as the expression cassette (e.g., ceDNA is not a double stranded circular molecule). The ceDNA vector has covalently closed ends, and thus is resistant to exonuclease digestion (e.g., exonuclease I or exonuclease III), e.g., for over an hour at 37° C.

In general, a ceDNA vector for expression of anti-CoV-2 S antibodies and antigen-binding fragments thereof as disclosed herein, comprises in the 5′ to 3′ direction: a first adeno-associated virus (AAV) inverted terminal repeat (ITR), a nucleic acid sequence of interest (for example an expression cassette as described herein) and a second AAV ITR. The ITR sequences selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization.

Encompassed herein are methods and compositions comprising the ceDNA vector for production of anti-CoV-2 S antibodies and antigen-binding fragments thereof, which may further include a delivery system, such as but not limited to, a liposome nanoparticle delivery system. Non-limiting exemplary liposome nanoparticle systems encompassed for use are disclosed herein. According to some aspects, the disclosure provides for a lipid nanoparticle comprising ceDNA and an ionizable lipid. For example, a lipid nanoparticle formulation that is made and loaded with a ceDNA vector obtained by the process is disclosed in International Application PCT/US2018/050042, filed on Sep. 7, 2018, which is incorporated herein.

The ceDNA vectors as disclosed herein have no packaging constraints imposed by the limiting space within the viral capsid. ceDNA vectors represent a viable eukaryotically-produced alternative to prokaryote-produced plasmid DNA vectors, as opposed to encapsulated AAV genomes. This permits the insertion of control elements, e.g., regulatory switches as disclosed herein, large transgenes, multiple transgenes etc.

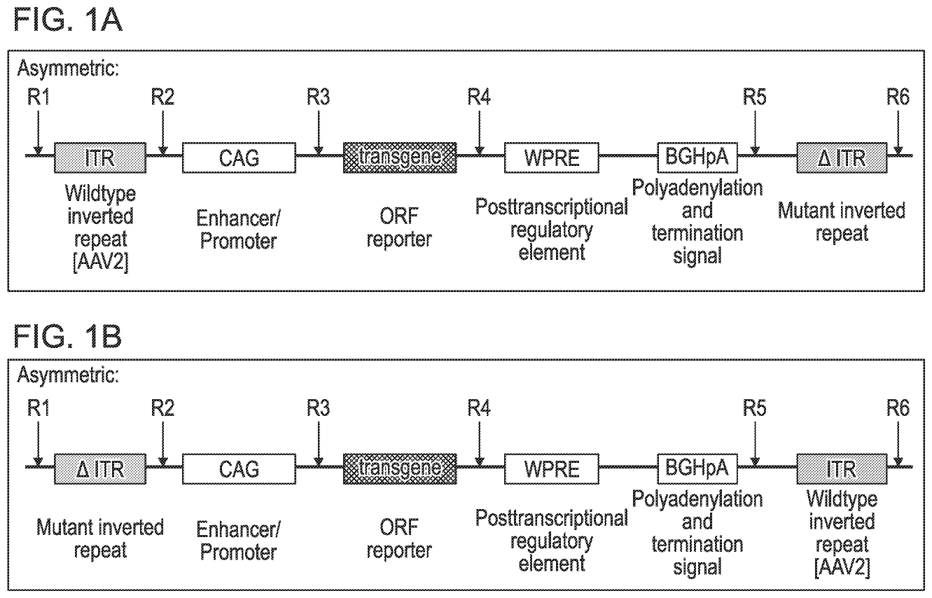

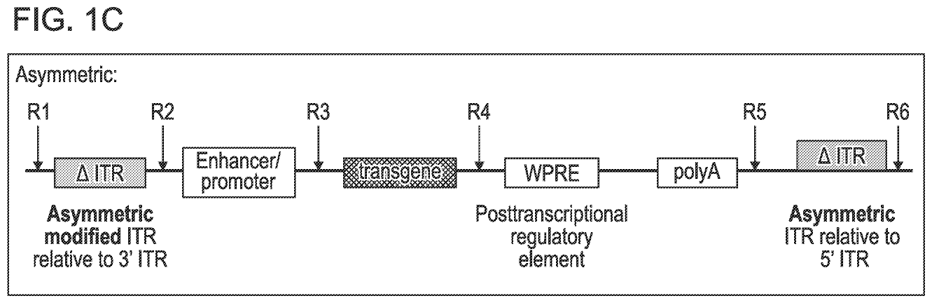

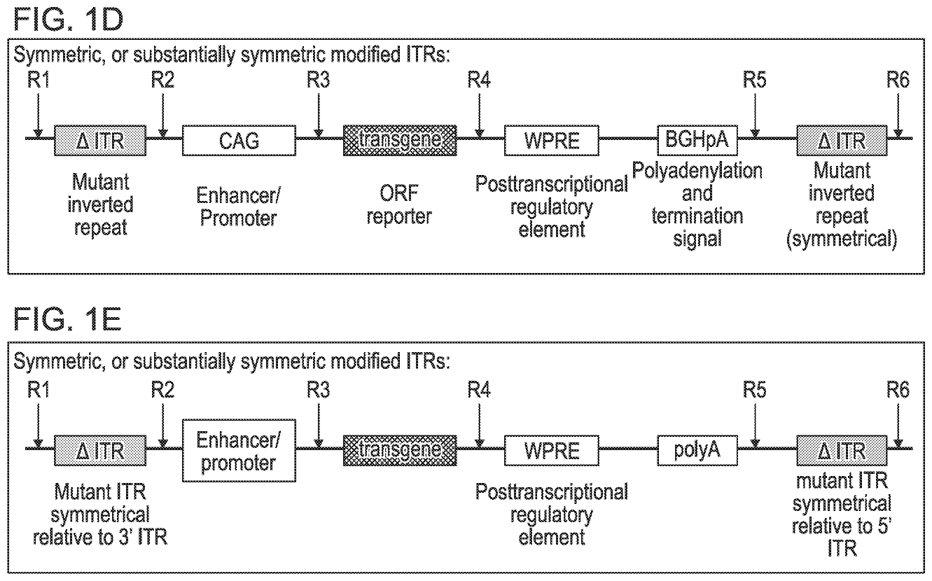

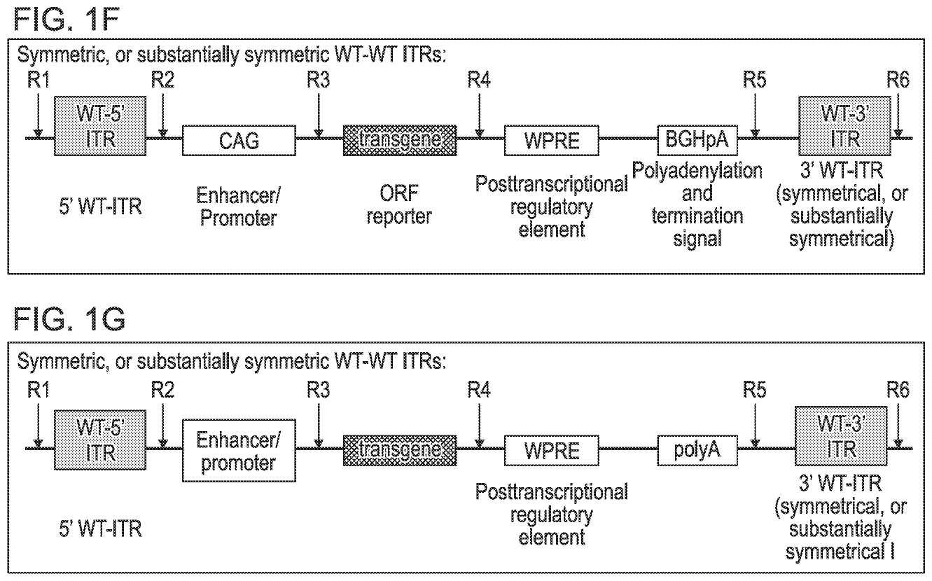

FIG. 1A-1E show schematics of non-limiting, exemplary ceDNA vectors for expression of anti-CoV-2 S antibodies and antigen-binding fragments thereof, or the corresponding sequence of ceDNA plasmids. ceDNA vectors for expression of anti-CoV-2 S antibodies and antigen-binding fragments thereof are capsid-free and can be obtained from a plasmid encoding in this order: a first ITR, an expression cassette comprising a transgene and a second ITR. The expression cassette may include one or more regulatory sequences that allows and/or controls the expression of the transgene, e.g., where the expression cassette can comprise one or more of, in this order: an enhancer/promoter, an ORF reporter (transgene), a post-transcription regulatory element (e.g., WPRE), and a polyadenylation and termination signal (e.g., BGH polyA).

The expression cassette can also comprise an internal ribosome entry site (IRES) and/or a 2A element. The cis-regulatory elements include, but are not limited to, a promoter, a riboswitch, an insulator, a mir-regulatable element, a post-transcriptional regulatory element, a tissue- and cell type-specific promoter and an enhancer. According to some embodiments the ITR can act as the promoter for the transgene. According to some embodiments, the ceDNA vector comprises additional components to regulate expression of the transgene, for example, a regulatory switch, for controlling and regulating the expression of the anti-CoV-2 S antibodies and antigen-binding fragments thereof, and can include if desired, a regulatory switch which is a kill switch to enable controlled cell death of a cell comprising a ceDNA vector.

The expression cassette can comprise more than 4000 nucleotides, 5000 nucleotides, 10,000 nucleotides or 20,000 nucleotides, or 30,000 nucleotides, or 40,000 nucleotides or 50,000 nucleotides, or any range between about 4000-10,000 nucleotides or 10,000-50,000 nucleotides, or more than 50,000 nucleotides. According to some embodiments, the expression cassette can comprise a transgene in the range of 500 to 50,000 nucleotides in length. According to some embodiments, the expression cassette can comprise a transgene in the range of 500 to 75,000 nucleotides in length. According to some embodiments, the expression cassette can comprise a transgene which is in the range of 500 to 10,000 nucleotides in length. According to some embodiments, the expression cassette can comprise a transgene which is in the range of 1000 to 10,000 nucleotides in length. According to some embodiments, the expression cassette can comprise a transgene which is in the range of 500 to 5,000 nucleotides in length. The ceDNA vectors do not have the size limitations of encapsidated AAV vectors, thus enable delivery of a large-size expression cassette to provide efficient transgene expression. According to some embodiments, the ceDNA vector is devoid of prokaryote-specific methylation.

Sequences provided in the expression cassette, expression construct of a ceDNA vector for expression of e.g., anti-CoV-2 S antibodies and antigen-binding fragments thereof, described herein can be codon optimized for the target host cell. As used herein, the term “codon optimized” or “codon optimization” refers to the process of modifying a nucleic acid sequence for enhanced expression in the cells of the vertebrate of interest, e.g., mouse or human, by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's GENE FORGE® codon optimization and custom gene synthesis platform (Aptagen, Inc., 2190 Fox Mill Rd. Suite 300, Herndon, Va. 20171) or another publicly available database. According to some embodiments, the nucleic acid is optimized for human expression.

A transgene expressed by the ceDNA vector for expression of anti-CoV-2 S antibodies and antigen-binding fragments thereof, as disclosed herein encodes anti-CoV-2 S antibodies and antigen-binding fragments thereof. There are many structural features of ceDNA vectors that differ from plasmid-based expression vectors. ceDNA vectors may possess one or more of the following features: the lack of original (i.e., not inserted) bacterial DNA, the lack of a prokaryotic origin of replication, being self-containing, i.e., they do not require any sequences other than the two ITRs, including the Rep binding and terminal resolution sites (RBS and TRS), and an exogenous sequence between the ITRs, the presence of ITR sequences that form hairpins, and the absence of bacterial-type DNA methylation or indeed any other methylation considered abnormal by a mammalian host. In general, it is preferred for the present vectors not to contain any prokaryotic DNA but it is contemplated that some prokaryotic DNA may be inserted as an exogenous sequence, as a non-limiting example in a promoter or enhancer region. Another important feature distinguishing ceDNA vectors from plasmid expression vectors is that ceDNA vectors are single-strand linear DNA having closed ends, while plasmids are always double-strand DNA.

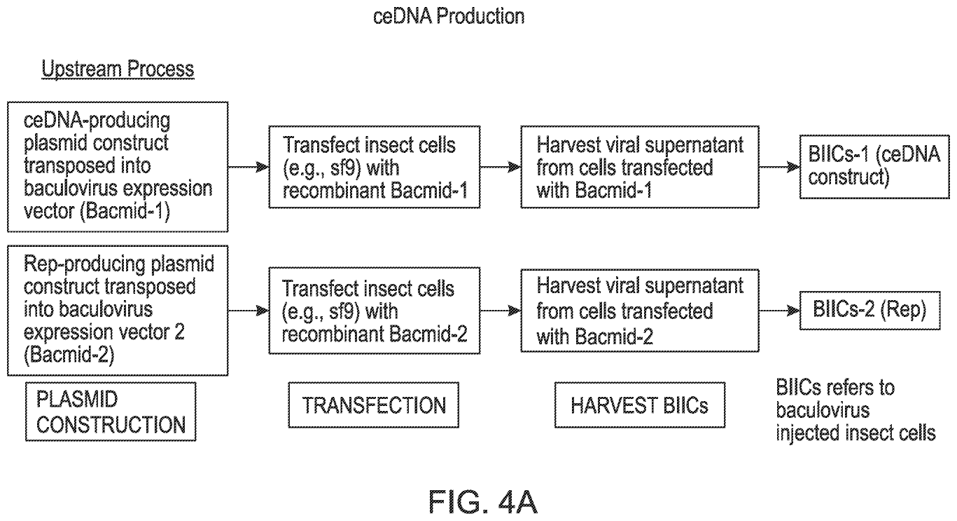

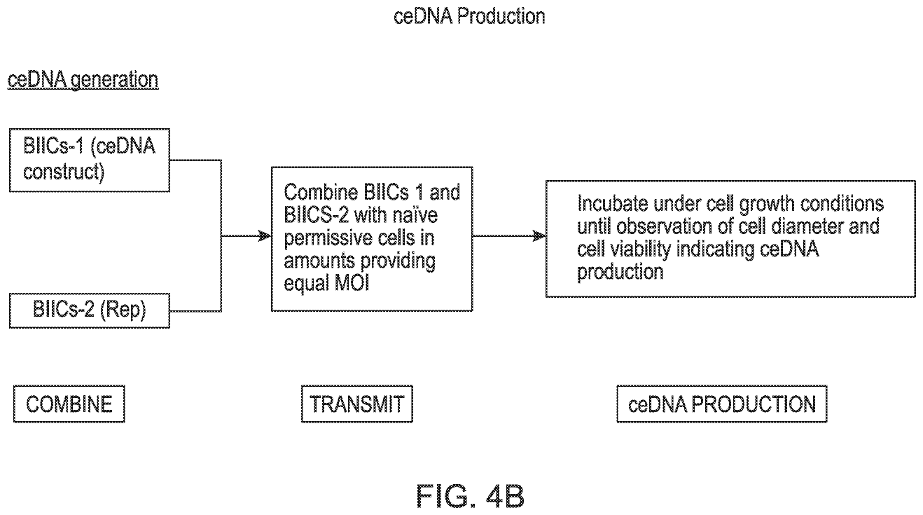

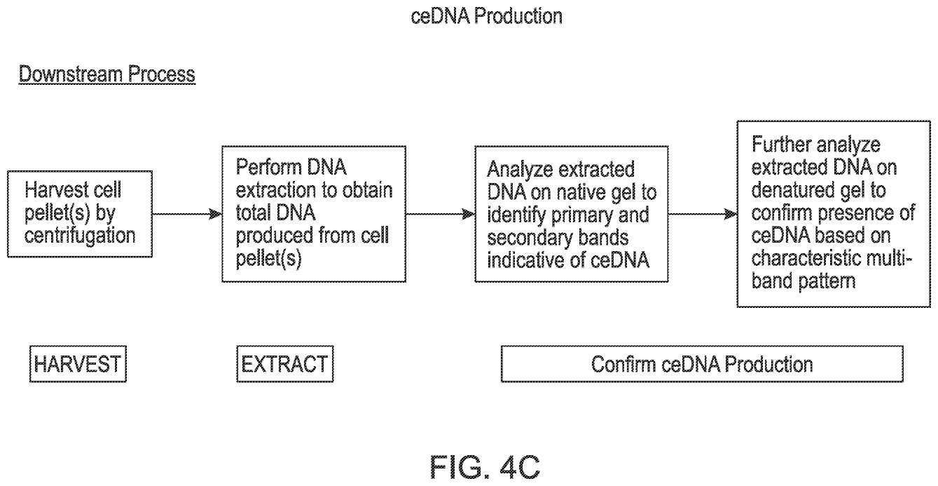

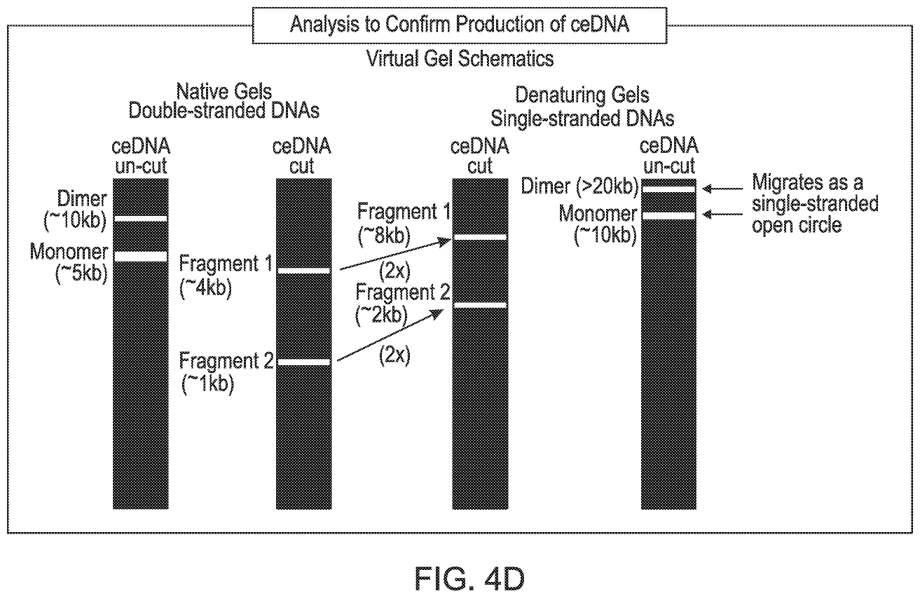

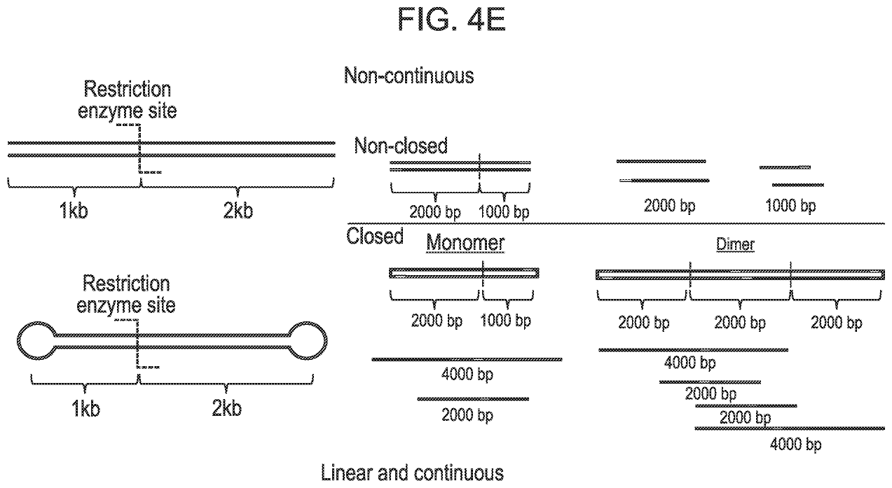

ceDNA vectors for expression of anti-CoV-2 S antibodies and antigen-binding fragments thereof produced by the methods provided herein preferably have a linear and continuous structure rather than a non-continuous structure, as determined by restriction enzyme digestion assay (FIG. 4D). The linear and continuous structure is believed to be more stable from attack by cellular endonucleases, as well as less likely to be recombined and cause mutagenesis. Thus, a ceDNA vector in the linear and continuous structure is a preferred embodiment. The continuous, linear, single strand intramolecular duplex ceDNA vector can have covalently bound terminal ends, without sequences encoding AAV capsid proteins. These ceDNA vectors are structurally distinct from plasmids (including ceDNA plasmids described herein), which are circular duplex nucleic acid molecules of bacterial origin. The complimentary strands of plasmids may be separated following denaturation to produce two nucleic acid molecules, whereas in contrast, ceDNA vectors, while having complimentary strands, are a single DNA molecule and therefore even if denatured, remain a single molecule. According to some embodiments, ceDNA vectors as described herein can be produced without DNA base methylation of prokaryotic type, unlike plasmids. Therefore, the ceDNA vectors and ceDNA-plasmids are different both in term of structure (in particular, linear versus circular) and also in view of the methods used for producing and purifying these different objects (see below), and also in view of their DNA methylation which is of prokaryotic type for ceDNA-plasmids and of eukaryotic type for the ceDNA vector.

There are several advantages of using a ceDNA vector for expression of anti-CoV-2 S antibodies and antigen-binding fragments thereof, as described herein over plasmid-based expression vectors, such advantages include, but are not limited to: (1) plasmids contain bacterial DNA sequences and are subjected to prokaryotic-specific methylation, e.g., 6-methyl adenosine and 5-methyl cytosine methylation, whereas capsid-free AAV vector sequences are of eukaryotic origin and do not undergo prokaryotic-specific methylation; as a result, capsid-free AAV vectors are less likely to induce inflammatory and immune responses compared to plasmids; (2) while plasmids require the presence of a resistance gene during the production process, ceDNA vectors do not; (3) while a circular plasmid is not delivered to the nucleus upon introduction into a cell and requires overloading to bypass degradation by cellular nucleases, ceDNA vectors contain viral cis-elements, i.e., ITRs, that confer resistance to nucleases and can be designed to be targeted and delivered to the nucleus. It is hypothesized that the minimal defining elements indispensable for ITR function are a Rep-binding site (RBS; 5′-GCGCGCTCGCTCGCTC-3′ for AAV2) and a terminal resolution site (TRS; 5′-AGTTGG-3′ for AAV2) plus a variable palindromic sequence allowing for hairpin formation; and 4) ceDNA vectors do not have the over-representation of CpG dinucleotides often found in prokaryote-derived plasmids that reportedly binds a member of the Toll-like family of receptors, eliciting a T cell-mediated immune response. In contrast, transductions with capsid-free AAV vectors disclosed herein can efficiently target cell and tissue-types that are difficult to transduce with conventional AAV virions using various delivery reagent.

### A. Inverted Terminal Repeats (ITRs)

As disclosed herein, ceDNA vectors for expression of anti-CoV-2 S antibodies and antigen-binding fragments thereof, contain a nucleic acid sequence positioned between two inverted terminal repeat (ITR) sequences, where the ITR sequences can be an asymmetrical ITR pair or a symmetrical- or substantially symmetrical ITR pair, as these terms are defined herein. A ceDNA vector as disclosed herein can comprise ITR sequences that are selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization, where the methods of the present disclosure may further include a delivery system, such as but not limited to a liposome nanoparticle delivery system.

According to some embodiments, the ITR sequence can be from viruses of the Parvoviridae family, which includes two subfamilies: Parvovirinae, which infect vertebrates, and Densovirinae, which infect insects. The subfamily Parvovirinae (referred to as the parvoviruses) includes the genus Dependovirus, the members of which, under most conditions, require coinfection with a helper virus such as adenovirus or herpes virus for productive infection. The genus Dependovirus includes adeno-associated virus (AAV), which normally infects humans (e.g., serotypes 2, 3A, 3B, 5, and 6) or primates (e.g., serotypes 1 and 4), and related viruses that infect other warm-blooded animals (e.g., bovine, canine, equine, and ovine adeno-associated viruses). The parvoviruses and other members of the Parvoviridae family are generally described in Kenneth I. Berns, “Parvoviridae: The Viruses and Their Replication.” Chapter 69 in FIELDS VIROLOGY (3d Ed. 1996).

While ITRs exemplified in herein are AAV2 WT-ITRs, one of ordinary skill in the art is aware that one can as stated above use ITRs from any known parvovirus, for example a dependovirus such as AAV (e.g., AAV1, AAV2, AAV3, AAV4, AAV5, AAV 5, AAV7, AAV8, AAV9, AAV10, AAV 11. AAV12, AAVrh8, AAVrh10, AAV-DJ, and AAV-DJ8 genome. E.g., NCBI: NC 002077; NC 001401; NC001729; NC001829; NC006152; NC 006260; NC 006261), chimeric ITRs, or ITRs from any synthetic AAV. According to some embodiments, the AAV can infect warm-blooded animals, e.g., avian (AAAV), bovine (BAAV), canine, equine, and ovine adeno-associated viruses. According to some embodiments the ITR is from B19 parvovirus (GenBank Accession No: NC 000883), Minute Virus from Mouse (MVM) (GenBank Accession No. NC 001510); goose parvovirus (GenBank Accession No. NC 001701); snake parvovirus 1 (GenBank Accession No. NC 006148). According to some embodiments, the 5′ WT-ITR can be from one serotype and the 3′ WT-ITR from a different serotype, as discussed herein.

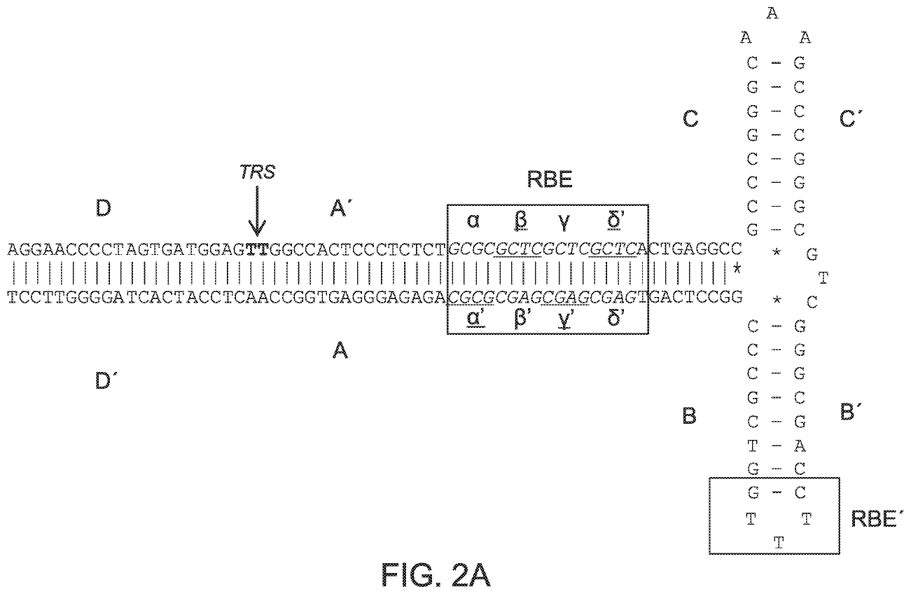

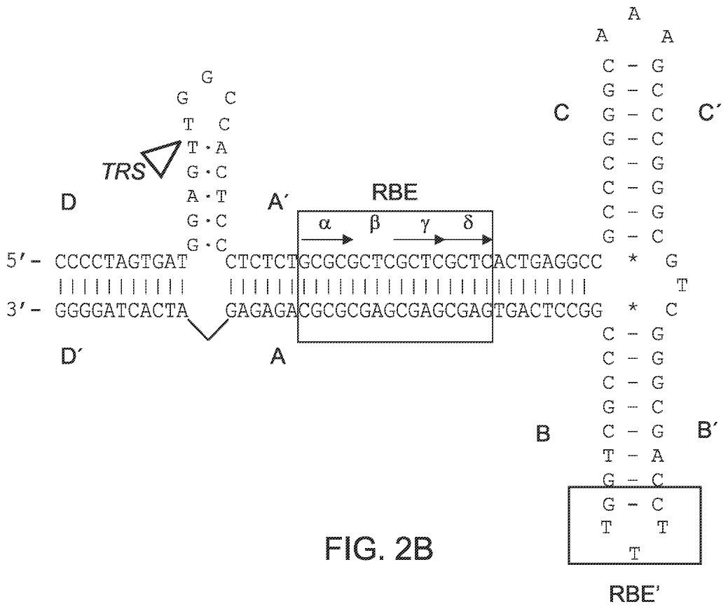

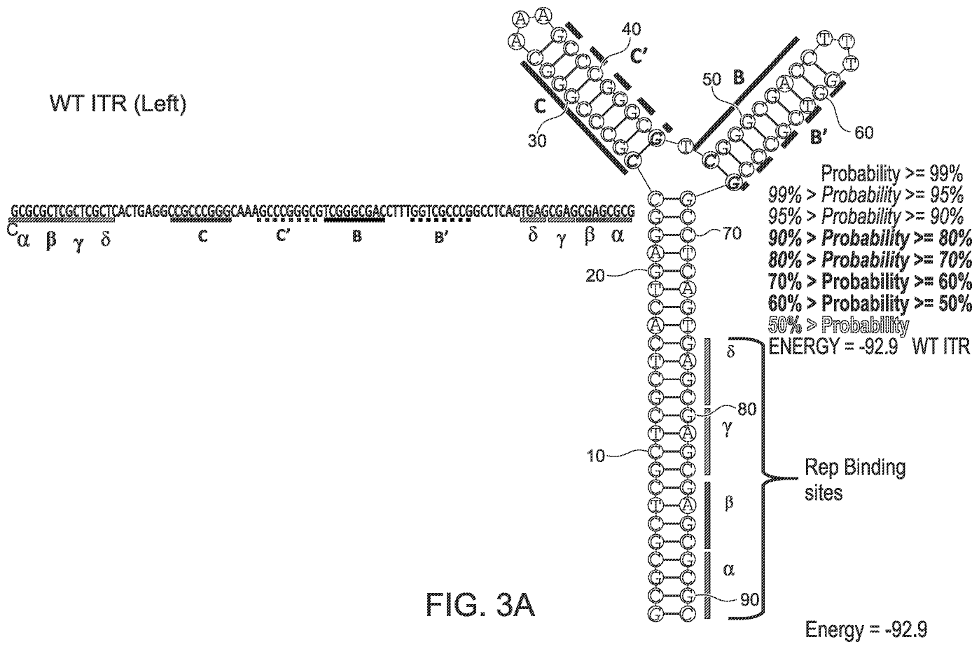

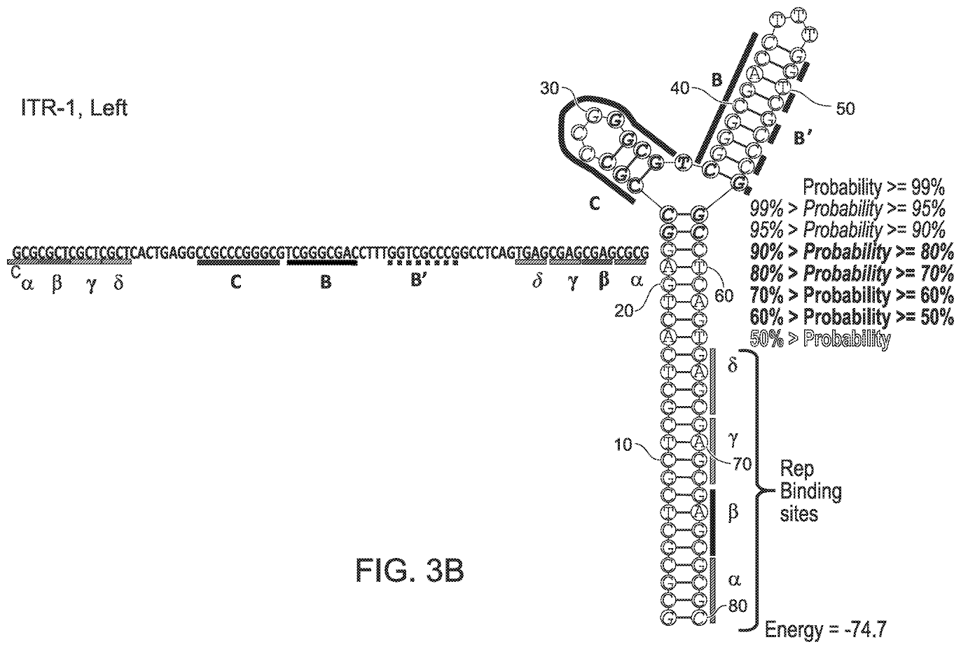

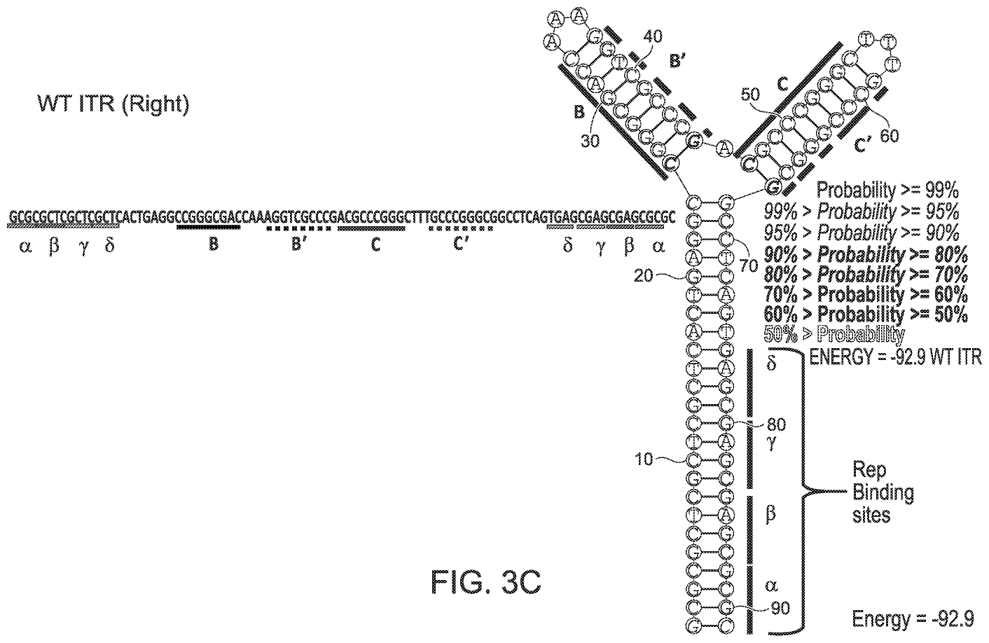

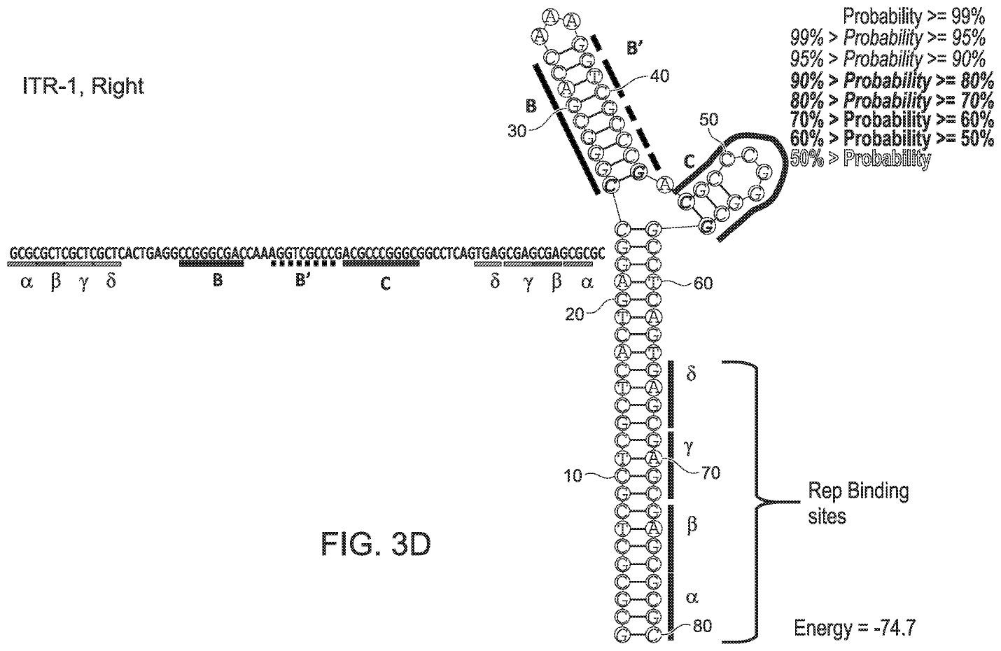

An ordinarily skilled artisan is aware that ITR sequences have a common structure of a double-stranded Holliday junction, which typically is a T-shaped or Y-shaped hairpin structure (see e.g., FIG. 2A and FIG. 3A), where each WT-ITR is formed by two palindromic arms or loops (B-B′ and C-C′) embedded in a larger palindromic arm (A-A′), and a single stranded D sequence, (where the order of these palindromic sequences defines the flip or flop orientation of the ITR). See, for example, structural analysis and sequence comparison of ITRs from different AAV serotypes (AAV1-AAV6) and described in Grimm et al., J. Virology, 2006; 80(1); 426-439; Yan et al., J. Virology, 2005; 364-379; Duan et al., Virology 1999; 261; 8-14. One of ordinary skill in the art can readily determine WT-ITR sequences from any AAV serotype for use in a ceDNA vector or ceDNA-plasmid based on the exemplary AAV2 ITR sequences provided herein. See, for example, the sequence comparison of ITRs from different AAV serotypes (AAV1-AAV6, and avian AAV (AAAV) and bovine AAV (BAAV)) described in Grimm et al., J. Virology, 2006; 80(1); 426-439; that show the % identity of the left ITR of AAV2 to the left ITR from other serotypes: AAV-1 (84%), AAV-3 (86%), AAV-4 (79%), AAV-5 (58%), AAV-6 (left ITR) (100%) and AAV-6 (right ITR) (82%).

### (i) Symmetrical ITR Pairs

According to some embodiments, a ceDNA vector for expression of anti-CoV-2 S antibodies and antigen-binding fragments thereof, as described herein comprises, in the 5′ to 3′ direction: a first adeno-associated virus (AAV) inverted terminal repeat (ITR), a nucleic acid sequence of interest (for example an expression cassette as described herein) and a second AAV ITR, where the first ITR (5′ ITR) and the second ITR (3′ ITR) are symmetric, or substantially symmetrical with respect to each other—that is, a ceDNA vector can comprise ITR sequences that have a symmetrical three-dimensional spatial organization such that their structure is the same shape in geometrical space, or have the same A, C-C′ and B-B′ loops in 3D space. In such an embodiment, a symmetrical ITR pair, or substantially symmetrical ITR pair can be modified ITRs (e.g., mod-ITRs) that are not wild-type ITRs. A mod-ITR pair can have the same sequence which has one or more modifications from wild-type ITR and are reverse complements (inverted) of each other. In alternative embodiments, a modified ITR pair are substantially symmetrical as defined herein, that is, the modified ITR pair can have a different sequence but have corresponding or the same symmetrical three-dimensional shape.

### (a) Wildtype ITRs

According to some embodiments, the symmetrical ITRs, or substantially symmetrical ITRs are wild type (WT-ITRs) as described herein. That is, both ITRs have a wild-type sequence, but do not necessarily have to be WT-ITRs from the same AAV serotype. That is, according to some embodiments, one WT-ITR can be from one AAV serotype, and the other WT-ITR can be from a different AAV serotype. In such an embodiment, a WT-ITR pair are substantially symmetrical as defined herein, that is, they can have one or more conservative nucleotide modification while still retaining the symmetrical three-dimensional spatial organization.

Accordingly, as disclosed herein, ceDNA vectors contain a transgene or nucleic acid sequence positioned between two flanking wild-type inverted terminal repeat (WT-ITR) sequences, that are either the reverse complement (inverted) of each other, or alternatively, are substantially symmetrical relative to each other—that is a WT-ITR pair have symmetrical three-dimensional spatial organization. According to some embodiments, a wild-type ITR sequence (e.g., AAV WT-ITR) comprises a functional Rep binding site (RBS; e.g., 5′-GCGCGCTCGCTCGCTC-3′ for AAV) and a functional terminal resolution site (TRS; e.g., 5′-AGTT-3′).

According to some aspect, ceDNA vectors for expression of anti-CoV-2 S antibodies and antigen-binding fragments thereof are obtainable from a vector polynucleotide that encodes a nucleic acid operatively positioned between two WT inverted terminal repeat sequences (WT-ITRs) (e.g., AAV WT-ITRs). That is, both ITRs have a wild-type sequence, but do not necessarily have to be WT-ITRs from the same AAV serotype. According to some embodiments, one WT-ITR can be from one AAV serotype, and the other WT-ITR can be from a different AAV serotype. In such an embodiment, the WT-ITR pair are substantially symmetrical as defined herein, that is, they can have one or more conservative nucleotide modification while still retaining the symmetrical three-dimensional spatial organization. According to some embodiments, the 5′ WT-ITR is from one AAV serotype, and the 3′ WT-ITR is from the same or a different AAV serotype. According to some embodiments, the 5′ WT-ITR and the 3′WT-ITR are mirror images of each other, that is they are symmetrical. According to some embodiments, the 5′ WT-ITR and the 3′ WT-ITR are from the same AAV serotype.

WT ITRs are well known. According to some embodiment the two ITRs are from the same AAV2 serotype. In certain embodiments one can use WT from other serotypes. There are a number of serotypes that are homologous, e.g., AAV2, AAV4, AAV6, AAV8. According to some embodiment, closely homologous ITRs (e.g., ITRs with a similar loop structure) can be used. In another embodiment, one can use AAV WT ITRs that are more diverse, e.g., AAV2 and AAV5, and still another embodiment, one can use an ITR that is substantially WT—that is, it has the basic loop structure of the WT but some conservative nucleotide changes that do not alter or affect the properties. When using WT-ITRs from the same viral serotype, one or more regulatory sequences may further be used. In certain embodiments, the regulatory sequence is a regulatory switch that permits modulation of the activity of the ceDNA, e.g., the expression of the encoded anti-CoV-2 S antibodies and antigen-binding fragments thereof.

According to some embodiments, one aspect of the technology described herein relates to a ceDNA vector for expression of anti-CoV-2 S antibodies and antigen-binding fragments thereof, wherein the ceDNA vector comprises at least one nucleic acid sequence encoding, e.g., a HC and/or a LC, operably positioned between two wild-type inverted terminal repeat sequences (WT-ITRs), wherein the WT-ITRs can be from the same serotype, different serotypes or substantially symmetrical with respect to each other (i.e., have the symmetrical three-dimensional spatial organization such that their structure is the same shape in geometrical space, or have the same A. C-C′ and B-B′ loops in 3D space). According to some embodiments, the symmetric WT-ITRs comprises a functional terminal resolution site and a Rep binding site. According to some embodiments, the nucleic acid sequence encodes an antibody, or antigen-binding fragment, as described herein, and wherein the vector is not in a viral capsid.

According to some embodiments, the WT-ITRs are the same but the reverse complement of each other. For example, the sequence AACG in the 5′ ITR may be CGTT (i.e., the reverse complement) in the 3′ ITR at the corresponding site. According to some example, the 5′ WT-ITR sense strand comprises the sequence of ATCGATCG and the corresponding 3′ WT-ITR sense strand comprises CGATCGAT (i.e., the reverse complement of ATCGATCG). According to some embodiments, the WT-ITRs ceDNA further comprises a terminal resolution site and a replication protein binding site (RPS) (sometimes referred to as a replicative protein binding site), e.g., a Rep binding site.

Exemplary WT-ITR sequences for use in the ceDNA vectors for expression of Anti-CoV S antibodies and antigen-binding fragments thereof (e.g., anti-CoV-2 S antibodies and antigen-binding fragments thereof) comprising WT-ITRs are shown in Table 2 herein, which shows pairs of WT-ITRs (5′ WT-ITR and the 3′ WT-ITR).

As an exemplary example, the present disclosure provides a ceDNA vector for expression of anti-CoV S antibodies and antigen-binding fragments thereof (e.g., anti-CoV-2 S antibodies and antigen-binding fragments thereof) comprising a promoter operably linked to a transgene (e.g., nucleic acid sequence), with or without the regulatory switch, where the ceDNA is devoid of capsid proteins and is: (a) produced from a ceDNA-plasmid (e.g., see FIGS. 1F-1G) that encodes WT-ITRs, where each WT-ITR has the same number of intramolecularly duplexed base pairs in its hairpin secondary configuration (preferably excluding deletion of any AAA or TTT terminal loop in this configuration compared to these reference sequences), and (b) is identified as ceDNA using the assay for the identification of ceDNA by agarose gel electrophoresis under native gel and denaturing conditions in Example 1.

According to some embodiments, the flanking WT-ITRs are substantially symmetrical to each other. In this embodiment the 5′ WT-ITR can be from one serotype of AAV, and the 3′ WT-ITR from a different serotype of AAV, such that the WT-ITRs are not identical reverse complements. For example, the 5′ WT-ITR can be from AAV2, and the 3′ WT-ITR from a different serotype (e.g., AAV1, 3, 4, 5, 6, 7, 8, 9, 10, 11, and 12. According to some embodiments, WT-ITRs can be selected from two different parvoviruses selected from any to of: AAV1, AAV2, AAV3, AAV4, AAV5, AAV6, AAV7, AAV8, AAV9, AAV10, AAV11, AAV12, AAV13, snake parvovirus (e.g., royal python parvovirus), bovine parvovirus, goat parvovirus, avian parvovirus, canine parvovirus, equine parvovirus, shrimp parvovirus, porcine parvovirus, or insect AAV. According to some embodiments, such a combination of WT ITRs is the combination of WT-ITRs from AAV2 and AAV6. According to some embodiment, the substantially symmetrical WT-ITRs are when one is inverted relative to the other ITR at least 90% identical, at least 95% identical, at least 96% . . . 97% . . . 98% . . . 99% . . . . 99.5% and all points in between, and has the same symmetrical three-dimensional spatial organization. According to some embodiments, a WT-ITR pair are substantially symmetrical as they have symmetrical three-dimensional spatial organization, e.g., have the same 3D organization of the A, C-C′. B-B′ and D arms. According to some embodiment, a substantially symmetrical WT-ITR pair are inverted relative to the other, and are at least 95% identical, at least 96% . . . 97% . . . 98% . . . 99% . . . . 99.5% and all points in between, to each other, and one WT-ITR retains the Rep-binding site (RBS) of 5′-GCGCGCTCGCTCGCTC-3′ and a terminal resolution site (trs). According to some embodiments, a substantially symmetrical WT-ITR pair are inverted relative to each other, and are at least 95% identical, at least 96% . . . 97% . . . 98% . . . 99% . . . . 99.5% and all points in between, to each other, and one WT-ITR retains the Rep-binding site (RBS) of 5′-GCGCGCTCGCTCGCTC-3′ and a terminal resolution site (trs) and in addition to a variable palindromic sequence allowing for hairpin secondary structure formation. Homology can be determined by standard means well known in the art such as BLAST (Basic Local Alignment Search Tool), BLASTN at default setting.

According to some embodiments, the structural element of the ITR can be any structural element that is involved in the functional interaction of the ITR with a large Rep protein (e.g., Rep 78 or Rep 68). In certain embodiments, the structural element provides selectivity to the interaction of an ITR with a large Rep protein, i.e., determines at least in part which Rep protein functionally interacts with the ITR. In other embodiments, the structural element physically interacts with a large Rep protein when the Rep protein is bound to the ITR. Each structural element can be, e.g., a secondary structure of the ITR, a nucleic acid sequence of the ITR, a spacing between two or more elements, or a combination of any of the above. According to some embodiment, the structural elements are selected from the group consisting of an A and an A′ arm, a B and a B′ arm, a C and a C′ arm, a D arm, a Rep binding site (RBE) and an RBE′ (i.e., complementary RBE sequence), and a terminal resolution sire (trs).

By way of example only, Table 4 indicates exemplary combinations of WT-ITRs.

Table 4: Exemplary combinations of WT-ITRs from the same serotype or different serotypes, or different parvoviruses. The order shown is not indicative of the ITR position, for example, “AAV1, AAV2” demonstrates that the ceDNA can comprise a WT-AAV1 ITR in the 5′ position, and a WT-AAV2 ITR in the 3′ position, or vice versa, a WT-AAV2 ITR the 5′ position, and a WT-AAV1 ITR in the 3′ position. Abbreviations: AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12); AAVrh8, AAVrh10, AAV-DJ, and AAV-DJ8 genome (E.g., NCBI: NC 002077; NC 001401; NC001729; NC001829; NC006152; NC 006260; NC 006261), ITRs from warm-blooded animals (avian AAV (AAAV), bovine AAV (BAAV), canine, equine, and ovine AAV), ITRs from B19 Parvovirus (GenBank Accession No: NC 000883), Minute Virus from Mouse (MVM) (GenBank Accession No. NC 001510); Goose: goose parvovirus (GenBank Accession No. NC 001701); snake: snake parvovirus 1 (GenBank Accession No. NC 006148).

TABLE 4
Exemplary combinations of WT-ITRs

| | | | | |
|---|---|---|---|---|
| AAV1, AAV1 | AAV2, AAV2 | AAV3, AAV3 | AAV4, AAV4 | AAV5, AAV5 |
| AAV1, AAV2 | AAV2, AAV3 | AAV3, AAV4 | AAV4, AAV5 | AAV5, AAV6 |
| AAV1, AAV3 | AAV2, AAV4 | AAV3, AAV5 | AAV4, AAV6 | AAV5, AAV7 |
| AAV1, AAV4 | AAV2, AAV5 | AAV3, AAV6 | AAV4, AAV7 | AAV5, AAV8 |
| AAV1, AAV5 | AAV2, AAV6 | AAV3, AAV7 | AAV4, AAV8 | AAV5, AAV9 |
| AAV1, AAV6 | AAV2, AAV7 | AAV3, AAV8 | AAV4, AAV9 | AAV5, AAV10 |
| AAV1, AAV7 | AAV2, AAV8 | AAV3, AAV9 | AAV4, AAV10 | AAV5, AAV11 |
| AAV1, AAV8 | AAV2, AAV9 | AAV3, AAV10 | AAV4, AAV11 | AAV5, AAV12 |
| AAV1, AAV9 | AAV2, AAV10 | AAV3, AAV11 | AAV4, AAV12 | AAV5, AAVRH8 |
| AAV1, AAV10 | AAV2, AAV11 | AAV3, AAV12 | AAV4, AAVRH8 | AAV5, AAVRH10 |
| AAV1, AAV11 | AAV2, AAV12 | AAV3, AAVRH8 | AAV4, AAVRH10 | AAV5, AAV13 |
| AAV1, AAV12 | AAV2, AAVRH8 | AAV3, AAVRH10 | AAV4, AAV13 | AAV5, AAVDJ |
| AAV1, AAVRH8 | AAV2, AAVRH10 | AAV3, AAV13 | AAV4, AAVDJ | AAV5, AAVDJ8 |
| AAV1, AAVRH10 | AAV2, AAV13 | AAV3, AAVDJ | AAV4, AAVDJ8 | AAV5, AVIAN |
| AAV1, AAV13 | AAV2, AAVDJ | AAV3, AAVDJ8 | AAV4, AVIAN | AAV5, BOVINE |
| AAV1, AAVDJ | AAV2, AAVDJ8 | AAV3, AVIAN | AAV4, BOVINE | AAV5, CANINE |
| AAV1, AAVDJ8 | AAV2, AVIAN | AAV3, BOVINE | AAV4, CANINE | AAV5, EQUINE |
| AAV1, AVIAN | AAV2, BOVINE | AAV3, CANINE | AAV4, EQUINE | AAV5, GOAT |
| AAV1, BOVINE | AAV2, CANINE | AAV3, EQUINE | AAV4, GOAT | AAV5, SHRIMP |
| AAV1, CANINE | AAV2, EQUINE | AAV3, GOAT | AAV4, SHRIMP | AAV5, PORCINE |
| AAV1, EQUINE | AAV2, GOAT | AAV3, SHRIMP | AAV4, PORCINE | AAV5, INSECT |
| AAV1, GOAT | AAV2, SHRIMP | AAV3, PORCINE | AAV4, INSECT | AAV5, OVINE |
| AAV1, SHRIMP | AAV2, PORCINE | AAV3, INSECT | AAV4, OVINE | AAV5, B19 |
| AAV1, PORCINE | AAV2, INSECT | AAV3, OVINE | AAV4, B19 | AAV5, MVM |
| AAV1, INSECT | AAV2, OVINE | AAV3, B19 | AAV4, MVM | AAV5, GOOSE |
| AAV1, OVINE | AAV2, B19 | AAV3, MVM | AAV4, GOOSE | AAV5, SNAKE |
| AAV1, B19 | AAV2, MVM | AAV3, GOOSE | AAV4, SNAKE | |
| AAV1, MVM | AAV2, GOOSE | AAV3, SNAKE | | |
| AAV1, GOOSE | AAV2, SNAKE | | | |
| AAV1, SNAKE | | | | |
| AAV6, AAV6 | AAV7, AAV7 | AAV8, AAV8 | AAV9, AAV9 | AAV10, AAV10 |
| AAV6, AAV7 | AAV7, AAV8 | AAV8, AAV9 | AAV9, AAV10 | AAV10, AAV11 |
| AAV6, AAV8 | AAV7, AAV9 | AAV8, AAV10 | AAV9, AAV11 | AAV10, AAV12 |
| AAV6, AAV9 | AAV7, AAV10 | AAV8, AAV11 | AAV9, AAV12 | AAV10, AAVRH8 |
| AAV6, AAV10 | AAV7, AAV11 | AAV8, AAV12 | AAV9, AAVRH8 | AAV10, AAVRH10 |
| AAV6, AAV11 | AAV7, AAV12 | AAV8, AAVRH8 | AAV9, AAVRH10 | AAV10, AAV13 |
| AAV6, AAV12 | AAV7, AAVRH8 | AAV8, AAVRH10 | AAV9, AAV13 | AAV10, AAVDJ |
| AAV6, AAVRH8 | AAV7, AAVRH10 | AAV8, AAV13 | AAV9, AAVDJ | AAV10, AAVDJ8 |
| AAV6, AAVRH10 | AAV7, AAV13 | AAV8, AAVDJ | AAV9, AAVDJ8 | AAV10, AVIAN |
| AAV6, AAV13 | AAV7, AAVDJ | AAV8, AAVDJ8 | AAV9, AVIAN | AAV10, BOVINE |
| AAV6, AAVDJ | AAV7, AAVDJ8 | AAV8, AVIAN | AAV9, BOVINE | AAV10, CANINE |
| AAV6, AAVDJ8 | AAV7, AVIAN | AAV8, BOVINE | AAV9, CANINE | AAV10, EQUINE |
| AAV6, AVIAN | AAV7, BOVINE | AAV8, CANINE | AAV9, EQUINE | AAV10, GOAT |
| AAV6, BOVINE | AAV7, CANINE | AAV8, EQUINE | AAV9, GOAT | AAV10, SHRIMP |
| AAV6, CANINE | AAV7, EQUINE | AAV8, GOAT | AAV9, SHRIMP | AAV10, PORCINE |
| AAV6, EQUINE | AAV7, GOAT | AAV8, SHRIMP | AAV9, PORCINE | AAV10, INSECT |
| AAV6, GOAT | AAV7, SHRIMP | AAV8, PORCINE | AAV9, INSECT | AAV10, OVINE |
| AAV6, SHRIMP | AAV7, PORCINE | AAV8, INSECT | AAV9, OVINE | AAV10, B19 |
| AAV6, PORCINE | AAV7, INSECT | AAV8, OVINE | AAV9, B19 | AAV10, MVM |
| AAV6, INSECT | AAV7, OVINE | AAV8, B19 | AAV9, MVM | AAV10, GOOSE |
| AAV6, OVINE | AAV7, B19 | AAV8, MVM | AAV9, GOOSE | AAV10, SNAKE |
| AAV6, B19 | AAV7, MVM | AAV8, GOOSE | AAV9, SNAKE | |
| AAV6, MVM | AAV7, GOOSE | AAV8, SNAKE | | |
| AAV6, GOOSE | AAV7, SNAKE | | | |
| AAV6, SNAKE | | | | |
| AAV11, AAV11 | AAV12, AAV12 | AAVRH8, AAVRH8 | AAVRH10, AAVRH10 | AAV13, AAV13 |
| AAV11, AAV12 | AAV12, AAVRH8 | AAVRH8, AAVRH10 | AAVRH10, AAV13 | AAV13, AAVDJ |
| AAV11, AAVRH8 | AAV12, AAVRH10 | AAVRH8, AAV13 | AAVRH10, AAVDJ | AAV13, AAVDJ8 |
| AAV11, AAVRH10 | AAV12, AAV13 | AAVRH8, AAVDJ | AAVRH10, AAVDJ8 | AAV13, AVIAN |
| AAV11, AAV13 | AAV12, AAVDJ | AAVRH8, AAVDJ8 | AAVRH10, AVIAN | AAV13, BOVINE |
| AAV11, AAVDJ | AAV12, AAVDJ8 | AAVRH8, AVIAN | AAVRH10, BOVINE | AAV13, CANINE |
| AAV11, AAVDJ8 | AAV12, AVIAN | AAVRH8, BOVINE | AAVRH10, CANINE | AAV13, EQUINE |
| AAV11, AVIAN | AAV12, BOVINE | AAVRH8, CANINE | AAVRH10, EQUINE | AAV13, GOAT |
| AAV11, BOVINE | AAV12, CANINE | AAVRH8, EQUINE | AAVRH10, GOAT | AAV13, SHRIMP |
| AAV11, CANINE | AAV12, EQUINE | AAVRH8, GOAT | AAVRH10, SHRIMP | AAV13, PORCINE |
| AAV11, EQUINE | AAV12, GOAT | AAVRH8, SHRIMP | AAVRH10, PORCINE | AAV13, INSECT |
| AAV11, GOAT | AAV12, SHRIMP | AAVRH8, PORCINE | AAVRH10, INSECT | AAV13, OVINE |
| AAV11, SHRIMP | AAV12, PORCINE | AAVRH8, INSECT | AAVRH10, OVINE | AAV13, B19 |
| AAV11, PORCINE | AAV12, INSECT | AAVRH8, OVINE | AAVRH10, B19 | AAV13, MVM |
| AAV11, INSECT | AAV12, OVINE | AAVRH8, B19 | AAVRH10, MVM | AAV13, GOOSE |
| AAV11, OVINE | AAV12, B19 | AAVRH8, MVM | AAVRH10, GOOSE | AAV13, SNAKE |
| AAV11, B19 | AAV12, MVM | AAVRH8, GOOSE | AAVRH10, SNAKE | |
| AAV11, MVM | AAV12, GOOSE | AAVRH8, SNAKE | | |
| AAV11, GOOSE | AAV12, SNAKE | | | |
| AAV11, SNAKE | | | | |
| AAVDJ, AAVDJ | AAVDJ8, AVVDJ8 | AVIAN, AVIAN | BOVINE, BOVINE | CANINE, CANINE |
| AAVDJ, AAVDJ8 | AAVDJ8, AVIAN | AVIAN, BOVINE | BOVINE, CANINE | CANINE, EQUINE |
| AAVDJ, AVIAN | AAVDJ8, BOVINE | AVIAN, CANINE | BOVINE, EQUINE | CANINE, GOAT |
| AAVDJ, BOVINE | AAVDJ8, CANINE | AVIAN, EQUINE | BOVINE, GOAT | CANINE, SHRIMP |
| AAVDJ, CANINE | AAVDJ8, EQUINE | AVIAN, GOAT | BOVINE, SHRIMP | CANINE, PORCINE |
| AAVDJ, EQUINE | AAVDJ8, GOAT | AVIAN, SHRIMP | BOVINE, PORCINE | CANINE, INSECT |
| AAVDJ, GOAT | AAVDJ8, SHRIMP | AVIAN, PORCINE | BOVINE, INSECT | CANINE, OVINE |
| AAVDJ, SHRIMP | AAVDJ8, PORCINE | AVIAN, INSECT | BOVINE, OVINE | CANINE, B19 |
| AAVDJ, PORCINE | AAVDJ8, INSECT | AVIAN, OVINE | BOVINE, B19 | CANINE, MVM |
| AAVDJ, INSECT | AAVDJ8, OVINE | AVIAN, B19 | BOVINE, MVM | CANINE, GOOSE |
| AAVDJ, OVINE | AAVDJ8, B19 | AVIAN, MVM | BOVINE, GOOSE | CANINE, SNAKE |
| AAVDJ, B19 | AAVDJ8, MVM | AVIAN, GOOSE | BOVINE, SNAKE | |
| AAVDJ, MVM | AAVDJ8, GOOSE | AVIAN, SNAKE | | |
| AAVDJ, GOOSE | AAVDJ8, SNAKE | | | |
| AAVDJ, SNAKE | | | | |
| EQUINE, EQUINE | GOAT, GOAT | SHRIMP, SHRIMP | PORCINE, PORCINE | INSECT, INSECT |
| EQUINE, GOAT | GOAT, SHRIMP | SHRIMP, PORCINE | PORCINE, INSECT | INSECT, OVINE |
| EQUINE, SHRIMP | GOAT, PORCINE | SHRIMP, INSECT | PORCINE, OVINE | INSECT, B19 |
| EQUINE, PORCINE | GOAT, INSECT | SHRIMP, OVINE | PORCINE, B19 | INSECT, MVM |
| EQUINE, INSECT | GOAT, OVINE | SHRIMP, B19 | PORCINE, MVM | INSECT, GOOSE |
| EQUINE, OVINE | GOAT, B19 | SHRIMP, MVM | PORCINE, GOOSE | INSECT, SNAKE |
| EQUINE, B19 | GOAT, MVM | SHRIMP, GOOSE | PORCINE, SNAKE | |
| EQUINE, MVM | GOAT, GOOSE | SHRIMP, SNAKE | | |
| EQUINE, GOOSE | GOAT, SNAKE | | | |
| EQUINE, SNAKE | | | | |
| OVINE, OVINE | B19, B19 | MVM, MVM | GOOSE, GOOSE | SNAKE, SNAKE |
| OVINE, B19 | B19, MVM | MVM, GOOSE | GOOSE, SNAKE | |
| OVINE, MVM | B19, GOOSE | MVM, SNAKE | | |
| OVINE, GOOSE | B19, SNAKE | | | |
| OVINE, SNAKE | | | | |

By way of example only. Table 5 shows the sequences of exemplary WT-ITRs from some different AAV serotypes.

TABLE 5
Exemplary WT-ITRs

| AAV | | |
|---|---|---|
| serotype | 5′ WT-ITR (LEFT) | 3′ WT-ITR (RIGHT) |
| AAV1 | 5′-TTGCCCACTCCCTCTCTGCGCGCTCGC | 5′-TTACCCTAGTGATGGAGTTGCCCACTC |
| | TCGCTCGGTGGGGCCTGCGGACCAAA | CCTCTCTGCGCGCGTCGCTCGCTCGGT |
| | GGTCCGCAGACGGCAGAGGTCTCCTC | GGGGCCGGCAGAGGAGACCTCTGCCG |
| | TGCCGGCCCCACCGAGCGAGCGACGC | TCTGCGGACCTTTGGTCCGCAGGCCCC |
| | GCGCAGAGAGGGAGTGGGCAACTCCA | ACCGAGCGAGCGAGCGCGCAGAGAGG |
| | TCACTAGGGTAA-3′ | GAGTGGGCAA-3′ |
| AAV2 | CCTGCAGGCAGCTGCGCGCTCGCTCG | AGGAACCCCTAGTGATGGAGTTGGCCA |
| | CTCACTGAGGCCGCCCGGGCAAAGCC | CTCCCTCTCTGCGCGCTCGCTCGCTCAC |
| | CGGGCGTCGGGCGACCTTTGGTCGCC | TGAGGCCGGGCGACCAAAGGTCGCCC |
| | CGGCCTCAGTGAGCGAGCGAGCGCGC | GACGCCCGGGCTTTGCCCGGGCGGCCT |
| | AGAGAGGGAGTGGCCAACTCCATCAC | CAGTGAGCGAGCGAGCGCGCAGCTGC |
| | TAGGGGTTCCT | CTGCAGG |
| AAV3 | 5′-TTGGCCACTCCCTCTATGCGCACTCGC | 5′-ATACCTCTAGTGATGGAGTTGGCCACT |
| | TCGCTCGGTGGGGCCTGGCGACCAAA | CCCTCTATGCGCACTCGCTCGCTCGGT |
| | GGTCGCCAGACGGACGTGGGTTTCCA | GGGGCCGGACGTGGAAACCCACGTCC |
| | CGTCCGGCCCCACCGAGCGAGCGAGT | GTCTGGCGACCTTTGGTCGCCAGGCCC |
| | GCGCATAGAGGGAGTGGCCAACTCCA | CACCGAGCGAGCGAGTGCGCATAGAG |
| | TCACTAGAGGTAT-3′ | GGAGTGGCCAA-3′ |
| AAV4 | 5′-TTGGCCACTCCCTCTATGCGCGCTCGC | 5′-AGTTGGCCACATTAGCTATGCGCGCTC |
| | TCACTCACTCGGCCCTGGAGACCAAA | GCTCACTCACTCGGCCCTGGAGACCAA |
| | GGTCTCCAGACTGCCGGCCTCTGGCC | AGGTCTCCAGACTGCCGGCCTCTGGCC |
| | GGCAGGGCCGAGTGAGTGAGCGAGC | GGCAGGGCCGAGTGAGTGAGCGAGCG |
| | GCGCATAGAGGGAGTGGCCAACT-3′ | CGCATAGAGGGAGTGGCCAA-3′ |
| AAV5 | 5′-TCCCCCCTGTCGCGTTCGCTCGCTCGC | 5′-CTTACAAAACCCCCTTGCTTGAGAGTG |
| | TGGCTCGTTTGGGGGGGCGACGGCCA | TGGCACTCTCCCCCCTGTCGCGTTCGCT |
| | GAGGGCCGTCGTCTGGCAGCTCTTTG | CGCTCGCTGGCTCGTTTGGGGGGGTGG |
| | AGCTGCCACCCCCCCAAACGAGCCAG | CAGCTCAAAGAGCTGCCAGACGACGG |
| | CGAGCGAGCGAACGCGACAGGGGGG | CCCTCTGGCCGTCGCCCCCCCAAACGA |
| | AGAGTGCCACACTCTCAAGCAAGGGG | GCCAGCGAGCGAGCGAACGCGACAGG |
| | GTTTTGTAAG -3′ | GGGGA-3′ |
| AAV6 | 5′-TTGCCCACTCCCTCTAATGCGCGCTCG | 5′-ATACCCCTAGTGATGGAGTTGCCCACT |
| | CTCGCTCGGTGGGGCCTGCGGACCAA | CCCTCTATGCGCGCTCGCTCGCTCGGT |
| | AGGTCCGCAGACGGCAGAGGTCTCCT | GGGGCCGGCAGAGGAGACCTCTGCCG |
| | CTGCCGGCCCCACCGAGCGAGCGAGC | TCTGCGGACCTTTGGTCCGCAGGCCCC |
| | GCGCATAGAGGGAGTGGGCAACTCCA | ACCGAGCGAGCGAGCGCGCATTAGAG |
| | TCACTAGGGGTAT-3′ | GGAGTGGGCAA |

According to some embodiments, the nucleic acid sequence of the WT-ITR sequence can be modified (e.g., by modifying 1, 2, 3, 4 or 5, or more nucleotides or any range therein), whereby the modification is a substitution for a complementary nucleotide, e.g., G for a C, and vice versa, and T for an A, and vice versa.

The ceDNA vector for expression of anti-CoV S antibodies and antigen-binding fragments thereof (e.g., anti-CoV-2 S antibodies and antigen-binding fragments thereof) as described herein can include WT-ITR structures that retains an operable RBE, trs and RBE′ portion. FIG. 2A and FIG. 2B, using wild-type ITRs for exemplary purposes, show one possible mechanism for the operation of a trs site within a wild type ITR structure portion of a ceDNA vector. According to some embodiments, the ceDNA vector for expression of anti-CoV-2 S antibodies and antigen-binding fragments thereof contains one or more functional WT-ITR polynucleotide sequences that comprise a Rep-binding site (RBS; 5′-GCGCGCTCGCTCGCTC-3′ for AAV2) and a terminal resolution site (TRS; 5′-AGTT). According to some embodiments, at least one WT-ITR is functional. In alternative embodiments, where a ceDNA vector for expression of anti-CoV S antibodies and antigen-binding fragments thereof (e.g., anti-CoV-2 S antibodies and antigen-binding fragments thereof) comprises two WT-ITRs that are substantially symmetrical to each other, at least one WT-ITR is functional and at least one WT-ITR is non-functional.

B. Modified ITRs (Mod-ITRs) in General for ceDNA Vectors Comprising Asymmetric ITR Pairs or Symmetric ITR Pairs

As discussed herein, a ceDNA vector for expression of anti-CoV-2 S antibodies and antigen-binding fragments thereof can comprise a symmetrical ITR pair or an asymmetrical ITR pair. In both instances, one or both of the ITRs can be modified ITRs—the difference being that in the first instance (i.e., symmetric mod-ITRs), the mod-ITRs have the same three-dimensional spatial organization (i.e., have the same A-A′, C-C′ and B-B′ arm configurations), whereas in the second instance (i.e., asymmetric mod-ITRs), the mod-ITRs have a different three-dimensional spatial organization (i.e., have a different configuration of A-A′, C-C′ and B-B′ arms).

According to some embodiments, a modified ITR is an ITRs that is modified by deletion, insertion, and/or substitution as compared to a wild-type ITR sequence (e.g., AAV ITR). According to some embodiments, at least one of the ITRs in the ceDNA vector comprises a functional Rep binding site (RBS; e.g., 5′-GCGCGCTCGCTCGCTC-3′ for AAV2) and a functional terminal resolution site (TRS; e.g., 5′-AGTT-3′) According to some embodiment, at least one of the ITRs is a non-functional ITR. According to some embodiment, the different or modified ITRs are not each wild type ITRs from different serotypes.

Specific alterations and mutations in the ITRs are described in detail herein, but in the context of ITRs, “altered” or “mutated” or “modified”, it indicates that nucleotides have been inserted, deleted, and/or substituted relative to the wild-type, reference, or original ITR sequence. The altered or mutated ITR can be an engineered ITR. As used herein. “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature.

According to some embodiments, a mod-ITR may be synthetic. According to some embodiment, a synthetic ITR is based on ITR sequences from more than one AAV serotype. In another embodiment, a synthetic ITR includes no AAV-based sequence. In yet another embodiment, a synthetic ITR preserves the ITR structure described above although having only some or no AAV-sourced sequence. According to some aspects, a synthetic ITR may interact preferentially with a wild type Rep or a Rep of a specific serotype, or According to some instances will not be recognized by a wild-type Rep and be recognized only by a mutated Rep.

The skilled artisan can determine the corresponding sequence in other serotypes by known means. For example, determining if the change is in the A, A′, B, B′, C, C′ or D region and determine the corresponding region in another serotype. One can use BLAST® (Basic Local Alignment Search Tool) or other homology alignment programs at default status to determine the corresponding sequence. The disclosure further provides populations and pluralities of ceDNA vectors comprising mod-ITRs from a combination of different AAV serotypes—that is, one mod-ITR can be from one AAV serotype and the other mod-ITR can be from a different serotype. Without wishing to be bound by theory, according to some embodiment one ITR can be from or based on an AAV2 ITR sequence and the other ITR of the ceDNA vector can be from or be based on any one or more ITR sequence of AAV serotype 1 (AAV1), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12).

Any parvovirus ITR can be used as an ITR or as a base ITR for modification. Preferably, the parvovirus is a dependovirus. More preferably AAV. The serotype chosen can be based upon the tissue tropism of the serotype. AAV2 has a broad tissue tropism, AAV1 preferentially targets to neuronal and skeletal muscle, and AAV5 preferentially targets neuronal, retinal pigmented epithelia, and photoreceptors. AAV6 preferentially targets skeletal muscle and lung. AAV8 preferentially targets liver, skeletal muscle, heart, and pancreatic tissues. AAV9 preferentially targets liver, skeletal and lung tissue. According to some embodiment, the modified ITR is based on an AAV2 ITR.

More specifically, the ability of a structural element to functionally interact with a particular large Rep protein can be altered by modifying the structural element. For example, the nucleic acid sequence of the structural element can be modified as compared to the wild-type sequence of the ITR. According to some embodiment, the structural element (e.g., A arm, A′ arm, B arm, B′ arm, C arm, C′ arm, D arm, RBE, RBE′, and trs) of an ITR can be removed and replaced with a wild-type structural element from a different parvovirus. For example, the replacement structure can be from AAV1, AAV2, AAV3, AAV4, AAV5, AAV6, AAV7, AAV8, AAV9, AAV10, AAV11, AAV12, AAV13, snake parvovirus (e.g., royal python parvovirus), bovine parvovirus, goat parvovirus, avian parvovirus, canine parvovirus, equine parvovirus, shrimp parvovirus, porcine parvovirus, or insect AAV. For example, the ITR can be an AAV2 ITR and the A or A′ arm or RBE can be replaced with a structural element from AAV5. In another example, the ITR can be an AAV5 ITR and the C or C′ arms, the RBE, and the trs can be replaced with a structural element from AAV2. In another example, the AAV ITR can be an AAV5 ITR with the B and B′ arms replaced with the AAV2 ITR B and B′ arms.

By way of example only, Table 6 indicates exemplary modifications of at least one nucleotide (e.g., a deletion, insertion and/or substitution) in regions of a modified ITR, where X is indicative of a modification of at least one nucleic acid (e.g., a deletion, insertion and/or substitution) in that section relative to the corresponding wild-type ITR. According to some embodiments, any modification of at least one nucleotide (e.g., a deletion, insertion and/or substitution) in any of the regions of C and/or C′ and/or B and/or B′ retains three sequential T nucleotides (i.e., TTT) in at least one terminal loop. For example, if the modification results in any of: a single arm ITR (e.g., single C-C′ arm, or a single B-B′ arm), or a modified C-B′ arm or C′-B arm, or a two arm ITR with at least one truncated arm (e.g., a truncated C-C′ arm and/or truncated B-B′ arm), at least the single arm, or at least one of the arms of a two arm ITR (where one arm can be truncated) retains three sequential T nucleotides (i.e., TTT) in at least one terminal loop. According to some embodiments, a truncated C-C′ arm and/or a truncated B-B′ arm has three sequential T nucleotides (i.e., TTT) in the terminal loop.

TABLE 6
Exemplary combinations of modifications of at least one nucleotide
(e.g., a deletion, insertion and/or substitution) to different
B-B′ and C-C′ regions or arms of ITRs (X indicates
a nucleotide modification, e.g., addition, deletion or substitution
of at least one nucleotide in the region).

| B region | B′ region | C region | C′ region |
|---|---|---|---|
| X | | | |
| | X | | |
| X | X | | |
| | | X | |
| | | | X |
| | | X | X |
| X | | X | |
| X | | | X |
| | X | X | |
| | X | | X |
| X | X | X | |
| X | X | | X |
| X | | X | X |
| | X | X | X |
| X | X | X | X |

According to some embodiments, mod-ITR for use in a ceDNA vector for expression of anti-CoV-2 S antibodies and antigen-binding fragments thereof) comprises an asymmetric ITR pair, or a symmetric mod-ITR pair as disclosed herein, can comprise any one of the combinations of modifications shown in Table 6, and also a modification of at least one nucleotide in any one or more of the regions selected from: between A′ and C, between C and C′, between C′ and B, between B and B′ and between B′ and A. According to some embodiments, any modification of at least one nucleotide (e.g., a deletion, insertion and/or substitution) in the C or C′ or B or B′ regions, still preserves the terminal loop of the stem-loop. According to some embodiments, any modification of at least one nucleotide (e.g., a deletion, insertion and/or substitution) between C and C′ and/or B and B′ retains three sequential T nucleotides (i.e., TTT) in at least one terminal loop. In alternative embodiments, any modification of at least one nucleotide (e.g., a deletion, insertion and/or substitution) between C and C′ and/or B and B′ retains three sequential A nucleotides (i.e., AAA) in at least one terminal loop. According to some embodiments, a modified ITR for use herein can comprise any one of the combinations of modifications shown in Table 6, and also a modification of at least one nucleotide (e.g., a deletion, insertion and/or substitution) in any one or more of the regions selected from: A′, A and/or D. For example, according to some embodiments, a modified ITR for use herein can comprise any one of the combinations of modifications shown in Table 6, and also a modification of at least one nucleotide (e.g., a deletion, insertion and/or substitution) in the A region. According to some embodiments, a modified ITR for use herein can comprise any one of the combinations of modifications shown in Table 6, and also a modification of at least one nucleotide (e.g., a deletion, insertion and/or substitution) in the A′ region. According to some embodiments, a modified ITR for use herein can comprise any one of the combinations of modifications shown in Table 6, and also a modification of at least one nucleotide (e.g., a deletion, insertion and/or substitution) in the A and/or A′ region. According to some embodiments, a modified ITR for use herein can comprise any one of the combinations of modifications shown in Table 6, and also a modification of at least one nucleotide (e.g., a deletion, insertion and/or substitution) in the D region.

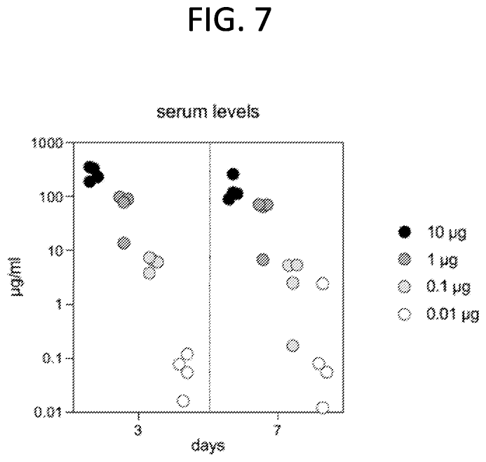

According to some embodiment, the nucleotide sequence of the structural element can be modified (e.g., by modifying 1, 2, 3, 4, 5, 6, 7, 8, 9, 10, 11, 12, 13, 14, 15, 16, 17, 18, 19, or 20 or more nucleotides or any range therein) to produce a modified structural element. According to some embodiment, the specific modifications to the ITRs are exemplified herein (e.g., SEQ ID NOS: 3, 4, 15-47, 101-116 or 165-187, or shown in FIG. 7A-7B of International Patent Application No. PCT/US2018/064242, filed on Dec. 6, 2018 (e.g., SEQ ID Nos 97-98, 101-103, 105-108, 111-112, 117-134, 545-54 in PCT/US2018/064242). According to some embodiments, an ITR can be modified (e.g., by modifying 1, 2, 3, 4, 5, 6, 7, 8, 9, 10, 11, 12, 13, 14, 15, 16, 17, 18, 19, or 20 or more nucleotides or any range therein). In other embodiments, the ITR can have at least 80%, at least 85%, at least 90%, at least 95%, at least 96%, at least 97%, at least 98%, at least 99%, or more sequence identity with one of the modified ITRs of SEQ ID NOS: 3, 4, 15-47, 101-116 or 165-187, or the RBE-containing section of the A-A′ arm and C-C′ and B-B′ arms of SEQ ID NO: 3, 4, 15-47, 101-116 or 165-187, or shown in Tables 2-9 (i.e., SEQ ID NO: 110-112, 115-190, 200-468) of International Patent Application No. PCT/US18/49996, which is incorporated herein in its entirety by reference.

According to some embodiments, a modified ITR can for example, comprise removal or deletion of all of a particular arm, e.g., all or part of the A-A′ arm, or all or part of the B-B′ arm or all or part of the C-C′ arm, or alternatively, the removal of 1, 2, 3, 4, 5, 6, 7, 8, 9 or more base pairs forming the stem of the loop so long as the final loop capping the stem (e.g., single arm) is still present (e.g., see ITR-21 in FIG. 7A of PCT/US2018/064242, filed Dec. 6, 2018). According to some embodiments, a modified ITR can comprise the removal of 1, 2, 3, 4, 5, 6, 7, 8, 9 or more base pairs from the B-B′ arm. According to some embodiments, a modified ITR can comprise the removal of 1, 2, 3, 4, 5, 6, 7, 8, 9 or more base pairs from the C-C′ arm (see, e.g., ITR-1 in FIG. 3B, or ITR-45 in FIG. 7A of International Patent Application No. PCT/US2018/064242, filed Dec. 6, 2018). According to some embodiments, a modified ITR can comprise the removal of 1, 2, 3, 4, 5, 6, 7, 8, 9 or more base pairs from the C-C′ arm and the removal of 1, 2, 3, 4, 5, 6, 7, 8, 9 or more base pairs from the B-B′ arm. Any combination of removal of base pairs is envisioned, for example, 6 base pairs can be removed in the C-C′ arm and 2 base pairs in the B-B′ arm. As an illustrative example, FIG. 3B shows an exemplary modified ITR with at least 7 base pairs deleted from each of the C portion and the C′ portion, a substitution of a nucleotide in the loop between C and C′ region, and at least one base pair deletion from each of the B region and B′ regions such that the modified ITR comprises two arms where at least one arm (e.g., C-C′) is truncated. According to some embodiments, the modified ITR also comprises at least one base pair deletion from each of the B region and B′ regions, such that the B-B′ arm is also truncated relative to WT ITR.

According to some embodiments, a modified ITR can have between 1 and 50 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) nucleotide deletions relative to a full-length wild-type ITR sequence. According to some embodiments, a modified ITR can have between 1 and 30 nucleotide deletions relative to a full-length WT ITR sequence. According to some embodiments, a modified ITR has between 2 and 20 nucleotide deletions relative to a full-length wild-type ITR sequence.

According to some embodiments, a modified ITR does not contain any nucleotide deletions in the RBE-containing portion of the A or A′ regions, so as not to interfere with DNA replication (e.g., binding to an RBE by Rep protein, or nicking at a terminal resolution site). According to some embodiments, a modified ITR encompassed for use herein has one or more deletions in the B, B′, C, and/or C region as described herein.

In another embodiment, the structure of the structural element can be modified. For example, the structural element a change in the height of the stem and/or the number of nucleotides in the loop. For example, the height of the stem can be about 2, 3, 4, 5, 6, 7, 8, or 9 nucleotides or more or any range therein. According to some embodiment, the stem height can be about 5 nucleotides to about 9 nucleotides and functionally interacts with Rep. In another embodiment, the stem height can be about 7 nucleotides and functionally interacts with Rep. In another example, the loop can have 3, 4, 5, 6, 7, 8, 9, or 10 nucleotides or more or any range therein.

In another embodiment, the number of GAGY binding sites or GAGY-related binding sites within the RBE or extended RBE can be increased or decreased. According to some example, the RBE or extended RBE, can comprise 1, 2, 3, 4, 5, or 6 or more GAGY binding sites or any range therein. Each GAGY binding site can independently be an exact GAGY sequence or a sequence similar to GAGY as long as the sequence is sufficient to bind a Rep protein.

In another embodiment, the spacing between two elements (such as but not limited to the RBE and a hairpin) can be altered (e.g., increased or decreased) to alter functional interaction with a large Rep protein. For example, the spacing can be about 1, 2, 3, 4, 5, 6, 7, 8, 9, 10, 11, 12, 13, 14, 15, 16, 17, 18, 19, 20, or 21 nucleotides or more or any range therein.

The ceDNA vector for expression of anti-CoV-2 S antibodies and antigen-binding fragments thereof as described herein can include an ITR structure that is modified with respect to the wild type AAV2 ITR structure disclosed herein, but still retains an operable RBE, trs and RBE′ portion. FIG. 2A and FIG. 2B show one possible mechanism for the operation of a trs site within a wild type ITR structure portion of a ceDNA vector for expression of anti-CoV-2 S antibodies and antigen-binding fragments thereof. According to some embodiments, the ceDNA vector for expression of anti-CoV-2 S antibodies and antigen-binding fragments thereof contains one or more functional ITR polynucleotide sequences that comprise a Rep-binding site (RBS; 5′-GCGCGCTCGCTCGCTC-3′ for AAV2) and a terminal resolution site (TRS). According to some embodiments, at least one ITR (wt or modified ITR) is functional. In alternative embodiments, where a ceDNA vector for expression of anti-CoV-2 S antibodies and antigen-binding fragments thereof comprises two modified ITRs that are different or asymmetrical to each other, at least one modified ITR is functional and at least one modified ITR is non-functional.

According to some embodiments, the modified ITR (e.g., the left or right ITR) of a ceDNA vector for expression of anti-CoV S antibodies and antigen-binding fragments thereof (e.g., anti-CoV-2 S antibodies and antigen-binding fragments thereof) as described herein has modifications within the loop arm, the truncated arm, or the spacer. Exemplary sequences of ITRs having modifications within the loop arm, the truncated arm, or the spacer are listed in Table 2 (i.e., SEQ ID NOS: 135-190, 200-233); Table 3 (e.g., SEQ ID Nos: 234-263); Table 4 (e.g., SEQ ID NOs: 264-293); Table 5 (e.g., SEQ ID Nos: 294-318 herein); Table 6 (e.g., SEQ ID NO: 319-468; and Tables 7-9 (e.g., SEQ ID Nos: 101-110, 111-112, 115-134) or Table 10A or 10B (e.g., SEQ ID Nos: 9, 100, 469-483, 484-499) of International Patent Application No. PCT/US18/49996, which is incorporated herein in its entirety by reference.

According to some embodiments, the modified ITR for use in a ceDNA vector for expression of anti-CoV-2 S antibodies and antigen-binding fragments thereof comprising an asymmetric ITR pair, or symmetric mod-ITR pair is selected from any or a combination of those shown in Tables 2, 3, 4, 5, 6, 7, 8, 9 and 10A-10B of International Patent Application No. PCT/US18/49996 which is incorporated herein in its entirety by reference.

According to some embodiment, a ceDNA vector for expression of anti-CoV-2 S antibodies and antigen-binding fragments thereof comprises, in the 5′ to 3′ direction: a first adeno-associated virus (AAV) inverted terminal repeat (ITR), a nucleic acid sequence of interest (for example an expression cassette as described herein) and a second AAV ITR, where the first ITR (5′ ITR) and the second ITR (3′ ITR) are asymmetric with respect to each other—that is, they have a different 3D-spatial configuration from one another. As an exemplary embodiment, the first ITR can be a wild-type ITR and the second ITR can be a mutated or modified ITR, or vice versa, where the first ITR can be a mutated or modified ITR and the second ITR a wild-type ITR. According to some embodiment, the first ITR and the second ITR are both mod-ITRs, but have different sequences, or have different modifications, and thus are not the same modified ITRs, and have different 3D spatial configurations. Stated differently, a ceDNA vector with asymmetric ITRs comprises ITRs where any changes According to some ITR relative to the WT-ITR are not reflected in the other ITR; or alternatively, where the asymmetric ITRs have a modified asymmetric ITR pair can have a different sequence and different three-dimensional shape with respect to each other.

In an alternative embodiment, a ceDNA vector for expression of anti-CoV-2 S antibodies and antigen-binding fragments thereof comprises two symmetrical mod-ITRs—that is, both ITRs have the same sequence, but are reverse complements (inverted) of each other. According to some embodiments, a symmetrical mod-ITR pair comprises at least one or any combination of a deletion, insertion, or substitution relative to wild type ITR sequence from the same AAV serotype. The additions, deletions, or substitutions in the symmetrical ITR are the same but the reverse complement of each other. For example, an insertion of 3 nucleotides in the C region of the 5′ ITR would be reflected in the insertion of 3 reverse complement nucleotides in the corresponding section in the C′ region of the 3′ ITR. Solely for illustration purposes only, if the addition is AACG in the 5′ ITR, the addition is CGTT in the 3′ ITR at the corresponding site. For example, if the 5′ ITR sense strand is ATCGATCG with an addition of AACG between the G and A to result in the sequence ATCGAACGATCG. The corresponding 3′ ITR sense strand is CGATCGAT (the reverse complement of ATCGATCG) with an addition of CGTT (i.e., the reverse complement of AACG) between the T and C to result in the sequence CGATCGTTCGAT (the reverse complement of ATCGAACGATCG).

In alternative embodiments, the modified ITR pair are substantially symmetrical as defined herein—that is, the modified ITR pair can have a different sequence but have corresponding or the same symmetrical three-dimensional shape. For example, one modified ITR can be from one serotype and the other modified ITR be from a different serotype, but they have the same mutation (e.g., nucleotide insertion, deletion or substitution) in the same region. Stated differently, for illustrative purposes only, a 5′ mod-ITR can be from AAV2 and have a deletion in the C region, and the 3′ mod-ITR can be from AAV5 and have the corresponding deletion in the C′ region, and provided the 5′ mod-ITR and the 3′ mod-ITR have the same or symmetrical three-dimensional spatial organization, they are encompassed for use herein as a modified ITR pair.

According to some embodiments, a substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR. By way of example only, substantially symmetrical ITRs can have a symmetrical spatial organization such that their structure is the same shape in geometrical space. This can occur, e.g., when a G-C pair is modified, for example, to a C-G pair or vice versa, or A-T pair is modified to a T-A pair, or vice versa. Therefore, using the exemplary example above of modified 5′ ITR as a ATCGAACGATCG, and modified 3′ ITR as CGATCGTTCGAT (i.e., the reverse complement of ATCGAACGATCG, these modified ITRs would still be symmetrical if, for example, the 5′ ITR had the sequence of ATCGAACCATCG, where G in the addition is modified to C, and the substantially symmetrical 3′ ITR has the sequence of CGATCGTTCGAT, without the corresponding modification of the T in the addition to a. According to some embodiments, such a modified ITR pair are substantially symmetrical as the modified ITR pair has symmetrical stereochemistry.

C. Exemplary ceDNA Vectors

As described above, the present disclosure relates to recombinant ceDNA expression vectors and ceDNA vectors that encodeanti-CoV-2 S antibodies and antigen-binding fragments thereof, comprising any one of: an asymmetrical ITR pair, a symmetrical ITR pair, or substantially symmetrical ITR pair as described above. In certain embodiments, the disclosure relates to recombinant ceDNA vectors for expression of anti-CoV-2 S antibodies and antigen-binding fragments thereof having flanking ITR sequences and a transgene, where the ITR sequences are asymmetrical, symmetrical or substantially symmetrical relative to each other as defined herein, and the ceDNA further comprises a nucleic acid sequence of interest located between the flanking ITRs, wherein said nucleic acid molecule is devoid of viral capsid protein coding sequences.

The ceDNA expression vector for expression of anti-CoV-2 S antibodies and antigen-binding fragments thereof may be any ceDNA vector that can be conveniently subjected to recombinant DNA procedures including nucleic acid sequence(s) as described herein, provided at least one ITR is altered. The ceDNA vectors for expression of anti-CoV-2 S antibodies and antigen-binding fragments thereof of the present disclosure are compatible with the host cell into which the ceDNA vector is to be introduced. In certain embodiments, the ceDNA vectors may be linear. As used herein “transgene”, “nucleic acid sequence” and “heterologous nucleic acid sequence” are synonymous, and encode anti-CoV-2 S antibodies and antigen-binding fragments thereof (e.g., antibody HC and/or antibody LC, as described herein.

Referring now to FIGS. 1A-1G, schematics of the functional components of two non-limiting plasmids useful in making a ceDNA vector for expression of anti-CoV S antibodies and antigen-binding fragments thereof (e.g., anti-CoV-2 S antibodies and antigen-binding fragments thereof) are shown. FIG. 1A, 1B, 1D, 1F show the construct of ceDNA vectors or the corresponding sequences of ceDNA plasmids for expression of anti-CoV-2 S antibodies and antigen-binding fragments thereof. ceDNA vectors are capsid-free and can be obtained from a plasmid encoding in this order: a first ITR, an expressible transgene cassette and a second ITR, where the first and second ITR sequences are asymmetrical, symmetrical or substantially symmetrical relative to each other as defined herein. ceDNA vectors for expression of anti-CoV-2 S antibodies and antigen-binding fragments thereof are capsid-free and can be obtained from a plasmid encoding in this order: a first ITR, an expressible transgene (protein or nucleic acid) and a second ITR, where the first and second ITR sequences are asymmetrical, symmetrical or substantially symmetrical relative to each other as defined herein. According to some embodiments, the expressible transgene cassette includes, as needed: an enhancer/promoter, one or more homology arms, a donor sequence, a post-transcription regulatory element (e.g., WPRE,), and a polyadenylation and termination signal (e.g., BGH polyA).

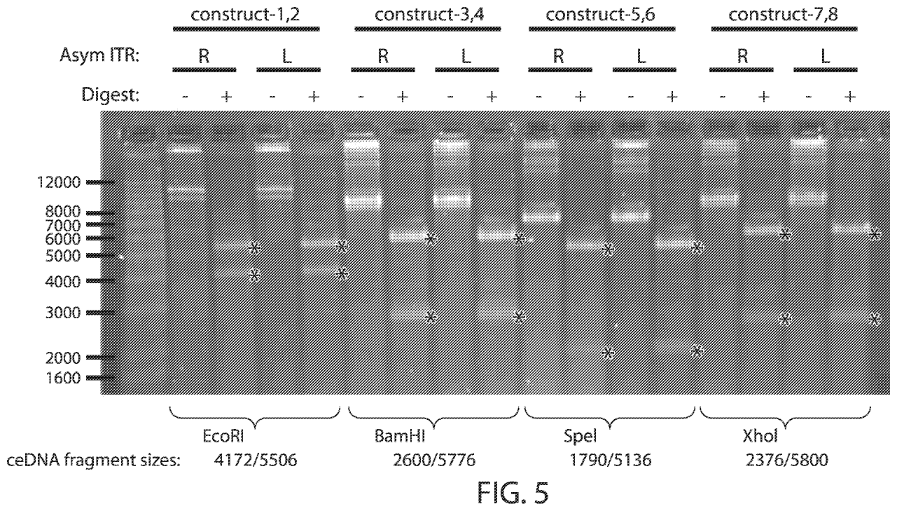

FIG. 5 is a gel confirming the production of ceDNA from multiple plasmid constructs using the method described in the Examples. The ceDNA is confirmed by a characteristic band pattern in the gel, as discussed with respect to FIG. 4A above and in the Examples.

### (i) Regulatory Elements

The ceDNA vectors for expression of anti-CoV-2 S antibodies and antigen-binding fragments thereof as described herein comprising an asymmetric ITR pair or symmetric ITR pair as defined herein, can further comprise a specific combination of cis-regulatory elements. Cis-regulatory elements include, but are not limited to, a promoter, a riboswitch, an insulator, a mir-regulatable element, a post-transcriptional regulatory element, a tissue- and cell type-specific promoter and an enhancer.

A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to the cell, tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter, as well as the promoters listed below. Such promoters and/or enhancers can be used for expression of any gene of interest, e.g., the gene editing molecules, donor sequence, therapeutic proteins etc.). For example, the vector may comprise a promoter that is operably linked to the nucleic acid sequence encoding a therapeutic protein. The promoter operably linked to the therapeutic protein coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metallothionein. The promoter may also be a tissue specific promoter, such as a liver specific promoter, such as human alpha 1-antitrypsin (HAAT), natural or synthetic.

In one embodiment, the promoter used is the native promoter of the gene encoding the therapeutic protein. The promoters and other regulatory sequences for the respective genes encoding the therapeutic proteins are known and have been characterized. The promoter region used may further include one or more additional regulatory sequences (e.g., native), e.g., enhancers.

### (ii). Polyadenylation Sequences:

A sequence encoding a polyadenylation sequence can be included in the ceDNA vector for expression of anti-CoV-2 S antibodies and antigen-binding fragments thereof to stabilize an mRNA expressed from the ceDNA vector, and to aid in nuclear export and translation. According to some embodiment, the ceDNA vector does not include a polyadenylation sequence. In other embodiments, the ceDNA vector for expression of anti-CoV-2 S antibodies and antigen-binding fragments thereof includes at least 1, at least 2, at least 3, at least 4, at least 5, at least 10, at least 15, at least 20, at least 25, at least 30, at least 40, least 45, at least 50 or more adenine dinucleotides. According to some embodiments, the polyadenylation sequence comprises about 43 nucleotides, about 40-50 nucleotides, about 40-55 nucleotides, about 45-50 nucleotides, about 35-50 nucleotides, or any range there between.

The expression cassettes can include any poly-adenylation sequence known in the art or a variation thereof. Some expression cassettes can also include SV40 late polyA signal upstream enhancer (USE) sequence. According to some embodiments, a USE sequence can be used in combination with SV40 pA or heterologous poly-A signal. PolyA sequences are located 3′ of the transgene encoding the anti-CoV-2 S antibodies and antigen-binding fragments thereof.

The expression cassettes can also include a post-transcriptional element to increase the expression of a transgene. According to some embodiments, Woodchuck Hepatitis Virus (WHP) posttranscriptional regulatory element (WPRE) is used to increase the expression of a transgene. Other posttranscriptional processing elements such as the post-transcriptional element from the thymidine kinase gene of herpes simplex virus, or hepatitis B virus (HBV) can be used.

According to some embodiments, one or more nucleic acid sequences that encode an anti-SARS-CoV-2 S antibody, or antigen-binding fragment thereof can also encode a secretory sequence so that the protein is directed to the Golgi Apparatus and Endoplasmic Reticulum and folded into the correct conformation by chaperone molecules as it passes through the ER and out of the cell. Exemplary secretory sequences include, but are not limited to VH-02 and VK-A26 and Igκ signal sequence, as well as a Gluc secretory signal that allows the tagged protein to be secreted out of the cytosol, TMD-ST secretory sequence, that directs the tagged protein to the golgi.

D. Exemplary ceDNA Anti-CoV-2 S Vectors

According to some embodiments, an exemplary capsid-free close-ended DNA (ceDNA) vector comprising at least one nucleic acid sequence comprising an anti-CoV-2 S antibody or antigen-binding fragment thereof, comprises an ORF selected from a sequence shown in Table 7, below.

TABLE 7

| Construct | Sequence Identifier |
|---|---|
| ceDNA-1856 | SEQ ID NO: 25 |
| ceDNA-1859 | SEQ ID NO: 26 |
| ceDNA-2157 | SEQ ID NO: 27 |

According to some embodiments, the exemplary ceDNA is ceDNA-1856, comprising an ORF comprising SEQ ID NO:25, shown below.

CCTGCAGGCA GCTGCGCGCT CGCTCGCTCA CTGAGGCCGC CCGGGCAAAG
CCCGGGCGTCGGGCGACCTT TGGTCGCCCG GCCTCAGTGA GCGAGCGAGC
GCGCAGAGAG GGAGTGGCCA ACTCCATCAC TAGGGGTTCC TTGTAGTTAA
TGATTAACCC GCCATGCTAC TTATCGCGGC CGCGGGGGAG GCTGCTGGTG
AATATTAACC AAGGTCACCC CAGTTATCGG AGGAGCAAAC AGGGGCTAAG
TCCACCGGGG GAGGCTGCTG GTGAATATTA ACCAAGGTCA CCCCAGTTAT
CGGAGGAGCA AACAGGGGCT AAGTCCACCG GGGGAGGCTG CTGGTGAATA
TTAACCAAGG TCACCCCAGT TATCGGAGGA GCAAACAGGG GCTAAGTCCA
CGGTACCCAC TGGGAGGATG TTGAGTAAGA TGGAAAACTA CTGATGACCC
TTGCAGAGAC AGAGTATTAG GACATGTTTG AACAGGGGCC GGGCGATCAG
CAGGTAGCTC TAGAGGATCC CCGTCTGTCT GCACATTTCG TAGAGCGAGT
GTTCCGATAC TCTAATCTCC CTAGGCAAGG TTCATATTTG TGTAGGTTAC
TTATTCTCCT TTTGTTGACT AAGTCAATAA TCAGAATCAG CAGGTTTGGA
GTCAGCTTGG CAGGGATCAG CAGCCTGGGT TGGAAGGAGG GGGTATAAAA
GCCCCTTCAC CAGGAGAAGC CGTCACACAG ATCCACAAGC TCCTGAAGAG
GTAAGGGTTT AAGGGATGGT TGGTTGGTGG GGTATTAATG TTTAATTACC
TGGAGCACCT GCCTGAAATC ACTTTTTTTC AGGTTGGGTT TAAACCGCAG
CCACCATGGA CATGAGAGTG CCCGCCCAGC TGCTGGGCCT GCTGCTGCTG
TGGCTGTCCG GAGCCAGATG CGAAATCGTG CTGACCCAGA GCCCTGGCAC
CCTGAGCCTGTCACCCGGCG AACGGGCTAC CCTGTCTTGT AGAGCCTCTC
AGACCGTGTC CAGCACCAGCCTGGCCTGGT ACCAGCAGAA ACCTGGACAG
GCTCCTAGAC TGCTGATCTA CGGAGCTTCTAGTAGAGCCA CCGGCATCCC
CGATAGATTC AGCGGCAGCG GCAGCGGCAC TGATTTCACCCTGACAATTA
GCCGGCTGGA ACCTGAGGAC TTTGCCGTGT ATTACTGCCA GCAACACGAC
ACCAGCCTGA CATTCGGCGG CGGAACCAAA GTTGAGATCA AGCGGACCGT
GGCCGCTCCATCTGTGTTCA TCTTTCCACC TAGCGACGAG CAGCTGAAGT
CCGGCACAGC CTCTGTGGTGTGCCTGCTCA ACAACTTCTA CCCTCGCGAG
GCCAAGGTGC AGTGGAAGGT GGACAACGCCCTGCAAAGCG GCAACAGCCA
GGAGAGCGTC ACAGAACAGG ACAGCAAGGA CTCTACATACAGCCTGAGCA
GCACACTGAC CCTCAGCAAG GCCGATTACG AGAAGCACAA GGTTTACGCCT
GCGAGGTGA CCCACCAGGG CCTGTCCAGC CCTGTGACAAAGAGCTTCAA
TAGAGGCGAATGTTGATAGT TAATTAAGAG CATCTTACCGC CATTTAT
TCCCATATTTGT TCTGTTTTTCTTGATTTGGG TATACATTTA AATGTTAATA
AAACAAAATG GTGGGGCAAT CATTTACATTTTTAGGGATA TGTAATTACTAG
TTCAGGTG TATTGCCACA AGACAAACAT GTTAAGAAACTTTCCCGTTA TTTACGCTCT
GTTCCTGTTA ATCAACCTCT GGATTACAAA ATTTGTGAA AGATTGACTGA
TATTCTTAAC TATGTTGCTC CTTTTACGCT GTGTGGATAT GCTGCTTTATA GCCTCTGTA
TCTAGCTAT TGCTTCCCGTA CGGCTTTCGT TTTCTCCTCC TTGTATAAATC CTGGTTGCT
GTCTCTTTTA GAGGAGTTGT GGCCCGTTGT CCGTCAACGT GGCGTGGTG
TGCTCTGTGTT TGCTGACGCAACCCCCACTG GCTGGGGCAT TGCCACCACC
TGTCAACTCCTTTCTGGGACTTTCGCTTTC CCCCTCCCGA TCGCCACGGC AGAACTCATC
GCCGCCTGCCTTGCCCGCTG CTGGACAGGG GCTAGGTTGC TGGGCACTGA
TAATTCCGTG GTGTTGTCTGTGCCTTCTAG TTGCCAGCCA TCTGTTGTTT GCCCCTCCCC
CGTGCCTTCC TTGACCCTGGAAGGTGCCAC TCCCACTGTC CTTTCCTAAT
AAAATGAGGA AATTGCATCG CATTGTCTGAGTAGGTGTCA TTCTATTCTG
GGGGGTGGGG TGGGGCAGGA CAGCAAGGGG GAGGATTG GGAAGACAATAG
CAGGCATGCT GGGGATGCGG TGGGCTCTAT GGCTCTAGAG
CATGGCTACGTAGATAAGTA GCATGGCGGG TTAATCATTA ACTACACCTG
CAGGAGGAAC CCCTAGTGATGGAGTTGGCC ACTCCCTCTC TGCGCGCTCG
CTCGCTCACT GAGGCCGGGC GACCAAAGGTCGCCCGACGC CCGGGCGGCC
TCAGTGAGCG AGCGAGCGCG CAGCTGCCTG CAGG

According to some embodiments, the ORF is at least 85%, at least 90%, at least 91%, at least 92%, at least 93%, at least 94%, at least 94%, at least 95%, at least 96%, at least 97%, at least 98% or at least 99% identical to SEQ ID NO:25.

According to some embodiments, the exemplary ceDNA vector is ceDNA-1859, comprising an ORF comprising SEQ ID NO:26, shown below.

CCTGCAGGCA GCTGCGCGCT CGCTCGCTCA CTGAGGCCGC CCGGGCAAAG CCCGGGCGTC GGGCGACCTT
TGGTCGCCCG GCCTCAGTGA GCGAGCGAGC GCGCAGAGAG GGAGTGGCCA ACTCCATCAC TAGGGGTTCC
TTGTAGTTAA TGATTAACCC GCCATGCTAC TTATCGCGGC CGCGGGGGAG GCTGCTGGTG AATATTAACC
AAGGTCACCC CAGTTATCGG AGGAGCAAAC AGGGGCTAAG TCCACCGGGG GAGGCTGCTG GTGAATATTA
ACCAAGGTCA CCCCAGTTAT CGGAGGAGCA AACAGGGGCT AAGTCCACCG GGGGAGGCTG CTGGTGAATA
TTAACCAAGG TCACCCCAGT TATCGGAGGA GCAAACAGGG GCTAAGTCCA CGGTACCCAC TGGGAGGATG
TTGAGTAAGA TGGAAAACTA CTGATGACCC TTGCAGAGAC AGAGTATTAG GACATGTTTG AACAGGGGCC
GGGCGATCAG CAGGTAGCTC TAGAGGATCC CCGTCTGTCT GCACATTTCG TAGAGCGAGT GTTCCGATAC
TCTAATCTCC CTAGGCAAGG TTCATATTTG TGTAGGTTAC TTATTCTCCT TTTGTTGACT AAGTCAATAA
TCAGAATCAG CAGGTTTGGA GTCAGCTTGG CAGGGATCAG CAGCCTGGGT TGGAAGGAGG GGGTATAAAA
GCCCCTTCAC CAGGAGAAGC CGTCACACAG ATCCACAAGC TCCTGAAGAG GTAAGGGTTT AAGGGATGGT
TGGTTGGTGG GGTATTAATG TTTAATTACC TGGAGCACCT GCCTGAAATC ACTTTTTTTC AGGTTGGGTT
TAAACCGCAG CCACCATGGA ATTCGGCCTG TCCTGGGTCT TTCTGGTGGC CATCCTGAAG GGCGTGCAGT
GCCAGGTCCA GCTGGTTCAG AGCGGCGCCG AGGTTAAGAA ACCTGGCGCC AGCGTGAAAG TGTCCTGCAA
GGCCAGCGGC TACCCCTTCA CCAGCTACGG CATCTCTTGG GTGCGGCAGG CCCCTGGACA AGGACTGGAG
TGGATGGGAT GGATCAGCAC TTACCAGGGC AATACCAACT ACGCCCAGAA ATTCCAGGGC AGAGTGACAA
TGACCACCGA CACCAGCACAACCACAGGCT ACATGGAACT GCGGAGACTG AGAAGCGATG ATACAGCCGT
GTACTACTGCGCCAGAGATT ATACAAGAGG TGCTTGGTTC GGCGAGAGCC TGATCGGCGG ATTCGACAAC
TGGGGACAAG GCACCCTGGT GACCGTGTCA TCCGCCTCTA CCAAGGGCCC TAGCGTGTTTCCACTGGCCC
CTAGCTCTAA AAGCACAAGC GGCGGCACCG CCGCTCTGGG ATGTCTGGTGAAGGACTACT TCCCAGAGCC
CGTGACCGTG AGCTGGAACA GCGGCGCTCT CACATCTGGGGTGCATACCT TTCCCGCCGT GCTGCAGTCT
TCTGGACTGT ACAGCCTGAG CAGCGTGGTGACCGTGCCCT CCAGCAGCCT GGGCACACAG ACCTACATCT
GCAACGTGAA CCACAAGCCA TCTAATACCA AGGTGGATAA GAAGGTGGAA CCTAAGAGCT GTGACAAGAC
ACACACATGC CCCCCCTGCC CTGCTCCTGA GCTGCTGGCC GGCCCCTCCG TGTTTCTCTT CCCTCCTAAA
CCCAAGGACA CACTGATGAT TAGCCGGACC CCAGAGGTGA CCTGTGTGGT GGTTGACGTG AGTCACGAAG
ATCCTGAAGT GAAGTTCAAC TGGTACGTGG ACGGCGTGGA GGTGCATAAC GCCAAAACCA AGCCTCGGGA
AGAGCAGTAC AACAGCACCT ATAGAGTGGT GAGCGTGCTT ACAGTGCTGC ATCAGGACTG GCTGAACGGC
AAGGAATACA AGTGCAAGGT GTCCAACAAA GCCCTGCCTC TGCCTGAAGA AAAGACCATC AGCAAGGCCA
AGGGCCAACC AAGAGAGCCT CAAGTGTACA CCCTGCCCCC CAGCAGAGAT GAGCTGACCA AGAATCAGGT
GTCCCTGACC TGCCTGGTCA AAGGCTTCTA CCCTAGCGAC ATCGCCGTCG AGTGGGAGAG CAATGGCCAG
CCTGAGAACA ACTACAAGAC CACCCCTCCT GTGCTGGACA GCGACGGCAG CTTCTTCCTG TATAGCAAGC
TGACCGTGGA CAAGTCCAGG TGGCAGCAGG GCAATGTGTT CAGCTGTAGC GTGCTGCACG AGGCCCTGCA
CAGCCACTAC ACACAGAAGT CTCTGAGCCT GTCTCCTGGC AAGTGATAGT TAATTAAGAG CATCTTACCG
CCATTTATTC CCATATTTGT TCTGTTTTTC TTGATTTGGG TATACATTTA AATGTTAATA AAACAAAATG
GTGGGGCAAT CATTTACATT TTTAGGGATA TGTAATTACT AGTTCAGGTG TATTGCCACA AGACAAACAT
GTTAAGAAAC TTTCCCGTTA TTTACGCTCT GTTCCTGTTA ATCAACCTCT GGATTACAAA
ATTTGTGAAAGATTGACTGA TATTCTTAAC TATGTTGCTC CTTTTACGCT GTGTGGATAT GCTGCTTTAT
AGCCTCTGTA TCTAGCTATT GCTTCCCGTA CGGCTTTCGT TTTCTCCTCC TTGTATAAAT CCTGGTTGCT
GTCTCTTTTA GAGGAGTTGT GGCCCGTTGT CCGTCAACGT GGCGTGGTGT GCTCTGTGTT TGCTGACGCA
ACCCCCACTG GCTGGGGCAT TGCCACCACC TGTCAACTCC TTTCTGGGAC TTTCGCTTTC CCCCTCCCGA
TCGCCACGGC AGAACTCATC GCCGCCTGCC TTGCCCGCTG CTGGACAGGG GCTAGGTTGC TGGGCACTGA
TAATTCCGTG GTGTTGTCTG TGCCTTCTAG TTGCCAGCCA TCTGTTGTTT GCCCCTCCCC CGTGCCTTCC
TTGACCCTGG AAGGTGCCAC TCCCACTGTC CTTTCCTAAT AAAATGAGGA AATTGCATCG CATTGTCTGA
GTAGGTGTCA TTCTATTCTG GGGGGTGGGG TGGGGCAGGA CAGCAAGGGG GAGGATTGGG AAGACAATAG
CAGGCATGCT GGGGATGCGG TGGGCTCTAT GGCTCTAGAG ATGGCTACG TAGATAAGTA GCATGGCGGG
TTAATCATTA ACTACACCTG CAGGAGGAAC CCCTAGTGAT GGAGTTGGCC ACTCCCTCTC TGCGCGCTCG
CTCGCTCACT GAGGCCGGGC GACCAAAGGT CGCCCGACGC CCGGGCGGCC TCAGTGAGCG AGCGAGCGCG
CAGCTGCCTG CAGG

According to some embodiments, the ORF is at least 85%, at least 90%, at least 91%, at least 92%, at least 93%, at least 94%, at least 94%, at least 95%, at least 96%, at least 97%, at least 98% or at least 99% identical to SEQ ID NO:26.

According to some embodiments, SEQ ID NO:25 comprises the following components, where the numbers indicate nucleic acid residues:
856 . . . 921=Immunoglobulin kappa variable 1D-33 Signal Peptide
856 . . . 1566=LC1 (Light Chain 1)-codon optimized
922 . . . 1242=S309_VL (variable region of the light chain)
1243 . . . 1563=hIgKappa Constant Region [P01834]
1243 . . . 1563=S309 Constant Region (Human Kappa)

According to some embodiments, SEQ ID NO:26 comprises the following components, where the numbers indicate nucleic acid residues:
856 . . . 912=Human IGHV3-43 heavy chain signal peptide
856 . . . 2286=HC1 Heavy Chain 1 (HC1)-codon optimized
913 . . . 1293=S309_VH (variable region of the heavy chain)
1294 . . . 2283=S309 Constant Region (hIgG1) with GAALIE and LS mutations

According to some embodiments, the exemplary ceDNA vector is ceDNA-2157, comprising SEQ ID NO: 27, shown below.

CCTGCAGGCA GCTGCGCGCT CGCTCGCTCA CTGAGGCCGC CCGGGCAAAG
CCCGGGCGTCGGGCGACCTT TGGTCGCCCG GCCTCAGTGA GCGAGCGAGC
GCGCAGAGAG GGAGTGGCCAACTCCATCAC TAGGGGTTCC TTGTAGTTAA
TGATTAACCC GCCATGCTAC TTATCTACGT AGCCATGCAT ATGTACCACA
TTTGTAGAGG TTTTACTTGC TTTAAAAAAC CTCCCACATCTCCCCCTGAA CCTGAAACAT
AAAATGAATG CAATTGTTGT TGTTAACTTG TTTATTGCAG CTTATAATGG
TTACAAATAA AGCAATAGCA TCACAAATTT CACAAATAAA GCATTTTTTT
CACTGCATTC TAGTTGTGGT TTGTCCAAAC TCATCAATGT ATCTTATCAT
GTCTGTAAAATACAGCATAG CAAAACTTTA ACCTCCAAAT CAAGCCTCTA
CTTGAATCCT TTTCTGAGGGATGAATAAGG CATAGGCATC AGGGGCTGTT
GCCAATGTGC ATTAGCTGTT TGCAGCCTCACCTTCTTTCA TGGAGTTTAA GATATAGTGT
ATTTTCCCAA GGTTTGAACT AGCTCTTCATTTCTTTATGT TTTAAATGCA CTGACCTCCC
ACATTCCCTT TTTAGTAAAA TATTCAGAAATAATTTAAAT ACATCATTGC
AATGAAAATA AATGTTTTTT ATTAGGCAGA ATCCAGATGCTCAAGGCCCT
TCATAATATC CCCCAGTTTA GTAGTTGGAC TTAGGGAACA AAGGAACCTT
TAATAGAAAT TGGACAGCAA GAAAGCGAGC TTAATTAACT ATCAACATTC
GCCTCTATTGAAGCTCTTTG TCACAGGGCT GGACAGGCCC TGGTGGGTCA
CCTCGCAGGC GTAAACCTTGTGCTTCTCGT AATCGGCCTT GCTGAGGGTC
AGTGTGCTGC TCAGGCTGTA TGTAGAGTCCTTGCTGTCCT GTTCTGTGAC GCTCTCCTGG
CTGTTGCCGC TTTGCAGGGC GTTGTCCACCTTCCACTGCA CCTTGGCCTC
GCGAGGGTAG AAGTTGTTGA GCAGGCACAC CACAGAGGCTGTGCCGGACT
TCAGCTGCTC GTCGCTAGGT GGAAAGATGA ACACAGATGG AGCGGCCACG
GTCCGCTTGA TCTCAACTTT GGTTCCGCCG CCGAATGTCA GGCTGGTGTC
GTGTTGCTGGCAGTAATACA CGGCAAAGTC CTCAGGTTCC AGCCGGCTAA
TTGTCAGGGT GAAATCAGTGCCGCTGCCGC TGCCGCTGAA TCTATCGGGG
ATGCCGGTGG CTCTACTAGA AGCTCCGTAGATCAGCAGTC TAGGAGCCTG
TCCAGGTTTC TGCTGGTACC AGGCCAGGCT GGTGCTGGACACGGTCTGAG
AGGCTCTACA AGACAGGGTA GCCCGTTCGC CGGGTGACAG GCTCAGGGTG
CCAGGGCTCT GGGTCAGCAC GATTTCGCAT CTGGCTCCGG ACAGCCACAG
CAGCAGCAGGCCCAGCAGCT GGGCGGGCAC TCTCATGTCC ATGGTGGCTG
CGGTTTAAAC TCAGTTCCAAAGGTTGGAAT CTAAAAGAGA GAAACAATTA
GAATCAGTAG TTTAACACAT TATACACTTAAAAATTTTAT ATTTACCTTA GAGTAATTAT
TCACTGTCCC AGGTCAGTGG TGGTGCCTGAAGCTGAGGAG ACAGGGCCCT
GTCCTCGTCC GTATTTAAGC AGTGGATCCA GAGGGGCAACGGGGGAGGCT
GCTGGTGAAT ATTAACCAAG GTCACCCCAG TTATCGGAGG AGCAAACAGG
GGCTAAGTCC ACTGGCTGGG ATCTGAGTCG CCCGCCTACG CTGCCCGGAC
GCTTTGCCTGGGCAGTGTAC AGCTTCCACT GCACTTACCG AAAGGAGTCA
TTGTACCTGG CTCAGAAACCACAGCGTCCT GTGTCCAAGG TGGAGGGGGT
GGCGTGAGTC AGACAGTCTC TGGGAGAGTACCACTTAGCT GGCCCTCTGC
TCTCACTGCA GAATCCTTAG TGGCTGTTCC ACTGGTAGCAAGATCCAGTA
CCTCTAGACC AGCCTGACCA ACATGGTGAA ACCCCGTCCC TGTCAAAAAT
AGAAAAAATT AGCCGGGTGT GGTGGCACAG GCCTGTAATC CCAGTTACTT
GGGAGGCTGAGGCTGACCAA CATGGAGAAA CCCGTCTCTA CTAAAAATAG
AAAATTAGCC AGGTGTGGTGGCACATGCTT GTAATCCCAG CTACTTGGGA
GGCTGAGGCA GGGGAAGGTT GTGGTGAGATGAGATTGTGT CACTGCACTC
CAGCCTGGGC GACAGAGCAA ACCTCCATCT CAAAAAACAA AACAAAACAA
AACAAAAAAA CCAAATGTTT ATTTGCCACA AAAACCCTAT CAGATGGGCG
TCTTTATCAT TTCCATTGTA CAGATGGGGA AACAGGCTTC GGGGTCGGGG
CATAGCCACT TACTGACGAC TCCCCACCCA GCAAGTGGTT TTGAACCCGG
ACCCTCTCAC ACTACCTAAACCACGCCAGG ACAACCTCTG CTCCTCTCCA
CCGAAATTCC AAGGGGTCGA GTGGATGTTGGAGGTGGCAT GGGCCCAGAG
AGGTCTCTGA CCTCTGCCCC AGCTCCAAGG TCAGCAGGCA GGGAGGGCTG
TGTGTTTGCT GTTTGCTGCT TGCAATGTTT GCCCATTTTA GGGACATGAG
TAGGCTGAAG TTTGTTCAGT GTGGACTTCA GAGGCAGCAC ACAAACAGCT
GCTGGAGGATGGGAACTGAG GGGTTGGAAG GGGGCAGGGT GAGCCCAGAA
ACTCCTGTGT GCCTCTGAGCCTACATTCTT AACTACCCTC CGCCCTACTC CTGTCCCTCC
CCCATTTCCT GTTTGCAGTACCCAAGGCAA ATATTAGTCT AAGTAGGACA
GAGGGACAAA GAGCAGGAAC ACGGGGAGGCACAAGTTCTC GCATCGATTG
TACCAAAGTA CAAGCGTTAA TGATTAACTG TACCAAAGTACAAGCGTTAA
TGATTAACTG TACCAAAGTA CAAGCGTTAA TGATTAACGG GGGAGGCTGC
TGGTGAATAT TAACCAAGGT CACCCCAGTT ATCGGAGGAG CAAACAGGGG
CTAAGTCCACCGGGGGAGGC TGCTGGTGAA TATTAACCAA GGTCACCCCA
GTTATCGGAG GAGCAAACAGGGGCTAAGTC CACCGGGGGA GGCTGCTGGT
GAATATTAAC CAAGGTCACC CCAGTTATCGGAGGAGCAAA CAGGGGCTAA
GTCCACGGTA CCCACTGGGA GGATGTTGAG TAAGATGGAAAACTACTGAT
GACCCTTGCA GAGACAGAGT ATTAGGACAT GTTTGAACAG GGGCCGGGCG
ATCAGCAGGT AGCTCTAGAG GATCCCCGTC TGTCTGCACA TTTCGTAGAG
CGAGTGTTCCGATACTCTAA TCTCCCTAGG CAAGGTTCAT ATTTGTGTAG GTTACTTATT
CTCCTTTTGTTGACTAAGTC AATAATCAGA ATCAGCAGGT TTGGAGTCAG
CTTGGCAGGG ATCAGCAGCCTGGGTTGGAA GGAGGGGGTA TAAAAGCCCC
TTCACCAGGA GAAGCCGTCA CACAGATCCACAAGCTCCTG AAGAGGTAAG
GGTTTAAGGG ATGGTTGGTT GGTGGGGTAT TAATGTTTAATTACCTGGAG
CACCTGCCTG AAATCACTTT TTTTCAGGTT GGACCAGGTC GCAGCCACCA
TGGAATTCGG CCTGTCCTGG GTCTTTCTGG TGGCCATCCT GAAGGGCGTG
CAGTGCCAGGTCCAGCTGGT TCAGAGCGGC GCCGAGGTTA AGAAACCTGG
CGCCAGCGTG AAAGTGTCCTGCAAGGCCAG CGGCTACCCC TTCACCAGCT
ACGGCATCTC TTGGGTGCGG CAGGCCCCTG GACAAGGACT GGAGTGGATG
GGATGGATCA GCACTTACCA GGGCAATACC AACTACGCCCAGAAATTCCA
GGGCAGAGTG ACAATGACCA CCGACACCAG CACAACCACA GGCTACATGG
AACTGCGGAG ACTGAGAAGC GATGATACAG CCGTGTACTA CTGCGCCAGA
GATTATACAAGAGGTGCTTG GTTCGGCGAG AGCCTGATCG GCGGATTCGA
CAACTGGGGA CAAGGCACCCTGGTGACCGT GTCATCCGCC TCTACCAAGG
GCCCTAGCGT GTTTCCACTG GCCCCTAGCTCTAAAAGCAC AAGCGGCGGC
ACCGCCGCTC TGGGATGTCT GGTGAAGGAC TACTTCCCAGAGCCCGTGAC
CGTGAGCTGG AACAGCGGCG CTCTCACATC TGGGGTGCAT ACCTTTCCCG
CCGTGCTGCA GTCTTCTGGA CTGTACAGCC TGAGCAGCGT GGTGACCGTG
CCCTCCAGCAGCCTGGGCAC ACAGACCTAC ATCTGCAACG TGAACCACAA
GCCATCTAAT ACCAAGGTGGATAAGAAGGT GGAACCTAAG AGCTGTGACA
AGACACACAC ATGCCCCCCC TGCCCTGCTCCTGAGCTGCT GGCCGGCCCC
TCCGTGTTTC TCTTCCCTCC TAAACCCAAG GACACACTGATGATTAGCCG
GACCCCAGAG GTGACCTGTG TGGTGGTTGA CGTGAGTCAC GAAGATCCTG
AAGTGAAGTT CAACTGGTAC GTGGACGGCG TGGAGGTGCA TAACGCCAAA
ACCAAGCCTCGGGAAGAGCA GTACAACAGC ACCTATAGAG TGGTGAGCGT
GCTTACAGTG CTGCATCAGG ACTGGCTGAA CGGCAAGGAA TACAAGTGCA
AGGTGTCCAA CAAAGCCCTG CCTCTGCCTGAAGAAAAGAC CATCAGCAAG
GCCAAGGGCC AACCAAGAGA GCCTCAAGTG TACACCCTGC CCCCCAGCAG
AGATGAGCTG ACCAAGAATC AGGTGTCCCT GACCTGCCTG GTCAAAGGCT
TCTACCCTAG CGACATCGCC GTCGAGTGGG AGAGCAATGG CCAGCCTGAG
AACAACTACAAGACCACCCC TCCTGTGCTG GACAGCGACG GCAGCTTCTT
CCTGTATAGC AAGCTGACCGTGGACAAGTC CAGGTGGCAG CAGGGCAATG
TGTTCAGCTG TAGCGTGCTG CACGAGGCCCTGCACAGCCA CTACACACAG
AAGTCTCTGA GCCTGTCTCC TGGCAAGTGA TAGCTTAAGGAGCATCTTAC
CGCCATTTAT TCCCATATTT GTTCTGTTTT TCTTGATTTG GGTATACATT
TAAATGTTAA TAAAACAAAA TGGTGGGGCA ATCATTTACA TTTTTAGGGA
TATGTAATTACTAGTTCAGG TGTATTGCCA CAAGACAAAC ATGTTAAGAA
ACTTTCCCGT TATTTACGCTCTGTTCCTGT TAATCAACCT CTGGATTACA AAATTTGTGA
AAGATTGACT GATATTCTTA ACTATGTTGC TCCTTTTACG CTGTGTGGAT ATGCTGCTTT
ATAGCCTCTG TATCTAGCTATTGCTTCCCG TACGGCTTTC GTTTTCTCCT CCTTGTATAA
ATCCTGGTTG CTGTCTCTTT TAGAGGAGTT GTGGCCCGTT GTCCGTCAAC
GTGGCGTGGT GTGCTCTGTG TTTGCTGACGCAACCCCCAC TGGCTGGGGC
ATTGCCACCA CCTGTCAACT CCTTTCTGGG ACTTTCGCTT TCCCCCTCCC GATCGCCACG
GCAGAACTCA TCGCCGCCTG CCTTGCCCGC TGCTGGACAGGGGCTAGGTT
GCTGGGCACT GATAATTCCG TGGTGTTGTC TGTGCCTTCT AGTTGCCAGC
CATCTGTTGT TTGCCCCTCC CCCGTGCCTT CCTTGACCCT GGAAGGTGCC
ACTCCCACTG TCCTTTCCTA ATAAAATGAG GAAATTGCAT CGCATTGTCT
GAGTAGGTGT CATTCTATTC TGGGGGGTGG GGTGGGGCAG GACAGCAAGG
GGGAGGATTG GGAAGACAAT AGCAGGCATG CTGGGGATGC GGTGGGCTCT
ATGGCTCTAG AGCATGGCTA CGTAGATAAG TAGCATGGCG GGTTAATCAT
TAACTACACC TGCAGGAGGA ACCCCTAGTG ATGGAGTTGG CCACTCCCTC
TCTGCGCGCT CGCTCGCTCA CTGAGGCCGG GCGACCAAAG GTCGCCCGAC
GCCCGGGCGG CCTCAGTGAG CGAGCGAGCG CGCAGCTGCC TGCAGG

According to some embodiments, the ceDNA vector is at least 85%, at least 90%, at least 91%, at least 92%, at least 93%, at least 94%, at least 94%, at least 95%, at least 96%, at least 97%, at least 98% or at least 99% identical to SEQ ID NO: 27.

According to some embodiments, SEQ ID NO: 27 comprises the following components, where the numbers indicate nucleic acid residues:
1 . . . 141=left-ITR_v1
142 . . . 193=spacer_left-ITR_536
complement(194 . . . 415)=SV40_polyA
416 . . . 810=HBB_3pUTR
complement(416 . . . 810)=HBBv2_3pUTR
complement(822 . . . 1532)=Translation 1532-822
complement(822 . . . 1532)=LC1 codon optimized
complement(825 . . . 1145)=hIgKappa Constant Region [P01834]
complement(825 . . . 1145)=S309 Constant Region (Human Kappa)
complement(1146 . . . 1466)=S309_VL (variable light chain)
complement(1467 . . . 1532)=Immunoglobulin kappa variable 1D-33 Signal Peptide [P01593.2]
complement(1551 . . . 2822)=CpG minimized_hAAT_Promoter Set
Spacer complement(2823 . . . 2832)=10mer_2A
complement(2823 . . . 3048) 3×HNF1-4_ProEnh_10 mer
3049 . . . 3266=3×SerpinEnhancer
3720 . . . 3776=Human IGHV3-43 heavy chain signal peptide [P0DP04]
3720 . . . 5150=HC1_codon optimized
3777 . . . 4157=S309_VH
4158 . . . 5147=S309 Constant Region (hIgG1) with GAALIE and LS mutations
5160 . . . 5740=WPRE_3pUTR
5741 . . . 5965=bGH
5966 . . . 6026=spacer_right-ITR_v1
6027 . . . 6156=right-ITR_v1

### IV. Method of Production of a ceDNA Vector

### A. Production in General

Certain methods for the production of a ceDNA vector for expression of anti-CoV-2 S antibodies and antigen-binding fragments thereof comprising an asymmetrical ITR pair or symmetrical ITR pair as defined herein is described in section IV of International application PCT/US18/49996 filed Sep. 7, 2018, which is incorporated herein in its entirety by reference. According to some embodiments, a ceDNA vector for expression of anti-CoV-2 S antibodies and antigen-binding fragments thereof as disclosed herein can be produced using insect cells, as described herein. In alternative embodiments, a ceDNA vector for expression of anti-CoV-2 S antibodies and antigen-binding fragments thereof as disclosed herein can be produced synthetically and According to some embodiments, in a cell-free method, as disclosed on International Application PCT/US19/14122, filed Jan. 18, 2019, which is incorporated herein in its entirety by reference.

As described herein, according to some embodiment, a ceDNA vector for expression of anti-CoV-2 S antibodies and antigen-binding fragments thereof can be obtained, for example, by the process comprising the steps of: (a) incubating a population of host cells (e.g., insect cells) harboring the polynucleotide expression construct template (e.g., a ceDNA-plasmid, a ceDNA-Bacmid, and/or a ceDNA-baculovirus), which is devoid of viral capsid coding sequences, in the presence of a Rep protein under conditions effective and for a time sufficient to induce production of the ceDNA vector within the host cells, and wherein the host cells do not comprise viral capsid coding sequences; and (b) harvesting and isolating the ceDNA vector from the host cells. The presence of Rep protein induces replication of the vector polynucleotide with a modified ITR to produce the ceDNA vector in a host cell. However, no viral particles (e.g., AAV virions) are expressed. Thus, there is no size limitation such as that naturally imposed in AAV or other viral-based vectors.

The presence of the ceDNA vector isolated from the host cells can be confirmed by digesting DNA isolated from the host cell with a restriction enzyme having a single recognition site on the ceDNA vector and analyzing the digested DNA material on a non-denaturing gel to confirm the presence of characteristic bands of linear and continuous DNA as compared to linear and non-continuous DNA.

In yet another aspect, the disclosure provides for use of host cell lines that have stably integrated the DNA vector polynucleotide expression template (ceDNA template) into their own genome in production of the non-viral DNA vector, e.g., as described in Lee, L. et al. (2013) Plos One 8(8): c69879. Preferably, Rep is added to host cells at an MOI of about 3. When the host cell line is a mammalian cell line, e.g., HEK293 cells, the cell lines can have polynucleotide vector template stably integrated, and a second vector such as herpes virus can be used to introduce Rep protein into cells, allowing for the excision and amplification of ceDNA in the presence of Rep and helper virus.

According to some embodiment, the host cells used to make the ceDNA vectors for expression of anti-CoV-2 S antibodies and antigen-binding fragments thereof as described herein are insect cells, and baculovirus is used to deliver both the polynucleotide that encodes Rep protein and the non-viral DNA vector polynucleotide expression construct template for ceDNA, e.g., as described in FIGS. 4A-4C and Example 1. According to some embodiments, the host cell is engineered to express Rep protein.

The ceDNA vector is then harvested and isolated from the host cells. The time for harvesting and collecting ceDNA vectors described herein from the cells can be selected and optimized to achieve a high-yield production of the ceDNA vectors. For example, the harvest time can be selected in view of cell viability, cell morphology, cell growth, etc. According to some embodiment, cells are grown and harvested a sufficient time after baculoviral infection to produce ceDNA vectors but before most cells start to die due to the baculoviral toxicity. The DNA vectors can be isolated using plasmid purification kits such as Qiagen Endo-Free Plasmid kits. Other methods developed for plasmid isolation can be also adapted for DNA vectors. Generally, any nucleic acid purification methods can be adopted.

The DNA vectors can be purified by any means known to those of skill in the art for purification of DNA. According to some embodiment, ceDNA vectors are purified as DNA molecules. In another embodiment, the ceDNA vectors are purified as exosomes or microparticles.

The presence of the ceDNA vector for expression of anti-CoV-2 S antibodies and antigen-binding fragments thereof can be confirmed by digesting the vector DNA isolated from the cells with a restriction enzyme having a single recognition site on the DNA vector and analyzing both digested and undigested DNA material using gel electrophoresis to confirm the presence of characteristic bands of linear and continuous DNA as compared to linear and non-continuous DNA. FIG. 4C and FIG. 4D illustrate one embodiment for identifying the presence of the closed ended ceDNA vectors produced by the processes herein.

According to some embodiments, the ceDNA is synthetically produced in a cell-free environment.

B. ceDNA Plasmid

A ceDNA-plasmid is a plasmid used for later production of a ceDNA vector for expression of anti-CoV-2 S antibodies and antigen-binding fragments thereof. According to some embodiments, a ceDNA-plasmid can be constructed using known techniques to provide at least the following as operatively linked components in the direction of transcription: (1) a modified 5′ ITR sequence; (2) an expression cassette containing a cis-regulatory element, for example, a promoter, inducible promoter, regulatory switch, enhancers and the like; and (3) a modified 3′ ITR sequence, where the 3′ ITR sequence is symmetric relative to the 5′ ITR sequence. According to some embodiments, the expression cassette flanked by the ITRs comprises a cloning site for introducing an exogenous sequence. The expression cassette replaces the rep and cap coding regions of the AAV genomes.

According to some aspect, a ceDNA vector for expression of anti-CoV-2 S antibodies and antigen-binding fragments thereof is obtained from a plasmid, referred to herein as a “ceDNA-plasmid” encoding in this order: a first adeno-associated virus (AAV) inverted terminal repeat (ITR), an expression cassette comprising a transgene, and a mutated or modified AAV ITR, wherein said ceDNA-plasmid is devoid of AAV capsid protein coding sequences. In alternative embodiments, the ceDNA-plasmid encodes in this order: a first (or 5′) modified or mutated AAV ITR, an expression cassette comprising a transgene, and a second (or 3′) modified AAV ITR, wherein said ceDNA-plasmid is devoid of AAV capsid protein coding sequences, and wherein the 5′ and 3′ ITRs are symmetric relative to each other. In alternative embodiments, the ceDNA-plasmid encodes in this order: a first (or 5′) modified or mutated AAV ITR, an expression cassette comprising a transgene, and a second (or 3′) mutated or modified AAV ITR, wherein said ceDNA-plasmid is devoid of AAV capsid protein coding sequences, and wherein the 5′ and 3′ modified ITRs have the same modifications (i.e., they are inverse complement or symmetric relative to each other).

In a further embodiment, the ceDNA-plasmid system is devoid of viral capsid protein coding sequences (i.e. it is devoid of AAV capsid genes but also of capsid genes of other viruses). In addition, in a particular embodiment, the ceDNA-plasmid is also devoid of AAV Rep protein coding sequences. Accordingly, in a preferred embodiment, ceDNA-plasmid is devoid of functional AAV cap and AAV rep genes GG-3′ for AAV2) plus a variable palindromic sequence allowing for hairpin formation.

A ceDNA-plasmid of the present disclosure can be generated using natural nucleic acid sequences of the genomes of any AAV serotypes well known in the art. According to some embodiment, the ceDNA-plasmid backbone is derived from the AAV1, AAV2, AAV3, AAV4, AAV5, AAV 5, AAV7, AAV8, AAV9, AAV10, AAV 11, AAV12, AAVrh8, AAVrh10, AAV-DJ, and AAV-DJ8 genome. E.g., NCBI: NC 002077; NC 001401; NC001729; NC001829; NC006152; NC 006260; NC 006261; Kotin and Smith, The Springer Index of Viruses, available at the URL maintained by Springer (at www web address: oesys.springer.de/viruses/database/mkchapter.asp?virID=42.04.) (note—references to a URL or database refer to the contents of the URL or database as of the effective filing date of this application) In a particular embodiment, the ceDNA-plasmid backbone is derived from the AAV2 genome. In another particular embodiment, the ceDNA-plasmid backbone is a synthetic backbone genetically engineered to include at its 5′ and 3′ ITRs derived from one of these AAV genomes.

A ceDNA-plasmid can optionally include a selectable or selection marker for use in the establishment of a ceDNA vector-producing cell line. According to some embodiment, the selection marker can be inserted downstream (i.e., 3′) of the 3′ ITR sequence. In another embodiment, the selection marker can be inserted upstream (i.e., 5′) of the 5′ ITR sequence. Appropriate selection markers include, for example, those that confer drug resistance. Selection markers can be, for example, a blasticidin S-resistance gene, kanamycin, geneticin, and the like. In a preferred embodiment, the drug selection marker is a blasticidin S-resistance gene.

An exemplary ceDNA (e.g., rAAV0) vector for expression of anti-CoV S antibodies and antigen-binding fragments thereof (e.g., anti-CoV-2 S antibodies and antigen-binding fragments thereof) is produced from an rAAV plasmid. A method for the production of a rAAV vector, can comprise: (a) providing a host cell with a rAAV plasmid as described above, wherein both the host cell and the plasmid are devoid of capsid protein encoding genes, (b) culturing the host cell under conditions allowing production of an ceDNA genome, and (c) harvesting the cells and isolating the AAV genome produced from said cells.

C. Exemplary Method of Making the ceDNA Vectors from ceDNA Plasmids

Methods for making capsid-less ceDNA vectors for expression of anti-CoV-2 S antibodies and antigen-binding fragments thereof are also provided herein, notably a method with a sufficiently high yield to provide sufficient vector for in vivo experiments.

According to some embodiments, a method for the production of a ceDNA vector for expression of anti-CoV-2 S antibodies and antigen-binding fragments thereof comprises the steps of: (1) introducing the nucleic acid construct comprising an expression cassette and two symmetric ITR sequences into a host cell (e.g., Sf9 cells), (2) optionally, establishing a clonal cell line, for example, by using a selection marker present on the plasmid, (3) introducing a Rep coding gene (either by transfection or infection with a baculovirus carrying said gene) into said insect cell, and (4) harvesting the cell and purifying the ceDNA vector. The nucleic acid construct comprising an expression cassette and two ITR sequences described above for the production of ceDNA vector can be in the form of a ceDNA plasmid, or Bacmid or Baculovirus generated with the ceDNA plasmid as described below. The nucleic acid construct can be introduced into a host cell by transfection, viral transduction, stable integration, or other methods known in the art.

### D. Cell Lines

Host cell lines used in the production of a ceDNA vector for expression of anti-CoV-2 S antibodies and antigen-binding fragments thereof can include insect cell lines derived from Spodoptera frugiperda, such as Sf9 Sf21, or Trichoplusia ni cell, or other invertebrate, vertebrate, or other eukaryotic cell lines including mammalian cells. Other cell lines known to an ordinarily skilled artisan can also be used, such as HEK293, Huh-7, HeLa, HepG2, HeplA, 911, CHO, COS, MeWo, NIH3T3, A549, HT1 180, monocytes, and mature and immature dendritic cells. Host cell lines can be transfected for stable expression of the ceDNA-plasmid for high yield ceDNA vector production.

CeDNA-plasmids can be introduced into Sf9 cells by transient transfection using reagents (e.g., liposomal, calcium phosphate) or physical means (e.g., electroporation) known in the art. Alternatively, stable Sf9 cell lines which have stably integrated the ceDNA-plasmid into their genomes can be established. Such stable cell lines can be established by incorporating a selection marker into the ceDNA-plasmid as described above. If the ceDNA-plasmid used to transfect the cell line includes a selection marker, such as an antibiotic, cells that have been transfected with the ceDNA-plasmid and integrated the ceDNA-plasmid DNA into their genome can be selected for by addition of the antibiotic to the cell growth media. Resistant clones of the cells can then be isolated by single-cell dilution or colony transfer techniques and propagated.

E. Isolating and Purifying ceDNA Vectors

Examples of the process for obtaining and isolating ceDNA vectors are described in FIGS. 4A-4E and the specific examples below. ceDNA-vectors for expression of anti-CoV-2 S antibodies and antigen-binding fragments thereof disclosed herein can be obtained from a producer cell expressing AAV Rep protein(s), further transformed with a ceDNA-plasmid, ceDNA-bacmid, or ceDNA-baculovirus. Plasmids useful for the production of ceDNA vectors include plasmids that encode anti-CoV-2 S antibodies and antigen-binding fragments thereof, or plasmids encoding one or more REP proteins.

According to some aspect, a polynucleotide encodes the AAV Rep protein (Rep 78 or 68) delivered to a producer cell in a plasmid (Rep-plasmid), a bacmid (Rep-bacmid), or a baculovirus (Rep-baculovirus). The Rep-plasmid, Rep-bacmid, and Rep-baculovirus can be generated by methods described above.

Methods to produce a ceDNA vector for expression of anti-CoV-2 S antibodies and antigen-binding fragments thereof are described herein. Expression constructs used for generating a ceDNA vector for expression of e.g., anti-CoV-2 S antibodies and antigen-binding fragments thereof as described herein can be a plasmid (e.g., ceDNA-plasmids), a Bacmid (e.g., ceDNA-bacmid), and/or a baculovirus (e.g., ceDNA-baculovirus). By way of an example only, a ceDNA-vector can be generated from the cells co-infected with ceDNA-baculovirus and Rep-baculovirus. Rep proteins produced from the Rep-baculovirus can replicate the ceDNA-baculovirus to generate ceDNA-vectors. Alternatively, ceDNA vectors for expression of anti-CoV-2 S antibodies and antigen-binding fragments thereof can be generated from the cells stably transfected with a construct comprising a sequence encoding the AAV Rep protein (Rep78/52) delivered in Rep-plasmids, Rep-bacmids, or Rep-baculovirus. CeDNA-Baculovirus can be transiently transfected to the cells, be replicated by Rep protein and produce ceDNA vectors.

The bacmid (e.g., ceDNA-bacmid) can be transfected into permissive insect cells such as Sf9, Sf21, Tni (Trichoplusia ni) cell, High Five cell, and generate ceDNA-baculovirus, which is a recombinant baculovirus including the sequences comprising the symmetric ITRs and the expression cassette. ceDNA-baculovirus can be again infected into the insect cells to obtain a next generation of the recombinant baculovirus. Optionally, the step can be repeated once or multiple times to produce the recombinant baculovirus in a larger quantity.

The time for harvesting and collecting ceDNA vectors for expression of anti-CoV-2 S antibodies and antigen-binding fragments thereof as described herein from the cells can be selected and optimized to achieve a high-yield production of the ceDNA vectors. For example, the harvest time can be selected in view of cell viability, cell morphology, cell growth, etc. Usually, cells can be harvested after sufficient time after baculoviral infection to produce ceDNA vectors but before majority of cells start to die because of the viral toxicity. The ceDNA-vectors can be isolated from the Sf9 cells using plasmid purification kits such as Qiagen ENDO-FREE PLASMID® kits. Other methods developed for plasmid isolation can be also adapted for ceDNA vectors. Generally, any art-known nucleic acid purification methods can be adopted, as well as commercially available DNA extraction kits.

Alternatively, purification can be implemented by subjecting a cell pellet to an alkaline lysis process, centrifuging the resulting lysate and performing chromatographic separation. As one non-limiting example, the process can be performed by loading the supernatant on an ion exchange column (e.g., SARTOBIND Q®) which retains nucleic acids, and then eluting (e.g., with a 1.2 M NaCl solution) and performing a further chromatographic purification on a gel filtration column (e.g., 6 fast flow GE). The capsid-free AAV vector is then recovered by, e.g., precipitation.

According to some embodiments, ceDNA vectors for expression of anti-CoV-2 S antibodies and antigen-binding fragments thereof can also be purified in the form of exosomes, or microparticles. It is known in the art that many cell types release not only soluble proteins, but also complex protein/nucleic acid cargoes via membrane microvesicle shedding (Cocucci et al., 2009; EP 10306226.1) Such vesicles include microvesicles (also referred to as microparticles) and exosomes (also referred to as nanovesicles), both of which comprise proteins and RNA as cargo. Microvesicles are generated from the direct budding of the plasma membrane, and exosomes are released into the extracellular environment upon fusion of multivesicular endosomes with the plasma membrane. Thus, ceDNA vector-containing microvesicles and/or exosomes can be isolated from cells that have been transduced with the ceDNA-plasmid or a bacmid or baculovirus generated with the ceDNA-plasmid.

Microvesicles can be isolated by subjecting culture medium to filtration or ultracentrifugation at 20,000×g, and exosomes at 100,000×g. The optimal duration of ultracentrifugation can be experimentally-determined and will depend on the particular cell type from which the vesicles are isolated. Preferably, the culture medium is first cleared by low-speed centrifugation (e.g., at 2000×g for 5-20 minutes) and subjected to spin concentration using, e.g., an AMICON® spin column (Millipore, Watford, UK). Microvesicles and exosomes can be further purified via FACS or MACS by using specific antibodies that recognize specific surface antigens present on the microvesicles and exosomes. Other microvesicle and exosome purification methods include, but are not limited to, immunoprecipitation, affinity chromatography, filtration, and magnetic beads coated with specific antibodies or aptamers. Upon purification, vesicles are washed with, e.g., phosphate-buffered saline. One advantage of using microvesicles or exosome to deliver ceDNA-containing vesicles is that these vesicles can be targeted to various cell types by including on their membranes proteins recognized by specific receptors on the respective cell types. (See also EP 10306226)

Another aspect of the disclosure herein relates to methods of purifying ceDNA vectors from host cell lines that have stably integrated a ceDNA construct into their own genome. According to some embodiment, ceDNA vectors are purified as DNA molecules. In another embodiment, the ceDNA vectors are purified as exosomes or microparticles.

FIG. 5 of International application PCT/US18/49996 shows a gel confirming the production of ceDNA from multiple ceDNA-plasmid constructs using the method described in the Examples. The ceDNA is confirmed by a characteristic band pattern in the gel, as discussed with respect to FIG. 4D in the Examples.

### V. Pharmaceutical Compositions

In another aspect, pharmaceutical compositions are provided. The pharmaceutical composition comprises a ceDNA vector for expression of anti-CoV-2 S antibodies and antigen-binding fragments thereof as described herein and a pharmaceutically acceptable carrier or diluent.

The ceDNA vectors for expression of anti-CoV-2 S antibodies and antigen-binding fragments thereof as disclosed herein can be incorporated into pharmaceutical compositions suitable for administration to a subject for in vivo delivery to cells, tissues, or organs of the subject. Typically, the pharmaceutical composition comprises a ceDNA-vector as disclosed herein and a pharmaceutically acceptable carrier. For example, the ceDNA vectors for expression of anti-CoV-2 S antibodies and antigen-binding fragments thereof as described herein can be incorporated into a pharmaceutical composition suitable for a desired route of therapeutic administration (e.g., parenteral administration). Passive tissue transduction via high pressure intravenous or intra-arterial infusion, as well as intracellular injection, such as intranuclear microinjection or intracytoplasmic injection, are also contemplated. Pharmaceutical compositions for therapeutic purposes can be formulated as a solution, microemulsion, dispersion, liposomes, or other ordered structure suitable to high ceDNA vector concentration. Sterile injectable solutions can be prepared by incorporating the ceDNA vector compound in the required amount in an appropriate buffer with one or a combination of ingredients enumerated above, as required, followed by filtered sterilization including a ceDNA vector can be formulated to deliver a transgene in the nucleic acid to the cells of a recipient, resulting in the therapeutic expression of the transgene or donor sequence therein. The composition can also include a pharmaceutically acceptable carrier.

Pharmaceutically active compositions comprising a ceDNA vector for expression of anti-CoV-2 S antibodies and antigen-binding fragments thereof can be formulated to deliver a transgene for various purposes to the cell, e.g., cells of a subject.

Pharmaceutical compositions for therapeutic purposes typically must be sterile and stable under the conditions of manufacture and storage. The composition can be formulated as a solution, microemulsion, dispersion, liposomes, or other ordered structure suitable to high ceDNA vector concentration. Sterile injectable solutions can be prepared by incorporating the ceDNA vector compound in the required amount in an appropriate buffer with one or a combination of ingredients enumerated above, as required, followed by filtered sterilization.

A ceDNA vector for expression of anti-CoV-2 S antibodies and antigen-binding fragments thereof as disclosed herein can be incorporated into a pharmaceutical composition suitable for topical, systemic, intra-amniotic, intrathecal, intracranial, intra-arterial, intravenous, intralymphatic, intraperitoneal, subcutaneous, tracheal, intra-tissue (e.g., intramuscular, intracardiac, intrahepatic, intrarenal, intracerebral), intrathecal, intravesical, conjunctival (e.g., extra-orbital, intraorbital, retroorbital, intraretinal, subretinal, choroidal, sub-choroidal, intrastromal, intracameral and intravitreal), intracochlear, and mucosal (e.g., oral, rectal, nasal) administration. Passive tissue transduction via high pressure intravenous or intraarterial infusion, as well as intracellular injection, such as intranuclear microinjection or intracytoplasmic injection, are also contemplated.

According to some embodiments, the ceDNA vectors are delivered to the lungs of a subject.

According to some aspects, the methods provided herein comprise delivering one or more ceDNA vectors for expression of anti-CoV-2 S antibodies and antigen-binding fragments thereof as disclosed herein to a host cell. Also provided herein are cells produced by such methods, and organisms (such as animals, plants, or fungi) comprising or produced from such cells. Methods of delivery of nucleic acids can include lipofection, nucleofection, microinjection, biolistics, liposomes, immunoliposomes, polycation or lipid:nucleic acid conjugates, naked DNA, and agent-enhanced uptake of DNA. Lipofection is described in e.g., U.S. Pat. Nos. 5,049,386, 4,946,787; and 4,897,355) and lipofection reagents are sold commercially (e.g., TRANSFECTAM™ and LIPOFECTIN™). Delivery can be to cells (e.g., in vitro or ex vivo administration) or target tissues (e.g., in vivo administration).

Various techniques and methods are known in the art for delivering nucleic acids to cells. For example, nucleic acids, such as ceDNA for expression of anti-CoV-2 S antibodies and antigen-binding fragments thereof can be formulated into lipid nanoparticles (LNPs), lipidoids, liposomes, lipid nanoparticles, lipoplexes, or core-shell nanoparticles. Typically, LNPs are composed of nucleic acid (e.g., ceDNA) molecules, one or more ionizable or cationic lipids (or salts thereof), one or more non-ionic or neutral lipids (e.g., a phospholipid), a molecule that prevents aggregation (e.g., PEG or a PEG-lipid conjugate), and optionally a sterol (e.g., cholesterol).

Another method for delivering nucleic acids, such as ceDNA for expression of anti-CoV-2 S antibodies and antigen-binding fragments thereof to a cell is by conjugating the nucleic acid with a ligand that is internalized by the cell. For example, the ligand can bind a receptor on the cell surface and internalized via endocytosis. The ligand can be covalently linked to a nucleotide in the nucleic acid. Exemplary conjugates for delivering nucleic acids into a cell are described, example, in WO2015/006740, WO2014/025805, WO2012/037254, WO2009/082606, WO2009/073809, WO2009/018332. WO2006/112872, WO2004/090108, WO2004/091515 and WO2017/177326.

Nucleic acids, such as ceDNA vectors for expression of anti-CoV-2 S antibodies and antigen-binding fragments thereof can also be delivered to a cell by transfection. Useful transfection methods include, but are not limited to, lipid-mediated transfection, cationic polymer-mediated transfection, or calcium phosphate precipitation. Transfection reagents are well known in the art and include, but are not limited to, TurboFect Transfection Reagent (Thermo Fisher Scientific), Pro-Ject Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293fectin, LIPOFECTAMINE™ 2000, LIPOFECTAMINE™ 3000 (Thermo Fisher Scientific), LIPOFECTAMINE™ (Thermo Fisher Scientific), LIPOFECTIN™ (Thermo Fisher Scientific), DMRIE-C, CELLFECTIN™ (Thermo Fisher Scientific), OLIGOFECTAMINE™ (Thermo Fisher Scientific), LIPOFECTACE™, FUGENE™ (Roche, Basel, Switzerland), FUGENE™ HD (Roche), TRANSFECTAM™ (Transfectam, Promega, Madison, Wis.), TFX-10™ (Promega), TFX-20™ (Promega), TFX-50™ (Promega), TRANSFECTIN™ (BioRad, Hercules, Calif.), SILENTFECT™ (Bio-Rad), Effectene™ (Qiagen, Valencia, Calif.), DC-chol (Avanti Polar Lipids), GENEPORTER™ (Gene Therapy Systems, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT 4™ (Dharmacon), ESCORT™ III (Sigma, St. Louis, Mo.), and ESCORT™ IV (Sigma Chemical Co.). Nucleic acids, such as ceDNA, can also be delivered to a cell via microfluidics methods known to those of skill in the art.

ceDNA vectors for expression of anti-CoV-2 S antibodies and antigen-binding fragments thereof as described herein can also be administered directly to an organism for transduction of cells in vivo. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

The ceDNA vectors for expression of anti-CoV-2 S antibodies and antigen-binding fragments thereof in accordance with the present disclosure can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids. Exemplary liposomes and liposome formulations, including but not limited to polyethylene glycol (PEG)-functional group containing compounds are disclosed in International Application PCT/US2018/050042, filed on Sep. 7, 2018 and in International application PCT/US2018/064242, filed on Dec. 6, 2018, e.g., see the section entitled “Pharmaceutical Formulations”.

Various delivery methods known in the art or modification thereof can be used to deliver ceDNA vectors in vitro or in vivo. For example, according to some embodiments, ceDNA vectors for expression of anti-CoV-2 S antibodies and antigen-binding fragments thereof are delivered by making transient penetration in cell membrane by mechanical, electrical, ultrasonic, hydrodynamic, or laser-based energy so that DNA entrance into the targeted cells is facilitated. For example, a ceDNA vector can be delivered by transiently disrupting cell membrane by squeezing the cell through a size-restricted channel or by other means known in the art. According to some cases, a ceDNA vector alone is directly injected as naked DNA into any one of: any one or more tissues selected from: lung, liver, kidneys, gallbladder, prostate, adrenal gland, heart, intestine, stomach, skin, thymus, cardiac muscle or skeletal muscle. According to some cases, a ceDNA vector is delivered by gene gun. Gold or tungsten spherical particles (1-3 μm diameter) coated with capsid-free AAV vectors can be accelerated to high speed by pressurized gas to penetrate into target tissue cells.

Compositions comprising a ceDNA vector for expression of anti-CoV-2 S antibodies and antigen-binding fragments thereof and a pharmaceutically acceptable carrier are specifically contemplated herein. According to some embodiments, the ceDNA vector is formulated with a lipid delivery system, for example, liposomes as described herein. According to some embodiments, such compositions are administered by any route desired by a skilled practitioner. The compositions may be administered to a subject by different routes including orally, parenterally, sublingually, transdermally, rectally, transmucosally, topically, via inhalation, via buccal administration, intrapleurally, intravenous, intra-arterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian may readily determine the dosing regimen and route of administration that is most appropriate for a particular animal. The compositions may be administered by traditional syringes, needleless injection devices, “microprojectile bombardment gene guns”, or other physical methods such as electroporation (“EP”), hydrodynamic methods, or ultrasound.

According to some cases, a ceDNA vector for expression of anti-CoV-2 S antibodies and antigen-binding fragments thereof is delivered by hydrodynamic injection, which is a simple and highly efficient method for direct intracellular delivery of any water-soluble compounds and particles into internal organs and skeletal muscle in an entire limb.

### A. Microparticle/Nanoparticles

According to some embodiments, a ceDNA vector for expression of anti-CoV-2 S antibodies and antigen-binding fragments thereof as disclosed herein is delivered by a lipid nanoparticle. Generally, lipid nanoparticles comprise an ionizable amino lipid (e.g., heptatriaconta-6,9,28,31-tetraen-19-yl 4-(dimethylamino)butanoate, DLin-MC3-DMA, a phosphatidylcholine (1,2-distearoyl-sn-glycero-3-phosphocholine, DSPC), cholesterol and a coat lipid (polyethylene glycol-dimyristoylglycerol, PEG-DMG), for example as disclosed by Tam et al. (2013). Advances in Lipid Nanoparticles for siRNA delivery. Pharmaceuticals 5(3): 498-507.

According to some embodiments, a lipid nanoparticle has a mean diameter between about 10 and about 1000 nm. According to some embodiments, a lipid nanoparticle has a diameter that is less than 300 nm. According to some embodiments, a lipid nanoparticle has a diameter between about 10 and about 300 nm. According to some embodiments, a lipid nanoparticle has a diameter that is less than 200 nm. According to some embodiments, a lipid nanoparticle has a diameter between about 25 and about 200 nm. According to some embodiments, a lipid nanoparticle preparation (e.g., composition comprising a plurality of lipid nanoparticles) has a size distribution in which the mean size (e.g., diameter) is about 70 nm to about 200 nm, and more typically the mean size is about 100 nm or less.

Various lipid nanoparticles known in the art can be used to deliver ceDNA vector for expression of anti-CoV S antibodies and antigen-binding fragments thereof (e.g., anti-CoV-2 S antibodies and antigen-binding fragments thereof) as disclosed herein. For example, various delivery methods using lipid nanoparticles are described in U.S. Pat. Nos. 9,404,127, 9,006,417 and 9,518,272.

### B. Liposomes

The ceDNA vectors for expression of anti-CoV S antibodies and antigen-binding fragments thereof (e.g., anti-CoV-2 S antibodies and antigen-binding fragments thereof) in accordance with the present disclosure can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

The formation and use of liposomes is generally known to those of skill in the art. Liposomes have been developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

### C. Exemplary Liposome and Lipid Nanoparticle (LNP) Compositions

The ceDNA vectors for expression of anti-CoV-2 S antibodies and antigen-binding fragments thereof in accordance with the present disclosure can be added to liposomes for delivery to a cell, e.g., a cell in need of expression of the transgene. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

Lipid nanoparticles (LNPs) comprising ceDNA vectors are disclosed in International Application PCT/US2018/050042, filed on Sep. 7, 2018, and International Application PCT/US2018/064242, filed on Dec. 6, 2018 which are incorporated herein in their entirety and envisioned for use in the methods and compositions for ceDNA vectors for expression of anti-CoV S antibodies and antigen-binding fragments thereof (e.g., anti-CoV-2 S antibodies and antigen-binding fragments thereof) as disclosed herein.

According to some aspects, the disclosure provides for a liposome formulation that includes one or more compounds with a polyethylene glycol (PEG) functional group (so-called “PEG-ylated compounds”) which can reduce the immunogenicity/antigenicity of, provide hydrophilicity and hydrophobicity to the compound(s) and reduce dosage frequency. Or the liposome formulation simply includes polyethylene glycol (PEG) polymer as an additional component. In such aspects, the molecular weight of the PEG or PEG functional group can be from 62 Da to about 5,000 Da.

According to some aspects, the disclosure provides for a liposome formulation that will deliver an API with extended release or controlled release profile over a period of hours to weeks. According to some related aspects, the liposome formulation may comprise aqueous chambers that are bound by lipid bilayers. In other related aspects, the liposome formulation encapsulates an API with components that undergo a physical transition at elevated temperature which releases the API over a period of hours to weeks.

According to some aspects, the liposome formulation comprises sphingomyelin and one or more lipids disclosed herein. According to some aspects, the liposome formulation comprises optisomes.

According to some aspects, the disclosure provides for a liposome formulation that includes one or more lipids selected from: N-(carbonyl-methoxypolyethylene glycol 2000)-1,2-distearoyl-sn-glycero-3-phosphoethanolamine sodium salt, (distearoyl-sn-glycero-phosphoethanolamine), MPEG (methoxy polyethylene glycol)-conjugated lipid, HSPC (hydrogenated soy phosphatidylcholine); PEG (polyethylene glycol); DSPE (distearoyl-sn-glycero-phosphoethanolamine); DSPC (distearoylphosphatidylcholine); DOPC (dioleoylphosphatidylcholine); DPPG (dipalmitoylphosphatidylglycerol); EPC (egg phosphatidylcholine); DOPS (dioleoylphosphatidylserine); POPC (palmitoyloleoylphosphatidylcholine); SM (sphingomyelin); MPEG (methoxy polyethylene glycol); DMPC (dimyristoyl phosphatidylcholine); DMPG (dimyristoyl phosphatidylglycerol); DSPG (distearoylphosphatidylglycerol); DEPC (dierucoylphosphatidylcholine); DOPE (diolcoly-sn-glycero-phophoethanolamine), cholesteryl sulphate (CS), dipalmitoylphosphatidylglycerol (DPPG), DOPC (diolcoly-sn-glycero-phosphatidylcholine) or any combination thereof.

According to some aspects, the disclosure provides for a liposome formulation comprising phospholipid, cholesterol and a PEG-ylated lipid in a molar ratio of 56:38:5. According to some aspects, the liposome formulation's overall lipid content is from 2-16 mg/mL. According to some aspects, the disclosure provides for a liposome formulation comprising a lipid containing a phosphatidylcholine functional group, a lipid containing an ethanolamine functional group and a PEG-ylated lipid. According to some aspects, the disclosure provides for a liposome formulation comprising a lipid containing a phosphatidylcholine functional group, a lipid containing an ethanolamine functional group and a PEG-ylated lipid in a molar ratio of 3:0.015:2 respectively. According to some aspects, the disclosure provides for a liposome formulation comprising a lipid containing a phosphatidylcholine functional group, cholesterol and a PEG-ylated lipid. According to some aspects, the disclosure provides for a liposome formulation comprising a lipid containing a phosphatidylcholine functional group and cholesterol. According to some aspects, the PEG-ylated lipid is PEG-2000-DSPE. According to some aspects, the disclosure provides for a liposome formulation comprising DPPG, soy PC, MPEG-DSPE lipid conjugate and cholesterol.

According to some aspects, the disclosure provides for a liposome formulation comprising one or more lipids containing a phosphatidylcholine functional group and one or more lipids containing an ethanolamine functional group. According to some aspects, the disclosure provides for a liposome formulation comprising one or more: lipids containing a phosphatidylcholine functional group, lipids containing an ethanolamine functional group, and sterols, e.g., cholesterol. According to some aspects, the liposome formulation comprises DOPC/DEPC; and DOPE.

According to some aspects, the disclosure provides for a liposome formulation further comprising one or more pharmaceutical excipients, e.g., sucrose and/or glycine.

According to some aspects, the disclosure provides for a liposome formulation that is either unilamellar or multilamellar in structure. According to some aspects, the disclosure provides for a liposome formulation that comprises multi-vesicular particles and/or foam-based particles. According to some aspects, the disclosure provides for a liposome formulation that are larger in relative size to common nanoparticles and about 150 to 250 nm in size. According to some aspects, the liposome formulation is a lyophilized powder.

According to some aspects, the disclosure provides for a liposome formulation that is made and loaded with ceDNA vectors disclosed or described herein, by adding a weak base to a mixture having the isolated ceDNA outside the liposome. This addition increases the pH outside the liposomes to approximately 7.3 and drives the API into the liposome. According to some aspects, the disclosure provides for a liposome formulation having a pH that is acidic on the inside of the liposome. In such cases the inside of the liposome can be at pH 4-6.9, and more preferably pH 6.5. In other aspects, the disclosure provides for a liposome formulation made by using intra-liposomal drug stabilization technology. In such cases, polymeric or non-polymeric highly charged anions and intra-liposomal trapping agents are utilized, e.g., polyphosphate or sucrose octasulfate.

According to some aspects, the disclosure provides for a lipid nanoparticle comprising ceDNA and an ionizable lipid. For example, a lipid nanoparticle formulation that is made and loaded with ceDNA obtained by the process as disclosed in International Application PCT/US2018/050042, filed on Sep. 7, 2018, which is incorporated herein. This can be accomplished by high energy mixing of ethanolic lipids with aqueous ceDNA at low pH which protonates the ionizable lipid and provides favorable energetics for ceDNA/lipid association and nucleation of particles. The particles can be further stabilized through aqueous dilution and removal of the organic solvent. The particles can be concentrated to the desired level.

Generally, the lipid nanoparticles are prepared at a total lipid to ceDNA (mass or weight) ratio of from about 10:1 to 60:1. According to some embodiments, the lipid to ceDNA ratio (mass/mass ratio; w/w ratio) can be in the range of from about 1:1 to about 60:1, from about 1:1 to about 55:1, from about 1:1 to about 50:1, from about 1:1 to about 45:1, from about 1:1 to about 40:1, from about 1:1 to about 35:1, from about 1:1 to about 30:1, from about 1:1 to about 25:1, from about 10:1 to about 14:1, from about 3:1 to about 15:1, from about 4:1 to about 10:1, from about 5:1 to about 9:1, about 6:1 to about 9:1; from about 30:1 to about 60:1. According to some embodiments, the lipid particles (e.g., lipid nanoparticles) are prepared at a ceDNA (mass or weight) to total lipid ratio of about 60:1. According to some embodiments, the lipid particles are prepared at a total lipid to ceDNA (mass or weight) ratio of from about 10:1 to 30:1. According to some embodiments, the lipid to ceDNA ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and ceDNA can be adjusted to provide a desired N/P ratio, for example, N/P ratio of 3, 4, 5, 6, 7, 8, 9, 10 or higher. Generally, the lipid particle formulation's overall lipid content can range from about 5 mg/ml to about 30 mg/mL.

The ionizable lipid is typically employed to condense the nucleic acid cargo, e.g., ceDNA at low pH and to drive membrane association and fusogenicity.

Generally, ionizable lipids are lipids comprising at least one amino group that is positively charged or becomes protonated under acidic conditions, for example at pH of 6.5 or lower. Ionizable lipids are also referred to as cationic lipids herein.

Exemplary ionizable lipids are described in International PCT patent publications WO2015/095340, WO2015/199952, WO2018/011633, WO2017/049245, WO2015/061467, WO2012/040184, WO2012/000104, WO2015/074085, WO2016/081029, WO2017/004143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO2012/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, and US patent publications US2016/0311759, US2015/0376115, US2016/0151284, US2017/0210697, US2015/0140070, US2013/0178541, US2013/0303587, US2015/0141678, US2015/0239926, US2016/0376224, US2017/0119904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, US2011/0076335, US2006/0083780, US2013/0123338, US2015/0064242, US2006/0051405, US2013/0065939, US2006/0008910, US2003/0022649, US2010/0130588, US2013/0116307, US2010/0062967, US2013/0202684, US2014/0141070, US2014/0255472, US2014/0039032, US2018/0028664, US2016/0317458, and US2013/0195920, the contents of all of which are incorporated herein by reference in their entirety.

According to some embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3) having the following structure:

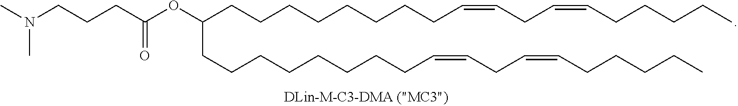 [SMILES: CCCCC/C=C\C/C=C\CCCCCCCCC(CCCCCCCC/C=C\C/C=C\CCCCC)OC(=O)CCCN(C)C]

The lipid DLin-MC3-DMA is described in Jayaraman et al., Angew. Chem. Int. Ed Engl. (2012), 51(34): 8529-8533, content of which is incorporated herein by reference in its entirety.

According to some embodiments, the ionizable lipid is the lipid ATX-002 as described in WO2015/074085, content of which is incorporated herein by reference in its entirety.

According to some embodiments, the ionizable lipid is (13Z,16Z)-N,N-dimethyl-3-nonyldocosa-13,16-dien-1-amine (Compound 32), as described in WO2012/040184, content of which is incorporated herein by reference in its entirety.

According to some embodiments, the ionizable lipid is Compound 6 or Compound 22 as described in WO2015/199952, content of which is incorporated herein by reference in its entirety.

Without limitations, ionizable lipid can comprise 20-90% (mol) of the total lipid present in the lipid nanoparticle. For example, ionizable lipid molar content can be 20-70% (mol), 30-60% (mol) or 40-50% (mol) of the total lipid present in the lipid nanoparticle. According to some embodiments, ionizable lipid comprises from about 50 mol % to about 90 mol % of the total lipid present in the lipid nanoparticle.

According to some aspects, the lipid nanoparticle can further comprise a non-cationic lipid. Non-ionic lipids include amphipathic lipids, neutral lipids and anionic lipids. Accordingly, the non-cationic lipid can be a neutral uncharged, zwitterionic, or anionic lipid. Non-cationic lipids are typically employed to enhance fusogenicity.

Exemplary non-cationic lipids envisioned for use in the methods and compositions as disclosed herein are described in International Application PCT/US2018/050042, filed on Sep. 7, 2018, and PCT/US2018/064242, filed on Dec. 6, 2018 which is incorporated herein in its entirety. Exemplary non-cationic lipids are described in International Application Publication WO2017/099823 and US patent publication US2018/0028664, the contents of both of which are incorporated herein by reference in their entirety.

The non-cationic lipid can comprise 0-30% (mol) of the total lipid present in the lipid nanoparticle. For example, the non-cationic lipid content is 5-20% (mol) or 10-15% (mol) of the total lipid present in the lipid nanoparticle. In various embodiments, the molar ratio of ionizable lipid to the neutral lipid ranges from about 2:1 to about 8:1.

According to some embodiments, the lipid nanoparticles do not comprise any phospholipids. According to some aspects, the lipid nanoparticle can further comprise a component, such as a sterol, to provide membrane integrity.

One exemplary sterol that can be used in the lipid nanoparticle is cholesterol and derivatives thereof. Exemplary cholesterol derivatives are described in International application WO2009/127060 and US patent publication US2010/0130588, contents of both of which are incorporated herein by reference in their entirety.

The component providing membrane integrity, such as a sterol, can comprise 0-50% (mol) of the total lipid present in the lipid nanoparticle. According to some embodiments, such a component is 20-50% (mol) 30-40% (mol) of the total lipid content of the lipid nanoparticle.

According to some aspects, the lipid nanoparticle can further comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. According to some embodiments, the conjugated lipid molecule is a PEG-lipid conjugate, for example, a (methoxy polyethylene glycol)-conjugated lipid. Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, and US2017/0119904, the contents of all of which are incorporated herein by reference in their entirety.

According to some embodiments, a PEG-lipid is a compound as defined in US2018/0028664, the content of which is incorporated herein by reference in its entirety. According to some embodiments, a PEG-lipid is disclosed in US20150376115 or in US2016/0376224, the content of both of which is incorporated herein by reference in its entirety.

The PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl] carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. According to some examples, the PEG-lipid can be selected from the group consisting of PEG-DMG, 1,2-dimyristoyl-sn-glycero-3-phosphoethanolamine-N-[methoxy(polyethylene glycol)-2000],

Lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (CPL) conjugates can be used in place of or in addition to the PEG-lipid. Exemplary conjugated lipids, i.e., PEG-lipids, (POZ)-lipid conjugates, ATTA-lipid conjugates and cationic polymer-lipids are described in the International patent application publications WO1996/010392, WO1998/051278, WO2002/087541, WO2005/026372, WO2008/147438, WO2009/086558, WO2012/000104, WO2017/117528, WO2017/099823, WO2015/199952, WO2017/004143, WO2015/095346, WO2012/000104, WO2012/000104, and WO2010/006282, US patent application publications US2003/0077829, US2005/0175682, US2008/0020058, US2011/0117125, US2013/0303587, US2018/0028664, US2015/0376115, US2016/0376224, US2016/0317458, US2013/0303587, US2013/0303587, and US20110123453, and U.S. Pat. Nos. 5,885,613, 6,287,591, 6,320,017, and 6,586,559, the contents of all of which are incorporated herein by reference in their entirety.

According to some embodiments, the one or more additional compound can be a therapeutic agent. The therapeutic agent can be selected from any class suitable for the therapeutic objective. In other words, the therapeutic agent can be selected from any class suitable for the therapeutic objective. In other words, the therapeutic agent can be selected according to the treatment objective and biological action desired.

According to some embodiments, the additional compound is another antibody, or antigen-binding fragment thereof, described herein.

According to some embodiments, the additional agent is an anti-viral drug or a vaccine. According to some embodiments, the additional agent is selected from the group consisting of: an anti-inflammatory agent, an antimalarial agent, and an antibody or antigen-binding fragment thereof that specifically binds to CoV-S. In further embodiments, the antimalarial agent is chloroquine or hydroxychloroquine. According to some embodiments, the anti-inflammatory agent is an antibody, such as for example, sarilumab, tocilizumab, or gimsilumab.

According to some embodiments, the additional compound is immune stimulatory agent. Also provided herein is a pharmaceutical composition comprising the lipid nanoparticle-encapsulated insect-cell produced, or a synthetically produced ceDNA vector for expression of anti-CoV S antibodies and antigen-binding fragments thereof (e.g., anti-CoV-2 S antibodies and antigen-binding fragments thereof) as described herein and a pharmaceutically acceptable carrier or excipient.

According to some aspects, the disclosure provides for a lipid nanoparticle formulation further comprising one or more pharmaceutical excipients. According to some embodiments, the lipid nanoparticle formulation further comprises sucrose, tris, trehalose and/or glycine.

The ceDNA vector can be complexed with the lipid portion of the particle or encapsulated in the lipid position of the lipid nanoparticle. According to some embodiments, the ceDNA can be fully encapsulated in the lipid position of the lipid nanoparticle, thereby protecting it from degradation by a nuclease, e.g., in an aqueous solution. According to some embodiments, the ceDNA in the lipid nanoparticle is not substantially degraded after exposure of the lipid nanoparticle to a nuclease at 37° C. for at least about 20, 30, 45, or 60 minutes. According to some embodiments, the ceDNA in the lipid nanoparticle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours.

In certain embodiments, the lipid nanoparticles are substantially non-toxic to a subject, e.g., to a mammal such as a human. According to some aspects, the lipid nanoparticle formulation is a lyophilized powder.

According to some embodiments, lipid nanoparticles are solid core particles that possess at least one lipid bilayer. In other embodiments, the lipid nanoparticles have a non-bilayer structure, i.e., a non-lamellar (i.e., non-bilayer) morphology. Without limitations, the non-bilayer morphology can include, for example, three dimensional tubes, rods, cubic symmetries, etc. For example, the morphology of the lipid nanoparticles (lamellar vs. non-lamellar) can readily be assessed and characterized using, e.g., Cryo-TEM analysis as described in US2010/0130588, the content of which is incorporated herein by reference in its entirety.

According to some further embodiments, the lipid nanoparticles having a non-lamellar morphology are electron dense. According to some aspects, the disclosure provides for a lipid nanoparticle that is either unilamellar or multilamellar in structure. According to some aspects, the disclosure provides for a lipid nanoparticle formulation that comprises multi-vesicular particles and/or foam-based particles.

By controlling the composition and concentration of the lipid components, one can control the rate at which the lipid conjugate exchanges out of the lipid particle and, in turn, the rate at which the lipid nanoparticle becomes fusogenic. In addition, other variables including, e.g., pH, temperature, or ionic strength, can be used to vary and/or control the rate at which the lipid nanoparticle becomes fusogenic. Other methods which can be used to control the rate at which the lipid nanoparticle becomes fusogenic will be apparent to those of ordinary skill in the art based on this disclosure. It will also be apparent that by controlling the composition and concentration of the lipid conjugate, one can control the lipid particle size.

The pKa of formulated cationic lipids can be correlated with the effectiveness of the LNPs for delivery of nucleic acids (see Jayaraman et al, Angewandte Chemie, International Edition (2012), 51(34), 8529-8533; Semple et al, Nature Biotechnology 28, 172-176 (2010), both of which are incorporated by reference in their entirety). The preferred range of pKa is ˜5 to ˜ 7. The pKa of the cationic lipid can be determined in lipid nanoparticles using an assay based on fluorescence of 2-(p-toluidino)-6-napthalene sulfonic acid (TNS).

### VI. Methods of Treatment

A ceDNA vector for expression of anti-CoV-2 S antibodies and antigen-binding fragments thereof as disclosed herein can also be used in a method for the delivery of a nucleic acid sequence of interest (e.g., encoding anti-CoV-2 S antibodies and antigen-binding fragments thereof) to a target cell (e.g., a host cell). The method may in particular be a method for delivering anti-CoV-2 S antibodies and antigen-binding fragments thereof to a cell of a subject in need thereof and treating COVID-19.

In addition, the disclosure provides a method for the delivery of anti-CoV-2 S antibodies and antigen-binding fragments thereof in a cell of a subject in need thereof, comprising multiple administrations of the ceDNA vector of the disclosure encoding said anti-CoV-2 S antibodies and antigen-binding fragments thereof. Since the ceDNA vector of the disclosure does not induce an immune response like that typically observed against encapsidated viral vectors, such a multiple administration strategy will likely have greater success in a ceDNA-based system. The ceDNA vector are administered in sufficient amounts to transfect the cells of a desired tissue and to provide sufficient levels of gene transfer and expression of the anti-CoV-2 S antibodies and antigen-binding fragments thereof without undue adverse effects.

The present disclosure provides methods for treating or preventing SARS-CoV-2 infection by administering a ceDNA vector for expression of anti-CoV-2 S antibodies and antigen-binding fragments thereof as described herein to a subject (e.g., a human) in need of such treatment or prevention.

According to some embodiments, COVID-19 may be treated or prevented, in a subject, by administering a ceDNA vector for expression of anti-CoV-2 S antibodies and antigen-binding fragments thereof as described herein to a subject.

An effective or therapeutically effective dose of a ceDNA vector for expression of anti-CoV-2 S antibodies and antigen-binding fragments thereof as described herein, for treating or preventing a viral infection refers to the amount of the ceDNA vector for expression of anti-CoV-2 S antibodies and antigen-binding fragments thereof as described herein that is sufficient to alleviate one or more signs and/or symptoms of the infection in the treated subject, whether by inducing the regression or elimination of such signs and/or symptoms or by inhibiting the progression of such signs and/or symptoms. The dose amount may vary depending upon the age and the size of a subject to be administered, target disease, conditions, route of administration, and the like. In an embodiment of the disclosure, an effective or therapeutically effective dose of antibody or antigen-binding fragment thereof of the present disclosure, for treating or preventing SARS-CoV-2 infection, e.g., in an adult human subject, is about 0.01 to about 200 mg/kg, e.g., up to about 150 mg/kg. In an embodiment of the disclosure, the dosage is up to about 10.8 or 11 grams (e.g., about 1, 2, 3, 4, 5, 6, 7, 8, 9, 10 or 11 grams). Depending on the severity of the infection, the frequency and the duration of the treatment can be adjusted. In certain embodiments, the ceDNA vector for expression of anti-CoV-2 S antibodies and antigen-binding fragments thereof as described herein can be administered at an initial dose, followed by one or more secondary doses. In certain embodiments, the initial dose may be followed by administration of a second or a plurality of subsequent doses of antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

The subject that is administered the ceDNA vector may have a viral infection, e.g., an influenza infection, or be predisposed to developing an infection. Subjects predisposed to developing an infection, or subjects who may be at elevated risk for contracting an infection (e.g., of coronavirus or influenza virus), include subjects with compromised immune systems because of autoimmune disease, subjects receiving immunosuppressive therapy (for example, following organ transplant), subjects afflicted with human immunodeficiency syndrome (HIV) or acquired immune deficiency syndrome (AIDS), subjects with forms of anemia that deplete or destroy white blood cells, subjects receiving radiation or chemotherapy, or subjects afflicted with an inflammatory disorder. Additionally, subjects of very young (e.g., 5 years of age or younger) or old age (e.g., 65 years of age or older) are at increased risk. Moreover, a subject may be at risk of contracting a viral infection due to proximity to an outbreak of the disease, e.g., subject resides in a densely-populated city or in close proximity to subjects having confirmed or suspected infections of a virus, or choice of employment, e.g., hospital worker, pharmaceutical researcher, traveler to infected area, or frequent flier.

The present disclosure also encompasses prophylactically administering a ceDNA vector for expression of anti-CoV-2 S antibodies and antigen-binding fragments thereof as described herein, to a subject who is at risk of viral infection so as to prevent such infection. “Prevent” or “preventing” means to administer a ceDNA vector for expression of anti-CoV-2 S antibodies and antigen-binding fragments thereof as described herein, to a subject to inhibit the manifestation of a disease or infection (e.g., viral infection) in the body of a subject, for which the ceDNA vector for expression of anti-CoV-2 S antibodies and antigen-binding fragments thereof as described herein is effective when administered to the subject at an effective or therapeutically effective amount or dose.

According to some embodiments, a sign or symptom of a viral infection in a subject is survival or proliferation of virus in the body of the subject, e.g., as determined by viral titer assay (e.g., coronavirus propagation in embryonated chicken eggs or coronavirus spike protein assay). Other signs and symptoms of viral infection are discussed herein.

As noted above, according to some embodiments the subject may be a non-human animal, and the antibodies and antigen-binding fragments discussed herein may be used in a veterinary context to treat and/or prevent disease in the non-human animals (e.g., cats, dogs, pigs, cows, horses, goats, rabbits, sheep, and the like).

The present disclosure provides a method for treating or preventing viral infection (e.g., SARS-CoV-2 infection) or for inducing the regression or elimination or inhibiting the progression of at least one sign or symptom of viral infection such as: fever or feeling feverish/chills; cough; sore throat; runny or stuffy nose; sneezing; muscle or body aches; headaches; fatigue (tiredness); vomiting; diarrhea; respiratory tract infection; chest discomfort; shortness of breath; bronchitis; and/or pneumonia, which sign or symptom is secondary to viral infection, in a subject in need thereof (e.g., a human), by administering a therapeutically effective amount of a ceDNA vector for expression of anti-CoV-2 S antibodies and antigen-binding fragments thereof as described herein to the subject.

### A. Ex Vivo Treatment

According to some embodiments, cells are removed from a subject, a ceDNA vector for expression of anti-CoV-2 S antibodies and antigen-binding fragments thereof as disclosed herein is introduced therein, and the cells are then replaced back into the subject. Methods of removing cells from subject for treatment ex vivo, followed by introduction back into the subject are known in the art (see, e.g., U.S. Pat. No. 5,399,346; the disclosure of which is incorporated herein in its entirety). Alternatively, a ceDNA vector is introduced into cells from another subject, into cultured cells, or into cells from any other suitable source, and the cells are administered to a subject in need thereof.

Cells transduced with a ceDNA vector for expression of anti-CoV-2 S antibodies and antigen-binding fragments thereof as disclosed herein are preferably administered to the subject in a “therapeutically-effective amount” in combination with a pharmaceutical carrier. Those skilled in the art will appreciate that the therapeutic effects need not be complete or curative, as long as some benefit is provided to the subject.

According to some embodiments, a ceDNA vector for expression of anti-CoV-2 S antibodies and antigen-binding fragments thereof as disclosed herein can encode an anti-CoV-2 S antibodies and antigen-binding fragments thereof as described herein that is to be produced in a cell in vitro, ex vivo, or in vivo. For example, in contrast to the use of the ceDNA vectors described herein in a method of treatment as discussed herein, according to some embodiments a ceDNA vector for expression of anti-CoV-2 S antibodies and antigen-binding fragments thereof may be introduced into cultured cells and the expressed anti-CoV-2 S antibodies and antigen-binding fragments thereof) isolated from the cells, e.g., for the production of antibodies and fusion proteins. According to some embodiments, the cultured cells comprising a ceDNA vector for expression of anti-CoV-2 S antibodies and antigen-binding fragments thereof as disclosed herein can be used for commercial production of antibodies or fusion proteins, e.g., serving as a cell source for small or large scale biomanufacturing of antibodies or fusion proteins. In alternative embodiments, a ceDNA vector for expression of anti-CoV-2 S antibodies and antigen-binding fragments thereof as disclosed herein is introduced into cells in a host non-human subject, for in vivo production of antibodies or fusion proteins, including small scale production as well as for commercial large scale anti-CoV-2 S antibodies and antigen-binding fragments thereof production.

The ceDNA vectors for expression of anti-CoV-2 S antibodies and antigen-binding fragments thereof as disclosed herein can be used in both veterinary and medical applications. Suitable subjects for ex vivo gene delivery methods as described above include both avians (e.g., chickens, ducks, geese, quail, turkeys and pheasants) and mammals (e.g., humans, bovines, ovines, caprines, equines, felines, canines, and lagomorphs), with mammals being preferred. Human subjects are most preferred. Human subjects include neonates, infants, juveniles, and adults.

### B. Dose Ranges

Provided herein are methods of treatment comprising administering to the subject an effective amount of a composition comprising a ceDNA vector encoding an anti-CoV S antibodies and antigen-binding fragments thereof (e.g., anti-CoV-2 S antibodies and antigen-binding fragments thereof) as described herein.

In vivo and/or in vitro assays can optionally be employed to help identify optimal dosage ranges for use. The precise dose to be employed in the formulation will also depend on the route of administration, and the seriousness of the condition, and should be decided according to the judgment of the person of ordinary skill in the art and each subject's circumstances. Effective doses can be extrapolated from dose-response curves derived from in vitro or animal model test systems

A ceDNA vector for expression of anti-CoV-2 S antibodies and antigen-binding fragments thereof as disclosed herein is administered in sufficient amounts to transfect the cells of a desired tissue and to provide sufficient levels of gene transfer and expression without undue adverse effects. Conventional and pharmaceutically acceptable routes of administration include, but are not limited to, those described above in the “Administration” section, such as direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration can be combined, if desired.

The dose of the amount of a ceDNA vectors for expression of anti-CoV-2 S antibodies and antigen-binding fragments thereof as disclosed herein required to achieve a particular “therapeutic effect.” will vary based on several factors including, but not limited to: the route of nucleic acid administration, the level of antibody expression required to achieve a therapeutic effect, the specific disease or disorder being treated, and the stability of the resulting expressed protein(s). One of skill in the art can readily determine a ceDNA vector dose range to treat a patient having a particular disease or disorder based on the aforementioned factors, as well as other factors that are well known in the art.

Dosage regime can be adjusted to provide the optimum therapeutic response. For example, the oligonucleotide can be repeatedly administered, e.g., several doses can be administered daily or the dose can be proportionally reduced as indicated by the exigencies of the therapeutic situation. One of ordinary skill in the art will readily be able to determine appropriate doses and schedules of administration of the subject oligonucleotides, whether the oligonucleotides are to be administered to cells or to subjects.

A “therapeutically effective dose” for clinical use will fall in a relatively broad range that can be determined through clinical trials and will depend on the particular application (e.g., neural cells will require very small amounts, while systemic injection would require large amounts). For example, for direct in vivo injection into skeletal or cardiac muscle of a human subject, a therapeutically effective dose will be on the order of from about 1 μg to 100 g of the ceDNA vector. If exosomes or microparticles are used to deliver the ceDNA vector, then a therapeutically effective dose can be determined experimentally, but is expected to deliver from 1 μg to about 100 g of vector. Moreover, a therapeutically effective dose is an amount ceDNA vector that expresses a sufficient amount of the transgene to have an effect on the subject that results in a reduction According to some or more symptoms of the disease, but does not result in significant off-target or significant adverse side effects. According to some embodiment, a “therapeutically effective amount” is an amount of an expressed anti-CoV-2 S antibodies and antigen-binding fragments thereof that is sufficient to produce a statistically significant, measurable change in reduction of a given disease symptom. Such effective amounts can be gauged in clinical trials as well as animal studies for a given ceDNA vector composition.

Treatment can involve administration of a single dose or multiple doses. According to some embodiments, more than one dose can be administered to a subject; in fact, multiple doses can be administered as needed, because the ceDNA vector does not elicit an anti-capsid host immune response due to the absence of a viral capsid. As such, one of skill in the art can readily determine an appropriate number of doses. The number of doses administered can, for example, be on the order of 1-100, preferably 2-20 doses.

Without wishing to be bound by any particular theory, the lack of typical anti-viral immune response elicited by administration of a ceDNA vector as described by the disclosure (i.e., the absence of capsid components) allows the ceDNA vector for expression of anti-CoV-2 S antibodies and antigen-binding fragments thereof to be administered to a host on multiple occasions. According to some embodiments, the number of occasions in which a nucleic acid is delivered to a subject is in a range of 2 to 10 times (e.g., 2, 3, 4, 5, 6, 7, 8, 9, or 10 times). According to some embodiments, a ceDNA vector is delivered to a subject more than 10 times.

According to some embodiments, a dose of a ceDNA vector is administered on day 0. Following the initial treatment at day 0, a second dosing (re-dose) can be performed in about 1 week, about 2 weeks, about 3 weeks, about 4 weeks, about 5 weeks, about 6 weeks, about 7 weeks, about 8 weeks, or about 3 months, about 4 months, about 5 months, about 6 months, about 7 months, about 8 months, about 9 months, about 10 months, about 11 months, or about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, about 25 years, about 26 years, about 27 years, about 28 years, about 29 years, about 30 years, about 31 years, about 32 years, about 33 years, about 34 years, about 35 years, about 36 years, about 37 years, about 38 years, about 39 years, about 40 years, about 41 years, about 42 years, about 43 years, about 44 years, about 45 years, about 46 years, about 47 years, about 48 years, about 49 years or about 50 years after the initial treatment with the ceDNA vector.

According to some embodiments, re-dosing of the therapeutic nucleic acid results in an increase in expression of the therapeutic nucleic acid. According to some embodiments, the increase of expression of the therapeutic nucleic acid after re-dosing, compared to the expression of the therapeutic nucleic acid after the first dose is about 0.5-fold to about 10-fold, about 1-fold to about 5-fold, about 1-fold to about 2-fold, or about 0.5-fold, about 1-fold, about 2-fold, about 3-fold, about 4-fold, about 5-fold, about 6-fold, about 7-fold, about 8-fold, about 9-fold or about 10-fold higher after re-dosing of the therapeutic nucleic acid.

In particular embodiments, more than one administration (e.g., two, three, four or more administrations) of a ceDNA vector for expression of anti-CoV-2 S antibodies and antigen-binding fragments thereof) as disclosed herein may be employed to achieve the desired level of antibody expression over a period of various intervals, e.g., daily, weekly, monthly, yearly, etc.

As described herein, according to some embodiments, a ceDNA vector expressing anti-CoV-2 S antibodies and antigen-binding fragments thereof can be administered in combination with an additional compound.

### C. Unit Dosage Forms

According to some embodiments, the pharmaceutical compositions comprising a ceDNA vector for expression of anti-CoV-2 S antibodies and antigen-binding fragments thereof as disclosed herein can conveniently be presented in unit dosage form. A unit dosage form will typically be adapted to one or more specific routes of administration of the pharmaceutical composition. According to some embodiments, the unit dosage form is adapted for intravenous, intramuscular, or subcutaneous administration. According to some embodiments, the unit dosage form is adapted for administration by inhalation. According to some embodiments, the unit dosage form is adapted for administration by a vaporizer. According to some embodiments, the unit dosage form is adapted for administration by a nebulizer. According to some embodiments, the unit dosage form is adapted for administration by an aerosolizer. According to some embodiments, the unit dosage form is adapted for oral administration, for buccal administration, or for sublingual administration.

D. Testing for Successful Antibody Expression Using a ceDNA Vector

Assays well known in the art can be used to test the efficiency of expression of anti-CoV-2 S antibodies and antigen-binding fragments thereof by a ceDNA vector can be performed in both in vitro and in vivo models. Levels of the expression of the anti-CoV-2 S antibodies and antigen-binding fragments thereof by ceDNA can be assessed by one skilled in the art by measuring protein levels of the anti-CoV-2 S antibodies and antigen-binding fragments thereof (e.g., western blot analysis, and enzyme-linked immunosorbent assay (ELISA)). For in vivo applications, protein function assays can be used to test the functionality of a given anti-CoV-2 S antibodies and antigen-binding fragments thereof to determine if expression has successfully occurred. One skilled in the art will be able to determine the best test for measuring functionality of an anti-CoV-2 S antibodies and antigen-binding fragments thereof expressed by the ceDNA vector in vitro or in vivo.

It is contemplated herein that the effects of expression of anti-CoV-2 S antibodies and antigen-binding fragments thereof from the ceDNA vector in a cell or subject can last for at least 1 month, at least 2 months, at least 3 months, at least four months, at least 5 months, at least six months, at least 10 months, at least 12 months, at least 18 months, at least 2 years, at least 5 years, at least 10 years, at least 20 years, or can be permanent.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

### EXAMPLES

The following examples are provided by way of illustration not limitation. It will be appreciated by one of ordinary skill in the art that ceDNA vectors can be constructed from any of the wild-type or modified ITRs described herein, and that the following exemplary methods can be used to construct and assess the activity of such ceDNA vectors. While the methods are exemplified with certain ceDNA vectors, they are applicable to any ceDNA vector in keeping with the description.

### Example 1: Constructing ceDNA Vectors Using an Insect Cell-Based Method

Production of the ceDNA vectors using a polynucleotide construct template is described in Example 1 of PCT/US18/49996, which is incorporated herein in its entirety by reference. For example, a polynucleotide construct template used for generating the ceDNA vectors of the present disclosure can be a ceDNA-plasmid, a ceDNA-Bacmid, and/or a ceDNA-baculovirus. Without being limited to theory, in a permissive host cell, in the presence of e.g., Rep, the polynucleotide construct template having two symmetric ITRs and an expression construct, where at least one of the ITRs is modified relative to a wild-type ITR sequence, replicates to produce ceDNA vectors. ceDNA vector production undergoes two steps: first, excision (“rescue”) of template from the template backbone (e.g., ceDNA-plasmid, ceDNA-bacmid, ceDNA-baculovirus genome etc.) via Rep proteins, and second, Rep mediated replication of the excised ceDNA vector.

An exemplary method to produce ceDNA vectors is from a ceDNA-plasmid as described herein. Referring to FIGS. 1A and 1B, the polynucleotide construct template of each of the ceDNA-plasmids includes both a left modified ITR and a right modified ITR with the following between the ITR sequences: (i) an enhancer/promoter; (ii) a cloning site for a transgene; (iii) a posttranscriptional response element (e.g., the woodchuck hepatitis virus posttranscriptional regulatory element (WPRE)); and (iv) a poly-adenylation signal (e.g., from bovine growth hormone gene (BGHpA). Unique restriction endonuclease recognition sites (R1-R6) (shown in FIG. 1A and FIG. 1B) were also introduced between each component to facilitate the introduction of new genetic components into the specific sites in the construct. R3 (PmeI) GTTTAAAC and R4 (PacI) TTAATTAA enzyme sites are engineered into the cloning site to introduce an open reading frame of a transgene. These sequences were cloned into a pFastBac HT B plasmid obtained from ThermoFisher Scientific.

Production of ceDNA-Bacmids:

DH10Bac competent cells (MAX EFFICIENCY® DH10Bac™ Competent Cells, Thermo Fisher) were transformed with either test or control plasmids following a protocol according to the manufacturer's instructions. Recombination between the plasmid and a baculovirus shuttle vector in the DH10Bac cells were induced to generate recombinant ceDNA-bacmids. The recombinant bacmids were selected by screening a positive selection based on blue-white screening in E. coli (Φ80dlacZΔM15 marker provides α-complementation of the β-galactosidase gene from the bacmid vector) on a bacterial agar plate containing X-gal and IPTG with antibiotics to select for transformants and maintenance of the bacmid and transposase plasmids. White colonies caused by transposition that disrupts the β-galactoside indicator gene were picked and cultured in 10 ml of media.

The recombinant ceDNA-bacmids were isolated from the E. coli and transfected into Sf9 or Sf21 insect cells using FugeneHD to produce infectious baculovirus. The adherent Sf9 or Sf21 insect cells were cultured in 50 ml of media in T25 flasks at 25° C. Four days later, culture medium (containing the P0 virus) was removed from the cells, filtered through a 0.45 μm filter, separating the infectious baculovirus particles from cells or cell debris.

Optionally, the first generation of the baculovirus (P0) was amplified by infecting naïve Sf9 or Sf21 insect cells in 50 to 500 ml of media. Cells were maintained in suspension cultures in an orbital shaker incubator at 130 rpm at 25° C., monitoring cell diameter and viability, until cells reach a diameter of 18-19 nm (from a naïve diameter of 14-15 nm), and a density of ˜4.0E+6 cells/mL. Between 3 and 8 days post-infection, the P1 baculovirus particles in the medium were collected following centrifugation to remove cells and debris then filtration through a 0.45 μm filter.

The ceDNA-baculovirus comprising the test constructs were collected and the infectious activity, or titer, of the baculovirus was determined. Specifically, four×20 mL Sf9 cell cultures at 2.5E+6 cells/ml were treated with P1 baculovirus at the following dilutions: 1/1000, 1/10,000, 1/50,000, 1/100,000, and incubated at 25-27° C. Infectivity was determined by the rate of cell diameter increase and cell cycle arrest and change in cell viability every day for 4 to 5 days.

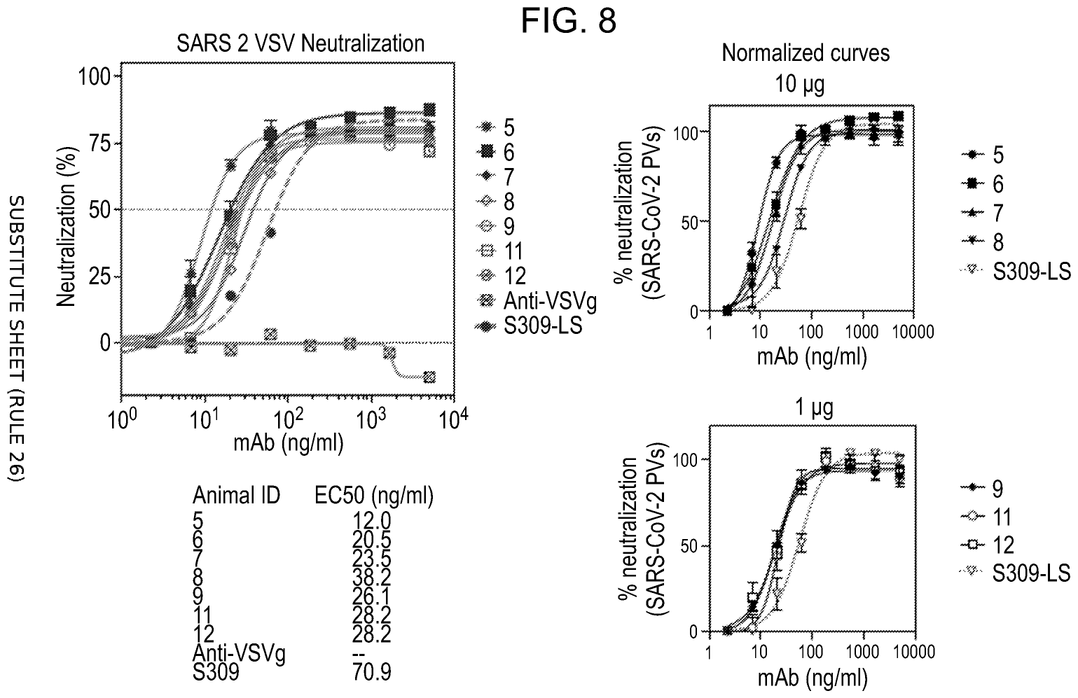

A “Rep-plasmid” as disclosed in FIG. 8A of PCT/US18/49996, which is incorporated herein in its entirety by reference, was produced in a pFASTBAC™-Dual expression vector (ThermoFisher) comprising both the Rep78 and Rep52 or Rep68 and Rep40. The Rep-plasmid was transformed into the DH10Bac competent cells (MAX EFFICIENCY® DH10Bac™ Competent Cells (Thermo Fisher) following a protocol provided by the manufacturer. Recombination between the Rep-plasmid and a baculovirus shuttle vector in the DH10Bac cells were induced to generate recombinant bacmids (“Rep-bacmids”). The recombinant bacmids were selected by a positive selection that included-blue-white screening in E. coli (Φ80dlacZΔM15 marker provides α-complementation of the β-galactosidase gene from the bacmid vector) on a bacterial agar plate containing X-gal and IPTG. Isolated white colonies were picked and inoculated in 10 ml of selection media (kanamycin, gentamicin, tetracycline in LB broth). The recombinant bacmids (Rep-bacmids) were isolated from the E. coli and the Rep-bacmids were transfected into Sf9 or Sf21 insect cells to produce infectious baculovirus.

The Sf9 or Sf21 insect cells were cultured in 50 ml of media for 4 days, and infectious recombinant baculovirus (“Rep-baculovirus”) were isolated from the culture. Optionally, the first generation Rep-baculovirus (P0) were amplified by infecting naïve Sf9 or Sf21 insect cells and cultured in 50 to 500 ml of media. Between 3 and 8 days post-infection, the P1 baculovirus particles in the medium were collected either by separating cells by centrifugation or filtration or another fractionation process. The Rep-baculovirus were collected and the infectious activity of the baculovirus was determined. Specifically, four×20 mL Sf9 cell cultures at 2.5×10⁶ cells/mL were treated with P1 baculovirus at the following dilutions, 1/1000, 1/10,000, 1/50,000, 1/100,000, and incubated. Infectivity was determined by the rate of cell diameter increase and cell cycle arrest, and change in cell viability every day for 4 to 5 days.

ceDNA Vector Generation and Characterization

With reference to FIG. 4B, Sf9 insect cell culture media containing either (1) a sample-containing a ceDNA-bacmid or a ceDNA-baculovirus, and (2) Rep-baculovirus described above were then added to a fresh culture of Sf9 cells (2.5E+6 cells/ml, 20 ml) at a ratio of 1:1000 and 1:10,000, respectively. The cells were then cultured at 130 rpm at 25° C. 4-5 days after the co-infection, cell diameter and viability are detected. When cell diameters reached 18-20 nm with a viability of ˜70-80%, the cell cultures were centrifuged, the medium was removed, and the cell pellets were collected. The cell pellets are first resuspended in an adequate volume of aqueous medium, either water or buffer. The ceDNA vector was isolated and purified from the cells using Qiagen MIDI PLUS™ purification protocol (Qiagen, 0.2 mg of cell pellet mass processed per column).

Yields of ceDNA vectors produced and purified from the Sf9 insect cells were initially determined based on UV absorbance at 260 nm.

ceDNA vectors can be assessed by identified by agarose gel electrophoresis under native or denaturing conditions as illustrated in FIG. 4D, where (a) the presence of characteristic bands migrating at twice the size on denaturing gels versus native gels after restriction endonuclease cleavage and gel electrophoretic analysis and (b) the presence of monomer and dimer (2×) bands on denaturing gels for uncleaved material is characteristic of the presence of ceDNA vector.

Structures of the isolated ceDNA vectors were further analyzed by digesting the DNA obtained from co-infected Sf9 cells (as described herein) with restriction endonucleases selected for a) the presence of only a single cut site within the ceDNA vectors, and b) resulting fragments that were large enough to be seen clearly when fractionated on a 0.8% denaturing agarose gel (>800 bp). As illustrated in FIGS. 4D and 4E, linear DNA vectors with a non-continuous structure and ceDNA vector with the linear and continuous structure can be distinguished by sizes of their reaction products—for example, a DNA vector with a non-continuous structure is expected to produce 1 kb and 2 kb fragments, while a non-encapsidated vector with the continuous structure is expected to produce 2 kb and 4 kb fragments.

Therefore, to demonstrate in a qualitative fashion that isolated ceDNA vectors are covalently closed-ended as is required by definition, the samples were digested with a restriction endonuclease identified in the context of the specific DNA vector sequence as having a single restriction site, preferably resulting in two cleavage products of unequal size (e.g., 1000 bp and 2000 bp). Following digestion and electrophoresis on a denaturing gel (which separates the two complementary DNA strands), a linear, non-covalently closed DNA will resolve at sizes 1000 bp and 2000 bp, while a covalently closed DNA (i.e., a ceDNA vector) will resolve at 2× sizes (2000 bp and 4000 bp), as the two DNA strands are linked and are now unfolded and twice the length (though single stranded). Furthermore, digestion of monomeric, dimeric, and n-meric forms of the DNA vectors will all resolve as the same size fragments due to the end-to-end linking of the multimeric DNA vectors (see FIG. 4D).

As used herein, the phrase “assay for the Identification of DNA vectors by agarose gel electrophoresis under native gel and denaturing conditions” refers to an assay to assess the close-endedness of the ceDNA by performing restriction endonuclease digestion followed by electrophoretic assessment of the digest products. One such exemplary assay follows, though one of ordinary skill in the art will appreciate that many art-known variations on this example are possible. The restriction endonuclease is selected to be a single cut enzyme for the ceDNA vector of interest that will generate products of approximately ⅓× and ⅔× of the DNA vector length. This resolves the bands on both native and denaturing gels. Before denaturation, it is important to remove the buffer from the sample. The Qiagen PCR clean-up kit or desalting “spin columns.” e.g., GE HEALTHCARE ILUSTRA™ MICROSPIN™ G-25 columns are some art-known options for the endonuclease digestion. The assay includes for example, i) digest DNA with appropriate restriction endonuclease(s), 2) apply to e.g., a Qiagen PCR clean-up kit, elute with distilled water, iii) adding 10× denaturing solution (10×=0.5 M NaOH, 10 mM EDTA), add 10× dye, not buffered, and analyzing, together with DNA ladders prepared by adding 10× denaturing solution to 4×, on a 0.8-1.0% gel previously incubated with 1 mM EDTA and 200 mM NaOH to ensure that the NaOH concentration is uniform in the gel and gel box, and running the gel in the presence of 1× denaturing solution (50 mM NaOH, 1 mM EDTA). One of ordinary skill in the art will appreciate what voltage to use to run the electrophoresis based on size and desired timing of results. After electrophoresis, the gels are drained and neutralized in 1×TBE or TAE and transferred to distilled water or 1×TBE/TAE with 1×SYBR Gold. Bands can then be visualized with e.g., Thermo Fisher, SYBR® Gold Nucleic Acid Gel Stain (10,000× Concentrate in DMSO) and epifluorescent light (blue) or UV (312 nm).

The purity of the generated ceDNA vector can be assessed using any art-known method. As one exemplary and non-limiting method, contribution of ceDNA-plasmid to the overall UV absorbance of a sample can be estimated by comparing the fluorescent intensity of ceDNA vector to a standard. For example, if based on UV absorbance 4 μg of ceDNA vector was loaded on the gel, and the ceDNA vector fluorescent intensity is equivalent to a 2 kb band which is known to be 1 μg, then there is 1 μg of ceDNA vector, and the ceDNA vector is 25% of the total UV absorbing material. Band intensity on the gel is then plotted against the calculated input that band represents—for example, if the total ceDNA vector is 8 kb, and the excised comparative band is 2 kb, then the band intensity would be plotted as 25% of the total input, which in this case would be 0.25 μg for 1.0 μg input. Using the ceDNA vector plasmid titration to plot a standard curve, a regression line equation is then used to calculate the quantity of the ceDNA vector band, which can then be used to determine the percent of total input represented by the ceDNA vector, or percent purity.

For comparative purposes, Example 1 describes the production of ceDNA vectors using an insect cell-based method and a polynucleotide construct template, and is also described in Example 1 of PCT/US18/49996, which is incorporated herein in its entirety by reference. For example, a polynucleotide construct template used for generating the ceDNA vectors of the present disclosure according to Example 1 can be a ceDNA-plasmid, a ceDNA-Bacmid, and/or a ceDNA-baculovirus. Without being limited to theory, in a permissive host cell, in the presence of e.g., Rep, the polynucleotide construct template having two symmetric ITRs and an expression construct, where at least one of the ITRs is modified relative to a wild-type ITR sequence, replicates to produce ceDNA vectors. ceDNA vector production undergoes two steps: first, excision (“rescue”) of template from the template backbone (e.g., ceDNA-plasmid, ceDNA-bacmid, ceDNA-baculovirus genome etc.) via Rep proteins, and second, Rep mediated replication of the excised ceDNA vector.

An exemplary method to produce ceDNA vectors in a method using insect cell is from a ceDNA-plasmid as described herein. Referring to FIGS. 1A and 1B, the polynucleotide construct template of each of the ceDNA-plasmids includes both a left modified ITR and a right modified ITR with the following between the ITR sequences: (i) an enhancer/promoter; (ii) a cloning site for a transgene; (iii) a posttranscriptional response element (e.g., the woodchuck hepatitis virus posttranscriptional regulatory element (WPRE)); and (iv) a poly-adenylation signal (e.g., from bovine growth hormone gene (BGHpA). Unique restriction endonuclease recognition sites (R1-R6) (shown in FIG. 1A and FIG. 1B) were also introduced between each component to facilitate the introduction of new genetic components into the specific sites in the construct. R3 (PmeI) GTTTAAAC and R4 (PacI) TTAATTAA enzyme sites are engineered into the cloning site to introduce an open reading frame of a transgene. These sequences were cloned into a pFastBac HT B plasmid obtained from ThermoFisher Scientific.

Production of ceDNA-Bacmids:

DH10Bac competent cells (MAX EFFICIENCY® DH10Bac™ Competent Cells, Thermo Fisher) were transformed with either test or control plasmids following a protocol according to the manufacturer's instructions. Recombination between the plasmid and a baculovirus shuttle vector in the DH10Bac cells were induced to generate recombinant ceDNA-bacmids. The recombinant bacmids were selected by screening a positive selection based on blue-white screening in E. coli (Φ80dlacZΔM15 marker provides α-complementation of the β-galactosidase gene from the bacmid vector) on a bacterial agar plate containing X-gal and IPTG with antibiotics to select for transformants and maintenance of the bacmid and transposase plasmids. White colonies caused by transposition that disrupts the β-galactoside indicator gene were picked and cultured in 10 ml of media.

The recombinant ceDNA-bacmids were isolated from the E. coli and transfected into Sf9 or Sf21 insect cells using FugeneHD to produce infectious baculovirus. The adherent Sf9 or Sf21 insect cells were cultured in 50 ml of media in T25 flasks at 25° C. Four days later, culture medium (containing the P0 virus) was removed from the cells, filtered through a 0.45 μm filter, separating the infectious baculovirus particles from cells or cell debris.

Optionally, the first generation of the baculovirus (P0) was amplified by infecting naïve Sf9 or Sf21 insect cells in 50 to 500 ml of media. Cells were maintained in suspension cultures in an orbital shaker incubator at 130 rpm at 25° C., monitoring cell diameter and viability, until cells reach a diameter of 18-19 nm (from a naïve diameter of 14-15 nm), and a density of ˜4.0E+6 cells/mL. Between 3 and 8 days post-infection, the P1 baculovirus particles in the medium were collected following centrifugation to remove cells and debris then filtration through a 0.45 μm filter.

The ceDNA-baculovirus comprising the test constructs were collected and the infectious activity, or titer, of the baculovirus was determined. Specifically, four×20 ml Sf9 cell cultures at 2.5E+6 cells/ml were treated with P1 baculovirus at the following dilutions: 1/1000, 1/10,000, 1/50,000, 1/100,000, and incubated at 25-27° C. Infectivity was determined by the rate of cell diameter increase and cell cycle arrest, and change in cell viability every day for 4 to 5 days.

A “Rep-plasmid” was produced in a pFASTBAC™-Dual expression vector (ThermoFisher) comprising both the Rep78 or Rep68 and Rep52 or Rep40. The Rep-plasmid was transformed into the DH10Bac competent cells (MAX EFFICIENCY® DH10Bac™ Competent Cells (Thermo Fisher) following a protocol provided by the manufacturer. Recombination between the Rep-plasmid and a baculovirus shuttle vector in the DH10Bac cells were induced to generate recombinant bacmids (“Rep-bacmids”). The recombinant bacmids were selected by a positive selection that included-blue-white screening in E. coli (Φ80dlacZΔM15 marker provides α-complementation of the β-galactosidase gene from the bacmid vector) on a bacterial agar plate containing X-gal and IPTG. Isolated white colonies were picked and inoculated in 10 ml of selection media (kanamycin, gentamicin, tetracycline in LB broth). The recombinant bacmids (Rep-bacmids) were isolated from the E. coli and the Rep-bacmids were transfected into Sf9 or Sf21 insect cells to produce infectious baculovirus.

The Sf9 or Sf21 insect cells were cultured in 50 ml of media for 4 days, and infectious recombinant baculovirus (“Rep-baculovirus”) were isolated from the culture. Optionally, the first generation Rep-baculovirus (P0) were amplified by infecting naïve Sf9 or Sf21 insect cells and cultured in 50 to 500 ml of media. Between 3 and 8 days post-infection, the P1 baculovirus particles in the medium were collected either by separating cells by centrifugation or filtration or another fractionation process. The Rep-baculovirus were collected and the infectious activity of the baculovirus was determined. Specifically, four×20 mL Sf9 cell cultures at 2.5×10⁶ cells/mL were treated with P1 baculovirus at the following dilutions, 1/1000, 1/10,000, 1/50,000, 1/100,000, and incubated. Infectivity was determined by the rate of cell diameter increase and cell cycle arrest, and change in cell viability every day for 4 to 5 days.

ceDNA Vector Generation and Characterization

Sf9 insect cell culture media containing either (1) a sample-containing a ceDNA-bacmid or a ceDNA-baculovirus, and (2) Rep-baculovirus described above were then added to a fresh culture of Sf9 cells (2.5E+6 cells/ml. 20 ml) at a ratio of 1:1000 and 1:10,000, respectively. The cells were then cultured at 130 rpm at 25° C. 4-5 days after the co-infection, cell diameter and viability are detected. When cell diameters reached 18-20 nm with a viability of ˜70-80%, the cell cultures were centrifuged, the medium was removed, and the cell pellets were collected. The cell pellets are first resuspended in an adequate volume of aqueous medium, either water or buffer. The ceDNA vector was isolated and purified from the cells using Qiagen MIDI PLUS™ purification protocol (Qiagen. 0.2 mg of cell pellet mass processed per column).

Yields of ceDNA vectors produced and purified from the Sf9 insect cells were initially determined based on UV absorbance at 260 nm. The purified ceDNA vectors can be assessed for proper closed-ended configuration using the electrophoretic methodology described in Example 5.

### Example 2: Synthetic ceDNA Production Via Excision from a Double-Stranded DNA Molecule

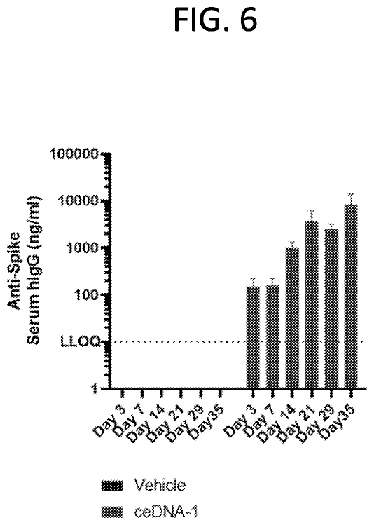

Synthetic production of the ceDNA vectors is described in Examples 2-6 of International Application PCT/US19/14122, filed Jan. 18, 2019, which is incorporated herein in its entirety by reference. One exemplary method of producing a ceDNA vector using a synthetic method that involves the excision of a double-stranded DNA molecule. In brief, a ceDNA vector can be generated using a double stranded DNA construct, e.g., see FIGS. 7A-8E of PCT/US19/14122. According to some embodiments, the double stranded DNA construct is a ceDNA plasmid, e.g., see, e.g., FIG. 6 in International patent application PCT/US2018/064242, filed Dec. 6, 2018).

According to some embodiments, a construct to make a ceDNA vector comprises a regulatory switch as described herein.

For illustrative purposes, Example 2 describes producing ceDNA vectors as exemplary closed-ended DNA vectors generated using this method. However, while ceDNA vectors are exemplified in this Example to illustrate in vitro synthetic production methods to generate a closed-ended DNA vector by excision of a double-stranded polynucleotide comprising the ITRs and expression cassette (e.g., nucleic acid sequence) followed by ligation of the free 3′ and 5′ ends as described herein, one of ordinary skill in the art is aware that one can, as illustrated above, modify the double stranded DNA polynucleotide molecule such that any desired closed-ended DNA vector is generated, including but not limited to, doggybone DNA, dumbbell DNA and the like. Exemplary ceDNA vectors for production of antibodies or fusion proteins that can be produced by the synthetic production method described in Example 2 are discussed in the sections entitled “III ceDNA vectors in general”.

Exemplary antibodies and fusion proteins expressed by the ceDNA vectors are described in the section entitled “IIC Exemplary antibodies and fusion proteins expressed by the ceDNA vectors”.

The method involves (i) excising a sequence encoding the expression cassette from a double-stranded DNA construct and (ii) forming hairpin structures at one or more of the ITRs and (iii) joining the free 5′ and 3′ ends by ligation, e.g., by T4 DNA ligase.

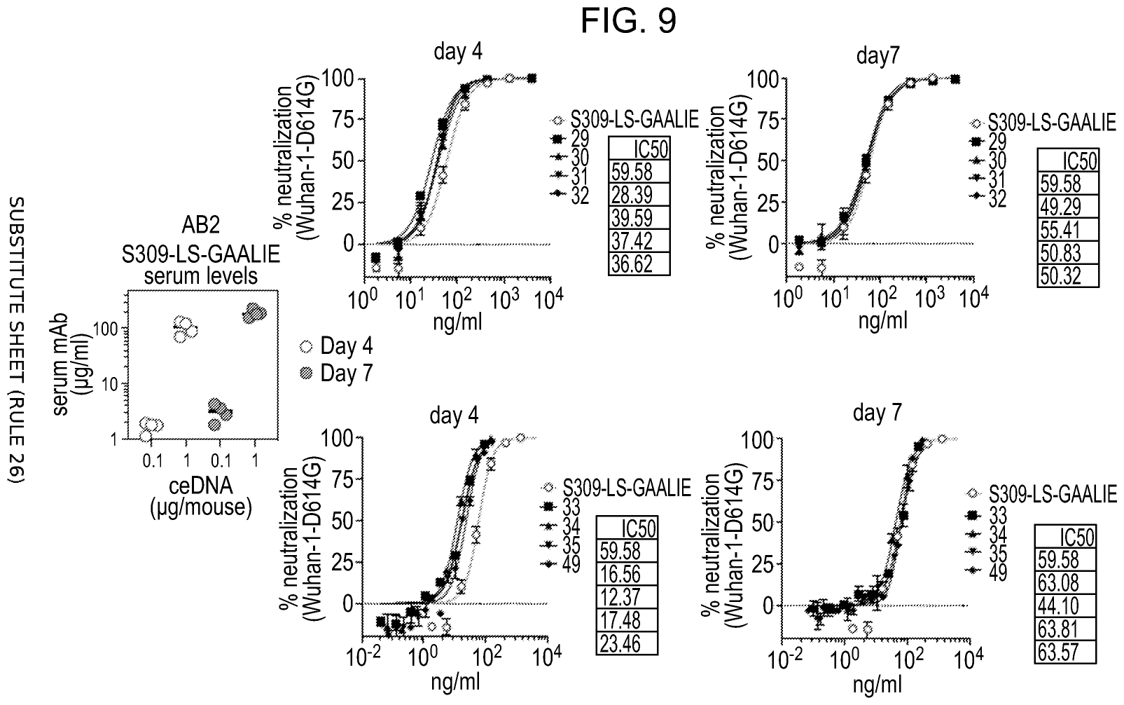

The double-stranded DNA construct comprises, in 5′ to 3′ order: a first restriction endonuclease site; an upstream ITR; an expression cassette; a downstream ITR; and a second restriction endonuclease site. The double-stranded DNA construct is then contacted with one or more restriction endonucleases to generate double-stranded breaks at both of the restriction endonuclease sites. One endonuclease can target both sites, or each site can be targeted by a different endonuclease as long as the restriction sites are not present in the ceDNA vector template. This excises the sequence between the restriction endonuclease sites from the rest of the double-stranded DNA construct (see FIG. 9 of PCT/US19/14122). Upon ligation a closed-ended DNA vector is formed.

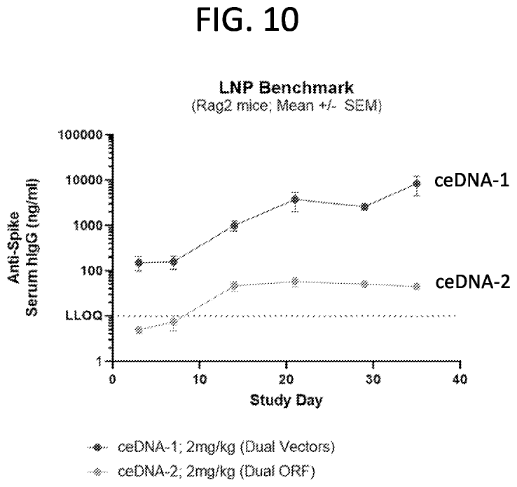

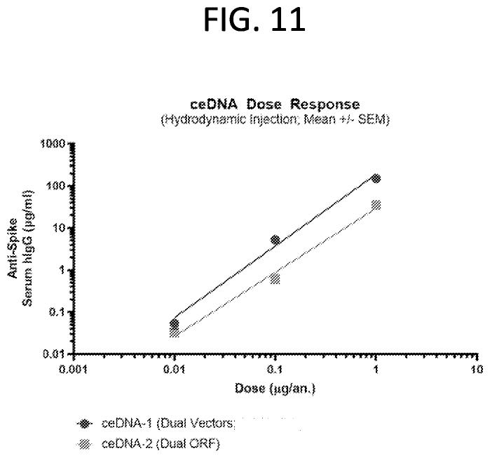

One or both of the ITRs used in the method may be wild-type ITRs. Modified ITRs may also be used, where the modification can include deletion, insertion, or substitution of one or more nucleotides from the wild-type ITR in the sequences forming B and B′ arm and/or C and C′ arm (see. e.g., FIGS. 6-8 and 10 FIG. 11B of PCT/US19/14122), and may have two or more hairpin loops (see, e.g., FIGS. 6-8 FIG. 11B of PCT/US19/14122) or a single hairpin loop (see, e.g., FIG. 10A-10B FIG. 11B of PCT/US19/14122). The hairpin loop modified ITR can be generated by genetic modification of an existing oligo or by de novo biological and/or chemical synthesis.

In a non-limiting example, ITR-6 Left and Right (SEQ ID NOS: 111 and 112), include 40 nucleotide deletions in the B-B′ and C-C′ arms from the wild-type ITR of AAV2. Nucleotides remaining in the modified ITR are predicted to form a single hairpin structure. Gibbs free energy of unfolding the structure is about −54.4 kcal/mol. Other modifications to the ITR may also be made, including optional deletion of a functional Rep binding site or a Trs site.

### Example 3: ceDNA Production Via Oligonucleotide Construction

Another exemplary method of producing a ceDNA vector using a synthetic method that involves assembly of various oligonucleotides, is provided in Example 3 of PCT/US19/14122, where a ceDNA vector is produced by synthesizing a 5′ oligonucleotide and a 3′ ITR oligonucleotide and ligating the ITR oligonucleotides to a double-stranded polynucleotide comprising an expression cassette. FIG. 11B of PCT/US19/14122 shows an exemplary method of ligating a 5′ ITR oligonucleotide and a 3′ ITR oligonucleotide to a double stranded polynucleotide comprising an expression cassette.

As disclosed herein, the ITR oligonucleotides can comprise WT-ITRs (e.g., see FIG. 3A, FIG. 3C), or modified ITRs (e.g., see, FIG. 3B and FIG. 3D). (See also, e.g., FIGS. 6A, 6B, 7A and 7B of PCT/US19/14122, which is incorporated herein in its entirety). Exemplary ITR oligonucleotides include, but are not limited to SEQ ID NOS: 134-145 (e.g., see Table 7 in of PCT/US19/14122). Modified ITRs can include deletion, insertion, or substitution of one or more nucleotides from the wild-type ITR in the sequences forming B and B′ arm and/or C and C′ arm. ITR oligonucleotides, comprising WT-ITRs or mod-ITRs as described herein, to be used in the cell-free synthesis, can be generated by genetic modification or biological and/or chemical synthesis. As discussed herein, the ITR oligonucleotides in Examples 2 and 3 can comprise WT-ITRs, or modified ITRs (mod-ITRs) in symmetrical or asymmetrical configurations, as discussed herein.

### Example 4: ceDNA Production Via a Single-Stranded DNA Molecule

Another exemplary method of producing a ceDNA vector using a synthetic method is provided in Example 4 of PCT/US19/14122, and uses a single-stranded linear DNA comprising two sense ITRs which flank a sense expression cassette sequence and are attached covalently to two antisense ITRs which flank an antisense expression cassette, the ends of which single stranded linear DNA are then ligated to form a closed-ended single-stranded molecule. One non-limiting example comprises synthesizing and/or producing a single-stranded DNA molecule, annealing portions of the molecule to form a single linear DNA molecule which has one or more base-paired regions of secondary structure, and then ligating the free 5′ and 3′ ends to each other to form a closed single-stranded molecule.

An exemplary single-stranded DNA molecule for production of a ceDNA vector comprises, from 5′ to 3′:
a sense first ITR;
a sense expression cassette sequence;
a sense second ITR;
an antisense second ITR;
an antisense expression cassette sequence; and
an antisense first ITR.

A single-stranded DNA molecule for use in the exemplary method of Example 4 can be formed by any DNA synthesis methodology described herein, e.g., in vitro DNA synthesis, or provided by cleaving a DNA construct (e.g., a plasmid) with nucleases and melting the resulting dsDNA fragments to provide ssDNA fragments.

Annealing can be accomplished by lowering the temperature below the calculated melting temperatures of the sense and antisense sequence pairs. The melting temperature is dependent upon the specific nucleotide base content and the characteristics of the solution being used, e.g., the salt concentration. Melting temperatures for any given sequence and solution combination are readily calculated by one of ordinary skill in the art.

The free 5′ and 3′ ends of the annealed molecule can be ligated to each other, or ligated to a hairpin molecule to form the ceDNA vector. Suitable exemplary ligation methodologies and hairpin molecules are described in Examples 2 and 3.

### Example 5: Purifying and/or Confirming Production of ceDNA

Any of the DNA vector products produced by the methods described herein, e.g., including the insect cell-based production methods described in Example 1, or synthetic production methods described in Examples 2-4 can be purified, e.g., to remove impurities, unused components, or byproducts using methods commonly known by a skilled artisan; and/or can be analyzed to confirm that DNA vector produced, (in this instance, a ceDNA vector) is the desired molecule. An exemplary method for purification of the DNA vector, e.g., ceDNA is using Qiagen Midi Plus purification protocol (Qiagen) and/or by gel purification,

The following is an exemplary method for confirming the identity of ceDNA vectors.

ceDNA vectors can be assessed by identified by agarose gel electrophoresis under native or denaturing conditions as illustrated in FIG. 4D, where (a) the presence of characteristic bands migrating at twice the size on denaturing gels versus native gels after restriction endonuclease cleavage and gel electrophoretic analysis and (b) the presence of monomer and dimer (2×) bands on denaturing gels for uncleaved material is characteristic of the presence of ceDNA vector.

Structures of the isolated ceDNA vectors were further analyzed by digesting the purified DNA with restriction endonucleases selected for a) the presence of only a single cut site within the ceDNA vectors, and b) resulting fragments that were large enough to be seen clearly when fractionated on a 0.8% denaturing agarose gel (>800 bp). As illustrated in FIGS. 4C and 4D, linear DNA vectors with a non-continuous structure and ceDNA vector with the linear and continuous structure can be distinguished by sizes of their reaction products—for example, a DNA vector with a non-continuous structure is expected to produce 1 kb and 2 kb fragments, while a ceDNA vector with the continuous structure is expected to produce 2 kb and 4 kb fragments.

Therefore, to demonstrate in a qualitative fashion that isolated ceDNA vectors are covalently closed-ended as is required by definition, the samples were digested with a restriction endonuclease identified in the context of the specific DNA vector sequence as having a single restriction site, preferably resulting in two cleavage products of unequal size (e.g., 1000 bp and 2000 bp). Following digestion and electrophoresis on a denaturing gel (which separates the two complementary DNA strands), a linear, non-covalently closed DNA will resolve at sizes 1000 bp and 2000 bp, while a covalently closed DNA (i.e., a ceDNA vector) will resolve at 2× sizes (2000 bp and 4000 bp), as the two DNA strands are linked and are now unfolded and twice the length (though single stranded). Furthermore, digestion of monomeric, dimeric, and n-meric forms of the DNA vectors will all resolve as the same size fragments due to the end-to-end linking of the multimeric DNA vectors (see FIG. 4E).

As used herein, the phrase “assay for the Identification of DNA vectors by agarose gel electrophoresis under native gel and denaturing conditions” refers to an assay to assess the close-endedness of the ceDNA by performing restriction endonuclease digestion followed by electrophoretic assessment of the digest products. One such exemplary assay follows, though one of ordinary skill in the art will appreciate that many art-known variations on this example are possible. The restriction endonuclease is selected to be a single cut enzyme for the ceDNA vector of interest that will generate products of approximately ⅓× and ⅔× of the DNA vector length. This resolves the bands on both native and denaturing gels. Before denaturation, it is important to remove the buffer from the sample. The Qiagen PCR clean-up kit or desalting “spin columns.” e.g., GE HEALTHCARE ILUSTRA™ MICROSPIN™ G-25 columns are some art-known options for the endonuclease digestion. The assay includes for example, i) digest DNA with appropriate restriction endonuclease(s), 2) apply to e.g., a Qiagen PCR clean-up kit, elute with distilled water, iii) adding 10× denaturing solution (10×=0.5 M NaOH, 10 mM EDTA), add 10× dye, not buffered, and analyzing, together with DNA ladders prepared by adding 10× denaturing solution to 4×, on a 0.8-1.0% gel previously incubated with 1 mM EDTA and 200 mM NaOH to ensure that the NaOH concentration is uniform in the gel and gel box, and running the gel in the presence of 1× denaturing solution (50 mM NaOH, 1 mM EDTA). One of ordinary skill in the art will appreciate what voltage to use to run the electrophoresis based on size and desired timing of results. After electrophoresis, the gels are drained and neutralized in 1×TBE or TAE and transferred to distilled water or 1×TBE/TAE with 1×SYBR Gold. Bands can then be visualized with e.g., Thermo Fisher, SYBR® Gold Nucleic Acid Gel Stain (10,000× Concentrate in DMSO) and epifluorescent light (blue) or UV (312 nm). The foregoing gel-based method can be adapted to purification purposes by isolating the ceDNA vector from the gel band and permitting it to renature.

The purity of the generated ceDNA vector can be assessed using any art-known method. As one exemplary and non-limiting method, contribution of ceDNA-plasmid to the overall UV absorbance of a sample can be estimated by comparing the fluorescent intensity of ceDNA vector to a standard. For example, if based on UV absorbance 4 μg of ceDNA vector was loaded on the gel, and the ceDNA vector fluorescent intensity is equivalent to a 2 kb band which is known to be 1 μg, then there is 1 μg of ceDNA vector, and the ceDNA vector is 25% of the total UV absorbing material. Band intensity on the gel is then plotted against the calculated input that band represents—for example, if the total ceDNA vector is 8 kb, and the excised comparative band is 2 kb, then the band intensity would be plotted as 25% of the total input, which in this case would be 0.25 μg for 1.0 μg input. Using the ceDNA vector plasmid titration to plot a standard curve, a regression line equation is then used to calculate the quantity of the ceDNA vector band, which can then be used to determine the percent of total input represented by the ceDNA vector, or percent purity.

### Example 6: A Study to Screen Covid Ab Constructs Via Intravenous Delivery in Male Rag2 Mice

ceDNA vectors were produced according to the methods described in Example 1 above.

The objective of the study was to determine protein expression after intravenous delivery of formulated ceDNA delivered via LNP and ruxolitinib (a Janus Associated Kinase (JAK) inhibitor) as an immunosuppressant TKI. A ceDNA comprising a nucleic acid encoding the heavy chain (HC) of anti-SARS-CoV-2 Ab and a ceDNA comprising a nucleic acid encoding the light chain (LC) of anti-SARS-CoV-2 Ab was dosed in mice (n=5) in combination with the immunosuppressant TKI (e.g., ruxolitinib). The study design and details were carried out as set forth below.

### Study Design

Table 8 sets forth the design of the kinase inhibitor administration component of the study. As shown in Table 8, two groups of male Rag2 mice (n=5) were orally (PO) administered either ruxolitinib (300 mg/kg) at a dose volume of 10 mL/kg or not dosed. For animals that were dosed, dosing was carried out at day 0, 30 minutes pre-dose and 5 hours post-dose.

TABLE 8
Study Design of Kinase Inhibitor Administration

| | | | | Dose | Dose | | |
|---|---|---|---|---|---|---|---|
| Group | No. of | | | Levels | Volume | | |
| No. | Animals | Strain | Inhibitor | (mg/kg) | (mL/kg) | ROA | Dosing Regimen |
| 1 | 5 | Rag2 | NA | NA | NA | NA | NA |
| 2 | 5 | Rag2 | | | | | Day 0, |
| | | | | | | | 30 min pre and 5 |
| | | | | | | | h post |

No. = Number; PO = oral gavage; ROA = route of administration; min = minutes; hrs = hours.
Vehicle for dosing and inhibitor preparation = 0.5% methylcellulose

Table 9 sets forth the design of the test material administration component of the study. As shown in Table 9, the two groups of Rag2 mice (n=5) were intravenously administered vehicle (Group 1) or test compound (Groups 2) at a dose level of 2 mg/kg and a dose volume of 5 mL/kg, on Day 0. Day 35 was the terminal time point of the study. Group 1 served as the vehicle control. In Group 2, the test composition comprised the ceDNA vector combination comprising the ceDNA1856 (LC) and the ceDNA 1859 (HC) constructs. The ORFs of ceDNA vectors 1856 and 1859 are set forth above in Table 7.

TABLE 9
Test Material Administration

| | | | | Dose | Dose | | |
|---|---|---|---|---|---|---|---|
| Group | No. of | | | Levels | Volume | Dosing Regimen | Terminal Time |
| No. | Animals | Strain | Test Material | (mg/kg) | (mL/kg) | ROA | Point |
| 1 | 5 | Rag2 | Vehicle | NA | 5 | Once on | Day 35 |
| 2 | 5 | | ceDNA1856 (LC) + | 2 | | Day 0 by IV | |
| | | | ceDNA1859 | | | | |
| | | | (HC) in LNP | | | | |

No. = Number;
IV = intravenous;
ROA = route of administration

### Test System

The test system was as follows:
Species: Mus musculus
Strain: Rag2 (B6.129S6-Rag2<tm1Fwa>)
Number of Males: 25, plus 2 spares
Age: 5 weeks of age at arrival
Source: Taconic

Housing: Animals were group housed in clear polycarbonate cages with contact bedding in a procedure room.

Food and Water: Animals were provided ad libitum Mouse Diet 5058 and filtered tap water acidified with 1N HCl to a targeted pH of 2.5-3.0.

### Test Material

Class of Compound: Recombinant DNA Vector: ceDNA

Dose Formulation: Test articles were supplied in ready to dose aliquots. Test article concentration was recorded at time of receipt.

Stock was warmed to room temperature and diluted with the provided PBS immediately, as necessary, prior to use. Prepared materials were stored at ˜4° C. if dosing was not performed immediately.

Inhibitor was supplied in daily ready to dose aliquots. Oral gavage dose solution was formulated in 0.5% methylcellulose. Oral gavage formulations were mixed (pipetting) and/or sonicated prior to administration to distribute particulates of oral gavage suspension.

Inhibitor Administration: Inhibitor was dosed on Day 0 per Table 8 above, by PO administration (oral gavage) at 10 mL/kg. Inhibitor was dosed 30 minutes (±5 minutes) prior to and 5 hours (±10 minutes) post the Day 0 ceDNA administration.

Test Material Administration: Doses of test material were administered on Day 0 by intravenous dosing into the lateral tail vein. Doses were administered at a dose volume of 5 mL/kg. Doses were rounded to the nearest 0.01 mL.

Residual Materials: All residual open stock was placed in the refrigerator and discarded after the completion of the in-life portion of the study. Prepared dose materials were discarded at the completion of dosing.

### In-Life Observations and Measurements

Cage Side Observations (Animal Health Checks): Cage side animal health checks were performed at least once daily to check for general health, mortality and morbidity.

Clinical Observations: Clinical observations were performed on Day 0: 60-120 minutes post dose and at the end of the work day (3-6 hours post) and on Day 1: 22-26 hours post the Day 0 Test Material dose. Additional observations were made per exception.

Body Weights: Body weights for all animals were recorded on Days 0, 1, 2, 3, 7, 14, 21, 29 and 35. Additional body weights were recorded as needed. Weights were rounded to the nearest 0.1 g.

### Blood Collection

All animals in Groups 1-2, had interim blood collected on Days 3, 7, 14, 21 and 29 according to Table 10 shown below.

TABLE 10
Blood Collection (Interim):

| | Sample Collection Times |
|---|---|
| | Whole Blood (Tail, saphenous or orbital) |
| Group Number | Serum^{a} |
| 1-2 | Day 3, 7, 14, 21, 29, 35 |
| Volume/Portion | ~150 μL whole blood |
| Processing | Two (2) aliquots of serum 25 μl each |
| Storage | Frozen at nominally −70° C. |

After collection animals received 0.5-1.0 mL lactated Ringer's; subcutaneously.

Whole blood for serum was collected by tail-vein nick, saphenous vein or orbital sinus puncture, under inhalant isoflurane. Whole blood was collected into a serum separator with clot activator tube and processed into two (2) aliquots of 25 μL of serum.

All samples were stored at nominally −70° C. until shipped to on dry ice.

Anesthesia Recovery: As applicable, animals were monitored continuously while under anesthesia, during recovery and until mobile.

### Terminal Procedures and Collections

TABLE 11
Terminal Collections

| | Sample Collection Times |
|---|---|
| | Terminal Whole Blood (cardiac) |
| Group Number | Serum^{a} |
| 1-2 | Day 35 |
| Volume/Portion | MOV |
| Processing | Three (3) aliquots of serum 2 × 50 μL each |
| | 1 × residual |
| Storage | Frozen at nominally −70° C. |

MOV = maximum obtainable volume
^{a}Whole blood will be collected into serum separator tubes, with clot activator

Terminal Blood: Whole blood for serum was collected into a serum separator with clot activator tube and processed into two (2) aliquots of 50 μL serum and one (1) aliquot of residual per facility SOPs. All samples were stored at nominally −70° C. until shipped to on dry ice.

### Results

Monoclonal anti-spike human IgG was used to detect antibody expression, which was quantified by ng/mL anti-spike hIgG detected up to 35 days after injection with the ceDNA construct. As shown in FIG. 6, when ceDNA constructs 1856+1859 LNP coformulation was employed, ˜1 ug/mL of protein was detected by day 14 and by day 35 up to ˜8 μg/mL of protein was detected (FIG. 6).

The results from this experiment show that functional expression with dual vectors in an LNP enables additional degrees of freedom to optimize mAb expression.

### Example 7: A Study to Demonstrate Expression of Covid Ab after LNP Delivery In Vivo

ceDNA vectors were produced according to the methods described in Example 1 above.

The objective of the study was to determine expression of covid Ab after intravenous delivery of LNP formulated ceDNA. A ceDNA comprising a nucleic acid encoding the light chain (LC; ceDNA-1856) and another ceDNA comprising a nucleic acid encoding the heavy chain (HC; ceDNA-1859) of anti-SARS-CoV-2 Ab was dosed in mice (5 groups). The study design and details were carried out as set forth below.

### Study Design

Table 12 sets forth the design of the test material administration component of the study. As shown in Table 12, there were 5 groups of C157B16 mice (4 mice per group) that were intravenously administered vehicle (Group 1) or test compound (Groups 2-5; coformulated ceDNA-1856 and 1859) at a dose level of 10, 1, 0.1, 0.01 μg of coformulated ceDNA1856 (LC) and ceDNA1859 (HC). Serum samples were taken at days 3 and 7. In Groups 2-5, the molecular ratio of HC to LC in the ceDNA1856 (LC) and ceDNA-1859 (HC) coformulation was approximately 1:1 (HC: LC). The ORFs of ceDNA vectors 1856 and 1859 are set forth above in Table 7.

Briefly, C57B6 mice were injected hydrodynamically (8-10% body weight volume) via the tail vein with vehicle containing the indicated amount of ceDNA (10, 1 0.1, 0.01 μg). For each animal, a sample of serum was obtained 3 and 7 days post ceDNA iv administration. The circulating levels of mAb were then quantified in the mouse sera by standard ELISA measuring human IgG1 antibodies.

TABLE 12

| | No. of | Mouse | | | | Serum |
|---|---|---|---|---|---|---|
| Group | Mice | strain | Test Article | Dose (ug) | | Samples |
| 1 | 4 | C57B16 | vehicle | — | | Day 3, 7 |
| 2 | 4 | C57B16 | Dual HC/LC vectors | 10 | ug | Day 3, 7 |
| | | | (1:1 mol/mol) | | | |
| 3 | 4 | C57B16 | Dual HC/LC vectors | 1 | ug | Day 3, 7 |
| | | | (1:1 mol/mol) | | | |
| 4 | 4 | C57B16 | Dual HC/LC vectors | 0.1 | ug | Day 3, 7 |
| | | | (1:1 mol/mol) | | | |
| 5 | 4 | C57B16 | Dual HC/LC vectors | 0.01 | ug | Day 3, 7 |
| | | | (1:1 mol/mol) | | | |

Serum samples were collected at days 3 and 7, and the amount of antibody in the serum was detected by ELISA. The results are shown in Table 13 below, and in FIG. 7.

TABLE 13

| | | | | | Day 3 Ab | Day 7 Ab |
|---|---|---|---|---|---|---|
| | | | | | concen- | concen- |
| Group | Test Article | Dose (ug) | | Animal | tration | tration |
| 1 | PBS | — | | 1 | 0 | 0 |
| | | | | 2 | 0 | 0 |
| | | | | 3 | 0 | 0 |
| | | | | 4 | 0 | 0 |
| 2 | Dual HC/LC | 10 | ug | 5 | 190 | 88.89 |
| | vectors (1:1 | | | 6 | 230.26 | 113.64 |
| | mol/mol) S309- | | | 7 | 331.29 | 119.46 |
| | LS/GAALIE | | | 8 | 351.91 | 261.29 |
| 3 | Dual HC/LC | 1 | ug | 9 | 97.9 | 65.1 |
| | vectors (1:1 | | | 10 | 13.61 | 6.65 |
| | mol/mol) S309- | | | 11 | 89.73 | 70.79 |
| | LS/GAALIE | | | 12 | 77.76 | 71.27 |
| 4 | Dual HC/LC | 0.1 | ug | 13 | 6.49 | 5.21 |
| | vectors (1:1 | | | 14 | 6.02 | 0.17 |
| | mol/mol) S309- | | | 15 | 3.74 | 2.47 |
| | LS/GAALIE | | | 16 | 7.3 | 5.33 |
| 5 | Dual HC/LC | 0.01 | ug | 17 | 0.119 | 2.405 |
| | vectors (1:1 | | | 18 | 0.054 | 0.055 |
| | mol/mol) S309- | | | 19 | 0.016 | 0.012 |
| | LS/GAALIE | | | 20 | 0.077 | 0.08 |

As shown in FIG. 7, there was a dose dependent increase in serum levels of the antibodies expressed when dual ceDNA constructs 1856 (LC) and 1859 (HC) LNP coformulation was employed.

In a further set of experiments, dual ceDNA vector constructs comprising ceDNA-1856 (encoding LC) and ceDNA-1859 (encoding HC) coformulation in LNP1 (“LNP formulation 1”) was employed. FIG. 10 shows the results of LNP encapsulating both ceDNA-1856 (encoding LC) and ceDNA-1859 (encoding HC) (“dual vector” format) delivery and the resulting robust expression of the anti-Spike huIgG up to Day 35. Further, as shown in FIG. 10. LNP delivery of dual vectors (ceDNA-1; ceDNA dual constructs 1856 (LC) and 1859 (HC) coformulated in LNP formulation 1) achieved persistent, therapeutically relevant, anti-Spike hIgG concentrations of 8 μg/mL in mice. FIG. 11 shows that there was a dose dependent increase in antibody expression in ceDNA-1 dual vector (ceDNA constructs of 1856 (LC) and 1859 (HC)) platform or ceDNA-2 single vector having dual ORFs designed to express the antibody HC and LC from a single ceDNA vector (ceDNA-2157: “ceDNA-2”), following hydrodynamic delivery.

In general, serum levels of anti-Spike hIgG concentrations were determined by ELISA. Briefly, purified SARS-CoV-2 spike protein (LakePharma®, Cat. No. 46328) was coated on 96-well assay plates (Greiner Bio-One®, Cat. No. 655085) at 2 μg/mL in DPBS (ThermoFisher) and plates were incubated overnight at 4° C. Plates were then blocked for non-specific binding using 300 μL of SuperBlock (PBS) Blocking Buffer (ThermoFisher®, Cat. No. 37515) at room temperature for 2 hours. These were washed three times with 300 μL per well of 1×PBST (ThermoFisher). Samples and reference Standard dilutions were prepared in General Serum Diluent (Immuno Chemistry Technologies®, Cat. No. 649) and 100 μL of these dilutions in duplicates were added to each well. The plates were incubated for 60 min at room temperature with gentle shaking at 500 rpm. Plates were washed 3 times with PBST and tapped on absorbent paper to remove excess liquid. Goat anti-human IgG (H+L) HRP enzyme conjugated secondary antibody (Invitrogen®, Cat. No. 31410) was diluted 1:5000 in General Serum Diluent and 100 μL was added to each well. The plates were incubated at room temperature for 60 minutes with shaking at 500 rpm and were then washed 3 times with PBST. Finally. 100 μL of substrate solution (KPLSUREBLUE™ TMB Microwell Substrate, SeraCare®, Cat. No. 5120-0077) was added to each well and plates were incubated for 15 min at room temperature. 100 μL of Stop solution (SERACARE®, Cat. No. 5150-0020) was added to each well and absorbance was measured at 450 nm. Results are interpolated from the standard curve.

### Example 8: Neutralization of SARS-CoV-2

FIG. 8 shows the results of an experiment to test the neutralization of SARS-CoV-2 by the antibodies expressed by the ceDNA constructs tested in Example 7, hydrodynamically delivered.

Mouse sera were tested for the capacity to neutralize SARS-CoV-2 Spike (D19) pseudotyped VSV-Luc. Briefly, VeroE6-TMPRSS2 cells were seeded in 96-well clear-bottom, black walled plates at 20K cells/well by HT and incubated overnight at 37° C. Mouse sera were heat inactivated at 56° C. for 30 min. A 1:1 mix of basal DMEM and normal mouse serum (MP Biologicals) was spiked with S309-LS or I1 anti-VSVg at 50 micrograms/ml to make a 10× working dilution. Final assay dilution was 5 μg/ml. Sera were diluted in basal media (DMEM without FBS) such that the human IgG concentration was 50 μg/ml to make a 10× working dilution. Final assay dilution was 5 μg/ml. CoV2 Spike (D19) pseudotyped VSV-Luc was diluted in basal 1:20, added to diluted sera and further incubated at 37° C. for 1 hour. VSV-serum mixes were added to VeroE6-TMPRSS2 cells in triplicate at 50 μl per well and incubated at 37° C. for 1 hour. 50 μl of complete media (DMEM with 10% FBS) was added to each well. After overnight incubation at 37° C., media was removed from cells and 100 μl of BioGlo (Promega): PBS (1:1) was added to each well and incubated for 5 min. Resulting luminescence was measured on the EnSight (Perkin Elmer). The light signal is directly proportional to the presence of VSV-infected cells. Data are expressed as percentage of neutralization. All the mouse sera tested showed a capacity to neutralize the SARS-CoV-2 Spike (D19) pseudotyped VSV-Luc. Comparable results were obtained when a second set of sera was tested, as shown in FIG. 9.

### Example 9: Comparative In Vitro Effector Function Studies of Recombinant Anti-SARS-Cov2 S Antibodies Produced from a Cell-Line Versus Anti-SARS-Cov2 S Antibodies Produced and Purified from Mice Treated with ceDNA Dual Vectors (HC/LC)

The objective of this study was to compare the binding affinities of recombinant cell-line-derived and ceDNA-derived, serum-purified anti-SARS-CoV2 antibodies. CHO (Chinese hamster ovary) cells were co-transfected with plasmids expressing an anti-SARS-CoV2 HC and LC, using a method as described in Stettler et al. (Science, 2016, 353(6301):823-826), to produce recombinant monocolonal antibodies. ceDNA derived anti-SARS-CoV2 antibodies were generated by hydrodynamic IV injection of naked dual ceDNA vectors encoding anti-SARS-CoV2 HC or LC as described herein, into the tailvein of C57/Bl6 mice. Delivery of DNA through this method resulted in the efficient in vivo transfection of foreign DNA (e.g., ceDNA vectors) primarily in the liver (see, e.g., Kim & Ahituv, Methods Mol Biol. 2013; 1015: 279-289). Thus, antibody was mainly produced from the liver of the ceDNA vectors (1856 (LC) and 1859 (HC)) treated mice and then purified from the serum of the treated mice, yielding a ceDNA generated antibody.

Antibody 1 and Antibody 2 are modified antibodies derived from a parent antibody identified from a 2003 SARS-CoV survivor. The variable region of both Antibody 1 and Antibody 2 have been developed to have an extended half-life, with Antibody 1 engineered with a single “LS” mutation, and Antibody 2 engineered with a double “LS” and “GAALIE” modifications. Specifically, both antibodies possess an Fc “LS” mutation as defined herein, that confers extended half-life by binding to the neonatal Fc receptor. Antibody 2 is identical to Antibody 1 with the exception of the additional “GAALIE” modifications, as defined herein, to the Fc. The “GAALIE” modification has been shown in vitro to, inter alia, enhance binding to the FcγRIIIa receptor and evoke protective CD8+ T-cells in context of viral respiratory infection in vivo.

The following antibodies were tested: Antibody 1 (cell-lined derived), a single “LS” mutant, recombinantly produced from transfected CHO cells in vitro; ceDNA Antibody 1, a single “LS” mutant, produced in mice treated with ceDNA (HC and LC as described herein) via hydrodynamic IV injection and purified from the serum of the treated mice 3 days post injection; Antibody 2 with a double “LS” and “GAALIE” modifications, recombinantly produced from CHO cells in vitro; ceDNA Antibody 2 with a double “LS” and “GAALIE” modifications, produced in mice treated with ceDNA HC/LC dual vector as described herein via hydrodynamic IV injection and purified from the serum of the treated mice 3 days post the hydrodynamic injection. To measure the dissociation constant (K_{D}), the baseline of the Octet® FAB2G Biosensor was first calibrated for 60 seconds in 2× buffer containing (0.02% BSA, 0.004% Tween-20 in PBS). Then, the antibody sample was loaded at 30 nM for 300 seconds. The biosensor was baselined again in the same type of buffer as mentioned above. The FcγRIIIa receptor (either polymorph designated as “V” or polymorph designated as “F”) at various concentrations (from about 3 nM to about 300 nM and applying 4-7 different concentrations in the range) was then associated with the tested antibody for 480 seconds, then dissociated for 480 seconds, and the dissociation constant (K_{D}) values were measured by the biosensor and calculated using the software OCTET® Analysis Studio.

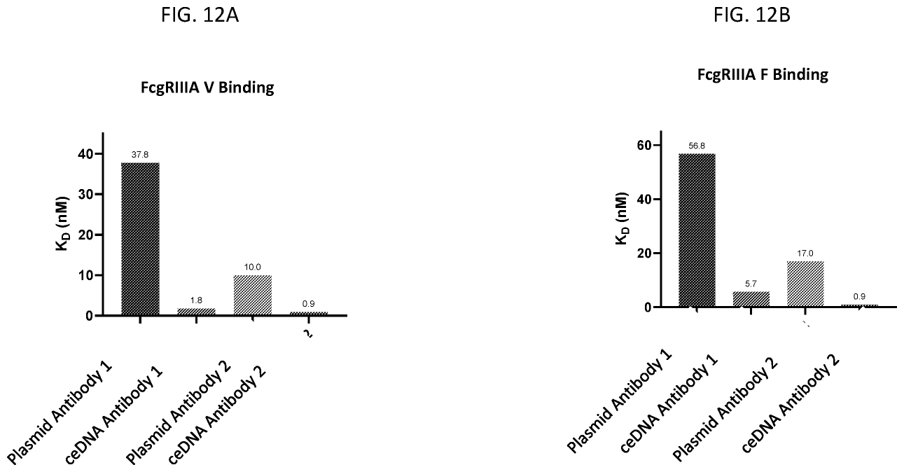

The dissociation constants (K_{D}) values are shown in Table 14 and also in FIG. 12A (for FcγRIIIa-V) and FIG. 12B (for FcγRIIIa-F). Table 14 also indicates that all of the measured and computed K_{D} values had a statistical regression value (R²) of >0.95, thereby indicating that all of the measured data fit the regression model extremely well.

TABLE 14

| FcγRIIIa | | | | |
|---|---|---|---|---|
| receptor | Antibody | Mutation | K_{D} (nM) | R² |
| V | Cell- line Ab1 | LS | 37.8 | 0.9915 |
| | ceDNA Ab1 | | 1.8 | 0.9940 |
| | Cell-line Ab2 | LS, | 10.0 | 0.9942 |
| | ceDNA Ab2 | GAALIE | 0.9 | 0.9933 |
| F | Cell-line Ab1 | LS | 56.8 | 0.9584 |
| | ceDNA Ab1 | | 5.7 | 0.9970 |
| | Cell-line Ab2 | LS, | 17.0 | 0.9898 |
| | ceDNA Ab2 | GAALIE | 0.9 | 0.9941 |

Lower K_{D} values are indicative of higher binding affinities. As can be seen in Table 14 and also in FIG. 12A and FIG. 12B, there was a dramatic increase in the binding affinity of antibody to receptor (FcγRIIIa-V and FcγRIIIa-F) when the antibody was produced from a mouse hydrodynamically injected with ceDNA compared to the recombinantly produced antibody (“Cell-line”).

Fucosylation is a typical terminal modification of proteins, and fucosyltransferase mediates the transfer of fucose residues to oligosaccharides and/or proteins. It was hypothesized that the antibodies produced as a result of hydrodynamic injection of ceDNA in the mice are afucosylated because they are produced in the liver of the ceDNA HC and LC treated mouse and the liver is an organ that may not express fucosyltransferase. Shields et al. (2002. J. Biol Chem. 277, 26733-26740) discovered antibodies lacking a prevalent post-translational modification to the conserved and essential Fc Asn297-linked carbohydrate (N-glycan), core fucosylation, bound CD16a with 50-fold greater affinity than typical human IgG1s (˜95% core fucosylated) and elicited ADCC at lower concentrations. Therefore, it was hypothesized that the resulting afucosylated monoclonal antibodies produced from the liver of the ceDNA HC/LC dual vector treated mice might demonstrate increased affinity toward the receptor FcγRIIIa than the recombinantly produced counterparts a mammalian from cell-line (e.g., CHO).

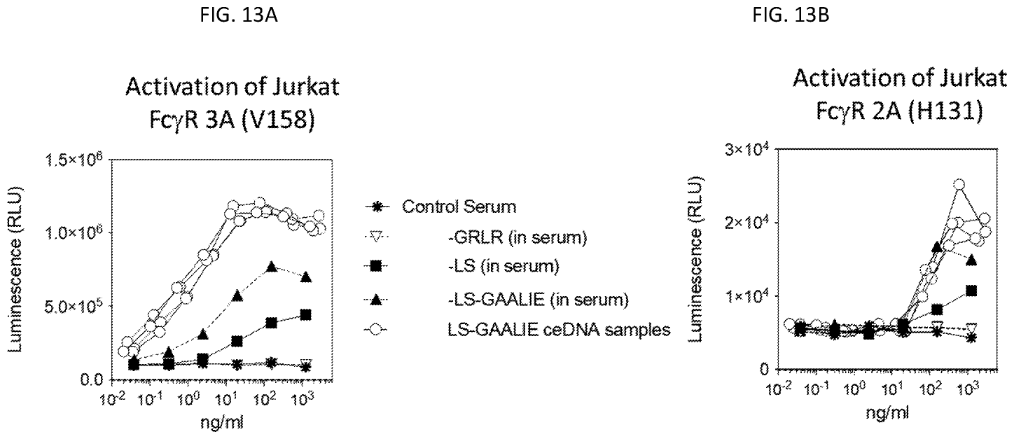

This hypothesis was confirmed in series of experiments below. Serum ceDNA derived Antibody 2 (LS-GAALIE) (“LS-GAALIE ceDNA”; FIGS. 13A and B) activated the Fc gamma receptor IIA, “FcγR 2A H131”, (FIG. 13B), but potently increased the activation of Fc gamma receptor IIIA, “FcγR 3A V158”, (FIG. 13A), on top of the already enhanced FcγR 3A activation of recombinant LS-GAALIE (“LS-GAALIE”, also referred to as “cell-line antibody 2”) when compared to recombinant LS (without the GAALIE mutations; “LS” or also referred to as “cell-line antibody 1”). The effect is most likely due to the afucosylation driven by the liver expression of ceDNA HC/LC dual vectors. This observation was confirmed by mass spec analyses of the glycan species present on these antibodies. Mass spec analysis of purified ceDNA LS-GAALIE (“ceDNA Antibody 2”) mAb and ceDNA LS (“ceDNA antibody 1”) mAb confirmed nearly complete afucosylation as shown in Table 15 below.

TABLE 15

| | Afucosylation (%) |
|---|---|
| LS ceDNA (ceDNA Ab1) | 98.4 |
| LS-GAALIE ceDNA (ceDNA Ab2) | 98.6 |

It is noted that Antibody 2, whether cell-line derived or ceDNA-derived and whether FcγRIIIa-V or FcγRIIIa-F, exhibited higher binding affinities than their respective Antibody 1 counterparts, confirming that the GAALIE modifications exerted a positive effect on the effector function as expected. However, in vivo liver expression of ceDNA derived antibodies (ceDNA HC/LC dual vectors as described herein) achieved far superior binding potency and in turn Fc gamma receptor activation levels as compared to those of monoclonal antibodies recombinantly produced from a traditional mammalian cell-line, mostly likely due to heightened afucosylation levels of the ceDNA-derived antibodies produced from the liver.

Taken together, these results demonstrate that the methods described above can generate antibodies with enhanced affinities and represents a new and improved platform for therapeutic antibody production for COVID-19.

### REFERENCES

All publications and references, including but not limited to patents and patent applications, cited in this specification and Examples herein are incorporated by reference in their entirety as if each individual publication or reference were specifically and individually indicated to be incorporated by reference herein as being fully set forth. Any patent application to which this application claims priority is also incorporated by reference herein in the manner described above for publications and references.

1. A capsid-free closed ended DNA (ceDNA) vector composition comprising a ceDNA vector comprising at least one nucleic acid sequence between flanking inverted terminal repeats (ITRs), wherein the at least one nucleic acid sequence encodes a heavy chain (HC) and/or a light chain (LC) of an anti-CoV-2 S antibody or an antigen-binding fragment thereof.

2. The ceDNA vector composition of claim 1, wherein the at least one nucleic acid sequence encodes the HC of the anti-SARS-CoV-2 S antibody, wherein the at least one nucleic acid sequence encoding the HC of the anti-SARS-CoV-2 S antibody is selected from the group consisting of SEQ ID NO: 1 and SEQ ID NO: 3.

3. The ceDNA vector composition of claim 1, wherein the at least one nucleic acid sequence encodes the LC of the anti-SARS-CoV-2 S antibody, wherein the at least one nucleic acid sequence encoding the LC of the anti-SARS-CoV-2 S antibody is selected from the group consisting of SEQ ID NO: 2 and SEQ ID NO: 4.

4. The ceDNA vector composition of claim 1, wherein the at least one nucleic acid sequence encodes both the HC and LC of the anti-SARS-CoV-2 S antibody, wherein the nucleic acid sequence encoding the HC is selected from SEQ ID NO: 1 and SEQ ID NO: 3, and the nucleic acid sequence encoding the LC is selected from SEQ ID NO: 2 and SEQ ID NO: 4.

5. A capsid-free closed-ended DNA (ceDNA) vector combination comprising:
a first ceDNA vector comprising at least one nucleic acid sequence between flanking inverted terminal repeats (ITRs), wherein the at least one nucleic acid sequence encodes a heavy chain (HC) of an anti-CoV-2 S antibody or an antigen-binding fragment thereof; and
a second ceDNA vector comprising at least one nucleic acid sequence between flanking inverted terminal repeats (ITRs), wherein the at least one nucleic acid sequence encodes a light chain (LC) of an anti-CoV-2 S antibody or an antigen-binding fragment thereof.

6. The ceDNA vector composition of any one of claims 2, 4 and 5, wherein the at least one nucleic acid sequence encodes an anti-SARS-CoV-2 S antibody HC comprising SEQ ID NO: 1 or SEQ ID NO: 3.

7. The ceDNA vector composition of any one of claims 3, 4 and 5, wherein the at least one nucleic acid sequence encodes an anti-SARS-CoV-2 S antibody LC comprising SEQ ID NO: 2 or SEQ ID NO: 4.

8. The ceDNA vector composition of claim 5, wherein the first ceDNA vector comprises at least one nucleic acid sequence encoding an anti-SARS-CoV-2 S antibody HC comprising SEQ ID NO: 1; and the second ceDNA vector comprises at least one nucleic acid sequence encoding an anti-SARS-CoV-2 S antibody LC comprising SEQ ID NO: 2.

9. The ceDNA vector composition of claim 5, wherein the first ceDNA vector comprises at least one nucleic acid sequence encoding an anti-SARS-CoV-2 S antibody HC comprising SEQ ID NO: 3; and the second ceDNA vector comprises at least one nucleic acid sequence encoding an anti-SARS-CoV-2 S antibody LC comprising SEQ ID NO: 4.

10. The ceDNA vector composition of any one of claims 5-9, wherein the first ceDNA vector comprises an open reading frame that is at least 85% identical to SEQ ID NO:25.

11. The ceDNA vector composition of any one of claims 5-9, wherein the second ceDNA vector comprises an ORF that is at least 85% identical to SEQ ID NO:26.

12. The ceDNA vector composition of any one of claims 5-11, wherein the first ceDNA vector and the second ceDNA vector are present at a molar ratio of 1:1.

13. The ceDNA vector composition of any one of claims 1-12, wherein the first ceDNA vector and the second ceDNA vector each comprise a promoter sequence, operatively linked to the least one nucleic acid sequence.

14. The ceDNA vector composition of claim 13, wherein the promoters are the same, or wherein the promoters are different.

15. The ceDNA vector composition of any one of claims 1-14, wherein at least one ITR comprises a functional terminal resolution site and a Rep binding site.

16. The ceDNA vector combination of claim 5, wherein the first and the second ceDNA vector are encapsulated in a lipid nanoparticle.

17. A capsid-free close-ended DNA (ceDNA) vector formulation comprising:
a first ceDNA vector comprising an open reading frame (ORF) at least 85% identical to SEQ ID NO:25; and
a second ceDNA vector comprising an ORF at least 85% identical to SEQ ID NO:26.

18. A capsid-free close-ended DNA (ceDNA) vector composition comprising:
a first ceDNA vector comprising an open reading frame (ORF) consisting of SEQ ID NO:26; and
a second ceDNA vector comprising an ORF consisting of SEQ ID NO:26.

19. A method of expressing an anti-CoV-2 S antibodies and antigen-binding fragments thereof in a cell comprising contacting the cell with the ceDNA vector formulation of any one of claims 1-18.

20. The method of claim 19, wherein the cell is in vitro or in vivo.

21. The method of claim 19 or 20, wherein the at least one nucleic acid sequence is codon optimized for expression in the eukaryotic cell.

22. A method of treating a subject with COVID-19, comprising administering to the subject a ceDNA vector composition of any one of claims 1-18, a cell of claim 33, or a composition of any one of claim 34-35.

23. A method of preventing infection of a subject with SARS-CoV-2, comprising administering to the subject a ceDNA vector composition of any one of claims 1-18, a cell of claim 33, or a composition of any one of claim 34-35.

24. The method of claim 22 or 23, wherein the subject is administered one or more additional therapeutic agents.

25. The method of any of claims 22-24, wherein the ceDNA vector formulation is administered by intravenous, subcutaneous or intramuscular injection.

26. An anti-SARS-CoV-2 S antibody, or an antigen binding fragment thereof, wherein the antibody, or the antigen binding fragment thereof, comprises a heavy chain (HC) and a light chain (LC), wherein the HC comprises an amino acid sequence that is at least 85% identical to SEQ ID NO: 1 or SEQ ID NO: 3; and the LC comprises an amino acid sequence that is at least 85% identical to SEQ ID NO: 2 or SEQ ID NO: 4, wherein the anti-SARS-CoV-2 S antibody is expressed from one or more ceDNA vectors containing a nucleic acid sequence encoding the HC and/or LC.

27. The antibody, or the antigen-binding fragment thereof, of claim 26, wherein:
the HC comprises SEQ ID NO: 1 and the LC comprises SEQ ID NO: 2; or
the HC comprises SEQ ID NO: 3 and the LC comprises SEQ ID NO: 4.

28. The anti-SARS-CoV-2 S antibody of claim 26, wherein the one or more ceDNA vectors are expressed in a cell.

29. The anti-SARS-CoV-2 S antibody of claim 26, wherein the anti-SARS-CoV-2 S antibody, or an antigen binding fragment thereof is expressed in the liver of a mammalian subject and has greater than 75%, 80%, 85%, 90%, 91%, 92%, 93%, 94%, 95%, 96%, 97%, or 98% afucosylation rate, or has FcγR IIIA or IIB activation rate that is greater than that of its counterpart recombinant antibody produced from a mammalian cell-line with the same amino acid sequences for the HC and the LC.

30. The method of claim 28, wherein the cell is in vitro or in vivo.

31. A pharmaceutical composition comprising the ceDNA vector composition of any one of claims 1-18.

32. The pharmaceutical composition of claim 30, further comprising an additional therapeutic agent.

33. A cell containing the ceDNA vector composition of any of claims 1-18.

34. A composition comprising the ceDNA vector composition of any of claims 1-18 and a lipid.

35. The composition of claim 34, wherein the lipid is a lipid nanoparticle (LNP).

36. A kit comprising the ceDNA vector composition of any one of claim 1-18 or 34-35, the pharmaceutical composition of claim 31 or 32, or the cell of claim 33, and instructions for use.
